(19) United States
(12) Patent Application Publication
(10) Pub. No.: US 2023/0000844 A1
(43) Pub. Date: Jan. 5, 2023

(54) BIOMARKERS FOR NANOPARTICLE COMPOSITIONS

(71) Applicant: Abraxis BioScience, LLC, Summit, NJ (US)

(72) Inventors: Neil P. DESAI, Pacific Palisades, CA (US); Anita N. SCHMID, Berkeley Heights, NJ (US); Shihe HOU, Millington, NJ (US); Andrew KWON, Hawthorne, CA (US)

(21) Appl. No.: 17/775,562

(22) PCT Filed: Nov. 11, 2020
(86) PCT No.: PCT/US2020/060070

§ 371 Date: May 9, 2022

Related U.S. Application Data
(60) Provisional application No. 62/991,469, filed on Mar. 18, 2020.
(60) Provisional application No. 62/933,820, filed on Nov. 11, 2019.

(51) Int. Cl. A61K 31/44; A61K 47/42; A61K 47/69; A61K 9/00; A61K 9/51; A61P 35/00

(52) U.S. Cl. CPC A61K 31/44; A61K 47/42; A61K 47/6929; A61K 9/0019; A61K 9/5169; A61P 35/00

(57) ABSTRACT

The present application provides methods and compositions for treating cancer by administering a composition comprising nanoparticles that comprise an mTOR inhibitor (such as a limus drug) and a carrier protein (such as an albumin) based upon the mutation status of TSC1 or TSC2 and one of VHL, RBI, PBRIM1, K.DM6A, RET, SETD2 ARID 1 A, BAP1, BRCA2, TP53, RBI, ATRX, FLT1, NTRK1, TLX3, KDM6A, CDH4, CDKN2C, DAXX, ERBB3, GNAS, IL7R, PDGFRB, PMS2, PTEN, SMARCA4, and YY1AP1.

### CROSS-REFERENCE TO RELATED APPLICATIONS

This application claims priority benefit of U.S. Provisional Patent Application No. 62/933,820 filed Nov. 11, 2019 and U.S. Provisional Patent Application No. 62/991,469 filed Mar. 18, 2020. The entire contents of those applications are hereby incorporated by reference herein.

### STATEMENT REGARDING FEDERALLY SPONSORED RESEARCH OR DEVELOPMENT

This subject matter of this application was supported in part by FDA Office of Orphan Products Development (OOPD) Grant R01FD005749. The Government has certain rights in this invention.

### FIELD OF THE INVENTION

The present invention relates to methods and compositions for treating cancer.

### BACKGROUND OF THE INVENTION

The mammalian target of rapamycin (mTOR) is a conserved serine/threonine kinase that serves as a central hub of signaling in the cell to integrate intracellular and extracellular signals and to regulate cellular growth and homeostasis. Activation of the mTOR pathway is associated with cell proliferation and survival, while inhibition of mTOR signaling leads to inflammation and cell death. Dysregulation of the mTOR signaling pathway has been implicated in an increasing number of human diseases, including cancer and autoimmune disorders. Consequently, mTOR inhibitors have found wide applications in treating diverse pathological conditions such as solid tumors, organ transplantation, restenosis, and rheumatoid arthritis. However, a pressing issue in the application of mTOR inhibitors is the variability of treatment response among different individuals having the same disease or condition. Given the large number of genes involved in the extended signaling network of mTOR, a reliable set of predictive biomarkers is much needed to guide selection of an effective treatment plan for individual patients.

Sirolimus (INN/USAN), also known as rapamycin, is an immunosuppressant drug used to prevent rejection in organ transplantation; it is especially useful in kidney transplants. Sirolimus-eluting stents were approved in the United States to treat coronary restenosis. Additionally, sirolimus has been demonstrated as an effective inhibitor of tumor growth in various cell lines and animal models. Other limus drugs, such as analogs of rapamycin, have been designed to improve the pharmacokinetic and pharmacodynamic properties of sirolimus. For example, Temsirolimus was approved in the United States and Europe for the treatment of renal cell carcinoma. Everolimus was approved in the U.S. for treatment of advanced breast cancer, pancreatic neuroendocrine tumors, advanced renal cell carcinoma, and subependymal giant cell astrocytoma (SEGA) associated with Tuberous Sclerosis. The mode of action of rapamycin is to bind the cytosolic protein FK-binding protein 12 (FKBP12), and the sirolimus-FKBP12 complex in turn inhibits the mTOR pathway by directly binding to the mTOR Complex 1 (mTORC1).

However, the roles of TSC1/2 and mTOR mutations in responding to rapalogs remain controversial. For example, although it has been reported that mutations in TSC1/2 and mTOR are more frequent in renal cell carcinoma (RCC) patients who respond well to rapalogs, the majority of rapalog responders have no mutations in mTOR pathway. In Kwiatkowski et al, only 2/32 (6.25%) patients with TSC1 mutations or copy number loss and 0% patients with TSC2 mutations or copy number loss that were treated with an mTOR inhibitor (e.g., temsirolimus or everolimus) responded. In addition, in another study (Kwiatkowski, NCT02201212) only 2/30 (7%) responses were seen in patients with TSC1 or TSC2 mutations that were treated with everolimus. See Kwiatkowski et al. Clin Cancer Res. 2016; 22:2445-52.

Moreover, rapalogs usually arrest cell proliferation but do not induce apoptosis.

Despite the initial response, tumors frequently develop resistance to these agents. See Hua et al., J Hematol Oncol 12, 71 (2019).

The disclosures of all publications, patents, patent applications and published patent applications referred to herein are hereby incorporated herein by reference in their entirety.

### BRIEF SUMMARY OF THE INVENTION

The present application provides methods of treating cancer in an individual comprising administering to the individual an effective amount of a composition comprising nanoparticles comprising an mTOR inhibitor (such as a limus drug) and a carrier protein (such as albumin), wherein the individual is selected for treatment on the basis of having an mTOR-activating aberration. In some embodiments, the mTOR-activating aberration comprises an aberration at one or more genes (such as 1, 2, 3, 4, 5, 6 or more) selected from the group consisting of TSC1, TSC2, RPS6, PTEN, TP53, RB1, ATRX, and FAT1.

In one aspect of the present application, there is provided a method of treating a cancer in an individual, comprising administering to the individual an effective amount of a composition comprising nanoparticles comprising an mTOR inhibitor and a carrier protein, wherein the individual is selected for treatment based on having an mTOR-activating aberration at TSC2 or RPS6. In some embodiments, the individual is selected for treatment based on having an mTOR-activating aberration at TSC2 and RPS6.

In some embodiments according to any one of the methods described above, the mTOR-activating aberration at TSC2 comprises a mutation in TSC2.

In some embodiments according to any one of the methods described above, the mTOR-activating aberration at TSC2 comprises a single-nucleotide variant (SNV). In some embodiments, the SNV comprises a mutation selected from the group consisting of C1503T, C2743G, C5383T, C3755G, G760T, C3442T, G880A, T707C, A4949G, or a deletion of any one or more of the amino acids at the position of 1405-1409, 1960-1970, 4999, 5002, 3521, 5208, 5238-5255.

In some embodiments according to any one of the methods described above, the mTOR-activating aberration at TSC2 comprises a copy number variation of TSC2.

In some embodiments according to any one of the methods described above, the mTOR-activating aberration at TSC2 is a loss of function mutation.

In some embodiments according to any one of the methods described above, the mTOR-activating aberration at TSC2 comprises an aberrant expression level of TSC2.

In some embodiments according to any one of the methods described above, the mTOR-activating aberration at TSC2 comprises an aberrant activity level of a protein encoded by TSC2.

In some embodiments according to any one of the methods described above, the mTOR-activating aberration at TSC2 comprises a loss of heterozygosity of TSC2.

The present application in another aspect provides a method of treating a cancer in an individual comprising administering to the individual an effective amount of a composition comprising nanoparticles comprising an mTOR inhibitor and a carrier protein, wherein the individual is selected for treatment on the basis of having an mTOR-activating aberration at TSC1 or RPS6.

In some embodiments according to any one of the methods described above, the mTOR-activating aberration at RPS6 comprises an aberrant phosphorylation level of the protein encoded by RPS6.

In some embodiments according to any one of the methods described above, the mTOR-activating aberration at RPS6 comprises an aberrant expression level of RPS6.

In some embodiments according to any one of the methods described above, the cancer is advanced and/or malignant.

In some embodiments according to any one of the methods described above, the cancer is a solid tumor.

In some embodiments according to any one of the methods described above, the cancer is a hematologic cancer.

In some embodiments according to any one of the methods described above, the cancer is selected from the group consisting of pancreatic neuroendocrine cancer, endometrial cancer, breast cancer, lymphangioleiomyomatosis (LAM), prostate cancer, hepatocellular carcinoma, melanoma, renal cell carcinoma, bladder cancer, endometrial cancer, ovary cancer, gynecologic cancer, sarcoma, perivascular epithelioid cell neoplasms (PEComa), Hodgkin's lymphoma and multiple myeloma.

In some embodiments according to any one of the methods described above, the nanoparticles in the composition comprises the mTOR inhibitor associated with the carrier protein.

In some embodiments according to any one of the methods described above, the nanoparticles in the composition have an average diameter of no greater than about 200 nm.

In some embodiments according to any one of the methods described above, the ratio of the mTOR inhibitor to the carrier protein in the nanoparticles is from about 1:1 to about 9:1.

In some embodiments according to any one of the methods described above, the carrier protein is an albumin. In some embodiments, the albumin is human serum albumin.

In some embodiments according to any one of the methods described above, the mTOR inhibitor is a limus drug. In some embodiments, the limus drug is rapamycin.

In some embodiments according to any one of the methods described above, the dose of the mTOR inhibitor in the composition for each administration is from about 10 mg/m² to about 100 mg/m².

In some embodiments according to any one of the methods described above, nanoparticle composition is administered at a frequency of about once a week to about once every two weeks.

In some embodiments according to any one of the methods described above, the method comprises administering the nanoparticle composition to the individual weekly for about two weeks followed by a rest period of about one week.

In some embodiments according to any one of the methods described above, the individual is resistant or refractory to a prior therapy.

In some embodiments according to any one of the methods described above, the method further comprises administering a second agent.

In some embodiments according to any one of the methods described above, the individual is a human.

In some embodiments according to any one of the methods described above, the individual does not comprise a mutation in TSC1.

In some embodiments according to any one of the methods described above, the method further comprises assessing the mTOR-activating aberration at TSC1, TSC2, or RPS6 in the individual.

In some embodiments according to any one of the methods described above, the method further comprises selecting the individual for treatment based on the individual having the mTOR-activating aberration at TSC1, TSC2 or RPS6.

In some embodiments according to any one of the methods described above, the composition comprises: (a) nanoparticles comprising rapamycin and albumin, and (b) a non-nanoparticle portion comprising albumin and rapamycin. In some embodiments, the nanoparticles comprise a core comprising rapamycin and a coating comprising albumin. In some embodiments, about 70% to about 85% of the albumin in the nanoparticles is in the form of monomeric albumin. In some embodiments, about 5% to about 15% of the albumin in the nanoparticles is in the form of polymeric albumin (or trimeric albumin). In some embodiments, about 9% to about 20% of the albumin in the nanoparticles is in the form of dimeric albumin. In some embodiments, about 0.5% to about 5% of the albumin in the non-nanoparticle portion is in the form of polymeric albumin (or trimeric albumin). In some embodiments, about 80% to about 95% of the albumin in the non-nanoparticle portion is in the form of monomeric albumin. In some embodiments, about 4% to about 14% of the albumin in the non-nanoparticle portion is in the form of dimeric albumin. In some embodiments, about 0.5% to about 5% of total albumin in the composition is in the form of polymeric albumin (or trimeric albumin). In some embodiments, about 80% to about 95% of total albumin in the composition is in the form of monomeric albumin. In some embodiments, about 4% to about 15% of total albumin in the composition is in the form of dimeric albumin. In some embodiments, the percentage of polymeric albumin (or trimeric albumin), dimeric albumin, or monomeric albumin is determined using size-exclusion chromatography. In some embodiments, the percentage of polymeric albumin (or trimeric albumin), dimeric albumin. or monomeric albumin is determined using size-exclusion chromatography using a saline mobile phase coupled with a multiple angle light scattering (MALS) detector. In some embodiments, the volume weighted mean particle size of the nanoparticles is about 200 nm or less. In some embodiments, the volume weighted mean particle size of the nanoparticles is about 50 nm to about 200 nm. In some embodiments, the Z-average particle size of the nanoparticles is about 200 nm or less. In some embodiments, the Z-average particle size of the nanoparticles is about 50 nm to about 200 nm. In some embodiments, the polydispersity index of the nanoparticles is less than 0.2. In some embodiments, the polydispersity index of the nanoparticles is about 0.03 to about 0.2. In some embodiments, the span of particle size distribution ((Dᵥ95−Dᵥ5)/Dᵥ50) of the nanoparticles is about 0.8 to about 1.2. In some embodiments, the weight percentage of the albumin in the nanoparticles is about 25% to about 45%. In some embodiments, the weight percentage of rapamycin in the nanoparticles is about 55% to about 75%. In some embodiments, the weight ratio of the albumin to the rapamycin in the nanoparticles is about 1:1 to about 1:4. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the nanoparticle composition is a nanoparticle suspension. In some embodiments, the concentration of albumin in the composition is about 30 mg/mL to about 100 mg/mL. In some embodiments, the concentration of albumin in the composition that is in the non-nanoparticle portion is about 30 mg/mL to about 100 mg/mL. In some embodiments, the concentration of albumin in the nanoparticle composition that is in the nanoparticles is about 1 mg/mL to about 5 mg/mL. In some embodiments, the concentration of rapamycin in the nanoparticle composition is about 1 mg/mL to about 100 mg/mL. In some embodiments, the concentration of rapamycin in the composition that is in the non-nanoparticle portion is about 20 μg/mL to about 55 μg/mL. In some embodiments, the concentration of rapamycin in the composition that is in the nanoparticles is about 1 mg/mL to about 15 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the composition is stable at 25° C. for at least 24 hours. In some embodiments, the composition is stable at 4° C. for at least 24 hours. In some embodiments, the nanoparticles had been resuspended from a dried composition. In some embodiments, the pH of the composition is about 6.0 to about 7.5. In some embodiments, the composition comprises less than 10 μg/mL tert-butanol. In some embodiments, the composition comprises tert-butanol. In some embodiments, the composition comprises less than 5 μg/mL chloroform. In some embodiments, the composition comprises chloroform. In some embodiments, the composition is a dried composition. In some embodiments, the zeta potential of the nanoparticles is about −25 mV to about −50 mV. In some embodiments, the composition has an amorphous morphology as determined by measuring crystallinity of a lyophilized form of the composition by X-ray diffraction. In some embodiments, the nanoparticles have an amorphous morphology as determined by separating the nanoparticles from the composition, lyophilizing the separated nanoparticles, and measuring crystallinity of the separated and lyophilized nanoparticles by X-ray diffraction. In some embodiments, the rapamycin in nanoparticles has an amorphous morphology as determined by Raman spectroscopy, polarized light microscopy, differential scanning calorimetry (DSC), modulated differential scanning calorimetry (mDSC). Fourier transform infrared (FTIR) spectroscopy, or nuclear magnetic resonance (NMR) spectroscopy. In some embodiments, the vinyl chain of the rapamycin in the nanoparticles interacts with the albumin in the nanoparticles. In some embodiments, at least a portion of the nanoparticles are non-spherical. In some embodiments, at least 20% of the nanoparticles in the composition are non-spherical. In some embodiments, seco-rapamycin is less than 3% by weight of the sum of seco-rapamycin and rapamycin in the nanoparticles. In some embodiments, seco-rapamycin is less than 3% by weight of the sum of seco-rapamycin and rapamycin in the composition. In some embodiments, seco-rapamycin is more than 0.2% by weight of the sum of seco-rapamycin and rapamycin in the nanoparticles. In some embodiments, seco-rapamycin is more than 0.2% by weight of the sum of seco-rapamycin and rapamycin in the composition.

In some embodiments according to any one of the methods described above, the composition comprises: (a) nanoparticles comprising rapamycin and albumin, and (b) a non-nanoparticle portion comprising albumin and rapamycin. In some embodiments, the nanoparticles comprise a core comprising rapamycin and a coating comprising albumin. In some embodiments, about 25% to about 50% of the albumin in the nanoparticles is in the form of monomeric albumin. In some embodiments, about 1% to about 4.5% of the albumin in the nanoparticles is in the form of oligomeric albumin. In some embodiments, about 42% to about 60% of the albumin in the nanoparticles is in the form of polymeric albumin (other than oligomeric albumin). In some embodiments, about 5% to about 16% of the albumin in the nanoparticles is in the form of dimeric albumin. In some embodiments, about 0.5% to about 3% of the albumin in the non-nanoparticle portion is in the form of polymeric albumin (other than oligomeric albumin). In some embodiments, about 0.5% to about 4% of the albumin in the non-nanoparticle portion is in the form of oligomeric albumin. In some embodiments, about 80% to about 95% of the albumin in the non-nanoparticle portion is in the form of monomeric albumin. In some embodiments, about 4% to about 14% of the albumin in the non-nanoparticle portion is in the form of dimeric albumin. In some embodiments, about 2% to about 7% of total albumin in the composition is in the form of polymeric albumin (other than oligomeric albumin). In some embodiments, about 0.3% to about 3% of the total albumin in the composition is in the form of oligomeric albumin. In some embodiments, about 80% to about 95% of total albumin in the composition is in the form of monomeric albumin. In some embodiments, about 4% to about 15% of total albumin in the composition is in the form of dimeric albumin. In some embodiments, the percentage of polymeric albumin (other than oligomeric albumin), oligomeric albumin, dimeric albumin, or monomeric albumin is determined using size-exclusion chromatography. In some embodiments, the percentage of polymeric albumin (other than oligomeric albumin), oligomeric albumin, dimeric albumin, or monomeric albumin is determined using size-exclusion chromatography using a mobile phase containing an aqueous portion and a miscible portion (such as an aqueous buffer containing 7.5% methanol) coupled with a UV detector. In some embodiments, the volume weighted mean particle size of the nanoparticles is about 200 nm or less. In some embodiments, the volume weighted mean particle size of the nanoparticles is about 50 nm to about 200 nm. In some embodiments, the Z-average particle size of the nanoparticles is about 200 nm or less. In some embodiments, the Z-average particle size of the nanoparticles is about 50 nm to about 200 nm. In some embodiments, the polydispersity index of the nanoparticles is less than 0.2. In some embodiments, the polydispersity index of the nanoparticles is about 0.03 to about 0.2. In some embodiments, the span of particle size distribution ((Dᵥ95−Dᵥ5)/Dᵥ50) of the nanoparticles is about 0.8 to about 1.2. In some embodiments, the weight percentage of the albumin in the nanoparticles is about 25% to about 45%. In some embodiments, the weight percentage of rapamycin in the nanoparticles is about 55% to about 75%. In some embodiments, the weight ratio of the albumin to the rapamycin in the nanoparticles is about 1:1 to about 1:4. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the nanoparticle composition is a nanoparticle suspension. In some embodiments, the concentration of albumin in the composition is about 30 mg/mL to about 100 mg/mL. In some embodiments, the concentration of albumin in the composition that is in the non-nanoparticle portion is about 30 mg/mL to about 100 mg/mL. In some embodiments, the concentration of albumin in the nanoparticle composition that is in the nanoparticles is about 1 mg/mL to about 5 mg/mL. In some embodiments, the concentration of rapamycin in the nanoparticle composition is about 1 mg/mL to about 100 mg/mL. In some embodiments, the concentration of rapamycin in the composition that is in the non-nanoparticle portion is about 20 μg/mL to about 55 μg/mL. In some embodiments, the concentration of rapamycin in the composition that is in the nanoparticles is about 1 mg/mL to about 15 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the composition is stable at 25° C. for at least 24 hours. In some embodiments, the composition is stable at 4° C. for at least 24 hours. In some embodiments, the nanoparticles had been resuspended from a dried composition. In some embodiments, the pH of the composition is about 6.0 to about 7.5. In some embodiments, the composition comprises less than 10 μg/mL tert-butanol. In some embodiments, the composition comprises tert-butanol. In some embodiments, the composition comprises less than 5 μg/mL chloroform. In some embodiments, the composition comprises chloroform. In some embodiments, the composition is a dried composition. In some embodiments, the zeta potential of the nanoparticles is about −25 mV to about −50 mV. In some embodiments, the composition has an amorphous morphology as determined by measuring crystallinity of a lyophilized form of the composition by X-ray diffraction. In some embodiments, the nanoparticles have an amorphous morphology as determined by separating the nanoparticles from the composition, lyophilizing the separated nanoparticles, and measuring crystallinity of the separated and lyophilized nanoparticles by X-ray diffraction. In some embodiments, the rapamycin in nanoparticles has an amorphous morphology as determined by Raman spectroscopy, polarized light microscopy, differential scanning calorimetry (DSC), modulated differential scanning calorimetry (mDSC), Fourier transform infrared (FTIR) spectroscopy, or nuclear magnetic resonance (NMR) spectroscopy. In some embodiments, the vinyl chain of the rapamycin in the nanoparticles interacts with the albumin in the nanoparticles. In some embodiments, at least a portion of the nanoparticles are non-spherical. In some embodiments, at least 20% of the nanoparticles in the composition are non-spherical. In some embodiments, seco-rapamycin is less than 3% by weight of the sum of seco-rapamycin and rapamycin in the nanoparticles. In some embodiments, seco-rapamycin is less than 3% by weight of the sum of seco-rapamycin and rapamycin in the composition. In some embodiments, seco-rapamycin is more than 0.2% by weight of the sum of seco-rapamycin and rapamycin in the nanoparticles. In some embodiments, seco-rapamycin is more than 0.2% by weight of the sum of seco-rapamycin and rapamycin in the composition.

In some embodiments, about 3% or less of the rapamycin in the nanoparticle composition is free rapamycin.

### BRIEF DESCRIPTION OF THE DRAWINGS

FIG. 1 depicts distributions of patients that have PEComa with various primary sites of diseases.

FIGS. 2A-2B depict duration of treatment, time-to-response, and progression-free survival of each evaluable individual patient up to May 2019.

FIG. 3 depicts longitudinal tumor size of each evaluable individual patient under independent radiology review up to May 2019.

FIG. 4 depicts maximum percentage of target lesion reduction of each evaluable individual patient. “+” or“−” indicates phosphorylation level of S6. Patients numbered 19-22, 26, 27, 29-31 had TSC2 mutation; patients numbered 4, 9, 14, 18, and 28 had TSC1 mutations; patients 1-3, 6, 8, 10, 11, 13, 16, 17, and 24 did not have either TSC1 or TSC2 mutation; patients numbered 5, 7, 12, 15, 23, and 25 had no evaluable sample for determining TSC1 or TSC2 mutational status. Patients' numbers in this Figure do not correspond to patients' numbers in Table 9.

FIGS. 5A-5B depict representative computed tomography images of tumors in patients with uterine primary PEComa before and after treatment. FIG. 5A is a representative image of a 67-year old female patient. She had uterine primary PEComa and the cancer had metastasized to spleen, colon, epigastric, and pulmonary area. Partial response occurred at the first restaging (6 weeks). The patient is currently on treatment (>1.5 years on therapy). FIG. 5B is a representative image of another 67-year old female patient. She also had uterine primary PEComa and the cancer had metastasized to pelvis and lung. Partial response occurred at the first restaging (6 weeks). The patient is currently on treatment (>2.5 years on therapy).

FIGS. 6A-6B depict representative computed tomography images of tumors in patients with retroperitoneal primary PEComa before and after treatment. FIG. 6A is a representative image of a 70-year old female patient with retroperitoneum primary PEComa.

The cancer had metastasized to lung and liver. Partial response occurred at the first restaging (6 weeks). The patient is currently on treatment (>2 years on therapy). FIG. 6B is a representative image of a 55-year old male patient with retroperitoneum primary PEComa. The cancer had metastasized to lung. Partial response occurred at the first restaging (6 weeks). The patient is currently on treatment (>2.5 years on therapy).

FIG. 7 depicts representative computed tomography images of tumors in a 47-year old male patient with kidney primary PEComa before and after treatment. The cancer had metastasized to kidney and pelvis. Partial response occurred at the first restaging (6 weeks). The patient had received twelve cycles of treatment.

FIG. 8 depicts computed tomography of chest, showing multiple pulmonary nodules (black arrows) prior to starting oral 10 mg everolimus.

FIG. 9 depicts computed tomography of chest showing significant progression of disease in lungs (black arrow) 2 months after starting everolimus and prior to starting nab-sirolimus.

FIG. 10 depicts computed tomography of chest showing decrease in size of pulmonary nodules (black arrow) 3 months after starting nab-sirolimus.

FIG. 11A depicts the tumor growth results of a human hepatocellular carcinoma mouse xenograft model after 0-15 days of treatment with saline (Group 1), ABI-009 (intravenous route; Group 2), Rapamune (oral administration; Group 3), and ABI-009 (subcutaneous route; Group 4).

FIG. 11B depicts body weight changes in a human hepatocellular carcinoma mouse xenograft model after 0-15 days of treatment with saline (Group 1), ABI-009 (intravenous route; Group 2), Rapamune (oral administration; Group 3), and ABI-009 (subcutaneous route; Group 4).

FIG. 12A depicts antitumor activity following ABI-009 treatment in a human hepatocellular carcinoma mouse xenograft model.

FIG. 12B depicts animal survival following ABI-009 treatment in a human hepatocellular carcinoma mouse xenograft model.

FIG. 13 depicts a Kaplan-Meier curve for PFS and OS for the mutation subtypes.

FIGS. 14A and 14B depict an algorithm for assessing whether a mutation is pathogenic.

### DETAILED DESCRIPTION OF THE INVENTION

The present application provides methods of treating a cancer in an individual comprising administering to the individual an effective amount of a composition comprising nanoparticles comprising an mTOR inhibitor (e.g., rapamycin or a derivative thereof) and a carrier protein (e.g., albumin), wherein the individual is selected for treatment on the basis of having an mTOR-activating aberration at one or more (such as one, two, three, four, five, or six) genes (such as TSC1, TSC2, RPS6, PTEN. TP53, RB1, ATRX, or FAT1). In some embodiments, the individual is selected for treatment on the basis of having an mTOR-activating aberration at TSC1, TSC2, TP53. ATRX, or RPS6. In some embodiments, the individual is selected for treatment on the basis of having an mTOR-activating aberration at TSC2 and RPS6.

The application is at least partly based upon the strikingly advantageous effects shown in a phase II study in which patients with advanced and malignant PEComa (“PEComa trial”) were treated with ABI-009 (a nanoparticle formulation of sirolimus coated with albumin, i.e., nab-sirolimus). Patients received ABI-009 at a dose of 100 mg/m² for two out of every three weeks a cycle for one or more cycles. Most of the patients had one or more mutations on one or more (such as one, two, three, four, five, or six) genes (such as TSC1, TSC2, PTEN, TP53, RB1, ATRX, or FAT1) and a positive status of phosphorylation of S6. Up to November 2020, the trial has at least achieved a) 90% of the patients achieved a partial response or a stable control; b) disease control (partial response and stable disease) in 71% of the patients; c) an independently assessed overall response rate (ORR) of 39% with durable responses (ongoing 30.7+ median months) and d) acceptable safety profile despite relatively high dose of nab-sirolimus.

Patients with mutation in TSC1, TSC2, TP53 and/or ATRX showed at least partial response to treatment, as well as those that had a positive status of phosphorylation of S6. Strikingly, the majority of patients (about 90%) with TSC2 mutation showed partial response to the treatment, while about 20% of the patients with TSC1 mutation showed partial response. Moreover, 58% of patients with a positive status of phosphorylated S6 (i.e., pS6) showed partial response to the treatment, while none of the patients (zero out of eight) without expression of pS6 showed partial response. Importantly, all patients with a TSC2 mutations and a positive pS6 responded to the treatment, which strongly suggests that cancer patients with aberration at TSC2 and RPS6 are particularly suitable for a treatment that comprise the administration of the nanoparticle composition described herein.

Moreover, the excellent responses observed in PEComa trial is not limited only to PEComa patients. Among the few patients consecutively enrolled under ABI-009 Expanded Access Protocol, all four non-PEComa cancer patients who satisfied the key inclusion criteria of the TSC1, TSC2 pan tumor registration study discussed in Example 5, i.e., must have pathologic inactivating TSC1 or TSC2 mutation; must have no satisfactory alternative treatments or have progressed following a standard treatment; must not be previously treated with an mTOR inhibitor, were all responding. See Example 6. In addition to having a TSC1 and TSC2 mutation, all these patients have one or more additional aberrations as discussed in further detail below. These combination of aberrations define patient populations who are particularly suitable for a treatment that comprises the administration of the nanoparticle composition described herein.

The nanoparticle compositions in some embodiments may have distinct characteristics for any one or more (in any combination) of the following: (1) the oligomeric status of the albumin associated with (such as in) the nanoparticles, such as the percentage of albumin monomers, dimers, and/or polymers (or timers) of the albumin associated with (such as in) the nanoparticles; (2) the oligomeric status of the albumin associated with (such as in) the non-nanoparticle portion of the composition, such as the percentage of albumin monomers, dimers, and/or polymers (or trimers) of the albumin associated with (such as in) the non-nanoparticle portion of the composition; (3) the oligomeric status of the total albumin in the composition, such as the percentage of albumin monomers, dimers, and/or polymers (or trimers) of the total albumin in the composition; (4) the particle size profile of the nanoparticles, such as the average particle size, polydispersity index, and/or size distribution; (5) the portion (e.g., weight percentage) of the nanoparticles that is albumin and/or the portion (e.g., weight percentage) of the nanoparticles that is rapamycin; (6) the weight ratio of the albumin to the rapamycin in the nanoparticles; (7) the weight ratio of the albumin to the rapamycin in the non-nanoparticle portion of the composition; (8) the weight ratio of the albumin to the rapamycin in the non-nanoparticle portion of the composition (9) the weight ratio of the total albumin to the total rapamycin in the composition; (10) the portion (e.g., weight percentage) of rapamycin that is in the nanoparticles (or the non-nanoparticle portion of the composition) compared to the total rapamycin in the composition; (11) the portion (e.g., weight percentage) of albumin that is in the non-nanoparticle portion (or in the nanoparticles) compared to the total albumin in the composition; (12) the concentration of albumin in the composition; (13) the concentration of albumin in the non-nanoparticle portion of the composition; (14) the concentration of albumin in the composition that is associated with (such as in) the nanoparticles; (15) the concentration of rapamycin in the composition; (16) the concentration of rapamycin in the non-nanoparticle portion of the composition; (17) the concentration of rapamycin in the composition that is associated with (such as in) the nanoparticles; (18) the osmolality of the composition; (19) the viscosity of the composition; (20) the pH of the composition; (21) the stability of the nanoparticles in the composition; (22) the amount of residual solvent in the composition; (23) the zeta potential of the nanoparticles in the composition; (24) the crystalline status of the rapamycin in the nanoparticles; (25) the particle morphology of the nanoparticles, such as the shape, sphericity, thickness of the coating, and/or surface-to-volume ratio; (26) the weight percentage of seco-rapamycin in the nanoparticles, as compared to the sum of seco-rapamycin and rapamycin, by weight; (27) the presence, percentage, or concentration of albumin stabilizer (such as sodium caprylate and N-acetyltryptophanate) in the composition; (28) the recovery of rapamycin following filtration; (29) in vitro release kinetics of the nanoparticles; (30) the portion of total rapamycin in the composition that is both in the non-nanoparticle portion of the composition and not bound to albumin; and/or (31) the weight percentage of seco-rapamycin in the composition, as compared to the sum of seco-rapamycin and rapamycin, by weight. The physicochemical parameters discussed above can affect drug release and delivery of the albumin-based rapamycin nanoparticle compositions (such as pharmaceutical compositions), and thus constitute unique properties to the compositions. Any method of assessing the crystalline state of the rapamycin in the nanoparticles has a limit of detection. For example, if the limit of detection of a method is about 1%, then if less than 1% of the rapamycin is crystalline the assay will not detect crystalline rapamycin and the composition will be assessed as non-crystalline or amorphous. In some embodiments, the crystalline state of the rapamycin in the nanoparticles is assessed by a method with a limit of detection of about 1% crystalline rapamycin or less. In some embodiments, if the crystalline state of the rapamycin in the nanoparticles is assessed by a method with a limit of detection of about 1% crystalline rapamycin or less, and the method detects no crystalline rapamycin, then the rapamycin is assessed to be amorphous or non-crystalline.

The nanoparticle compositions in some embodiments may have distinct characteristics for any one or more (in any combination) of the following: (1) the oligomeric status of the albumin associated with (such as in) the nanoparticles, such as the percentage of albumin monomers, dimers, oligomers, and/or polymers (other than oligomers) of the albumin associated with (such as in) the nanoparticles; (2) the oligomeric status of the albumin associated with (such as in) the non-nanoparticle portion of the composition, such as the percentage of albumin monomers, dimers, oligomers, and/or polymers (other than oligomers) of the albumin associated with (such as in) the non-nanoparticle portion of the composition; (3) the oligomeric status of the total albumin in the composition, such as the percentage of albumin monomers, dimers, oligomers, and/or polymers (other than oligomers) of the total albumin in the composition; (4) the particle size profile of the nanoparticles, such as the average particle size, polydispersity index, and/or size distribution; (5) the portion (e.g., weight percentage) of the nanoparticles that is albumin and/or the portion (e.g., weight percentage) of the nanoparticles that is rapamycin; (6) the weight ratio of the albumin to the rapamycin in the nanoparticles; (7) the weight ratio of the albumin to the rapamycin in the non-nanoparticle portion of the composition; (8) the weight ratio of the albumin to the rapamycin in the non-nanoparticle portion of the composition (9) the weight ratio of the total albumin to the total rapamycin in the composition; (10) the portion (e.g., weight percentage) of rapamycin that is in the nanoparticles (or the non-nanoparticle portion of the composition) compared to the total rapamycin in the composition; (11) the portion (e.g., weight percentage) of albumin that is in the non-nanoparticle portion (or in the nanoparticles) compared to the total albumin in the composition; (12) the concentration of albumin in the composition; (13) the concentration of albumin in the non-nanoparticle portion of the composition; (14) the concentration of albumin in the composition that is associated with (such as in) the nanoparticles; (15) the concentration of rapamycin in the composition; (16) the concentration of rapamycin in the non-nanoparticle portion of the composition; (17) the concentration of rapamycin in the composition that is associated with (such as in) the nanoparticles; (18) the osmolality of the composition; (19) the viscosity of the composition; (20) the pH of the composition; (21) the stability of the nanoparticles in the composition; (22) the amount of residual solvent in the composition; (23) the zeta potential of the nanoparticles in the composition; (24) the crystalline status of the rapamycin in the nanoparticles; (25) the particle morphology of the nanoparticles, such as the shape, sphericity, thickness of the coating, and/or surface-to-volume ratio; (26) the weight percentage of seco-rapamycin in the nanoparticles, as compared to the sum of seco-rapamycin and rapamycin, by weight; (27) the presence, percentage, or concentration of albumin stabilizer (such as sodium caprylate and N-acetyltryptophanate) in the composition; (28) the recovery of rapamycin following filtration; (29) in vitro release kinetics of the nanoparticles; (30) the portion of total rapamycin in the composition that is both in the non-nanoparticle portion of the composition and not bound to albumin; and/or (31) the weight percentage of seco-rapamycin in the composition, as compared to the sum of seco-rapamycin and rapamycin, by weight. The physicochemical parameters discussed above can affect drug release and delivery of the albumin-based rapamycin nanoparticle compositions (such as pharmaceutical compositions), and thus constitute unique properties to the compositions. Any method of assessing the crystalline state of the rapamycin in the nanoparticles has a limit of detection. For example, if the limit of detection of a method is about 1%, then if less than 1% of the rapamycin is crystalline the assay will not detect crystalline rapamycin and the composition will be assessed as non-crystalline or amorphous. In some embodiments, the crystalline state of the rapamycin in the nanoparticles is assessed by a method with a limit of detection of about 1% crystalline rapamycin or less. In some embodiments, if the crystalline state of the rapamycin in the nanoparticles is assessed by a method with a limit of detection of about 1% crystalline rapamycin or less, and the method detects no crystalline rapamycin, then the rapamycin is assessed to be amorphous or non-crystalline.

The present application also provides a kit comprising a composition comprising nanoparticles comprising an mTOR inhibitor and an albumin; and an agent for assessing an mTOR-activating aberration at one or more (such as one, two, three, four, five, or six) of the genes described herein (such as TSC2, TSC1, RPS6). Also provided are compositions (such as pharmaceutical compositions), and medicine useful for methods described herein.

### Definitions

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of a pathological consequence of a cancer. The methods of the invention contemplate any one or more of these aspects of treatment.

The term “individual” refers to a mammal and includes, but is not limited to, human, bovine, horse, feline, canine, rodent, or primate. In some embodiments, the individual is a mammal. In some embodiments, the individual is a human.

“Adjuvant setting” refers to a clinical setting in which an individual has had a history of a hyperplasia (e.g. cancer, restenosis, or pulmonary hypertension), and generally (but not necessarily) been responsive to therapy, which includes, but is not limited to, surgery (e.g., surgery resection), radiotherapy, and chemotherapy. However, because of their history of a hyperplasia (e.g. cancer, restenosis, or pulmonary hypertension), these individuals are considered at risk of development of the disease. Treatment or administration in the “adjuvant setting” refers to a subsequent mode of treatment. The degree of risk (e.g., when an individual in the adjuvant setting is considered as “high risk” or “low risk”) depends upon several factors, most usually the extent of disease when first treated.

“Neoadjuvant setting” refers to a clinical setting in which the method is carried out before the primary/definitive therapy.

As used herein, “delaying” the development of a cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of a cancer is a method that reduces probability of disease development in a given time frame and/or reduces the extent of the disease in a given time frame, when compared to not using the method. Such comparisons are typically based on clinical studies, using a statistically significant number of subjects. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The term “effective amount” used herein refers to an amount of a compound or composition sufficient to treat a specified disorder, condition or disease such as ameliorate, palliate, lessen, and/or delay one or more of its symptoms. For therapeutic use, beneficial or desired results include, e.g., decreasing one or more symptoms resulting from the disease (biochemical, histologic and/or behavioral), including its complications and intermediate pathological phenotypes presenting during development of the disease, increasing the quality of life of those suffering from the disease, decreasing the dose of other medications required to treat the disease, enhancing effect of another medication, delaying the progression of the disease, and/or prolonging survival of patients. In reference to a cancer, an effective amount comprises an amount sufficient to cause a tumor tissue to shrink and/or to decrease the growth rate of the tumor tissue or to prevent or delay other unwanted cell proliferation in the tumor. In some embodiments, an effective amount is an amount sufficient to delay development of a cancer. In some embodiments, an effective amount is an amount sufficient to prevent or delay recurrence. An effective amount can be administered in one or more administrations. In the case of cancer, the effective amount of the drug or composition may: (i) reduce the number of tumor cells; (ii) reduce the tumor size; (iii) inhibit, retard, slow to some extent and preferably stop a tumor cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

An “adverse event” or “AE” as used herein refers to any untoward medical occurrence in an individual receiving a marketed pharmaceutical product or in an individual who is participating on a clinical trial who is receiving an investigational or non-investigational pharmaceutical agent. The AE does not necessarily have a causal relationship with the individual's treatment. Therefore, an AE can be any unfavorable and unintended sign, symptom, or disease temporally associated with the use of a medicinal product, whether or not considered to be related to the medicinal product. An AE includes, but is not limited to: an exacerbation of a pre-existing illness; an increase in frequency or intensity of a pre-existing episodic event or condition; a condition detected or diagnosed after study drug administration even though it may have been present prior to the start of the study; and continuously persistent disease or symptoms that were present at baseline and worsen following the start of the study. An AE generally does not include: medical or surgical procedures (e.g., surgery, endoscopy, tooth extraction, or transfusion); however, the condition that leads to the procedure is an adverse event; pre-existing diseases, conditions, or laboratory abnormalities present or detected at the start of the study that do not worsen; hospitalizations or procedures that are done for elective purposes not related to an untoward medical occurrence (e.g., hospitalizations for cosmetic or elective surgery or social/convenience admissions); the disease being studied or signs/symptoms associated with the disease unless more severe than expected for the individual's condition; and overdose of study drug without any clinical signs or symptoms.

A “serious adverse event” or (SAE) as used herein refers to any untoward medical occurrence at any dose including, but not limited to, that: a) is fatal; b) is life-threatening (defined as an immediate risk of death from the event as it occurred); c) results in persistent or significant disability or incapacity; d) requires in-patient hospitalization or prolongs an existing hospitalization (exception: Hospitalization for elective treatment of a pre-existing condition that did not worsen during the study is not considered an adverse event. Complications that occur during hospitalization are AEs and if a complication prolongs hospitalization, then the event is serious); e) is a congenital anomaly/birth defect in the offspring of an individual who received medication; or f) conditions not included in the above definitions that may jeopardize the individual or may require intervention to prevent one of the outcomes listed above unless clearly related to the individual's underlying disease. “Lack of efficacy” (progressive disease) is not considered an AE or SAE. The signs and symptoms or clinical sequelae resulting from lack of efficacy should be reported if they fulfill the AE or SAE definitions.

The following definitions may be used to evaluate response based on target lesions: “complete response” or “CR” refers to disappearance of all target lesions; “partial response” or “PR” refers to at least a 30% decrease in the sum of the longest diameters (SLD) of target lesions, taking as reference the baseline SLD; “stable disease” or “SD” refers to neither sufficient shrinkage of target lesions to qualify for PR, nor sufficient increase to qualify for PD, taking as reference the nadir SLD since the treatment started; and “progressive disease” or “PD” refers to at least a 20% increase in the SLD of target lesions, taking as reference the nadir SLD recorded since the treatment started, or, the presence of one or more new lesions.

The following definitions of response assessments may be used to evaluate a non-target lesion: “complete response” or “CR” refers to disappearance of all non-target lesions; “stable disease” or “SD” refers to the persistence of one or more non-target lesions not qualifying for CR or PD; and “progressive disease” or “PD” refers to the “unequivocal progression” of existing non-target lesion(s) or appearance of one or more new lesion(s) is considered progressive disease (if PD for the subject is to be assessed for a time point based solely on the progression of non-target lesion(s), then additional criteria are required to be fulfilled.

“Progression free survival” (PFS) indicates the length of time during and after treatment that the cancer does not grow. Progression-free survival includes the amount of time individuals have experienced a complete response or a partial response, as well as the amount of time individuals have experienced stable disease.

“Correlate” or “correlating” is meant comparing, in any way, the performance and/or results of a first analysis or protocol with the performance and/or results of a second analysis or protocol. For example one may use the results of a first analysis or protocol to determine whether a second analysis or protocol should be performed. With respect to the embodiment of gene expression analysis or protocol, one may use the results of the gene expression analysis or protocol to determine whether a specific therapeutic regimen should be performed.

“Predicting” or “prediction” is used herein to refer to the likelihood that an individual is likely to respond either favorably or unfavorably to a treatment regimen.

As used herein, “at the time of starting treatment” or “baseline” refers to the time period at or prior to the first exposure to the treatment.

A method of “aiding assessment” as used herein refers to methods that assist in making a clinical determination and may or may not be conclusive with respect to the assessment.

“Likely to respond” or “responsiveness” as used herein refers to any kind of improvement or positive response either clinical or non-clinical selected from, but not limited to, measurable reduction in tumor size or evidence of disease or disease progression, complete response, partial response, stable disease, increase or elongation of progression free survival, or increase or elongation of overall survival.

As used herein, “sample” refers to a composition which contains a molecule which is to be characterized and/or identified, for example, based on physical, biochemical, chemical, physiological, and/or genetic characteristics.

“Cells,” as used herein, is understood to refer not only to the particular subject cell, but to the progeny or potential progeny of such a cell. Because certain modifications may occur in succeeding generations due to either mutation or environmental influences, such progeny may not, in fact, be identical to the parent cell, but are still included within the scope of the term as used herein.

The mTOR-activing aberration determined “before or upon initiation of treatment” is the mTOR-activing aberration determined in an individual before or upon the individual receives the first administration of a treatment modality described herein.

An individual who “may be suitable”, which includes an individual who is “suitable” for treatment(s) described herein, is an individual who is more likely than not to benefit from administration of said treatments. Conversely, an individual who “may not be suitable” or “may be unsuitable”, which includes an individual who is “unsuitable” for treatment(s) described herein, is an individual who is more likely than not to fail to benefit from administration of said treatments.

As used herein, “mTOR inhibitor nanoparticle composition” refers to a composition comprising nanoparticles comprising an mTOR inhibitor (such as a limus drug) and an albumin. “Limus nanoparticle composition” refers to a composition comprising nanoparticles comprising a limus drug (such as Sirolimus) and an albumin.

It is understood that aspect and embodiments of the invention described herein include “consisting” and/or “consisting essentially of” aspects and embodiments.

Reference to “about” a value or parameter herein includes (and describes) variations that are directed to that value or parameter per se. For example, description referring to “about X” includes description of “X”.

The term “about X-Y” used herein has the same meaning as “about X to about Y.”

As used herein and in the appended claims, the singular forms “a,” “or,” and “the” include plural referents unless the context clearly dictates otherwise.

As is apparent to one skilled in the art, an individual assessed, selected for, and/or receiving treatment is an individual in need of such activities.

### Methods of Treating Cancer

In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual comprising administering (e.g., intravenously or subcutaneously administering) to the individual an effective amount of a composition comprising nanoparticles comprising an mTOR inhibitor and a carrier protein, wherein the individual is selected for treatment on the basis of having an mTOR-activating aberration at TSC2. In some embodiments, the mTOR-activating aberration at TSC2 comprises a mutation in TSC2. In some embodiments, the mutation is selected from the group consisting of splice site mutation, nonsense mutation, frameshift mutation, and missense mutation. In some embodiments, the mTOR-activating aberration at TSC2 comprises a single-nucleotide variant (SNV). In some embodiments, the SNV comprises a mutation selected from the group consisting of C1503T, C2743G, C5383T, C3755G, G760T. C3442T, G880A, T707C, A4949G, or a deletion of any one or more of the amino acids at the position of 1405-1409, 1960-1970, 4999, 5002, 3521, 5208, 5238-5255. In some embodiments, the mTOR-activating aberration at TSC2 comprises a copy number variation of TSC2. In some embodiments, the mTOR-activating aberration at TSC2 is a loss of function mutation. In some embodiments, the mTOR-activating aberration in TSC2 comprises an aberrant expression level of TSC2. In some embodiments, the mTOR-activating aberration in TSC2 comprises an aberrant activity level of a protein encoded by TSC2. In some embodiments, the mTOR-activating aberration in TSC2 comprises a loss of heterozygosity of TSC2. In some embodiments, the mTOR inhibitor is a limus drug. In some embodiments, the mTOR inhibitor is rapamycin or a derivative thereof. In some embodiments, the mTOR inhibitor is rapamycin. In some embodiments, the carrier protein is albumin (such as human serum albumin). In some embodiments, the dose of the mTOR inhibitor in the composition for each administration is from about 10 mg/m² to about 100 mg/m² (e.g., about 50 mg/m² to about 100 mg/m², about 75 mg/m² to about 100 mg/m²). In some embodiments, the method comprises administering the nanoparticle composition to the individual weekly for about two weeks followed by a rest period of about one week. In some embodiments, the cancer is selected from the group consisting of pancreatic neuroendocrine cancer, endometrial cancer, breast cancer, lymphangioleiomyomatosis (LAM), prostate cancer, hepatocellular carcinoma, melanoma, renal cell carcinoma, bladder cancer, endometrial cancer, ovary cancer, gynecologic cancer, sarcoma, perivascular epithelioid cell neoplasms (PEComa), Hodgkin's lymphoma and multiple myeloma. In some embodiments, the cancer is a PEComa. In some embodiments, the individual is selected for treatment based on having a TSC2 aberration (e.g., a TSC2 mutation), regardless of the nature of the cancer. In some embodiments, the individual does not have a TSC1 aberration (e.g., a TSC1 mutation). In some embodiments, the method further comprises administering an anti-PD-1 antibody into the individual. In some embodiments, the anti-PD-1 antibody is administered at a dose of about 1 mg/kg to about 5 mg/kg (such as about 3 mg/kg) once every three weeks. In some embodiments, the individual fails to respond to one or more prior therapy (such as a different mTOR inhibitor, e.g., everolimus, such as an immune checkpoint inhibitor, e.g., an anti-PD-1 antibody).

In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual (e.g., an individual having a TSC2 aberration in cancer tissue) comprising administering (e.g., intravenously or subcutaneously administering) to the individual an effective amount of a composition comprising nanoparticles comprising an mTOR inhibitor and a carrier protein, wherein the individual is selected for treatment on the basis of having an mTOR-activating aberration at RPS6. In some embodiments, the mTOR-activating aberration at RPS6 comprises an aberrant phosphorylation level of the protein encoded by RPS6 (e.g., phosphorylation at residue S235, S236, S240, and/or S244). In some embodiments, the mTOR-activating aberration at RPS6 comprises a positive status of phosphorylated S6 (pS6) (e.g., phosphorylation at residue S235, S236, S240, and/or S244). In some embodiments, the expression level of RPS6 is assessed by immunohistochemistry. In some embodiments, the mTOR-activating aberration at RPS6 comprises an aberrant expression level of RPS6. In some embodiments, the mTOR inhibitor is a limus drug. In some embodiments, the mTOR inhibitor is rapamycin or a derivative thereof. In some embodiments, the mTOR inhibitor is rapamycin. In some embodiments, the carrier protein is albumin (such as human serum albumin). In some embodiments, the dose of the mTOR inhibitor in the composition for each administration is from about 10 mg/m² to about 100 mg/m² (e.g., about 50 mg/m² to about 100 mg/m², about 75 mg/m² to about 100 mg/m²). In some embodiments, the method comprises administering the nanoparticle composition to the individual weekly for about two weeks followed by a rest period of about one week. In some embodiments, the cancer is selected from the group consisting of pancreatic neuroendocrine cancer, endometrial cancer, breast cancer, lymphangioleiomyomatosis (LAM), prostate cancer, hepatocellular carcinoma, melanoma, renal cell carcinoma, bladder cancer, endometrial cancer, ovary cancer, gynecologic cancer, sarcoma, perivascular epithelioid cell neoplasms (PEComa), Hodgkin's lymphoma and multiple myeloma. In some embodiments, the cancer is a PEComa. In some embodiments, the individual is selected for treatment based on having a RPS6 aberration (e.g., a positive status of phosphorylated S6), regardless of the nature of the cancer. In some embodiments, the method further comprises administering an anti-PD-1 antibody into the individual. In some embodiments, the anti-PD-1 antibody is administered at a dose of about 1 mg/kg to about 5 mg/kg (such as about 3 mg/kg) once every three weeks. In some embodiments, the individual fails to respond to one or more prior therapy (such as a different mTOR inhibitor, e.g., everolimus, such as an immune checkpoint inhibitor, e.g., an anti-PD-1 antibody).

In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual (e.g., an individual having a TSC2 aberration in cancer tissue) comprising administering (e.g., intravenously or subcutaneously administering) to the individual an effective amount of a composition comprising nanoparticles comprising an mTOR inhibitor and a carrier protein, wherein the individual is selected for treatment on the basis of having an mTOR-activating aberration at TSC1. In some embodiments, the mTOR-activating aberration at TSC1 comprises a mutation in TSC1. In some embodiments, the mutation is selected from the group consisting of a splice site mutation, a nonsense mutation, a frameshift mutation, a missense mutation and a loss or deletion of the gene. In some embodiments, the mTOR-activating aberration at TSC1 comprises a single-nucleotide variant (SNV). In some embodiments, the mTOR-activating aberration at TSC1 comprises a copy number variation of TSC1. In some embodiments, the mTOR-activating aberration at TSC1 is a loss of function mutation. In some embodiments, the mTOR-activating aberration in TSC1 comprises an aberrant expression level of TSC1. In some embodiments, the mTOR-activating aberration in TSC2 comprises an aberrant activity level of a protein encoded by TSC1. In some embodiments, the mTOR-activating aberration in TSC1 comprises a loss of heterozygosity of TSC1. In some embodiments, the mTOR inhibitor is a limus drug. In some embodiments, the mTOR inhibitor is rapamycin or a derivative thereof. In some embodiments, the mTOR inhibitor is rapamycin. In some embodiments, the carrier protein is albumin (such as human serum albumin). In some embodiments, the dose of the mTOR inhibitor in the composition for each administration is from about 10 mg/m² to about 100 mg/m² (e.g., about 50 mg/m² to about 100 mg/m², about 75 mg/m² to about 100 mg/m²). In some embodiments, the method comprises administering the nanoparticle composition to the individual weekly for about two weeks followed by a rest period of about one week. In some embodiments, the cancer is selected from the group consisting of pancreatic neuroendocrine cancer, endometrial cancer, breast cancer, lymphangioleiomyomatosis (LAM), prostate cancer, hepatocellular carcinoma, melanoma, renal cell carcinoma, bladder cancer, endometrial cancer, ovary cancer, gynecologic cancer, sarcoma, perivascular epithelioid cell neoplasms (PEComa), Hodgkin's lymphoma and multiple myeloma. In some embodiments, the cancer is a PEComa. In some embodiments, the individual is selected for treatment based on having a TSC1 aberration (e.g., a TSC1 mutation), regardless of the nature of the cancer. In some embodiments, the method further comprises administering an anti-PD-1 antibody into the individual. In some embodiments, the anti-PD-1 antibody is administered at a dose of about 1 mg/kg to about 5 mg/kg (such as about 3 mg/kg) once every three weeks. In some embodiments, the individual fails to respond to one or more prior therapy (such as a different mTOR inhibitor. e.g., everolimus, such as an immune checkpoint inhibitor, e.g., an anti-PD-1 antibody).

In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual (e.g., an individual having a TSC2 aberration in cancer tissue) comprising administering (e.g., intravenously or subcutaneously administering) to the individual an effective amount of a composition comprising nanoparticles comprising an mTOR inhibitor and a carrier protein, wherein the individual is selected for treatment on the basis of having an mTOR-activating aberration at PTEN. In some embodiments, the mTOR-activating aberration at PTEN comprises a mutation in PTEN. In some embodiments, the mutation is selected from the group consisting of a splice site mutation, a nonsense mutation, a frameshift mutation, a missense mutation and a loss or deletion of the gene. In some embodiments, the mTOR-activating aberration at PTEN comprises a single-nucleotide variant (SNV). In some embodiments, the mTOR-activating aberration at PTEN comprises a copy number variation of PTEN. In some embodiments, the mTOR-activating aberration at PTEN is a loss of function mutation. In some embodiments, the mTOR-activating aberration in PTEN comprises an aberrant expression level of PTEN. In some embodiments, the mTOR-activating aberration in PTEN comprises an aberrant activity level of a protein encoded by PTEN. In some embodiments, the mTOR-activating aberration in PTEN comprises a loss of heterozygosity of PTEN. In some embodiments, the mTOR inhibitor is a limus drug. In some embodiments, the mTOR inhibitor is rapamycin or a derivative thereof. In some embodiments, the mTOR inhibitor is rapamycin. In some embodiments, the carrier protein is albumin (such as human serum albumin). In some embodiments, the dose of the mTOR inhibitor in the composition for each administration is from about 10 mg/m² to about 100 mg/m² (e.g., about 50 mg/m² to about 100 mg/m², about 75 mg/m² to about 100 mg/m²). In some embodiments, the method comprises administering the nanoparticle composition to the individual weekly for about two weeks followed by a rest period of about one week. In some embodiments, the cancer is selected from the group consisting of pancreatic neuroendocrine cancer, endometrial cancer, breast cancer, lymphangioleiomyomatosis (LAM), prostate cancer, hepatocellular carcinoma, melanoma, renal cell carcinoma, bladder cancer, endometrial cancer, ovary cancer, gynecologic cancer, sarcoma, perivascular epithelioid cell neoplasms (PEComa), Hodgkin's lymphoma and multiple myeloma. In some embodiments, the cancer is a PEComa. In some embodiments, the individual is selected for treatment based on having a PTEN aberration (e.g., a PTEN mutation, e.g., a PTEN loss), regardless of the nature of the cancer. In some embodiments, the method further comprises administering an anti-PD-1 antibody into the individual. In some embodiments, the anti-PD-1 antibody is administered at a dose of about 1 mg/kg to about 5 mg/kg (such as about 3 mg/kg) once every three weeks. In some embodiments, the individual fails to respond to one or more prior therapy (such as a different mTOR inhibitor, e.g., everolimus, such as an immune checkpoint inhibitor, e.g., an anti-PD-1 antibody).

In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual (e.g., an individual having a TSC2 aberration in cancer tissue) comprising administering (e.g., intravenously or subcutaneously administering) to the individual an effective amount of a composition comprising nanoparticles comprising an mTOR inhibitor and a carrier protein, wherein the individual is selected for treatment on the basis of having an mTOR-activating aberration at ATRX. In some embodiments, the mTOR-activating aberration at ATRX comprises a mutation in ATRX In some embodiments, the mutation is selected from the group consisting of a splice site mutation, a nonsense mutation, a frameshift mutation, a missense mutation and a loss or deletion of the gene. In some embodiments, the mTOR-activating aberration at ATRX comprises a single-nucleotide variant (SNV). In some embodiments, the mTOR-activating aberration at ATRX comprises a copy number variation of ATRX. In some embodiments, the mTOR-activating aberration at ATRX is a loss of function mutation. In some embodiments, the mTOR-activating aberration in ATRX comprises an aberrant expression level of ATRX. In some embodiments, the mTOR-activating aberration in ATRX comprises an aberrant activity level of a protein encoded by ATRX. In some embodiments, the mTOR-activating aberration in ATRX comprises a loss of heterozygosity of ATRX. In some embodiments, the mTOR inhibitor is a limus drug. In some embodiments, the mTOR inhibitor is rapamycin or a derivative thereof. In some embodiments, the mTOR inhibitor is rapamycin. In some embodiments, the carrier protein is albumin (such as human serum albumin). In some embodiments, the dose of the mTOR inhibitor in the composition for each administration is from about 10 mg/m² to about 100 mg/m² (e.g., about 50 mg/m² to about 100 mg/m², about 75 mg/m² to about 100 mg/m²). In some embodiments, the method comprises administering the nanoparticle composition to the individual weekly for about two weeks followed by a rest period of about one week. In some embodiments, the cancer is selected from the group consisting of pancreatic neuroendocrine cancer, endometrial cancer, breast cancer, lymphangioleiomyomatosis (LAM), prostate cancer, hepatocellular carcinoma, melanoma, renal cell carcinoma, bladder cancer, endometrial cancer, ovary cancer, gynecologic cancer, sarcoma, perivascular epithelioid cell neoplasms (PEComa), Hodgkin's lymphoma and multiple myeloma. In some embodiments, the cancer is a PEComa. In some embodiments, the individual is selected for treatment based on having a ATRX aberration (e.g., a ATRX mutation, e.g., a ATRX loss), regardless of the nature of the cancer. In some embodiments, the method further comprises administering an anti-PD-1 antibody into the individual. In some embodiments, the anti-PD-1 antibody is administered at a dose of about 1 mg/kg to about 5 mg/kg (such as about 3 mg/kg) once every three weeks. In some embodiments, the individual fails to respond to one or more prior therapy (such as a different mTOR inhibitor, e.g., everolimus, such as an immune checkpoint inhibitor, e.g., an anti-PD-1 antibody).

In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual (e.g., an individual having a TSC2 aberration in cancer tissue) comprising administering (e.g., intravenously or subcutaneously administering) to the individual an effective amount of a composition comprising nanoparticles comprising an mTOR inhibitor and a carrier protein, wherein the individual is selected for treatment on the basis of having an mTOR-activating aberration at RB1. In some embodiments, the mTOR-activating aberration at RB1 comprises a mutation in RB1. In some embodiments, the mutation is selected from the group consisting of a splice site mutation, a nonsense mutation, a frameshift mutation, a missense mutation and a loss or deletion of the gene. In some embodiments, the mTOR-activating aberration at RB1 comprises a single-nucleotide variant (SNV). In some embodiments, the mTOR-activating aberration at RB1 comprises a copy number variation of RB1. In some embodiments, the mTOR-activating aberration at RB) is a loss of function mutation. In some embodiments, the mTOR-activating aberration in RB) comprises an aberrant expression level of RB1. In some embodiments, the mTOR-activating aberration in RB1 comprises an aberrant activity level of a protein encoded by RB1. In some embodiments, the mTOR-activating aberration in RB1 comprises a loss of heterozygosity of RB). In some embodiments, the mTOR inhibitor is a limus drug. In some embodiments, the mTOR inhibitor is rapamycin or a derivative thereof. In some embodiments, the mTOR inhibitor is rapamycin. In some embodiments, the carrier protein is albumin (such as human serum albumin). In some embodiments, the dose of the mTOR inhibitor in the composition for each administration is from about 10 mg/m² to about 100 mg/m² (e.g., about 50 mg/m² to about 100 mg/m², about 75 mg/m² to about 100 mg/m²). In some embodiments, the method comprises administering the nanoparticle composition to the individual weekly for about two weeks followed by a rest period of about one week. In some embodiments, the cancer is selected from the group consisting of pancreatic neuroendocrine cancer, endometrial cancer, breast cancer, lymphangioleiomyomatosis (LAM), prostate cancer, hepatocellular carcinoma, melanoma, renal cell carcinoma, bladder cancer, endometrial cancer, ovary cancer, gynecologic cancer, sarcoma, perivascular epithelioid cell neoplasms (PEComa), Hodgkin's lymphoma and multiple myeloma. In some embodiments, the cancer is a PEComa. In some embodiments, the individual is selected for treatment based on having a RB) aberration (e.g., a RB) mutation, e.g., a RB) loss), regardless of the nature of the cancer. In some embodiments, the method further comprises administering an anti-PD-1 antibody into the individual. In some embodiments, the anti-PD-1 antibody is administered at a dose of about 1 mg/kg to about 5 mg/kg (such as about 3 mg/kg) once every three weeks. In some embodiments, the individual fails to respond to one or more prior therapy (such as a different mTOR inhibitor, e.g., everolimus, such as an immune checkpoint inhibitor, e.g., an anti-PD-1 antibody).

In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual (e.g., an individual having a TSC2 aberration in cancer tissue) comprising administering (e.g., intravenously or subcutaneously administering) to the individual an effective amount of a composition comprising nanoparticles comprising an mTOR inhibitor and a carrier protein, wherein the individual is selected for treatment on the basis of having an mTOR-activating aberration at TP53. In some embodiments, the mTOR-activating aberration at TP53 comprises a mutation in TP53. In some embodiments, the mutation is selected from the group consisting of a splice site mutation, a nonsense mutation, a frameshift mutation, a missense mutation and a loss or deletion of the gene. In some embodiments, the mTOR-activating aberration at TP53 comprises a single-nucleotide variant (SNV). In some embodiments, the mTOR-activating aberration at TP53 comprises a copy number variation of TP53. In some embodiments, the mTOR-activating aberration at TP53 is a loss of function mutation. In some embodiments, the mTOR-activating aberration in TP53 comprises an aberrant expression level of TP53. In some embodiments, the mTOR-activating aberration in TP53 comprises an aberrant activity level of a protein encoded by TP53. In some embodiments, the mTOR-activating aberration in TP53 comprises a loss of heterozygosity of TP53. In some embodiments, the mTOR inhibitor is a limus drug. In some embodiments, the mTOR inhibitor is rapamycin or a derivative thereof. In some embodiments, the mTOR inhibitor is rapamycin. In some embodiments, the carrier protein is albumin (such as human serum albumin). In some embodiments, the dose of the mTOR inhibitor in the composition for each administration is from about 10 mg/m² to about 100 mg/m² (e.g., about 50 mg/m² to about 100 mg/m², about 75 mg/m² to about 100 mg/m²). In some embodiments, the method comprises administering the nanoparticle composition to the individual weekly for about two weeks followed by a rest period of about one week. In some embodiments, the cancer is selected from the group consisting of pancreatic neuroendocrine cancer, endometrial cancer, breast cancer, lymphangioleiomyomatosis (LAM), prostate cancer, hepatocellular carcinoma, melanoma, renal cell carcinoma, bladder cancer, endometrial cancer, ovary cancer, gynecologic cancer, sarcoma, perivascular epithelioid cell neoplasms (PEComa), Hodgkin's lymphoma and multiple myeloma. In some embodiments, the cancer is a PEComa. In some embodiments, the individual is selected for treatment based on having a TP53 aberration (e.g., a TP53 mutation, e.g., a TP53 loss), regardless of the nature of the cancer. In some embodiments, the method further comprises administering an anti-PD-1 antibody into the individual. In some embodiments, the anti-PD-1 antibody is administered at a dose of about 1 mg/kg to about 5 mg/kg (such as about 3 mg/kg) once every three weeks. In some embodiments, the individual fails to respond to one or more prior therapy (such as a different mTOR inhibitor. e.g., everolimus, such as an immune checkpoint inhibitor, e.g., an anti-PD-1 antibody).

In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual (e.g., an individual having a TSC2 aberration in cancer tissue) comprising administering (e.g., intravenously or subcutaneously administering) to the individual an effective amount of a composition comprising nanoparticles comprising an mTOR inhibitor and a carrier protein, wherein the individual is selected for treatment on the basis of having two or more (such as two, three, four, five, six or seven) mTOR-activating aberration selected from the group consisting of an mTOR-activating aberration at TSC1, an mTOR-activating aberration at TSC2, an mTOR-activating aberration at PTEN, an mTOR-activating aberration at ATRX, an mTOR-activating aberration at RB1, an mTOR-activating aberration at TP53. In some embodiments, the individual has both an mTOR-activating aberration at PTEN (such as a PTEN loss) and mTOR-activating aberration at TSC2 (such as a TSC2 mutation). In some embodiments, the individual further has an mTOR-activating aberration at RB1, ATRX, and/or TP53. In some embodiments, the mTOR-activating aberration comprises a mutation. In some embodiments, the mutation is selected from the group consisting of a splice site mutation, a nonsense mutation, a frameshift mutation, a missense mutation and a loss or deletion of the gene. In some embodiments, the mTOR-activating aberration comprises a single-nucleotide variant (SNV). In some embodiments, the mTOR-activating aberration comprises a copy number variation. In some embodiments, the mTOR-activating aberration is a loss of function mutation. In some embodiments, the mTOR-activating aberration comprises an aberrant expression level of the gene. In some embodiments, the mTOR-activating aberration comprises an aberrant activity level of a protein encoded by the gene. In some embodiments, the mTOR-activating aberration comprises a loss of heterozygosity of the gene. In some embodiments, the mTOR inhibitor is a limus drug. In some embodiments, the mTOR inhibitor is rapamycin or a derivative thereof. In some embodiments, the mTOR inhibitor is rapamycin. In some embodiments, the carrier protein is albumin (such as human serum albumin). In some embodiments, the dose of the mTOR inhibitor in the composition for each administration is from about 10 mg/m² to about 100 mg/m² (e.g., about 50 mg/m² to about 100 mg/m², about 75 mg/m² to about 100 mg/m²). In some embodiments, the method comprises administering the nanoparticle composition to the individual weekly for about two weeks followed by a rest period of about one week. In some embodiments, the cancer is selected from the group consisting of pancreatic neuroendocrine cancer, endometrial cancer, breast cancer, lymphangioleiomyomatosis (LAM), prostate cancer, hepatocellular carcinoma, melanoma, renal cell carcinoma, bladder cancer, endometrial cancer, ovary cancer, gynecologic cancer, sarcoma, perivascular epithelioid cell neoplasms (PEComa), Hodgkin's lymphoma and multiple myeloma. In some embodiments, the cancer is a PEComa. In some embodiments, the individual is selected for treatment based on having the one or more mTOR-activating aberrations, regardless of the nature of the cancer. In some embodiments, the method further comprises administering an anti-PD-1 antibody into the individual. In some embodiments, the anti-PD-1 antibody is administered at a dose of about 1 mg/kg to about 5 mg/kg (such as about 3 mg/kg) once every three weeks. In some embodiments, the individual fails to respond to one or more prior therapy (such as a different mTOR inhibitor, e.g., everolimus, such as an immune checkpoint inhibitor, e.g., an anti-PD-1 antibody).

In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual comprising administering (e.g., intravenously or subcutaneously administering) to the individual an effective amount of a composition comprising nanoparticles comprising an mTOR inhibitor and a carrier protein, wherein the individual is selected for treatment on the basis of having an mTOR-activating aberration at a) RPS6 and b) one other gene selected from the group consisting of TSC1, TSC2, PTEN, TP53, RB1, ATRX, and FAT1. In some embodiments, the individual is selected for treatment on the basis of having an mTOR-activating aberration at a) RPS6 and b) one other gene selected from the group consisting of PTEN, TSC1 or TSC2. In some embodiments, the individual is selected for treatment on the basis of having an mTOR-activating aberration at a) RPS6 and b) TSC1 or TSC2. In some embodiments, the mTOR-activating aberration at TSC1 or TSC2 comprises a mutation in TSC1 or TSC2. In some embodiments, the mutation is selected from the group consisting of a splice site mutation, a nonsense mutation, a frameshift mutation, a missense mutation and a loss or deletion of the gene. In some embodiments, the mTOR-activating aberration at RPS6 comprises an aberrant phosphorylation level of the protein encoded by RPS6 (e.g., phosphorylation at residue S235, S236, S240, and/or S244). In some embodiments, the mTOR-activating aberration at RPS6 comprises a positive status of phosphorylated S6 (pS6) (e.g., phosphorylation at residue S235, S236, S240, and/or S244). In some embodiments, the mTOR inhibitor is a limus drug. In some embodiments, the mTOR inhibitor is rapamycin or a derivative thereof. In some embodiments, the mTOR inhibitor is rapamycin. In some embodiments, the carrier protein is albumin (such as human serum albumin). In some embodiments, the dose of the mTOR inhibitor in the composition for each administration is from about 10 mg/m² to about 100 mg/m² (e.g., about 50 mg/m² to about 100 mg/m², about 75 mg/m² to about 100 mg/m²). In some embodiments, the method comprises administering the nanoparticle composition to the individual weekly for about two weeks followed by a rest period of about one week. In some embodiments, the cancer is selected from the group consisting of pancreatic neuroendocrine cancer, endometrial cancer, breast cancer, lymphangioleiomyomatosis (LAM), prostate cancer, hepatocellular carcinoma, melanoma, renal cell carcinoma, bladder cancer, endometrial cancer, ovary cancer, gynecologic cancer, sarcoma, perivascular epithelioid cell neoplasms (PEComa), Hodgkin's lymphoma and multiple myeloma. In some embodiments, the cancer is a PEComa. In some embodiments, the method further comprises administering an anti-PD-1 antibody into the individual. In some embodiments, the anti-PD-1 antibody is administered at a dose of about 1 mg/kg to about 5 mg/kg (such as about 3 mg/kg) once every three weeks. In some embodiments, the individual fails to respond to one or more prior therapy (such as a different mTOR inhibitor, e.g., everolimus, such as an immune checkpoint inhibitor, e.g., an anti-PD-1 antibody).

In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual comprising administering (e.g., intravenously or subcutaneously administering) to the individual an effective amount of a composition comprising nanoparticles comprising an mTOR inhibitor and a carrier protein, wherein the individual is selected for treatment on the basis of a) having a TSC2 aberration (e.g., a TSC2 mutation), and b) having a RPS6 aberration (e.g., aberrant phosphorylation level of the protein encoded by RPS6 (e.g., phosphorylation at residue S235, S236, S240, and/or S244). In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual comprising administering (e.g., intravenously or subcutaneously administering) to the individual an effective amount of a composition comprising nanoparticles comprising an mTOR inhibitor and a carrier protein, wherein the individual is selected for treatment on the basis of a) having a TSC2 aberration (e.g., a TSC2 mutation), b) not having a TSC1 mutation, and c) having a RPS6 aberration (e.g., aberrant phosphorylation level of the protein encoded by RPS6 (e.g., phosphorylation at residue S235, 5236, S240, and/or S244). In some embodiments, the mTOR-activating aberration at RPS6 comprises a positive status of phosphorylated S6 (pS6) (e.g., phosphorylation at residue S235, S236, S240, and/or S244). In some embodiments, the mutation is selected from the group consisting of a splice site mutation, a nonsense mutation, a frameshift mutation, a missense mutation and a loss or deletion of the gene. In some embodiments, the mTOR inhibitor is a limus drug. In some embodiments, the mTOR inhibitor is rapamycin or a derivative thereof. In some embodiments, the mTOR inhibitor is rapamycin. In some embodiments, the carrier protein is albumin (such as human serum albumin). In some embodiments, the dose of the mTOR inhibitor in the composition for each administration is from about 10 mg/m² to about 100 mg/m² (e.g., about 50 mg/m² to about 100 mg/m², about 75 mg/m² to about 100 mg/m²). In some embodiments, the method comprises administering the nanoparticle composition to the individual weekly for about two weeks followed by a rest period of about one week. In some embodiments, the cancer is selected from the group consisting of pancreatic neuroendocrine cancer, endometrial cancer, breast cancer, lymphangioleiomyomatosis (LAM), prostate cancer, hepatocellular carcinoma, melanoma, renal cell carcinoma, bladder cancer, endometrial cancer, ovary cancer, gynecologic cancer, sarcoma, perivascular epithelioid cell neoplasms (PEComa), Hodgkin's lymphoma and multiple myeloma. In some embodiments, the cancer is a PEComa. In some embodiments, the individual is selected for treatment based on having a TSC2 aberration and a RPS6 aberration, regardless of the nature of the cancer. In some embodiments, the method further comprises administering an anti-PD-1 antibody into the individual. In some embodiments, the anti-PD-1 antibody is administered at a dose of about 1 mg/kg to about 5 mg/kg (such as about 3 mg/kg) once every three weeks. In some embodiments, the individual fails to respond to one or more prior therapy (such as a different mTOR inhibitor, e.g., everolimus, such as an immune checkpoint inhibitor, e.g., an anti-PD-1 antibody).

In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual comprising administering (e.g., intravenously or subcutaneously administering) to the individual an effective amount of a composition comprising nanoparticles comprising an mTOR inhibitor and a carrier protein, wherein the individual is selected for treatment on the basis of a) having a mutation in TSC1, and b) having an aberrant phosphorylation level of the protein encoded by RPS6 (e.g., phosphorylation at residue S235, S236, S240, and/or S244). In some embodiments, the dose of the mTOR inhibitor in the composition for each administration is from about 10 mg/m² to about 100 mg/m² (e.g., about 50 mg/m² to about 100 mg/m², about 75 mg/m² to about 100 mg/m²). In some embodiments, the method comprises administering the nanoparticle composition to the individual weekly for about two weeks followed by a rest period of about one week. In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual comprising administering (e.g., intravenously or subcutaneously administering) to the individual an effective amount of a composition comprising nanoparticles comprising an mTOR inhibitor (e.g., rapamycin) and a carrier protein (e.g., albumin), wherein the individual is selected for treatment on the basis of a) having a mutation in TSC1, and b) having an aberrant phosphorylation level of the protein encoded by RPS6 (e.g., phosphorylation at residue S235, S236, S240, and/or S244), wherein the dose of the mTOR inhibitor in the composition for each administration is from about 10 mg/m² to about 100 mg/m² (e.g., about 50 mg/m² to about 100 mg/m², about 75 mg/m² to about 100 mg/m²), and optionally wherein the composition is administered weekly for about two weeks followed by a rest period of about one week. In some embodiments, the mTOR-activating aberration at RPS6 comprises a positive status of phosphorylated S6 (pS6) (e.g., phosphorylation at residue S235, S236, S240, and/or S244). In some embodiments, the mutation is selected from the group consisting of a splice site mutation, a nonsense mutation, a frameshift mutation, a missense mutation and a loss or deletion of the gene. In some embodiments, the mTOR inhibitor is a limus drug. In some embodiments, the mTOR inhibitor is rapamycin or a derivative thereof. In some embodiments, the mTOR inhibitor is rapamycin. In some embodiments, the carrier protein is albumin (such as human serum albumin). In some embodiments, the cancer is selected from the group consisting of pancreatic neuroendocrine cancer, endometrial cancer, breast cancer, lymphangioleiomyomatosis (LAM), prostate cancer, hepatocellular carcinoma, melanoma, renal cell carcinoma, bladder cancer, endometrial cancer, ovary cancer, gynecologic cancer, sarcoma, perivascular epithelioid cell neoplasms (PEComa). Hodgkin's lymphoma and multiple myeloma. In some embodiments, the cancer is a PEComa. In some embodiments, the individual is selected for treatment based on having a TSC1 aberration and a RPS6 aberration, regardless of the nature of the cancer. In some embodiments, the method further comprises administering an anti-PD-1 antibody into the individual. In some embodiments, the anti-PD-1 antibody is administered at a dose of about 1 mg/kg to about 5 mg/kg (such as about 3 mg/kg) once every three weeks. In some embodiments, the individual fails to respond to one or more prior therapy (such as a different mTOR inhibitor, e.g., everolimus, such as an immune checkpoint inhibitor, e.g., an anti-PD-1 antibody).

In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual comprising administering (e.g., intravenously or subcutaneously administering) to the individual an effective amount of a composition comprising nanoparticles comprising an mTOR inhibitor and a carrier protein, wherein the individual is selected for treatment on the basis of a) having a mutation in TP53 or ATRX, and b) having an aberrant phosphorylation level of the protein encoded by RPS6 (e.g., phosphorylation at residue S235, S236, S240, and/or S244). In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual comprising administering (e.g., intravenously or subcutaneously administering) to the individual an effective amount of a composition comprising nanoparticles comprising an mTOR inhibitor and a carrier protein, wherein the individual is selected for treatment on the basis of a) having a mutation in TP53 or ATRX, and b) having an aberrant phosphorylation level of the protein encoded by RPS6 (e.g., phosphorylation at residue S235, S236, S240, and/or S244). In some embodiments, the dose of the mTOR inhibitor in the composition for each administration is from about 10 mg/m² to about 100 mg/m² (e.g., about 50 mg/m² to about 100 mg/m², about 75 mg/m² to about 100 mg/m²). In some embodiments, the method comprises administering the nanoparticle composition to the individual weekly for about two weeks followed by a rest period of about one week. In some embodiments, there is provided a method of treating a cancer (e.g., an advanced and/or malignant cancer, e.g., PEComa. e.g., an advanced and/or malignant cancer, e.g., locally advanced inoperable cancer, e.g., a solid tumor) in an individual comprising administering (e.g., intravenously or subcutaneously administering) to the individual an effective amount of a composition comprising nanoparticles comprising an mTOR inhibitor (e.g., rapamycin) and a carrier protein (e.g., albumin), wherein the individual is selected for treatment on the basis of a) having a mutation in TP53 or ATRX, and b) having an aberrant phosphorylation level of the protein encoded by RPS6 (e.g., phosphorylation at residue S235, S236, S240, and/or S244), wherein the dose of the mTOR inhibitor in the composition for each administration is from about 10 mg/m² to about 100 mg/m² (e.g., about 50 mg/m² to about 100 mg/m², about 75 mg/m² to about 100 mg/m²), and optionally wherein the composition is administered weekly for about two weeks followed by a rest period of about one week. In some embodiments, the mutation is selected from the group consisting of a splice site mutation, a nonsense mutation, a frameshift mutation, a missense mutation and a loss or deletion of the gene. In some embodiments, the mTOR inhibitor is a limus drug. In some embodiments, the mTOR inhibitor is rapamycin or a derivative thereof. In some embodiments, the mTOR inhibitor is rapamycin. In some embodiments, the carrier protein is albumin (such as human serum albumin). In some embodiments, the cancer is selected from the group consisting of pancreatic neuroendocrine cancer, endometrial cancer, breast cancer, lymphangioleiomyomatosis (LAM), prostate cancer, hepatocellular carcinoma, melanoma, renal cell carcinoma, bladder cancer, endometrial cancer, ovary cancer, gynecologic cancer, sarcoma, perivascular epithelioid cell neoplasms (PEComa), Hodgkin's lymphoma and multiple myeloma. In some embodiments, the cancer is a PEComa. In some embodiments, the individual is selected for treatment based on having a TP53 or ATRX aberration and a RPS6 aberration, regardless of the nature of the cancer. In some embodiments, the method further comprises administering an anti-PD-1 antibody into the individual. In some embodiments, the anti-PD-1 antibody is administered at a dose of about 1 mg/kg to about 5 mg/kg (such as about 3 mg/kg) once every three weeks. In some embodiments, the individual fails to respond to one or more prior therapy (such as a different mTOR inhibitor. e.g., everolimus, such as an immune checkpoint inhibitor, e.g., an anti-PD-1 antibody).

In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual comprising administering (e.g., intravenously or subcutaneously administering) to the individual a composition comprising nanoparticles comprising rapamycin or a derivative thereof and an albumin, wherein the individual is selected for treatment on the basis of a) having a TSC2 aberration (e.g., a TSC2 mutation), and b) having an aberrant phosphorylation level of the protein encoded by RPS6 (e.g., phosphorylation at residue S235, S236, S240, and/or S244), wherein the dose of rapamycin or a derivative thereof in the composition for each administration is from about 10 mg/m² to about 100 mg/m² (e.g., about 25 mg/m² to about 100 mg/m², about 50 mg/m² to about 100 mg/m², about 75 mg/m² to about 100 mg/m²), and wherein the composition is administered weekly for about two weeks followed by a rest period of about one week.

In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual comprising administering (e.g., intravenously or subcutaneously administering) to the individual a composition comprising nanoparticles comprising rapamycin or a derivative thereof and an albumin, wherein the individual is selected for treatment on the basis of a) having a TSC1 aberration (e.g., a TSC1 mutation), and b) having an aberrant phosphorylation level of the protein encoded by RPS6 (e.g., phosphorylation at residue S235, S236, S240, and/or S244), wherein the dose of rapamycin or a derivative thereof in the composition for each administration is from about 10 mg/m² to about 100 mg/m² (e.g., about 25 mg/m² to about 100 mg/m², about 50 mg/m to about 100 mg/m², about 75 mg/m² to about 100 mg/m²), and wherein the composition is administered weekly for about two weeks followed by a rest period of about one week. In some embodiments, the method further comprises administering an anti-PD-1 antibody into the individual. In some embodiments, the anti-PD-1 antibody is administered at a dose of about 1 mg/kg to about 5 mg/kg (such as about 3 mg/kg) once every three weeks. In some embodiments, the individual fails to respond to one or more prior therapy (such as a different mTOR inhibitor. e.g., everolimus, such as an immune checkpoint inhibitor, e.g., an anti-PD-1 antibody).

In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual comprising administering (e.g., intravenously or subcutaneously administering) to the individual a composition comprising nanoparticles comprising rapamycin or a derivative thereof and an albumin, wherein the individual is selected for treatment on the basis of a) having a TSC2 aberration (e.g., a TSC2 mutation), b) does not have a TSC1 mutation, and c) having an aberrant phosphorylation level of the protein encoded by RPS6 (e.g., phosphorylation at residue S235, S236, S240, and/or S244), wherein the dose of rapamycin or a derivative thereof in the composition for each administration is from about 10 mg/m² to about 100 mg/m² (e.g., about 25 mg/m² to about 100 mg/m², about 50 mg/m² to about 100 mg/m², about 75 mg/m² to about 100 mg/m²), and wherein the composition is administered weekly for about two weeks followed by a rest period of about one week. In some embodiments, the method further comprises administering an anti-PD-1 antibody into the individual. In some embodiments, the anti-PD-1 antibody is administered at a dose of about 1 mg/kg to about 5 mg/kg (such as about 3 mg/kg) once every three weeks. In some embodiments, the individual fails to respond to one or more prior therapy (such as a different mTOR inhibitor, e.g., everolimus, such as an immune checkpoint inhibitor, e.g., an anti-PD-1 antibody).

In some embodiments, the aberrant phosphorylation level of the protein encoded by RPS6 is a positive status of phosphorylated S6 (pS6). In some embodiments, the aberrant phosphorylation level of the protein encoded by RPS6 is an increased phosphorylation of S6 in the cancer as compared to a reference tissue. In some embodiments, the reference tissue is derived from a non-cancerous tissue in the individual. In some embodiments, the reference tissue is derived from a corresponding tissue in another individual that does not have the cancer.

In some embodiments, there is provided a method of treating a population of individuals having different cancers (e.g. advanced and/or malignant cancer, e.g., locally advanced inoperable cancer, e.g., a solid tumor), comprising administering (e.g., intravenously or subcutaneously administering) to the population of individuals an effective amount of a composition comprising nanoparticles comprising an mTOR inhibitor (e.g., rapamycin) and a carrier protein (e.g., albumin), wherein each of the individuals has a TSC2 aberration (e.g., TSC2 mutation). In some embodiments, each of the individuals does not have a TSC1 mutation. In some embodiments, the method further comprises administering an anti-PD-1 antibody into the population of individual.

In some embodiments, there is provided a method of treating a population of individuals having different cancers (e.g. advanced and/or malignant cancer, e.g., locally advanced inoperable cancer, e.g., a solid tumor), comprising administering (e.g., intravenously or subcutaneously administering) to the population of individuals an effective amount of a composition comprising nanoparticles comprising an mTOR inhibitor (e.g., rapamycin) and a carrier protein (e.g., albumin), wherein each of the individuals has a RPS6 aberration (e.g., an aberrant phosphorylation level of the protein encoded by RPS6). In some embodiments, the individual has one or more mTOR-activating aberration at one or more (such as one, two, three, four, five, or six) genes selected from the group consisting of TSC1, TSC2, PTEN, TP53, RB1, ATRX, and FAT1. In some embodiments, the individual has one or more mTOR-activating aberration at one or more (such as one, two, three, four, five, or six) genes selected from the group consisting of TSC1, TSC2, ATRX, and TP53. In some embodiments, the individual has one or more mTOR-activating aberration at TSC1 or TSC2. In some embodiments, the method further comprises administering an anti-PD-1 antibody into the population of individual. In some embodiments, the population of individuals fails to respond to one or more prior therapy (such as a different mTOR inhibitor, e.g., everolimus, such as an immune checkpoint inhibitor, e.g., an anti-PD-1 antibody).

In some embodiments, there is provided a method of selecting an individual for a treatment on the basis of having a cancer that harbors a TSC2 mutation, wherein the treatment comprises administering to the individual a composition comprising nanoparticles comprising rapamycin or a derivative thereof and an albumin, wherein optionally the dose of rapamycin or a derivative thereof in the composition for each administration is from about 10 mg/m² to about 100 mg/m² (e.g., about 25 mg/m² to about 100 mg/m², about 50 mg/m² to about 100 mg/m², about 75 mg/m² to about 100 mg/m²), and wherein optionally the composition is administered weekly for about two weeks followed by a rest period of about one week. In some embodiments, the individual does not have a TSC1 mutation.

In some embodiments, there is provided a method of selecting an individual for a treatment on the basis of having a cancer characterized in an aberrant phosphorylation level of a protein encoded by RPS6, wherein the treatment comprises administering to the individual a composition comprising nanoparticles comprising rapamycin or a derivative thereof and an albumin, wherein optionally the dose of rapamycin or a derivative thereof in the composition for each administration is from about 10 mg/m² to about 100 mg/m² (e.g., about 25 mg/m² to about 100 mg/m², about 50 mg/m² to about 100 mg/m², about 75 mg/m² to about 100 mg/m²), and wherein optionally the composition is administered weekly for about two weeks followed by a rest period of about one week. In some embodiments, the individual has one or more mTOR-activating aberration at one or more (such as one, two, three, four, five, or six) genes selected from the group consisting of TSC1, TSC2, PTEN, TP53, RB1, ATRX, and FAT1. In some embodiments, the individual has one or more mTOR-activating aberration at one or more (such as one, two, three, or four) genes selected from the group consisting of TSC1, TSC2, ATRX, and TP53. In some embodiments, the individual has one or more mTOR-activating aberration at TSC1 or TSC2.

In some embodiments, there is provided a method of treating a cancer (e.g., an advanced and/or malignant cancer, e.g., PEComa, e.g., an advanced and/or malignant cancer, e.g., locally advanced inoperable cancer, e.g., a solid tumor) in an individual comprising administering (e.g., intravenously or subcutaneously administering) to the individual an effective amount of a composition comprising nanoparticles comprising an mTOR inhibitor (such as rapamycin) and a carrier protein (such as albumin) for at least about 6 months (such as at least about one year, one and a half years, or two years), wherein the individual is selected for treatment on the basis of having an mTOR-activating aberration at a) RPS6 and b) one other gene selected from the group consisting of TSC1, TSC2, PTEN, TP53, RB1, ATRX, and FAT1. In some embodiments, the dose of the mTOR inhibitor in the composition for each administration is from about 10 mg/m² to about 100 mg/m² (e.g., about 50 mg/m² to about 100 mg/m², about 75 mg/m² to about 100 mg/m²). In some embodiments, the method comprises administering the nanoparticle composition to the individual weekly for about two weeks followed by a rest period of about one week. In some embodiments, the individual is selected for treatment on the basis of a) having a TSC2 aberration (e.g., a TSC2 mutation), and b) having a RPS6 aberration (e.g., aberrant phosphorylation level of the protein encoded by RPS6 (e.g., phosphorylation at residue S235, S236, S240, and/or S244). In some embodiments, the individual is selected for treatment on the basis of a) having a mutation in TSC1, and b) having an aberrant phosphorylation level of the protein encoded by RPS6 (e.g., phosphorylation at residue S235, S236, S240, and/or S244). In some embodiments, the individual is selected for treatment on the basis of a) having a mutation in TP53 or ATRX, and b) having an aberrant phosphorylation level of the protein encoded by RPS6 (e.g., phosphorylation at residue S235, S236, S240, and/or S244). In some embodiments, the method further comprises administering an anti-PD-1 antibody into the individual. In some embodiments, the individual fails to respond to one or more prior therapy (such as a different mTOR inhibitor, e.g., everolimus, such as an immune checkpoint inhibitor, e.g., an anti-PD-1 antibody).

In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of having an mTOR aberration (e.g., inactivating mutation) at TSC1 or TSC2, wherein the individual has not been treated with an mTOR inhibitor. In some embodiments, the individual has failed (e.g., is refractory or resistant to) a prior therapy. In some embodiments, the prior therapy is a standard therapy for the cancer. In some embodiments, the individual is unlikely to tolerate or derive clinically meaningful benefit from appropriate standard of care therapy, or has no satisfactory alternative treatment (e.g., in the opinion of the investigator (e.g., a doctor treating the patient)). Prior therapy includes and is not limited to platinum-based therapy (e.g., cisplatin or carboplatin) an angiogenesis inhibitor (e.g., anti-VEGF antibody (e.g., bevacizumab)), a chemotherapeutic agent (e.g., gemcitabine, doxorubicin, vinorelbine, pazopanib, ifosfamide, Adriamycin, a taxane (e.g., paclitaxel), a checkpoint inhibitor (e.g., anti-PD-1 antibody, e.g., pembrolizumab), a RANKL ligand inhibitor (e.g., denosumab). In some embodiments, the inactivating mutation in TSC1 or TSC2 comprises a homozygous deletion, bi-allelic mutations, a splice site mutation, a frameshift mutation, nonsense mutation in coding region, missense mutation with confirmed impact, or a loss or deletion of TSC1 or TSC2. In some embodiments, the mTOR aberration at TSC1 or TSC2 comprises bi-allelic mutations. In some embodiments, the individual is a human and is administered (e.g., via an intravenous bolus administration) the composition at a dose of about 30 mg/m² to about 100 mg/m² (e.g., about 30 mg/m². 45 mg/m². 60 mg/m². 75 mg/m², 100 mg/m²) for two out of every three weeks a cycle for one or more cycles. In some embodiments, the individual receives at least 2, 3, 4, 5, 6, 7, 8, 9, 10, I1, 12, 13, 14, 15, 16, 17, 18, 19, 20, 21, 22, 23, or 24 cycles of treatment. In some embodiments, the individual receives administration of the composition a dose of about 30 mg/m² to about 100 mg/m² (e.g., about 30 mg/m², 45 mg/m², 60 mg/m², 75 mg/m², 100 mg/m²) for two out of every three weeks a cycle for at least about 6 months, 9 months, 12 months, 15 months, 18 months, 21 months, or 24 months. In some embodiments, the individual has a perivascular epithelioid cell neoplasms (PEComa), an ovarian cancer (e.g., epithelial ovarian cancer), an endometrial cancer, or a sarcoma (e.g., a high grade sarcoma, e.g., endometrial stromal sarcoma).

In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1 or TSC2, b) having an aberration (e.g., inactivating mutation) at any one or more (such as 1, 2, 3, 4, 5, 6, or more) of the genes selected from the group consisting of APH1A, AR, ARID1A, ARID1B, ASMTL, ATR, ARX, BAP1, BCL2L11, BLM, BRD4, BRIP1, BUB1B, BRCA2, CIC, CARM1, CCNE1, CD22, CDH4, C17orf70, CDKN1A, CDKN1B, CDKN2C, CEBPA, CHEK1, CKS1B, CRLF2, CTCF, CYLD, DAXX, DICER1, DMC1, DNMT1, DNMT3A, EPCAM, EP300, EPHA5, ERBB3, ERCC5, ETS1, ETV1, ETV4, EXT1, EZH2, FANCA, FANCL, FAT1, FGFR3, FGFR4, FLCN, FAM123B, FANCB, FANCD2, FANCF, FAS, FLT1, FLT3, FLT4, FOXO1, FOXL2, GATA2, GEN1, GLI2, GNAS, H19, HELQ, IL7R, JAK2, JAZF1, KAT6B, KDM6A, KDR, KEAP1, KIT, KLF4, KMT2A, KMT2D, KRAS, MAP3K1, MAP3K6, MCL1, MCM8, MEF2B, MGA, MTOR, MUTYH, MYCN, NBN, NF1, NF2, NPM1, NSD1, NRG1, NOTCH3, NR0B1, NTRK1, PBRM1, PDGFRA, PDGFRB, PIK3C2B, PTCH1, PTEN, POT1, PMS2, PRKDC, POLQ, PTCH1, PVRL4, RAD21, RAD50, RAF1, RB1, RBBP8, RET, RIF1, RIT1, RNF43, ROS1, RSPO2, RPTOR, SETD2, SMARCA4, SOCS1, STED2, SUFU, TCEB1, TET2, TGFBR2, TLX3, TP53, TP53BP1, TRIM37, TSHR, UIMC1, VHL, WHSC1L1, WRN, XPA, YY1AP1, and ZNF217. In some embodiments, the individual has an aberration (e.g., inactivating mutation) at any one or more (such as 1, 2, 3, 4, 5, 6, or more) of the genes selected from the group consisting of APH1A, AR, ASMTL, ATRX, BCL2L11, CARM1, CD22, CDKN1B, CKS1B, CRLF2, DAXX, DNMT1, EPHA5, ERBB3, ETS1, FAT1 FAM123B, FANCD2, FAS, FLT1, FOXO1, IL7R, KDM6A, KDR, KEAP1, MAP3K6, MEF2B, NF1, NTRK1, PDGFRB, PTEN, POT1, RAD21, RAF1, RB1, SMARCA4, TGFBR2, TP53, YY1AP1, and ZNF217. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1 or TSC2, b) having an aberration (e.g., inactivating mutation) at any one or more (such as 1, 2, 3, 4, 5, or more) of the genes selected from the group consisting of FLT1, IL7R, RB1, TP53, PTEN, and YY1AP1. In some embodiments, the individual has not been treated with an mTOR inhibitor. In some embodiments, the individual has failed (e.g., is refractory or resistant to) a prior therapy. In some embodiments, the prior therapy is a standard therapy for the cancer. In some embodiments, the individual is unlikely to tolerate or derive clinically meaningful benefit from appropriate standard of care therapy, or has no satisfactory alternative treatment (e.g., in the opinion of the investigator (e.g., a doctor treating the patient)). Prior therapy includes and is not limited to platinum-based therapy (e.g., cisplatin or carboplatin) an angiogenesis inhibitor (e.g., anti-VEGF antibody (e.g., bevacizumab)), a chemotherapeutic agent (e.g., gemcitabine, doxorubicin, vinorelbine, pazopanib, ifosfamide, Adriamycin, a taxane (e.g., paclitaxel), a checkpoint inhibitor (e.g., anti-PD-1 antibody. e.g., pembrolizumab), a RANKL ligand inhibitor (e.g., denosumab). In some embodiments, the inactivating mutation in TSC1 or TSC2 comprises a homozygous deletion, bi-allelic mutations, a splice site mutation, a frameshift mutation, nonsense mutation in coding region, missense mutation with confirmed impact, or a loss or deletion of TSC1 or TSC2. In some embodiments, the mTOR aberration at TSC1 or TSC2 comprises bi-allelic mutations. In some embodiments, the individual is a human and is administered (e.g., via an intravenous bolus administration) the composition at a dose of about 30 mg/m² to about 100 mg/m² (e.g., about 30 mg/m², 45 mg/m², 60 mg/m², 75 mg/m², 100 mg/m²) for two out of every three weeks a cycle for one or more cycles. In some embodiments, the individual receives at least 2, 3, 4, 5, 6, 7, 8, 9, 10, 11, 12, 13, 14, 15, 16, 17, 18, 19, 20, 21, 22, 23, or 24 cycles of treatment. In some embodiments, the individual receives administration of the composition a dose of about 30 mg/m² to about 100 mg/m² (e.g., about 30 mg/m², 45 mg/m², 60 mg/m², 75 mg/m², 100 mg/m²) for two out of every three weeks a cycle for at least about 6 months, 9 months, 12 months, 15 months, 18 months, 21 months, or 24 months. In some embodiments, the individual has a perivascular epithelioid cell neoplasms (PEComa), an ovarian cancer (e.g., epithelial ovarian cancer), an endometrial cancer, or a sarcoma (e.g., a high grade sarcoma. e.g., endometrial stromal sarcoma).

In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1 or TSC2, b) having an aberration (e.g., inactivating mutation) at any one or more (such as 1, 2, 3, 4, 5, 6, or more) of the genes selected from the group consisting of APH1A, ASXL1, BCL2L11, BRD4, BUB1B, C17orf70, C19orf40, CARM1, CCNE1, CD22, CDKN1A, CDKN1B, CDKN2C, CEBPA, CHEK1, CIC, CKS1B, CRLF2, CTCF, CYLD, DAXX, DMC1, DNMT, EPCAM, ERBB3, ETS, ETV1, ETV4, EXO1, EXT1, FAM123B, FANCA, FANCB, FGFR4, FLT1, FLT4, FOXO1, GATA2, GEN1, GLI1, GLI2, H19, HELQ, 1L7R JAK3, JAZF1, KAT6B, KDR KEAP1, KMT2A, MAP3K6, MCL1, MCM8, MEF2B, MEN1, MYCN, NF1, NPM1, NRG1, NR0B1, NSD1, NTRK1, PRKDC, PDGFRA, POLQ, POT1, PRKDC, PVRL4, RAD21, RAF1, RIT1, RNF43, ROS1, RPTOR, SDHA, SETBP1, SMARCA4, SOCS1, TCEB1, TET2, TSHR, UIMC1, WHSC1L1, XPA, YY1AP1, and ZNF21.

In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC2, b) having an aberration (e.g., inactivating mutation) at any one or more (such as 1, 2, 3, 4, 5, 6, or more) of the genes selected from the group consisting of ATR, AR, ASMTL, ASXL1, BCL2L11, BLM, BRCA2, BRIP1, BUB1B, CARM1, C17orf70, C19orf40, CIC, CCNE1, CDH4, CDKN2C, CDKN1A, CDKN1B, DAXX, DNMT1, EPHA5, EPCAM, ERBB3, ETV1, EXO1, EXT1, EZH2, FAT1, FAN), FANCA, FANCL, FANCD2, FGFR3, FGFR4, FAS, FAT1, FLT7, FOXO1, FLT4, GNAS, GLI2, H19, HELQ, IL7R, JAK2, JAZF1, KAT6B, KDM6A, KEAP1, KIT KLF4, MAP3K, MCM8, MGA, NPM1, NRG1, NR0B1, NTRK1, PDGFRA, PDGFRB, PIK3C2B, PMS2, POLQ, PRKDC PTEN, PTCH1, PRKDC, RAD21, RAD50, RB1, RET, RIF, RSPO2, SETBP1, SETD2, SMARCA4, SOCS1, TLX3, TP53, TRIM37, UIMC1, VHL, WHSC1L1, XPA, WRN, and YY1AP1. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC2, b) having an aberration (e.g., inactivating mutation) at any one or more (such as 1, 2, 3, 4, 5, 6, or more) of the genes selected from the group consisting of ASMTL, ASXL1, BCL2L11, BUB1B, CARM1, C17orf70, C19orf40, CIC, CCNE1, CDKN2C, CDKN1A, CDKN1B, DAXX DNMT1, EPCAM, ERBB3, ETV1, EXO1, EXT1, FANCA, FGFR4, FLT1, FOXO1, FLT4, GLI2, H19, HELQ, IL7R, JAK2, JAZF1, KAT6B, KEAP1, MCM8, NPM1, NRG1, NR0B1, NTRK1, PDGFRA, POLQ, PRKDC, RAD21, SETBP1, SMARCA4, SOCS, UIMC1, WHSC1L1, XPA, and YY1AP1. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC2, b) having an aberration (e.g., inactivating mutation) at any one or more (such as 1, 2, 3, 4, 5, 6, or more) of the genes selected from the group consisting of AR, ASMTL, BCL2L11, CARM1, CDKN1B. DAXX, DNMT1, EPHA5, ERBB3, FAS, FAT1, FLT1, FOXO1, IL7R, KDM6A, KEAP1, NTRK1, PTEN, RAD21, RB1, SMARCA4, TP53, and YY1AP1. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC2, b) having an aberration (e.g., inactivating mutation) at any one or more (such as 1, 2, or 3) of the genes selected from the group consisting of AR, IL7R, and NTRK1. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC2, b) having an aberration (e.g., inactivating mutation) at any one or more (such as 1, 2, 3, 4, 5, 6, or more) of the genes selected from the group consisting of BCL2L11, CARM1, CDKN1B, DNMT1, EPHA5, FOXO1, KEAP1, SMARCA4, and TP53. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC2, b) having an aberration (e.g., inactivating mutation) at any one or more (such as 1, 2, 3, 4, 5, 6, or more) of the genes selected from the group consisting of ASMTL, DAXX, ERBB3, FLT1, RAD21, RB1, TP53, and YY1AP1. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC2, b) having an aberration (e.g., inactivating mutation) at TP53. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC2, b) having an aberration (e.g., inactivating mutation) at FAT1. In some embodiments, the individual has not been treated with an mTOR inhibitor. In some embodiments, the individual has failed (e.g., is refractory or resistant to) a prior therapy. In some embodiments, the prior therapy is a standard therapy for the cancer. In some embodiments, the individual is unlikely to tolerate or derive clinically meaningful benefit from appropriate standard of care therapy, or has no satisfactory alternative treatment (e.g., in the opinion of the investigator (e.g., a doctor treating the patient)). Prior therapy includes and is not limited to platinum-based therapy (e.g., cisplatin or carboplatin) an angiogenesis inhibitor (e.g., anti-VEGF antibody (e.g., bevacizumab)), a chemotherapeutic agent (e.g., gemcitabine, doxorubicin, vinorelbine, pazopanib, ifosfamide, Adriamycin, a taxane (e.g., paclitaxel), a checkpoint inhibitor (e.g., anti-PD-1 antibody, e.g., pembrolizumab), a RANKL ligand inhibitor (e.g., denosumab). In some embodiments, the inactivating mutation in TSC1 or TSC2 comprises a homozygous deletion, bi-allelic mutations, a splice site mutation, a frameshift mutation, nonsense mutation in coding region, missense mutation with confirmed impact, or a loss or deletion of TSC1 or TSC2. In some embodiments, the mTOR aberration at TSC1 or TSC2 comprises bi-allelic mutations. In some embodiments, the individual is a human and is administered (e.g., via an intravenous bolus administration) the composition at a dose of about 30 mg/m² to about 100 mg/m² (e.g., about 30 mg/m², 45 mg/m², 60 mg/m². 75 mg/m², 100 mg/m²) for two out of every three weeks a cycle for one or more cycles. In some embodiments, the individual receives at least 2, 3, 4, 5, 6, 7, 8, 9, 10, 11, 12, 13, 14, 15, 16, 17, 18, 19, 20, 21, 22, 23, or 24 cycles of treatment. In some embodiments, the individual receives administration of the composition a dose of about 30 mg/m² to about 100 mg/m² (e.g., about 30 mg/m², 45 mg/m², 60 mg/m², 75 mg/m², 100 mg/m²) for two out of every three weeks a cycle for at least about 6 months, 9 months, 12 months, 15 months, 18 months, 21 months, or 24 months. In some embodiments, the individual has a perivascular epithelioid cell neoplasms (PEComa), an ovarian cancer (e.g., epithelial ovarian cancer), an endometrial cancer, or a sarcoma (e.g., a high grade sarcoma, e.g., endometrial stromal sarcoma).

In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1, b) having an aberration (e.g., inactivating mutation) at any one or more (such as 1, 2, 3, 4, 5, 6, or more) of the genes selected from the group consisting of AR, APH1A, ATRX, ARID1B, BRD4, BRCA2, BUB1B, CCNE1, C19orf40, CDH4, CDKN2C, CD22, CEBPA, CHEK1, CKS1B, CRLF2, CTCF, CYLD, DICER1, DMC1, DNMT3A, EP300, ERCC5, ERBB3, ETV4, ETS1, EXO1, EXT1, FAM123B, FANCB, FANCF, FANCD2, FAN1, FLT1, FOXL2, GATA2, GEN1, GLI1, GLI2, IL7R, KAT6B, KDR, KIT, KMT2A, KMT2D, MAP3K6, MCL1, MAP3K1, MCM8, MEF2B, MEN1, MSH2, MUTYH, MYCN, NOTCH3, NSD1, NF1, NTRK1, PDGFRB, POT1, POLQ, PVRL4, RAF1, RB1, RBBP8, RIF1, RIT1, RNF43, RPTOR, ROS1, SDHA, SMARCA4, SUFU, TCEB1, TET2, TGFBR2, TLX3, TP53, TP53BPL, TSHR, WHSC1L1, XPA, YY1AP1, and ZNF217. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1, b) having an aberration (e.g., inactivating mutation) at any one or more (such as 1, 2, 3, 4, 5, 6, or more) of the genes selected from the group consisting of APH1A, BRD4, BUB1B, CCNE1, C19orf40, CDKN2C, CD22, CEBPA, CHEK1, CKS1B, CRLF2, CTCF, CYLD, DMC1, ERBB3, ETV4, ETS1, EXO1, EXT1, FAM123R, FANCB, FLT1, GATA2, GEN1, GLI1, G12, IL7R, KAT6B, KDR, KMT2A, MAP3K6, MCL1, MCM8, MEF2B, MEN1, MYCN, NSD1, NF1, NTRK1, POT1 POLQ, PVRL4, RAF1, RIT1, RNF43, RPTOR, ROS1, SDHA, SMARCA4, TCEB1, TET2, TSHR, WHSC1L1, XPA, YY1AP1, and ZNF217. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1, b) having an aberration (e.g., inactivating mutation) at any one or more (such as 1, 2, 3, 4, 5, 6, or more) of the genes selected from the group consisting of APH1A, ATRX, CD22, CKS1B, CRLF2, ETS1, FAM123B, FANCD2, FLT1, IL7R, KDR, MAP3K6, MEF2B, NF1, NTRK1, PDGFRB, POT1, RAF1, RB1, TGFBR2, TP53, and YY1AP1. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1, b) having an aberration (e.g., inactivating mutation) at any of the genes selected from the group consisting of MEF2B, NF1, RAF1, RB1, and TP53. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1, b) having an aberration (e.g., inactivating mutation) at any one or more (such as 1, 2, 3, 4, 5, 6, or more) of the genes selected from the group consisting of APH1A, CD22, CKS1B, CRLF2, ETS1, FAM123B, FANCD2, FLT1, IL7R, KDR, MAP3K6, NTRK1, PDGFRB, POT1, TGFBR2, and YY1AP1. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1, b) having an aberration (e.g., inactivating mutation) at ATRX. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1, b) having an aberration (e.g., inactivating mutation) at any one or more (such as one, two or three) of the genes selected from the group consisting of TP53, RB1, and FAT1. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1, b) having an aberration (e.g., inactivating mutation) at any one or more (such as one, two or three) of the genes selected from the group consisting of TP53, RB1, and PTEN. In some embodiments, the individual has not been treated with an mTOR inhibitor. In some embodiments, the individual has failed (e.g., is refractory or resistant to) a prior therapy. In some embodiments, the prior therapy is a standard therapy for the cancer. In some embodiments, the individual is unlikely to tolerate or derive clinically meaningful benefit from appropriate standard of care therapy, or has no satisfactory alternative treatment (e.g., in the opinion of the investigator (e.g., a doctor treating the patient)). Prior therapy includes and is not limited to platinum-based therapy (e.g., cisplatin or carboplatin) an angiogenesis inhibitor (e.g., anti-VEGF antibody (e.g., bevacizumab)), a chemotherapeutic agent (e.g., gemcitabine, doxorubicin, vinorelbine, pazopanib, ifosfamide, Adriamycin, a taxane (e.g., paclitaxel), a checkpoint inhibitor (e.g., anti-PD-1 antibody. e.g., pembrolizumab), a RANKL ligand inhibitor (e.g., denosumab). In some embodiments, the inactivating mutation in TSC1 or TSC2 comprises a homozygous deletion, bi-allelic mutations, a splice site mutation, a frameshift mutation, nonsense mutation in coding region, missense mutation with confirmed impact or a loss or deletion of TSC1 or TSC2. In some embodiments, the mTOR aberration at TSC1 or TSC2 comprises bi-allelic mutations. In some embodiments, the individual is a human and is administered (e.g., via an intravenous bolus administration) the composition at a dose of about 30 mg/in to about 100 mg/m² (e.g., about 30 mg/m², 45 mg/m², 60 mg/m², 75 mg/m², 100 mg/m²) for two out of every three weeks a cycle for one or more cycles. In some embodiments, the individual receives at least 2, 3, 4, 5, 6, 7, 8, 9, 10, 11, 12, 13, 14, 15, 16, 17, 18, 19, 20, 21, 22, 23, or 24 cycles of treatment. In some embodiments, the individual receives administration of the composition a dose of about 30 mg/in to about 100 mg/m² (e.g., about 30 mg/m², 45 mg/in, 60 mg/m², 75 mg/m², 100 mg/m²) for two out of every three weeks a cycle for at least about 6 months, 9 months, 12 months, 15 months, 18 months, 21 months, or 24 months. In some embodiments, the individual has a perivascular epithelioid cell neoplasms (PEComa), an ovarian cancer (e.g., epithelial ovarian cancer), an endometrial cancer, or a sarcoma (e.g., a high grade sarcoma, e.g., endometrial stromal sarcoma).

In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1 or TSC2, b) having an aberration (e.g., inactivating mutation) at any one or more (such as 1, 2, 3, 4, 5, 6, or more) of the genes selected from the group consisting of TP53. RB1, VHL, PBRM1, PTEN, SETD2, BAP1, BRCA2, FANCD2, ARID1A, ARID1B, CDKN2A, FAT1, KDM6A, KIT, PDGFRB, RIF1. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1 or TSC2, b) having an aberration (e.g., inactivating mutation) at any one or more (such as 1, 2, 3, 4, 5, 6, or more) of the genes selected from the group consisting of TP53, RB1, TLX3, SMARCA4, RIF1, PTEN, NTRK1, FLT1, ERBB3, CDKN2C, ATRX, YY1AP1, XPA, WRN, PTCH1, PMS2, PDGFRB, NSD1, KMT2A, KDM6A, IL7R, GNAS, GLI2, GLI1, FLT4, FAT1, FANCD2, EXT1, DNMT3A, DAXX, CDH4, CCNE1, and BUB1B. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1 or TSC2, b) having an aberration (e.g., inactivating mutation) at any one or more (such as 1, 2, 3, 4, 5, 6, or more) of the genes selected from the group consisting of TP53, RB1, TLX3, SMARCA4, RIF1, PTEN, NTRK1, FLT1, ERBB3, CDKN2C, and ATRX. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1 or TSC2, b) having an aberration (e.g., inactivating mutation) at any one or more (such as 1, 2, 3, 4, 5, 6, or more) of the genes selected from the group consisting of TP53, VHL, RB1, PBRM1, ATRX, KDM6A, RET, SETD2, ARID1A, BAP1, FLT1, NTRK1, TLX3, and BRCA2. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1 or TSC2, b) having an aberration (e.g., inactivating mutation) at any one or more (such as 1, 2, 3, 4, 5, 6, or more) of the genes selected from the group consisting of TP53, RB1, ATRX, FLT1, NTRK1, TLX3, KDM6A, CDH4, CDKN2C, DAXX, ERBB3, GNAS, IL7R, PDGFRB, PMS2, PTEN, SMARCA4, and YY1AP1. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1 or TSC2, b) having an aberration (e.g., inactivating mutation) at any one or more (such as 1, 2, 3, 4, 5, 6, or more) of the genes selected from the group consisting of TP53, RB1, ATRX, FLT1, NTRK1, and TLX3. In some embodiments, the individual does not have an aberration (e.g., a mutation) at any one or more (such as 1, 2, 3, 4, or 5) of the genes selected from the group consisting of GLI1, KMT2A, NSD1, RIF1, and XPA. In some embodiments, the individual has not been treated with an mTOR inhibitor. In some embodiments, the individual has failed (e.g., is refractory or resistant to) a prior therapy. In some embodiments, the prior therapy is a standard therapy for the cancer. In some embodiments, the individual is unlikely to tolerate or derive clinically meaningful benefit from appropriate standard of care therapy, or has no satisfactory alternative treatment (e.g., in the opinion of the investigator (e.g., a doctor treating the patient)). Prior therapy includes and is not limited to platinum-based therapy (e.g., cisplatin or carboplatin) an angiogenesis inhibitor (e.g., anti-VEGF antibody (e.g., bevacizumab)), a chemotherapeutic agent (e.g., gemcitabine, doxorubicin, vinorelbine, pazopanib, ifosfamide, Adriamycin, a taxane (e.g., paclitaxel), a checkpoint inhibitor (e.g., anti-PD-1 antibody, e.g., pembrolizumab), a RANKL ligand inhibitor (e.g., denosumab). In some embodiments, the inactivating mutation in TSC1 or TSC2 comprises a homozygous deletion, bi-allelic mutations, a splice site mutation, a frameshift mutation, nonsense mutation in coding region, missense mutation with confirmed impact or a loss or deletion of TSC1 or TSC2. In some embodiments, the mTOR aberration at TSC1 or TSC2 comprises bi-allelic mutations. In some embodiments, the individual is a human and is administered (e.g., via an intravenous bolus administration) the composition at a dose of about 30 mg/m² to about 100 mg/m² (e.g., about 30 mg/m², 45 mg/m², 60 mg/m², 75 mg/m², 100 mg/m²) for two out of every three weeks a cycle for one or more cycles. In some embodiments, the individual receives at least 2, 3, 4, 5, 6, 7, 8, 9, 10, 11, 12, 13, 14, 15, 16, 17, 18, 19, 20, 21, 22, 23, or 24 cycles of treatment. In some embodiments, the individual receives administration of the composition a dose of about 30 mg/m² to about 100 mg/m² (e.g., about 30 mg/m², 45 mg/m², 60 mg/m², 75 mg/m², 100 mg/m²) for two out of every three weeks a cycle for at least about 6 months, 9 months, 12 months, 15 months, 18 months, 21 months, or 24 months. In some embodiments, the individual has a perivascular epithelioid cell neoplasms (PEComa), an ovarian cancer (e.g., epithelial ovarian cancer), an endometrial cancer, or a sarcoma (e.g., a high grade sarcoma, e.g., endometrial stromal sarcoma).

In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1, b) having an aberration (e.g., inactivating mutation) at any one or more (such as 1, 2, 3, 4, 5, 6, or more) of the genes selected from the group consisting of TP53, RB1, GLI1, KMT2A, NSD1, NTRK1, SMARCA4 and XPA. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1, b) having an aberration (e.g., inactivating mutation) at any one or more (such as 1, 2, 3, or more) of the genes selected from the group consisting of TP53, RB1, VHL, and PBRM1. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1, b) having an aberration (e.g., inactivating mutation) at any one or more (such as 1, 2, 3, 4, 5, 6, or more) of the genes selected from the group consisting of VHL, TP53, PBRM1, BAP1, NTRK1, RB1, ATRX, FANCD2, ARID1A, KDM6A. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC1, b) having an aberration (e.g., inactivating mutation) at any one or more (such as 1, 2, 3, 4, 5, 6, or more) of the genes selected from the group consisting of NTRK1, RB1, TP53, APH1A, ATRX, BUB1B, CD22, CDH4, CDKN2C, CEBPA, CKS1B, CRLF2, ETS, FAM123B, FANCD2, FLT1, IL7R, KDR, MAP3K6, MCL1, MEF2B, MUTYH, NF1, NOTCH3, PDGFRB, POT1, PVRL4, RAF1, RBBP8, RIT1, SDHA, SMARCA4, TET2, TGFBR2, TLX3, YY1AP1, and ZNF217. In some embodiments, the individual does not have an aberration (e.g., a mutation) at any one or more (such as 1, 2, 3, 4, or 5) of the genes selected from the group consisting of GLI1, KMT2A, NSD1, and XPA. In some embodiments, the individual has not been treated with an mTOR inhibitor. In some embodiments, the individual has failed (e.g., is refractory or resistant to) a prior therapy. In some embodiments, the prior therapy is a standard therapy for the cancer. In some embodiments, the individual is unlikely to tolerate or derive clinically meaningful benefit from appropriate standard of care therapy, or has no satisfactory alternative treatment (e.g., in the opinion of the investigator (e.g., a doctor treating the patient)). Prior therapy includes and is not limited to platinum-based therapy (e.g., cisplatin or carboplatin) an angiogenesis inhibitor (e.g., anti-VEGF antibody (e.g., bevacizumab)), a chemotherapeutic agent (e.g., gemcitabine, doxorubicin, vinorelbine, pazopanib, ifosfamide, Adriamycin, a taxane (e.g., paclitaxel), a checkpoint inhibitor (e.g., anti-PD-1 antibody, e.g., pembrolizumab), a RANKL ligand inhibitor (e.g., denosumab). In some embodiments, the inactivating mutation in TSC1 comprises a homozygous deletion, bi-allelic mutations, a splice site mutation, a frameshift mutation, nonsense mutation in coding region, missense mutation with confirmed impact or a loss or deletion of TSC1. In some embodiments, the mTOR aberration at TSC1 comprises bi-allelic mutations. In some embodiments, the individual is a human and is administered (e.g., via an intravenous bolus administration) the composition at a dose of about 30 mg/m² to about 100 mg/m² (e.g., about 30 mg/m², 45 mg/m², 60 mg/m², 75 mg/m², 100 mg/m²) for two out of every three weeks a cycle for one or more cycles. In some embodiments, the individual receives at least 2, 3, 4, 5, 6, 7, 8, 9, 10, 11, 12, 13, 14, 15, 16, 17, 18, 19, 20, 21, 22, 23, or 24 cycles of treatment. In some embodiments, the individual receives administration of the composition a dose of about 30 mg/m² to about 100 mg/m² (e.g., about 30 mg/m², 45 mg/m², 60 mg/m², 75 mg/m², 100 mg/m²) for two out of every three weeks a cycle for at least about 6 months, 9 months, 12 months, 15 months, 18 months, 21 months, or 24 months. In some embodiments, the individual has a perivascular epithelioid cell neoplasms (PEComa), an ovarian cancer (e.g., epithelial ovarian cancer), an endometrial cancer, or a sarcoma (e.g., a high grade sarcoma, e.g., endometrial stromal sarcoma).

In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC2, b) having an aberration (e.g., inactivating mutation) at any one or more (such as 1, 2, 3, 4, 5, 6, or more) of the genes selected from the group consisting of TP53, RB1, PTEN, BRCA2 and CDKN2A. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC2, b) having an aberration (e.g., inactivating mutation) at any one or more (such as 1, 2, 3, 4, 5, 6, or more) of the genes selected from the group consisting of TP53, MSS, ATRX, CDKN2C, DAXX, ERBB3, FLT1, FLT4, GNAS, KDM6A, PMS2, PTCH1, PTEN, RB1, RIF1, TLX3, and WRN. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC2, b) having an aberration (e.g., inactivating mutation) at any one or more (such as 1, 2, 3, 4, 5, 6, or more) of the genes selected from the group consisting of TP53, RB1, BRCA2, RET and SETD2. In some embodiments, there is provided a method of treating a cancer (e.g., metastatic cancer) in an individual, comprising administering an effective amount of a composition comprising nanoparticles comprising an mTOR inhibitor (e.g., rapamycin) and a carrier protein (e.g., albumin), wherein the individual is selected for treatment on the basis of a) having an mTOR aberration (e.g., inactivating mutation) at TSC2, b) having an aberration (e.g., inactivating mutation) at any one or more (such as 1, 2, 3, 4, 5, 6, or more) of the genes selected from the group consisting of TP53, ATRX, DAXX, ERBB3, FLT1. GNAS, KDM6A, PMS2, PTEN, RB1, and TLX. In some embodiments, the individual does not have an aberration (e.g., a mutation) at any one or more (such as 1, 2, 3, 4, or 5) of the genes selected from the group consisting of BRIP1, BUB1B, CDKN2C, FANCD2, FLT4, PDGFRA, PTCH1, RIF1, VHL, and WRN. In some embodiments, the individual has not been treated with an mTOR inhibitor. In some embodiments, the individual has failed (e.g., is refractory or resistant to) a prior therapy. In some embodiments, the prior therapy is a standard therapy for the cancer. In some embodiments, the individual is unlikely to tolerate or derive clinically meaningful benefit from appropriate standard of care therapy, or has no satisfactory alternative treatment (e.g., in the opinion of the investigator (e.g., a doctor treating the patient)). Prior therapy includes and is not limited to platinum-based therapy (e.g., cisplatin or carboplatin) an angiogenesis inhibitor (e.g., anti-VEGF antibody (e.g., bevacizumab)), a chemotherapeutic agent (e.g., gemcitabine, doxorubicin, vinorelbine, pazopanib, ifosfamide, Adriamycin, a taxane (e.g., paclitaxel), a checkpoint inhibitor (e.g., anti-PD-1 antibody. e.g., pembrolizumab), a RANKL ligand inhibitor (e.g., denosumab). In some embodiments, the inactivating mutation in TSC2 comprises a homozygous deletion, bi-allelic mutations, a splice site mutation, a frameshift mutation, nonsense mutation in coding region, missense mutation with confirmed impact or a loss or deletion of TSC2. In some embodiments, the mTOR aberration at TSC2 comprises bi-allelic mutations. In some embodiments, the individual is a human and is administered (e.g., via an intravenous bolus administration) the composition at a dose of about 30 mg/m² to about 100 mg/m² (e.g., about 30 mg/m², 45 mg/m², 60 mg/m², 75 mg/m², 100 mg/m²) for two out of every three weeks a cycle for one or more cycles. In some embodiments, the individual receives at least 2, 3, 4, 5, 6, 7, 8, 9, 10, 11, 12, 13, 14, 15, 16, 17, 18, 19, 20, 21, 22, 23, or 24 cycles of treatment. In some embodiments, the individual receives administration of the composition a dose of about 30 mg/m² to about 100 mg/m² (e.g., about 30 mg/m², 45 mg/m², 60 mg/m², 75 mg/m², 100 mg/m²) for two out of every three weeks a cycle for at least about 6 months, 9 months, 12 months, 15 months, 18 months, 21 months, or 24 months. In some embodiments, the individual has a perivascular epithelioid cell neoplasms (PEComa), an ovarian cancer (e.g., epithelial ovarian cancer), an endometrial cancer, or a sarcoma (e.g., a high grade sarcoma, e.g., endometrial stromal sarcoma).

In some embodiments, the individual has a stable microsatellite status.

In some embodiments, the individual has a low tumor mutational burden (e.g., less than about 10, 9, 8, 7, 6, 5, 4, or 3).

In some embodiments, methods described herein are not for treating a cancer that involve a driver mutation. Exemplary driver mutations include e.g., a deletion mutation in EGFR exon 19 in a lung cancer, e.g., a ERBB2 amplification in a breast cancer. In some embodiments, the individual does not have 1, 2, 3, 4, 5 or any of the following mutations: a) a deletion mutation in EGFR exon 19 (e.g., in a lung cancer (e.g., NSCLC)); b) EGFR exon 21 L858R alteration (e.g., in a lung cancer (e.g., NSCLC)); c) EGFR exon 20 T790M alteration (e.g., in a lung cancer (e.g., NSCLC)); d) AIX rearrangement (e.g., in a lung cancer (e.g., NSCLC)); e) BRAF V600E or V600K (e.g., in a lung cancer (e.g., NSCLC) or a melanoma); f) MET single nucleotide variant or indel that leads to MET exon 14 skipping (e.g., in a lung cancer (e.g., NSCLC)); g) ERBB2 (HER2) amplification (e.g., in a breast cancer); h) any of C420R, E542K, E545A, E545D [1635G>T only], E545G, E545K, Q546E, Q546R, H1047L, H1047R, and H1047Y in PIK3CA (e.g., in a breast cancer); i) BRCA1/2 alteration (e.g., in an ovarian cancer); j) a FGFR2 fusion and/or rearrangement (e.g., in cholangiocarcinoma); k) a mutation in any of BRCA1, BRCA2, ATM, BARD1, BRIP1, CDK12, CHEK1, CHEK2, FANCL, PALB2, RAD51B, RAD51C, RAD51D and RAD54L (e.g., in prostate cancer); 1) has a tumor mutation burden of at least 10 mutations per megabase in a solid tumor. In some embodiments, the individual does not have a mutation in 1, 2, 3, 4, 5, 6, 7, or any of EGFR, ALK, BRAF, MET, ERBB2, PIK3CA, FGFR2, BRCA1, BRCA2, A7M, BARD1, BRIP1, CDK12, CHEK1, CHEK2, FANCL, PALB2, RAD51B, RAD5L1C, RAD51D and RAD54L.

The methods provided herein can be used to treat an individual (e.g., human) who has been diagnosed with or is suspected of having a cancer. In some embodiments, the individual is human. In some embodiments, the individual is at least about any of 20, 25, 30, 35, 40, 45, 50, 55, 60, 65, 70, 75, 80, or 85 years old. In some embodiments, the individual is male. In some embodiments, the individual is female. In some embodiments, the individual has undergone a resection of the tumor. In some embodiments, the individual has refused surgery.

In some embodiments, the individual is medically inoperable. In some of embodiments, the individual is genetically or otherwise predisposed (e.g., having a risk factor) to developing a cancer. These risk factors include, but are not limited to, age, sex, race, diet, history of previous disease, presence of precursor disease, genetic considerations, and environmental exposure. In some embodiments, the individuals at risk for the cancer include, e.g., those having relatives who have experienced the cancer, and those whose risk is determined by analysis of genetic or biochemical markers.

In some embodiments, the composition is administered intravenously.

In some embodiments, the composition is administered subcutaneously.

The methods provided herein may be practiced in an adjuvant setting. In some embodiments, the method is practiced in a neoadjuvant setting, i.e., the method may be carried out before the primary/definitive therapy. In some embodiments, the method is used to treat an individual who has previously been treated. In some embodiments, the individual is resistant, non-responsive, partially responsive, initially responsive, or refractory to a prior therapy. In some embodiments, the individual has progressed on the prior therapy at the time of treatment. In some embodiments, the individual is unsuitable to continue with the prior therapy, for example, due to failure to respond and/or due to toxicity. In some embodiments, the individual has not previously been treated. In some embodiments, the method is used as a first line therapy. In some embodiments, the method is used as a second line therapy.

The methods described herein for treating cancer can be used in monotherapy as well as in combination therapy with another agent. In some embodiments, the composition comprising nanoparticles comprising the mTOR inhibitor (such as a limus drug) and the albumin is administered as a single agent. In some embodiments, the method further comprises administering to the individual an effective amount of at least another therapeutic agent. The other therapeutic agent may be a chemotherapeutic agent or an antibody. In some embodiments, the other therapeutic agent is selected from the group consisting of an alkylating agent, an anthracycline antibiotic, a DNA crosslinking agent, an antimetabolite, an indolequinone, a taxane, or a platinum-based agent.

An “aberration” at a gene refers to a genetic aberration of a gene, an aberrant expression level and/or an aberrant activity level of the gene that may lead to abnormal function of the protein encoded by the gene. An aberration at a gene comprises a mutation of the gene which includes, but not limited to, deletion, frameshift, insertion, indel, missense mutation, nonsense mutation, point mutation, silent mutation, splice site mutation, splice variant, and translocation. In some embodiments, the mutation may be a loss or deletion of the gene.

“MTOR-activating aberration” refers to a genetic aberration, an aberrant expression level and/or an aberrant activity level of one or more mTOR-associated gene that may lead to hyperactivation of the mTOR signaling pathway. “Hyperactivate” refers to increase of an activity level of a molecule (such as a protein or protein complex) or a signaling pathway (such as the mTOR a signaling pathway) to a level that is above a reference activity level or range, such as at least about any of 10%, 20%, 30%, 40%, 60%, 70%, 80%, 90%, 100%, 200%, 500% or more above the reference activity level or the median of the reference activity range. In some embodiments, the reference activity level is a clinically accepted normal activity level in a standardized test, or an activity level in a healthy individual (or tissue or cell isolated from the individual) free of the mTOR-activating aberration.

The mTOR-activating aberration contemplated herein may include one type of aberration at one mTOR-associated gene, more than one type (such as at least about any of 2, 3, 4, 5, 6, or more) of aberrations in one mTOR-associated gene, one type of aberration at more than one (such as at least about any of 2, 3, 4, 5, 6, or more) mTOR-associated genes, or more than one type (such as at least about any of 2, 3, 4, 5, 6, or more) of aberration at more than one (such as at least about any of 2, 3, 4, 5, 6, or more) mTOR-associated genes. Different types of mTOR-activating aberration may include, but are not limited to, genetic aberrations, aberrant expression levels (e.g. overexpression or under-expression), aberrant activity levels (e.g. high or low activity levels), and aberrant protein phosphorylation levels. In some embodiments, a genetic aberration comprises a change to the nucleic acid (such as DNA or RNA) or protein sequence (i.e. mutation) or an aberrant epigenetic feature associated with an mTOR-associated gene, including, but not limited to, coding, non-coding, regulatory, enhancer, silencer, promoter, intron, exon, and untranslated regions of the mTOR-associated gene. In some embodiments, the mTOR-activating aberration comprises a mutation of an mTOR-associated gene, including, but not limited to, deletion, frameshift, insertion, indel, missense mutation, nonsense mutation, point mutation, silent mutation, splice site mutation, splice variant, and translocation. In some embodiments, the mutation may be a loss of function mutation for a negative regulator of the mTOR signaling pathway or again of function mutation of a positive regulator of the mTOR signaling pathway. In some embodiments, the genetic aberration comprises a copy number variation of an mTOR-associated gene. In some embodiments, the copy number variation of the mTOR-associated gene is caused by structural rearrangement of the genome, including deletions, duplications, inversion, and translocations. In some embodiments, the genetic aberration comprises an aberrant epigenetic feature of an mTOR-associated gene, including, but not limited to, DNA methylation, hydroxymethylation, increased or decreased histone binding, chromatin remodeling, and the like.

The mTOR-activating aberration is determined in comparison to a control or reference, such as a reference sequence (such as a nucleic acid sequence or a protein sequence), a control expression (such as RNA or protein expression) level, a control activity (such as activation or inhibition of downstream targets) level, or a control protein phosphorylation level. The aberrant expression level or the aberrant activity level in an mTOR-associated gene may be above the control level (such as about any of 10%, 20%, 30%, 40%, 60%, 70%, 80%, 90%, 100%, 200%, 500% or more above the control level) if the mTOR-associated gene is a positive regulator (i.e. activator) of the mTOR signaling pathway, or below the control level (such as about any of 10%, 20%, 30%, 40%, 60%, 70%, 80%, 90% or more below the control level) if the mTOR-associated gene is a negative regulator (i.e. inhibitor) of the mTOR signaling pathway. In some embodiments, the control level (e.g. expression level or activity level) is the median level (e.g. expression level or activity level) of a control population. In some embodiments, the control population is a population having the same cancer as the individual being treated. In some embodiments, the control population is a healthy population that does not have the cancer, and optionally with comparable demographic characteristics (e.g. gender, age, ethnicity, etc.) as the individual being treated. In some embodiments, the control level (e.g. expression level or activity level) is a level (e.g. expression level or activity level) of a healthy tissue from the same individual. A genetic aberration may be determined by comparing to a reference sequence, including epigenetic patterns of the reference sequence in a control sample. In some embodiments, the reference sequence is the sequence (DNA, RNA or protein sequence) corresponding to a fully functional allele of an mTOR-associated gene, such as an allele (e.g. the prevalent allele) of the mTOR-associated gene present in a healthy population of individuals that do not have the cancer, but may optionally have similar demographic characteristics (such as gender, age, ethnicity etc.) as the individual being treated. Exemplary mTOR-associated genes and their reference sequences (i.e. wildtype sequences) are described in the section for the individual genes (such as TSC1, TSC2, RPS6, PTEN, TP53, ATRX, and FAT1).

The “status” of an mTOR-activating aberration may refer to the presence or absence of the mTOR-activating aberration at one or more mTOR-associated genes, or the aberrant level (expression or activity level, including phosphorylation level of a protein) of one or more mTOR-associated genes. In some embodiments, the presence of a genetic aberration (such as a mutation or a copy number variation) in one or more mTOR-associated genes as compared to a control indicates that (a) the individual is more likely to respond to treatment or (b) the individual is selected for treatment. In some embodiments, the absence of a genetic aberration at an mTOR-associated gene, or a wild-type mTOR-associated gene compared to a control, indicates that (a) the individual is less likely to respond to treatment or (b) the individual is not selected for treatment. In some embodiments, an aberrant level (such as expression level or activity level, including phosphorylation level of a protein) of one or more mTOR-associated genes is correlated with the likelihood of the individual to respond to treatment. For example, a larger deviation of the level (e.g. expression or activity level, including phosphorylation level of a protein) of one or more mTOR-associated genes in the direction of hyperactivating the mTOR signaling pathway indicates that the individual is more likely to respond to treatment. In some embodiments, a prediction model based on the level(s) (e.g. expression level or activity level, including phosphorylation level of a protein) of one or more mTOR-associated genes is used to predict (a) the likelihood of the individual to respond to treatment and (b) whether to select the individual for treatment. The prediction model, including, for example, coefficient for each level, may be obtained by statistical analysis, such as regression analysis, using clinical trial data.

The expression level, and/or activity level of the one or more mTOR-associated genes, and/or phosphorylation level of one or more proteins encoded by the one or more mTOR-associated genes, and/or the presence or absence of one or more genetic aberrations of the one or more mTOR-associated genes can be useful for determining any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits.

In some embodiments, the mutational status, expression level, or activity level of one or more resistance biomarker (such as TFE3) is further used for selecting an individual for any of the methods of treatment described herein, and/or for determining any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits. In some embodiments, the resistance biomarker is a gene selected from the ONCOPANEL™ test. See, for example, Wagle N. et al. Cancer discovery 2.1 (2012): 82-93.

In some embodiments according to any one of the methods of treatment described herein, the mutational status of TFE3 in an individual is used as a basis for selecting the individual. In some embodiments, the mutational status of TFE3 is used in combination with one or more mTOR-activating aberration at an individual as a basis for selecting the individual for the treatment. In some embodiments, the mutational status of TFE3 comprises translocation of TFE3. In some embodiments, translocation of TFE3 is used to exclude an individual from the treatment. In some embodiments, translocation of TFE3 in a sample of the individual is assessed by fluorescence in situ hybridization (FISH). In some embodiments, the sample is a blood sample. In some embodiments, the sample is a tumor biopsy. In some embodiments, the sample is obtained prior to initiation of the treatment methods described herein. In some embodiments, the sample is obtained after initiation of the treatment methods described herein.

As used herein, “based upon” includes assessing, determining, or measuring the individual's characteristics as described herein (and preferably selecting an individual suitable for receiving treatment). When the status of an mTOR-activating aberration is “used as a basis” for selection, assessing, measuring, or determining method of treatment as described herein, the mTOR-activating aberration at one or more mTOR-associated genes is determined before and/or during treatment, and the status (including presence, absence, expression level, activity level and/or phosphorylation level of the mTOR-activating aberration) obtained is used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; or (g) predicting likelihood of clinical benefits.

### Pathogenic/Inactivating Mutations

In some embodiments, the individual has a pathogenic (i.e., inactivating) mutation in any of the genes described herein. Pathogenic inactivating mutations (loss-of-function) of certain gene (e.g., TSC1 or TSC2) can be determined by review of experimental evidence within the published scientific literature and review of critical regions that may be disrupted, including but not limited to frameshift, missense mutations, truncating mutations, deletions, copy number variations, nonsense mutations, and loss or deletion of the gene. A pathogenic mutation is inferred as inactivating.

Pathogenic or inactivating mutation includes but not limited to homozygous deletions, bi-allelic (double hit) mutations, splice site mutations (e.g., a 2^{nd} or an additional splice site mutation), frameshift mutations, and nonsense mutations in coding region, missense mutations with confirmed impact.

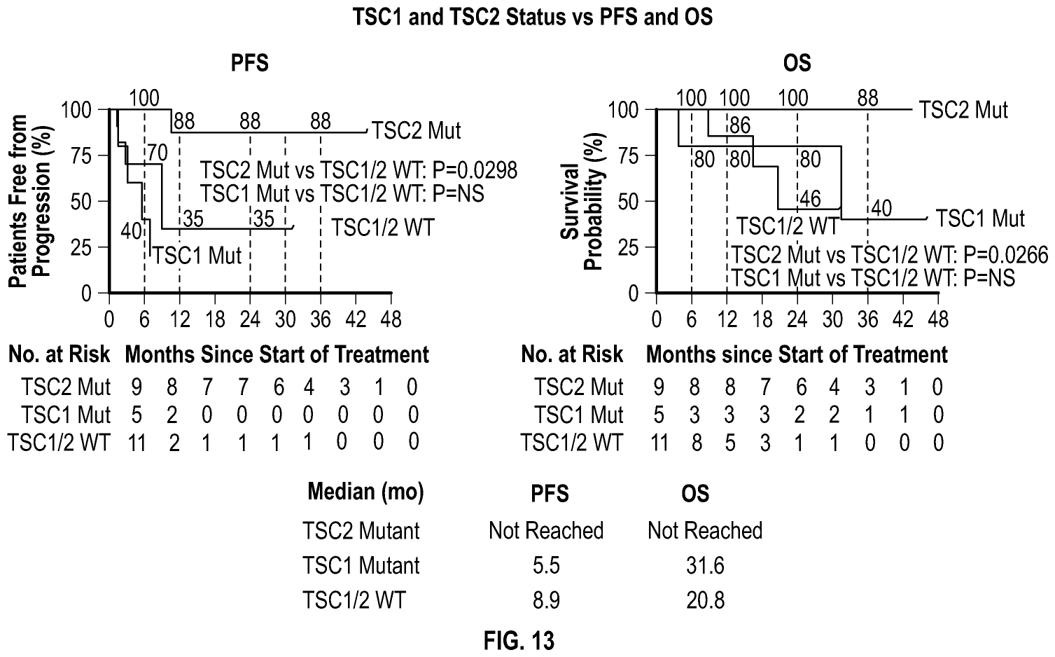

In some embodiments, the methods described herein comprises a step of determining if a mutation in TSC1 or TSC2 is a pathogenic mutation. In some embodiments, whether a mutation in TSC1 or TSC2 is determined according to the table in FIGS. 13A-13B or as described below.

In some embodiments, the inactivating mutation comprises a nonsense mutation, an out-of-frame insertion, a deletion mutation, or a mutation that affects canonical splice site in TSC1 or TSC2. In some embodiments, the allele frequency of mutated TSC1 or TSC2 is similar to or higher than a reference cancer gene in the tumor sample. In some embodiments, there is a second hit or loss of the other allele of mutated TSC1. In some embodiments, there is a mutation occurring in the last nucleotide position of an exon (i.e., 3′ end of an exon, e.g., a G).

In some embodiments, the inactivating mutation comprises an in-frame deletion mutation in TSC1 or TSC2. In some embodiments, the in-frame deletion mutation has been reported in the LOVD database (e.g., https://database.lovd.nl/shared/genes/TSC2). In some embodiments, the in-frame deletion mutation in TSC1 or TSC2 deletes a size of more than one amino acids.

In some embodiments, the inactivating mutation comprises a missense mutation in TSC1. In some embodiments, the missense mutation in TSC1 comprises a non-conservative substitution within amino acids 34-224 or exons 4-8 of TSC1.

In some embodiments, the inactivating mutation comprises a missense mutation in TSC2. In some embodiments, the missense mutation in TSC2 comprises a non-conservative substitution and/or has been reported in the LOVD database (https://databases.lovd.nl/shared/genes/TSC2).

In some embodiments, the inactivating mutation comprises a homozygous deletion mutation. In some embodiments, the homozygous deletion mutation affects one or more exons of TSC1 or TSC2.

### Methods of Assessing if a Mutation in TSC1 or TSC2 is Pathogenic

In some embodiments, there is provided a method of assessing if a mutation in TSC1 or TSC2 is pathogenic, comprising determining if the mutation is
i) a nonsense mutation, an out-of-frame insertion, a deletion mutation, or a mutation that affects canonical splice site in TSC1 or TSC2,
ii) an in-frame deletion mutation in TSC1 or TSC2,
iii) a missense mutation in TSC1 or TSC2, or
iv) a homozygous deletion in TSC1 or TSC2.

In some embodiments, the mutation is a nonsense mutation, an out-of-frame insertion, a deletion mutation, or a mutation that affects canonical splice site in TSC1 or TSC2, and the method further comprises determining if:

a) the allele frequency of mutated TSC1 or TSC2 is similar to or higher than a reference cancer gene in the tumor sample,

b) there is a second hit or loss of the other allele of mutated TSC1, or

c) there is a mutation occurring in the last nucleotide position of an exon (i.e., 3′ end of an exon, e.g., a G);

wherein the method further comprises determining that the mutation is a pathogenic mutation if the answer to any of a)-c) above is yes.

In some embodiments, the mutation is a nonsense mutation, an out-of-frame insertion, a deletion mutation, or a mutation that affects canonical splice site in TSC1 or TSC2, and the method further comprises determining if:

a) the allele frequency of mutated TSC1 or TSC2 is significantly lower (e.g., at least about 10%, 20%, 30%, 40%, 50% lower) than a reference cancer gene examined in the tumor sample,

b) the mutation is in 3′ half of exon 22 and all of exon 23 of TSC1

c) the mutation affects i) amino acids 947-989 of exon 26 of TSC2 or ii) amino acids 1272-1295 of exon 32 of TSC2, or

d) the individual has a tumor mutation burden of more than 10/Mb;

wherein the method further comprises determining that the mutation is not pathogenic if the answer is yes to any of a)-d) above is yes.

In some embodiments, the mutation is an in-frame deletion mutation in TSC1 or TSC2, and the method further comprises determining if: a) the deletion mutation is previously seen and/or reported in LOVD database (e.g., https://databases.lovd.nl/shared/genes/TSC2); or b) the if the deletion mutation comprises a deletion of size more than one amino acid; wherein the method further comprises determining that the mutation is pathogenic if the answer is yes to a) or b).

In some embodiments, the mutation is an in-frame deletion mutation in TSC1 or TSC2, and the method further comprises determining if a) the deletion mutation affects a single amino acid and b) the deletion mutation has not been reported in LOVD database (e.g., https://databases.lovd.nl/shared/genes/TSC2); and the method further comprises determining that the mutation is not pathogenic if the answer is yes to both a) and b).

In some embodiments, the mutation is a missense mutation in TSC1, and the method further comprises determining if a) the missense mutation comprises a mutation in amino acids 34-224 of exons 4-8 of TSC1 and the mutation is non-conservative substitute; and/or b) the missense mutation comprises a mutation in amino acids 34-224 of exons 4-8 of TSC1 and the mutation is a conservative substitute (e.g., L->V), wherein the method further comprises determining that 1) the mutation is pathogenic if answer is yes to a), or 2) the mutation is not pathogenic if the answer is yes to b).

In some embodiments, the mutation is a missense mutation in TSC2, and the method further comprises determining if a) the missense mutation is a non-conservative substitution and/or is confirmed in LOVD database, b) the missense mutation is a conservative substitution; wherein optionally the method further comprises determining that 1) the mutation is pathogenic if answer is yes to a), or 2) the mutation is not pathogenic if the answer is yes to b).

In some embodiments, the mutation is a homozygous deletion in TSC1 or TSC2, wherein the method further comprises determining if the homozygous deletion affects one or more than one exons, wherein optionally the method further comprises determining that the mutation is pathogenic if answer is yes to the above question.

### TSC2

TSC2 is also known as Tuberin, Tuberous sclerosis 2 protein, protein phosphatase 1 regulatory subunit 160, TSC4, PPP1R160, and LAM. TSC2 protein functions as part of a complex with TSC1 by negatively regulating mTORC1 signaling. In some embodiments, the nucleic acid sequence of a wildtype TSC2 gene is identified by the Genbank accession number NC_000016.10, from nucleotide 2047936 to nucleotide 2088712 on the forward strand of chromosome 16 according to the GRCh38.p2 assembly of the human genome. The wildtype TSC2 gene comprises 42 exons. A mutation of the TSC2 gene may occur in any one or any combination of the 42 exons, or in any intron or noncoding regions of the TSC2 gene.

In some embodiments, the amino acid sequence of a wildtype TSC2 protein is identified by the Genbank accession number NP_000539.2. In some embodiments, the amino acid sequence of a wildtype TSC2 protein is identified by the Genbank accession number NP_001070651.1. In some embodiments, the amino acid sequence of a wildtype TSC2 protein is identified by the Genbank accession number NP_001107854.1.

In some embodiments, the nucleic acid sequence of a cDNA encoding a wildtype TSC2 protein is identified by the Genbank accession number NM_000548.3. In some embodiments, the nucleic acid sequence of a cDNA encoding a wildtype TSC2 protein is identified by the Genbank accession number NM_001077183.1. In some embodiments, the nucleic acid sequence of a cDNA encoding a wildtype TSC2 protein is identified by the Genbank accession number NM_001114382.1.

In some embodiments, the individual is selected for treatment based on having an mTOR-activating aberration at TSC2. In some embodiments, the mTOR-activating aberration at TSC2 comprises a mutation (e.g., inactivating mutation) in TSC2. In some embodiments, the mutation is selected from the group consisting of a splice site mutation, a nonsense mutation, a frameshift mutation, a missense mutation, and a loss or deletion of the gene. In some embodiments, the mTOR-activating aberration at TSC2 comprises a single-nucleotide variant (SNV). In some embodiments, the SNV comprises a mutation selected from the group consisting of C1503T, C2743G, C5383T, C3755G, G760T, C3442T, G880A. T707C, A4949G, or a deletion of any one or more of the amino acids at the position of 1405-1409, 1960-1970, 4999, 5002, 3521, 5208, 5238-5255.

In some embodiments, the mutation is a two-point mutation (i.e., bi-allelic mutations). In some embodiments, the mutation comprises three-point mutation or four-point mutation. In some embodiments, the mTOR-activating aberration at TSC2 is a loss of function mutation. In some embodiments, the mTOR-activating aberration at TSC2 comprises a homozygous deletion. In some embodiments, the mTOR-activating aberration at TSC2 comprises a copy number variation of TSC2. In some embodiments, the mTOR-activating aberration at TSC2 comprises an aberrant expression level of TSC2. In some embodiments, the mTOR-activating aberration at TSC2 comprises an aberrant activity level of a protein encoded by TSC2.

In some embodiments, the individual has a mutation (e.g., inactivating mutation) in any one or more of exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, and 44 according to Genbank accession number NM_000548. In some embodiments, the individual has bi-allelic mutations (e.g., bi-allelic inactivating mutation) in two of exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, and 44 according to Genbank accession number NM_000548. In some embodiments, the individual has an inactivating mutation in any of exons 18, 22, 27, 30, and 42 of TSC2. In some embodiments, the individual has bi-allelic mutations in any two of exons 18, 22, 27, 30, and 42 of TSC2. In some embodiments, the individual has bi-allelic mutations in exons 18 and 30 of TSC2. In some embodiments, the individual has bi-allelic mutations in exons 22 and 27 of TSC2.

In some embodiments, the mutation is not within amino acids 947-989 or exon 26. In some embodiments, the mutation is not within amino acids 1272-1295 or exon 32.

In some embodiments, the mutation comprises a non-conservative substitution.

In some embodiments, the mutation has been reported by the LOVD database (https://databases.lovd.nl/shared/genes/TSC2)

TSC1 and TSC2 gene mutations were described in e.g., Rosset et al., Genetics and Molecular Biology, 40, 1, 69-79 (2017), which is incorporated herein by its entirety. In some embodiments, the individual has a continuous deletion (e.g., TSC2-PKD1 deletion). See e.g., Boronat el al., Brain Dev. 36:801-806. In some embodiments, the individual has a c.5238-5255 del in TSC2. See e.g., Rok et al. Med Sci Monit 11:230-234. In some embodiments, the individual has a proximal region mutation (e.g., in any of exons 1-22) and/or a distal region mutation (e.g., in any of exons 23-41). See e.g., van Eeghena et al. Epilepsy Res 103:83-87.

### TSC1

TSC1 is also known as Hamartin, Tuberous sclerosis 1 protein, TSC, KIAA0243, and LAM. TSC1 protein functions as part of a complex with TSC2 by negatively regulating mTORC1 signaling. In some embodiments, the nucleic acid sequence of a wildtype TSC1 gene is identified by the Genbank accession number NC_000009.12, from nucleotide 132891348 to nucleotide 132945370 on the reverse strand of chromosome 9 according to the GRCh38.p2 assembly of the human genome. The wildtype TSC1 gene comprises 25 exons. A mutation of the TSC1 gene may occur in any one or any combination of the 25 exons, or in any intron or noncoding regions of the TSC1 gene.

In some embodiments, the amino acid sequence of a wildtype TSC1 protein is identified by the Genbank accession number NP_000359.1. In some embodiments, the amino acid sequence of a wildtype TSC1 protein is identified by the Genbank accession number NP_001155898.1. In some embodiments, the amino acid sequence of a wildtype TSC1 protein is identified by the Genbank accession number NP_001155899.1.

In some embodiments, the nucleic acid sequence of a cDNA encoding a wildtype TSC1 protein is identified by the Genbank accession number NM_000368.4. In some embodiments, the nucleic acid sequence of a cDNA encoding a wildtype TSC1 protein is identified by the Genbank accession number NM_001162426.1. In some embodiments, the nucleic acid sequence of a cDNA encoding a wildtype TSC1 protein is identified by the Genbank accession number NM_001162427.1.

In some embodiments, the individual is selected for treatment on the basis of having an mTOR-activating aberration at TSC1. In some embodiments, the mTOR-activating aberration at TSC1 comprises a mutation (e.g., an inactivating mutation) in TSC1. In some embodiments, the mutation is selected from the group consisting of a splice site mutation, a nonsense mutation, a frameshift mutation, a missense mutation and a loss or deletion of the gene. In some embodiments, the mTOR-activating aberration at TSC1 comprises a single-nucleotide variant (SNV). In some embodiments, the mutation is a two-point mutation. In some embodiments, the mTOR-activating aberration at TSC1 is a loss of function mutation.

In some embodiments, the mTOR-activating aberration at TSC1 comprises a homozygous deletion. In some embodiments, the mTOR-activating aberration at TSC1 comprises a copy number variation of TSC1. In some embodiments, the mTOR-activating aberration at TSC1 comprises an aberrant expression level of TSC1. In some embodiments, the mTOR-activating aberration at TSC1 comprises an aberrant activity level of a protein encoded by TSC1.

In some embodiments, the individual has a mutation (e.g., inactivating mutation) in any one or more of exon 1, 2, 3, 4, 5, 6, 7, 8, 9, 10, 11, 12, 13, 14, 15, 16, 17, 18, 19, 20, 21, 22, 23, 24, and 25 according to Genbank accession number NM_000368. In some embodiments, the individual has bi-allelic mutations (e.g., bi-allelic inactivating mutation) in two of exon 1, 2, 3, 4, 5, 6, 7, 8, 9, 10, 11, 12, 13, 14, 15, 16, 17, 18, 19, 20, 21, 22, 23, 24, and 25 according to Genbank accession number NM_000368. In some embodiments, the mutation is not in exon 23. In some embodiments, the mutation is not in 3′ half of exon 22.

In some embodiments, the mutation comprises a non-conservative substitution.

In some embodiments, the mutation has been reported by the LOVD database (https://databases.lovd.nl/shared/genes/TSC1)

In some embodiments, the individual has a TSC1 loss or deletion.

### RPS6

Ribosomal protein S6 (RPS6) is also known as S6. Ribosomes, the organelles that catalyze protein synthesis, consist of a small 40S subunit and a large 60S subunit. Together these subunits are composed of 4 RNA species and approximately 80 structurally distinct proteins. This gene encodes a cytoplasmic ribosomal protein that is a component of the 40S subunit. The protein belongs to the S6E family of ribosomal proteins. It is the major substrate of protein kinases in the ribosome, with subsets of five C-terminal serine residues phosphorylated by different protein kinases. Phosphorylation is induced by a wide range of stimuli, including growth factors, tumor-promoting agents, and mitogens. Dephosphorylation occurs at growth arrest. The protein may contribute to the control of cell growth and proliferation through the selective translation of particular classes of mRNA. As is typical for genes encoding ribosomal proteins, there are multiple processed pseudogenes of this gene dispersed through the genome.

In some embodiments, the nucleic acid sequence of a wildtype RPS6 gene is identified by the Genbank accession number NC_000009.12, from nucleotide 19375715 to nucleotide 19380236 on the forward strand of chromosome 9 according to the GRCh38.p13 assembly of the human genome. The wildtype RPS6 gene comprises 6 exons. A mutation of the RPS6 gene may occur in any one or any combination of the 6 exons, or in any intron or noncoding regions of the RPS6 gene.

In some embodiments, the amino acid sequence of a wildtype RPS6 protein is identified by the Genbank accession number NM_001010.3.

In some embodiments, the individual is selected for treatment on the basis of having an mTOR-activating aberration at RPS6. In some embodiments, the mTOR-activating aberration at RPS6 comprises an aberrant phosphorylation level of the protein encoded by RPS6 (e.g., phosphorylation at residue S235, S236, S240, and/or S244). In some embodiments, the aberrant phosphorylation level of the protein encoded by RPS6 is a positive status of phosphorylated S6 (pS6). In some embodiments, the aberrant phosphorylation level of the protein encoded by RPS6 is an increased phosphorylation of S6 in the cancer as compared to a reference tissue. In some embodiments, the reference tissue is derived from a non-cancerous tissue in the individual. In some embodiments, the reference tissue is derived from a corresponding tissue in another individual that does not have the cancer. The status of, phosphorylated S6 can be assessed via IHC staining with an antibody that binds to phosphorylated residue(s) in S6 (e.g., an antibody that detects endogenous levels of ribosomal protein S6 only when phosphorylated at Ser235 and 236). In some embodiments, the expression level of RPS6 is assessed by immunohistochemistry. In some embodiments, the mTOR-activating aberration at RPS6 comprises an aberrant expression level of RPS6.

### TP53

Tumor protein 53 (TP53), also known as tumor protein p53, P53, BCC7, LFS1 or TRP53, is a tumor suppressor protein that responds to diverse cellular stresses to regulate expression of target genes, thereby inducing cell cycle arrest, apoptosis, senescence, DNA repair, or changes in metabolism. TP53 crosstalks with the mTOR signaling pathway by inhibiting mTOR activity. In some embodiments, the nucleic acid sequence of a wildtype TP53 gene is identified by the Genbank accession number NC_000017.11 from nucleotide 7668402 to nucleotide 7687550 of the complement strand of chromosome 17 according to the GRCh38.p2 assembly of the human genome. The wildtype TP53 gene comprises 12 exons. A mutation of the TP53 gene may occur in any one or any combination of the 12 exons, or in any intron or noncoding regions of the TP33 gene. The wildtype protein encoded by TP53 includes multiple isoforms, such as isoforms a-l. A mutation may affect any of the of TP53 isoforms. In some embodiments, the amino acid sequence of a wildtype TP53 protein is identified by the Genbank accession number NP_000537.3. In some embodiments, the nucleic acid sequence of a cDNA encoding a wildtype TP53 protein is identified by the Genbank accession number NM_000546.5.

In some embodiments, the individual is selected for treatment based on having an mTOR-activating aberration at TP53. In some embodiments, the mTOR-activating aberration at TP53 comprises a mutation in TP53. In some embodiments, the mutation is selected from the group consisting of a splice site mutation, a nonsense mutation, a frameshift mutation, a missense mutation and a loss or deletion of the gene. In some embodiments, the mTOR-activating aberration at TP53 comprises a single-nucleotide variant (SNV). In some embodiments, the mutation is a two-point mutation. In some embodiments, the mTOR-activating aberration at TP53 is a loss of function mutation. In some embodiments, the mTOR-activating aberration at TP53 comprises a homozygous deletion. In some embodiments, the mTOR-activating aberration at TP53 comprises a copy number variation of TP53. In some embodiments, the mTOR-activating aberration at TP53 comprises an aberrant expression level of TP53. In some embodiments, the mTOR-activating aberration at TP53 comprises an aberrant activity level of a protein encoded by TP53.

### ATRX

ATRX chromatin remodeler (ATRX), also known as JMS, XH2, XNP, MRX52, RAD54, RAD54L, or ZNF-HX. The protein encoded by this gene contains an ATPase/helicase domain, and thus it belongs to the SWI/SNF family of chromatin remodeling proteins. This protein is found to undergo cell cycle-dependent phosphorylation, which regulates its nuclear matrix and chromatin association, and suggests its involvement in the gene regulation at interphase and chromosomal segregation in mitosis. Mutations in this gene are associated with X-linked syndromes exhibiting cognitive disabilities as well as alpha-thalassemia (ATRA) syndrome. These mutations have been shown to cause diverse changes in the pattern of DNA methylation, which may provide a link between chromatin remodeling, DNA methylation, and gene expression in developmental processes. Multiple alternatively spliced transcript variants encoding distinct isoforms have been reported.

In some embodiments, the nucleic acid sequence of a wildtype ATRX gene is identified by the Genbank accession number NC 000023.11, from nucleotide 77504878 to nucleotide 77786235 on the forward strand of chromosome X according to the GRCh38.p13 assembly of the human genome. The wildtype ATRX gene comprises 38 exons. A mutation of the ATRX gene may occur in any one or any combination of the 38 exons, or in any intron or noncoding regions of the ATRX gene.

In some embodiments, the amino acid sequence of a wildtype ATRX protein is identified by the Genbank accession number of NM_000489.5. In some embodiments, the amino acid sequence of a wildtype ATRX protein is identified by the Genbank accession number of NM_138270.4. In some embodiments, the amino acid sequence of a wildtype ATRX protein is identified by the Genbank accession number selected from the group consisting of NM_000489.5, NM_138270.4, XM_017029611.1, XM_006724667.3, XM_017029603.1, XM_005262156.4, XM_017029610.1, XM_017029609.1, XM_017029605.1, XM_005262155.4, XM_005262157.5, XM_006724666.4, XM_017029604.2, XM_017029601.2. XM 005262154.5, XM_017029606.2, XM_005262153.5, XM_017029607.2, XM 017029602.1, XM_017029608.2, and XM_006724668.3.

In some embodiments, the individual is selected for treatment on the basis of having an mTOR-activating aberration at ATRX In some embodiments, the mTOR-activating aberration at ATRX comprises a mutation in ATRX. In some embodiments, the mutation is selected from the group consisting of a splice site mutation, a nonsense mutation, a frameshift mutation, a missense mutation and a loss or deletion of the gene. In some embodiments, the mTOR-activating aberration at ATRX comprises a single-nucleotide variant (SNV). In some embodiments, the mutation is a two-point mutation. In some embodiments, the mTOR-activating aberration at ATRX is a loss of function mutation. In some embodiments, the mTOR-activating aberration at ATRX comprises a homozygous deletion. In some embodiments, the mTOR-activating aberration at ATRX comprises a copy number variation of ATRX. In some embodiments, the mTOR-activating aberration at ATRX comprises an aberrant expression level of ATRX In some embodiments, the mTOR-activating aberration at ATRX comprises an aberrant activity level of a protein encoded by ATRX

### PTEN

Phosphatase and tensin homolog (PTEN) is also known as the phosphatidylinositol 3,4,5-triphosphate 3-phosphatase and dual-specificity phosphatase PTEN, mutated in multiple advanced cancers 1, phosphatase and tensin homolog, MMAC1, TEP1, BZS, DEC, CWS1, GLM2, MHAM, and PTEN1. In some embodiments, the nucleic acid sequence of a wildtype PTEN gene is identified by the Genbank accession number NC_000010.11 from nucleotide 87,863,625 to nucleotide 87971930 of the forward strand of chromosome 10 according to the GRCh38.p2 assembly of the human genome. The wildtype PTEN gene comprises 16 exons. A mutation of the PTEN gene may occur in any one or any combination of the 16 exons, or in any intron or noncoding regions of the PTEN gene.

In some embodiments, the amino acid sequence of a wildtype PTEN protein is identified by the Genbank accession number NP_000305.3. In some embodiments, the amino acid sequence of a wildtype PTEN protein is identified by the Genbank accession number NP_001291646.2. In some embodiments, the amino acid sequence of a wildtype PTEN protein is identified by the Genbank accession number NP_001291647.1. The wildtype PTEN protein comprises a phosphatase tensin-type domain, and a C2 tensin-type domain. A mutation in the PTEN protein may occur in either one or both protein domains.

In some embodiments, the nucleic acid sequence of a cDNA encoding a wildtype PTEN protein is identified by the Genbank accession number NM_000314.6. In some embodiments, the nucleic acid sequence of a cDNA encoding a wildtype PTEN protein is identified by the Genbank accession number NM_001304717.2. In some embodiments, the nucleic acid sequence of a cDNA encoding a wildtype PTEN protein is identified by the Genbank accession number NM_001304718.1.

In some embodiments, the individual is selected for treatment based on having an mTOR-activating aberration at PTEN. In some embodiments, the mTOR-activating aberration at PTEN comprises a mutation in PTEN. In some embodiments, the mutation is selected from the group consisting of a splice site mutation, a nonsense mutation, a frameshift mutation, a missense mutation and a loss or deletion of the gene. In some embodiments, the mTOR-activating aberration at PTEN comprises a single-nucleotide variant (SNV). In some embodiments, the mutation is a two-point mutation. In some embodiments, the mTOR-activating aberration at PTEN is a loss of function mutation. In some embodiments, the mTOR-activating aberration at PTEN comprises a homozygous deletion. In some embodiments, the mTOR-activating aberration at PTEN comprises a copy number variation of PTEN. In some embodiments, the mTOR-activating aberration at PTEN comprises an aberrant expression level of PTEN. In some embodiments, the mTOR-activating aberration at PTEN comprises an aberrant activity level of a protein encoded by PTEN.

### RB1

RB transcriptional corepressor 1 (RB1), also known as RB, pRb, OSRC, pp110, p105-Rb, or PPP1R130. The protein encoded by this gene is a negative regulator of the cell cycle and was the first tumor suppressor gene found. The encoded protein also stabilizes constitutive heterochromatin to maintain the overall chromatin structure. The active, hypophosphorylated form of the protein binds transcription factor E2F1. Defects in this gene are a cause of childhood cancer retinoblastoma (RB), bladder cancer, and osteogenic sarcoma.

In some embodiments, the nucleic acid sequence of a wildtype RB1 gene is identified by the Genbank accession number NC_000013.11, from nucleotide 48303747 to nucleotide 48481890 on the forward strand of chromosome 13 according to the GRCh38.p13 assembly of the human genome. The wildtype RB1 gene comprises 28 exons. A mutation of the RB1 gene may occur in any one or any combination of the 28 exons, or in any intron or noncoding regions of the RB1 gene.

In some embodiments, the amino acid sequence of a wildtype RB1 protein is identified by the Genbank accession number of NM_000321.2. In some embodiments, the amino acid sequence of a wildtype RB1 protein is identified by the Genbank accession number of XM_011535171.2.

In some embodiments, the individual is selected for treatment on the basis of having an mTOR-activating aberration at RB1. In some embodiments, the mTOR-activating aberration at RB1 comprises a mutation in RB1. In some embodiments, the mutation is selected from the group consisting of a splice site mutation, a nonsense mutation, a frameshift mutation, a missense mutation and a loss or deletion of the gene. In some embodiments, the mTOR-activating aberration at RB1 comprises a single-nucleotide variant (SNV). In some embodiments, the mutation is a two-point mutation. In some embodiments, the mTOR-activating aberration at RB) is a loss of function mutation. In some embodiments, the mTOR-activating aberration at RB1 comprises a homozygous deletion. In some embodiments, the mTOR-activating aberration at RB1 comprises a copy number variation of RB). In some embodiments, the mTOR-activating aberration at RB) comprises an aberrant expression level of RB1. In some embodiments, the mTOR-activating aberration at RB1 comprises an aberrant activity level of a protein encoded by RB1.

### FAT1

FAT1 atypical cadherin 1 (FAT1), was also known as AT, ME5, CDHF7, CDHR8, or hFAT1. This gene is an ortholog of the Drosophila fat gene, which encodes a tumor suppressor essential for controlling cell proliferation during Drosophila development. The gene product is a member of the cadherin superfamily, a group of integral membrane proteins characterized by the presence of cadherin-type repeats. In addition to containing 34 tandem cadherin-type repeats, the gene product has five epidermal growth factor (EGF)-like repeats and one laminin A-G domain. This gene is expressed at high levels in a number of fetal epithelia. Its product probably functions as an adhesion molecule and/or signaling receptor, and is likely to be important in developmental processes and cell communication. Transcript variants derived from alternative splicing and/or alternative promoter usage exist, but they have not been fully described.

In some embodiments, the nucleic acid sequence of a wildtype FAT1 gene is identified by the Genbank accession number NC_000004.12, from nucleotide 186587789 to nucleotide 186726696 on the forward strand of chromosome 4 according to the GRCh38.p13 assembly of the human genome. The wildtype FAT1 gene comprises 29 exons. A mutation of the FAT1 gene may occur in any one or any combination of the 29 exons, or in any intron or noncoding regions of the FAT1 gene.

In some embodiments, the amino acid sequence of a wildtype FAT1 protein is identified by the Genbank accession number of XM_006714139.3. In some embodiments, the amino acid sequence of a wildtype FAT1 protein is identified by the Genbank accession number of XM_005262834.3. In some embodiments, the amino acid sequence of a wildtype FAT1/protein is identified by the Genbank accession number of XM_005262835.2.

In some embodiments, the individual is selected for treatment on the basis of having an mTOR-activating aberration at FAT1. In some embodiments, the mTOR-activating aberration at FAT1 comprises a mutation in FA T1. In some embodiments, the mutation is selected from the group consisting of a splice site mutation, a nonsense mutation, a frameshift mutation, a missense mutation and a loss or deletion of the gene. In some embodiments, the mTOR-activating aberration at FAT1 comprises a single-nucleotide variant (SNV). In some embodiments, the mutation is a two-point mutation. In some embodiments, the mTOR-activating aberration at FAT1 is a loss of function mutation. In some embodiments, the mTOR-activating aberration at FAT1 comprises a homozygous deletion. In some embodiments, the mTOR-activating aberration at FAT1 comprises a copy number variation of FAT1. In some embodiments, the mTOR-activating aberration at FAT1 comprises an aberrant expression level of FAT1. In some embodiments, the mTOR-activating aberration at FAT1 comprises an aberrant activity level of a protein encoded by FAT1.

### Nanoparticle Compositions

The mTOR inhibitor nanoparticle compositions described herein comprise nanoparticles comprising (in various embodiments consisting essentially of or consisting of) an mTOR inhibitor (such as a limus drug, e.g., rapamycin or a derivative thereof) and an albumin (such as human serum albumin). Nanoparticles of poorly water soluble drugs (such as macrolides) have been disclosed in, for example, U.S. Pat. Nos. 5,916,596; 6,506,405; 6,749,868, 6,537,579, 7,820,788, and 8,911,786, and also in U. S. Pat. Pub. Nos. 2006/0263434, and 2007/0082838; PCT Patent Application WO08/137148, U.S. Patent Application No. 62/927,047, each of which is incorporated herein by reference in their entirety.

In some embodiments, the composition comprises nanoparticles with an average or mean diameter of no greater than about 1000 nanometers (nm), such as no greater than about any of 900, 800, 700, 600, 500, 400, 300, 200, and 100 nm. In some embodiments, the average or mean diameters of the nanoparticles is no greater than about 200 nm. In some embodiments, the average or mean diameters of the nanoparticles is no greater than about 150 nm. In some embodiments, the average or mean diameters of the nanoparticles is no greater than about 100 nm. In some embodiments, the average or mean diameter of the nanoparticles is about 10 to about 400 nm. In some embodiments, the average or mean diameter of the nanoparticles is about 10 to about 150 nm. In some embodiments, the average or mean diameter of the nanoparticles is about 40 to about 120 nm. In some embodiments, the average or mean diameter of the nanoparticles are no less than about 50 nm. In some embodiments, the nanoparticles are sterile-filterable.

In some embodiments, the particles (such as nanoparticles) described herein have an average or mean diameter of no greater than about any of 1000, 900, 800, 700, 600, 500, 400, 300, 200, 150, 120, and 100 nm. In some embodiments, the average or mean diameter of the particles is no greater than about 200 nm. In some embodiments, the average or mean diameter of the particles is between about 20 nm to about 400 nm. In some embodiments, the average or mean diameter of the particles is between about 40 nm to about 200 nm. In some embodiments, the average or mean diameter of the nanoparticles is about 100-120 nm, for example about 100 nm. In some embodiments, the average mean diameter of the particles is less than or equal to 120 nm. In some embodiments, the average mean diameter of the particles is about 100-120 nm, for example about 100 nm. In some embodiments, the particles are sterile-filterable.

In some embodiments, the nanoparticles in the composition described herein have an average diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least about any one of 60%, 70%, 80%, 90%, 95%, or 99%) of the nanoparticles in the composition have a diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least any one of 60%, 70%, 80%, 90%, 95%, or 99%) of the nanoparticles in the composition fall within the range of about 10 nm to about 400 nm, including for example about 10 nm to about 200 nm, about 20 nm to about 200 nm, about 30 nm to about 180 nm, about 40 nm to about 150 nm, about 40 nm to about 120 nm, and about 60 nm to about 100 nm.

Methods of determining average particle sizes are known in the art, for example, dynamic light scattering (DLS) has been routinely used in determining the size of submicrometre-sized particles based. International Standard IS022412 Particle Size Analysis—Dynamic Light Scattering. International Organisation for Standardisation (ISO) 2008 and Dynamic Light Scattering Common Terms Defined, Malvem Instruments Limited, 2011. In some embodiments, the particle size is measured as the volume-weighted mean particle size (Dv50) of the nanoparticles in the composition.

In some embodiments, the nanoparticles comprise the mTOR inhibitor associated with the albumin. In some embodiments, the nanoparticles comprise the mTOR inhibitor coated with the albumin.

In some embodiments, the albumin has sulfhydryl groups that can form disulfide bonds. In some embodiments, at least about 5% (including for example at least about any one of 10%, 15%, 20%, 25%, 30%, 40%, 50%, 60%, 70%, 80%, or 90%) of the albumin in the nanoparticle portion of the composition are crosslinked (for example crosslinked through one or more disulfide bonds).

In some embodiments, the nanoparticles comprising the mTOR inhibitor (such as a limus drug, e.g., rapamycin or a derivative thereof) are associated (e.g., coated) with an albumin (such as human albumin or human serum albumin). In some embodiments, the composition comprises an mTOR inhibitor (such as a limus drug, e.g., rapamycin or a derivative thereof) in both nanoparticle and non-nanoparticle forms (e.g., in the form of solutions or in the form of soluble albumin/nanoparticle complexes), wherein at least about any one of 50%, 60%, 70%, 80%, 90%, 95%, or 99% of the mTOR inhibitor in the composition are in nanoparticle form. In some embodiments, the mTOR inhibitor (such as a limus drug, e.g., rapamycin or a derivative thereof) in the nanoparticles constitutes more than about any one of 50%, 60%, 70%, 80%, 90%, 95%, or 99% of the nanoparticles by weight. In some embodiments, the nanoparticles have a non-polymeric matrix. In some embodiments, the nanoparticles comprise a core of an mTOR inhibitor (such as a limus drug, e.g., rapamycin or a derivative thereof) that is substantially free of polymeric materials (such as polymeric matrix).

In some embodiments, the composition comprises an albumin in both nanoparticle and non-nanoparticle portions of the composition, wherein at least about any one of 50%, 60%, 70%, 80%, 90%, 95%, or 99% of the albumin in the composition are in non-nanoparticle portion of the composition.

In some embodiments, the weight ratio of the albumin to the mTOR inhibitor (such as a limus drug, e.g., rapamycin or a derivative thereof) in the mTOR inhibitor nanoparticle composition is such that a sufficient amount of mTOR inhibitor binds to, or is transported by, the cell. While the weight ratio of an albumin to an mTOR inhibitor (such as a limus drug, e.g., rapamycin or a derivative thereof) will have to be optimized for different albumin and mTOR inhibitor combinations, generally the weight ratio of an albumin to an mTOR inhibitor (such as a limus drug, e.g., rapamycin or a derivative thereof) (w/w) is about 0.01:1 to about 100:1, about 0.02:1 to about 50:1, about 0.05:1 to about 20:1, about 0.1:1 to about 20:1, about 1:1 to about 18:1, about 2:1 to about 15:1, about 3:1 to about 12:1, about 4:1 to about 10:1, about 5:1 to about 9:1, or about 9:1. In some embodiments, the albumin to mTOR inhibitor (such as a limus drug, e.g., rapamycin or a derivative thereof) weight ratio is about any of 18:1 or less, 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, and 3:1 or less. In some embodiments, the weight ratio of the albumin (such as human albumin or human serum albumin) to the mTOR inhibitor (such as a limus drug, e.g., rapamycin or a derivative thereof) in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the composition comprises nanoparticles comprising an mTOR inhibitor and an albumin, wherein the weight ratio of the albumin to the mTOR inhibitor in the composition is about 0.01:1 to about 100:1. In some embodiments, the composition comprises nanoparticles comprising an mTOR inhibitor (such as rapamycin) and an albumin, wherein the weight ratio of the albumin to the mTOR inhibitor (such as rapamycin) in the composition is about 18:1 or less (including for example any of about 1:1 to about 18:1, about 2:1 to about 15:1, about 3:1 to about 12:1, about 4:1 to about 10:1, about 5:1 to about 9:1, and about 9:1). In some embodiments, the composition comprises nanoparticles comprising rapamycin, or a derivative thereof, and an albumin, wherein the weight ratio of the albumin to the rapamycin or derivative thereof in the composition is about 18:1 or less (including for example any of about 1:1 to about 18:1, about 2:1 to about 15:1, about 3:1 to about 12:1, about 4:1 to about 10:1, about 5:1 to about 9:1, and about 9:1). In some embodiments, the mTOR inhibitor (such as rapamycin) is coated with albumin.

In some embodiments, the mTOR inhibitor nanoparticle composition (such as rapamycin/albumin nanoparticle composition) comprises one or more of the above characteristics.

The nanoparticles described herein may be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

In some embodiments, the pharmaceutically acceptable carrier comprises an albumin (such as human albumin or human serum albumin). The albumin may either be natural in origin or synthetically prepared. In some embodiments, the albumin is human albumin or human serum albumin. In some embodiments, the albumin is a recombinant albumin.

Human serum albumin (HSA) is a highly soluble globular protein of Mᵣ 65K and consists of 585 amino acids. HSA is the most abundant protein in the plasma and accounts for 70-80% of the colloid osmotic pressure of human plasma. The amino acid sequence of HSA contains a total of 17 disulfide bridges, one free thiol (Cys 34), and a single tryptophan (Trp 214). Intravenous use of HSA solution has been indicated for the prevention and treatment of hypovolemic shock (see, e.g., Tullis, JAMA, 237: 355-360, 460-463, (1977)) and Houser et al., Surgery Gynecology and Obstetrics. 150: 811-816 (1980)) and in conjunction with exchange transfusion in the treatment of neonatal hyperbilirubinemia (see, e.g., Finlayson, Seminars in Thrombosis and Hemostasis, 6, 85-120, (1980)). Other albumins are contemplated, such as bovine serum albumin. Use of such non-human albumins could be appropriate, for example, in the context of use of these compositions in non-human mammals, such as the veterinary (including domestic pets and agricultural context). Human serum albumin (HSA) has multiple hydrophobic binding sites (a total of eight for fatty acids, an endogenous ligand of HSA) and binds a diverse set of drugs, especially neutral and negatively charged hydrophobic compounds (Goodman et al., The Pharmacological Basis of Therapeutics, 9^{th} ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (198a), Vorum, Dan. Med. Bull., 46, 379-99 (1999), Kragh-Hansen, Dan. Med Bull., 1441, 13140 (1990). Curry et al., Nat. Struct. Biol., 5, 827-35 (1998). Sugio et al., Protein. Eng., 12, 43946 (1999). He et al., Nature, 358, 209-15 (199b), and Carter et al., Adv. Protein. Chem., 45, 153-203 (1994)). Rapamycin and propofol have been shown to bind HSA (see, e.g., Paal et al., Eur. J. Biochem., 268(7), 2187-91 (200a), Purcell et al., Biochem. Biophys. Acta, 1478(a), 61-8 (2000), Altmayer et al., Arzneimittelforschung, 45, 1053-6 (1995), and Garrido et al., Rev. Esp. Anestestiol. Reanim., 41, 308-12 (1994)). In addition, docetaxel has been shown to bind to human plasma proteins (see, e.g., Urien et al., Invest. New Drugs, 14(b), 147-51 (1996)).

In some embodiments, the composition described herein is substantially free (such as free) of surfactants, such as Cremophor (or polyoxyethylated castor oil, including Cremophor EL9® (BASF) or Tween 80). In some embodiments, the mTOR inhibitor nanoparticle composition (such as rapamycin/albumin nanoparticle composition) is substantially free (such as free) of surfactants. A composition is “substantially free of Cremophor” or “substantially free of surfactant” if the amount of Cremophor or surfactant in the composition is not sufficient to cause one or more side effect(s) in an individual when the mTOR inhibitor nanoparticle composition (such as rapamycin/albumin nanoparticle composition) is administered to the individual. In some embodiments, the mTOR inhibitor nanoparticle composition (such as rapamycin/albumin nanoparticle composition) contains less than about any one of 20%, 15%, 10%, 7.5%, 5%, 2.5%, or 1% organic solvent or surfactant. In some embodiments, the albumin is human albumin or human serum albumin. In some embodiments, the albumin is recombinant albumin.

The amount of an albumin in the composition described herein will vary depending on other components in the composition. In some embodiments, the composition comprises an albumin in an amount that is sufficient to stabilize the mTOR inhibitor (such as a limus drug, e.g., rapamycin or a derivative thereof) in an aqueous suspension, for example, in the form of a stable colloidal suspension (such as a stable suspension of nanoparticles). In some embodiments, the albumin is in an amount that reduces the sedimentation rate of the mTOR inhibitor (such as a limus drug, e.g., rapamycin or a derivative thereof) in an aqueous medium. For particle-containing compositions, the amount of the albumin also depends on the size and density of nanoparticles of the mTOR inhibitor.

An mTOR inhibitor (such as a limus drug, e.g., rapamycin or a derivative thereof) is “stabilized” in an aqueous suspension if it remains suspended in an aqueous medium (such as without visible precipitation or sedimentation) for an extended period of time, such as for at least about any of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to an individual (such as a human). Stability of the suspension is generally (but not necessarily) evaluated at a storage temperature (such as room temperature (such as 20-25° C.) or refrigerated conditions (such as 4° C.)). For example, a suspension is stable at a storage temperature if it exhibits no flocculation or particle agglomeration visible to the naked eye or when viewed using an optical microscope at 1000 times, at about fifteen minutes after preparation of the suspension. Stability can also be evaluated under accelerated testing conditions, such as at a temperature that is about 40° C., or higher.

The compositions described herein may be a stable aqueous suspension of the mTOR inhibitor, such as a stable aqueous suspension of the mTOR inhibitor at a concentration of any of about 0.1 to about 200 mg/ml, about 0.1 to about 150 mg/ml, about 0.1 to about 100 mg/ml, about 0.1 to about 50 mg/ml, about 0.1 to about 20 mg/ml, about 1 to about 10 mg/ml, about 2 mg/ml to about 8 mg/ml, about 4 to about 6 mg/ml, and about 5 mg/ml. In some embodiments, the concentration of the mTOR inhibitor is at least about any of 0.2 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, 50 mg/ml, 100 mg/ml, 150 mg/ml, or 200 mg/ml.

In some embodiments, the albumin is present in an amount that is sufficient to stabilize the mTOR inhibitor (such as a limus drug, e.g., rapamycin or a derivative thereof) in an aqueous suspension at a certain concentration. For example, the concentration of the mTOR inhibitor (such as a limus drug, e.g., rapamycin or a derivative thereof) in the composition is about 0.1 to about 100 mg/ml, including for example about any of 0.1 to about 50 mg/ml, about 0.1 to about 20 mg/ml, about 1 to about 10 mg/ml, about 2 mg/ml to about 8 mg/ml, about 4 to about 6 mg/ml, or about 5 mg/ml. In some embodiments, the concentration of the mTOR inhibitor (such as a limus drug, e.g., rapamycin or a derivative thereof) is at least about any of 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the albumin is present in an amount that avoids use of surfactants (such as Cremophor), so that the composition is free or substantially free of surfactant (such as Cremophor).

In some embodiments, the composition, in liquid form, comprises from about 0.1% to about 50% (w/v) (e.g., about 0.5% (w/v), about 5% (w/v), about 10% (w/v), about 15% (w/v), about 20% (w/v), about 30% (w/v), about 40% (w/v), or about 50% (w/v)) of an albumin. In some embodiments, the composition, in liquid form, comprises about 0.5% to about 5% (w/v) of albumin.

In some embodiments, the albumin allows the composition to be administered to an individual (such as a human) without significant side effects. In some embodiments, the albumin (such as human serum albumin or human albumin) is in an amount that is effective to reduce one or more side effects of administration of the mTOR inhibitor (such as a limus drug, e.g., rapamycin or a derivative thereof) to a human. The term “reducing one or more side effects” of administration of the mTOR inhibitor (such as a limus drug, e.g., rapamycin or a derivative thereof) refers to reduction, alleviation, elimination, or avoidance of one or more undesirable effects caused by the mTOR inhibitor, as well as side effects caused by delivery vehicles (such as solvents that render the limus drugs suitable for injection) used to deliver the mTOR inhibitor. Such side effects include, for example, myelosuppression, neurotoxicity, hypersensitivity, inflammation, venous irritation, phlebitis, pain, skin irritation, peripheral neuropathy, neutropenic fever, anaphylactic reaction, venous thrombosis, extravasation, and combinations thereof. These side effects, however, are merely exemplary and other side effects, or combination of side effects, associated with limus drugs (such as a limus drug, e.g., rapamycin or a derivative thereof) can be reduced.

In some embodiments, the mTOR inhibitor nanoparticle compositions described herein comprise nanoparticles comprising an mTOR inhibitor (such as a limus drug, e.g., rapamycin or a derivative thereof) and an albumin (such as human albumin or human serum albumin), wherein the nanoparticles have an average diameter of no greater than about 200 nm. In some embodiments, the mTOR inhibitor nanoparticle compositions described herein comprise nanoparticles comprising an mTOR inhibitor (such as a limus drug, e.g., rapamycin or a derivative thereof) and an albumin (such as human albumin or human serum albumin), wherein the nanoparticles have an average diameter of no greater than about 150 nm. In some embodiments, the mTOR inhibitor nanoparticle compositions described herein comprise nanoparticles comprising an mTOR inhibitor (such as a limus drug, e.g., rapamycin or a derivative thereof) and an albumin (such as human albumin or human serum albumin), wherein the nanoparticles have an average diameter of no greater than about 150 nm (for example about 100-120 nm, for example about 100 nm). In some embodiments, the mTOR inhibitor nanoparticle compositions described herein comprise nanoparticles comprising rapamycin and human albumin (such as human serum albumin), wherein the nanoparticles have an average diameter of no greater than about 150 nm (for example about 100-120 nm, for example about 100 nm). In some embodiments, the mTOR inhibitor nanoparticle compositions described herein comprise nanoparticles comprising rapamycin and human albumin (such as human serum albumin), wherein the average or mean diameter of the nanoparticles is about 10 to about 150 nm. In some embodiments, the mTOR inhibitor nanoparticle compositions described herein comprise nanoparticles comprising rapamycin and human albumin (such as human serum albumin), wherein the average or mean diameter of the nanoparticles is about 40 to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as a limus drug, e.g., rapamycin or a derivative thereof) and an albumin (such as human albumin or human serum albumin), wherein the nanoparticles have an average diameter of no greater than about 200 nm, wherein the weight ratio of the albumin and the mTOR inhibitor in the composition is no greater than about 9:1 (for example, from about 3:1 to about 9:1, such as about 9:1 or about 8:1). In some embodiments, the mTOR inhibitor nanoparticle compositions described herein comprise nanoparticles comprising an mTOR inhibitor (such as a limus drug, e.g., rapamycin or a derivative thereof) and an albumin (such as human albumin or human serum albumin), wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., rapamycin or a derivative thereof) and an albumin (such as human albumin or human serum albumin), wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising rapamycin and human albumin (such as human serum albumin), wherein the nanoparticles have an average diameter of no greater than about 150 nm (for example about 100-120 nm, for example about 100 nm), wherein the weight ratio of albumin and mTOR inhibitor in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as a limus drug, e.g., rapamycin or a derivative thereof) associated (e.g., coated) with an albumin (such as human albumin or human serum albumin). In some embodiments, the mTOR inhibitor nanoparticle compositions described herein comprise nanoparticles comprising an mTOR inhibitor (such as a limus drug, e.g., rapamycin or a derivative thereof) associated (e.g., coated) with an albumin (such as human albumin or human serum albumin), wherein the nanoparticles have an average diameter of no greater than about 200 nm. In some embodiments, the mTOR inhibitor nanoparticle compositions described herein comprise nanoparticles comprising an mTOR inhibitor (such as a limus drug, e.g., rapamycin or a derivative thereof) associated (e.g., coated) with an albumin (such as human albumin or human serum albumin), wherein the nanoparticles have an average diameter of no greater than about 150 nm. In some embodiments, the mTOR inhibitor nanoparticle compositions described herein comprise nanoparticles comprising an mTOR inhibitor (such as a limus drug, e.g., rapamycin or a derivative thereof) associated (e.g., coated) with an albumin (such as human albumin or human serum albumin), wherein the nanoparticles have an average diameter of about 10 nm to about 150 nm. In some embodiments, the mTOR inhibitor nanoparticle compositions described herein comprise nanoparticles comprising an mTOR inhibitor (such as a limus drug, e.g., rapamycin or a derivative thereof) associated (e.g., coated) with an albumin (such as human albumin or human serum albumin), wherein the nanoparticles have an average diameter of about 40 nm to about 120 nm. In some embodiments, the mTOR inhibitor nanoparticle compositions described herein comprise nanoparticles comprising rapamycin associated (e.g., coated) with human albumin (such as human serum albumin), wherein the nanoparticles have an average diameter of no greater than about 150 nm (for example about 100-120 nm, for example about 100 nm). In some embodiments, the mTOR inhibitor nanoparticle compositions described herein comprise nanoparticles comprising rapamycin associated (e.g., coated) with human albumin (such as human serum albumin), wherein the nanoparticles have an average diameter of about 10 nm to about 150 nm. In some embodiments, the mTOR inhibitor nanoparticle compositions described herein comprise nanoparticles comprising rapamycin associated (e.g., coated) with human albumin (such as human serum albumin), wherein the nanoparticles have an average diameter of about 40 nm to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as a limus drug, e.g., rapamycin or a derivative thereof) associated (e.g., coated) with an albumin (such as human albumin or human serum albumin), wherein the weight ratio of the albumin and the mTOR inhibitor in the composition is no greater than about 9:1 (for example, from about 3:1 to about 9:1, such as about 9:1 or about 8:1). In some embodiments, the mTOR inhibitor nanoparticle compositions described herein comprise nanoparticles comprising an mTOR inhibitor (such as a limus drug, e.g., rapamycin or a derivative thereof) associated (e.g., coated) with an albumin (such as human albumin or human serum albumin), wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., rapamycin or a derivative thereof) associated (e.g., coated) with an albumin (such as human albumin or human serum albumin), wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., rapamycin or a derivative thereof) associated (e.g., coated) with an albumin (such as human albumin or human serum albumin), wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising rapamycin associated (e.g., coated) with human albumin (such as human serum albumin), wherein the nanoparticles have an average diameter of no greater than about 150 nm (for example about 100-120 nm, for example about 100 nm), wherein the weight ratio of albumin and the rapamycin in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as a limus drug, e.g., rapamycin or a derivative thereof) stabilized by an albumin (such as human albumin or human serum albumin). In some embodiments, the mTOR inhibitor nanoparticle compositions described herein comprise nanoparticles comprising an mTOR inhibitor (such as a limus drug. e.g., rapamycin or a derivative thereof) stabilized by an albumin (such as human albumin or human serum albumin), wherein the nanoparticles have an average diameter of no greater than about 200 nm. In some embodiments, the mTOR inhibitor nanoparticle compositions described herein comprise nanoparticles comprising an mTOR inhibitor (such as a limus drug, e.g., rapamycin or a derivative thereof) stabilized by an albumin (such as human albumin or human serum albumin), wherein the nanoparticles have an average diameter of no greater than about 150 nm. In some embodiments, the mTOR inhibitor nanoparticle compositions described herein comprise nanoparticles comprising an mTOR inhibitor (such as a limus drug, e.g., rapamycin or a derivative thereof) stabilized by an albumin (such as human albumin or human serum albumin), wherein the nanoparticles have an average diameter of no greater than about 150 nm (for example about 100-120 nm, for example about 100 nm). In some embodiments, the mTOR inhibitor nanoparticle compositions described herein comprise nanoparticles comprising rapamycin stabilized by human albumin (such as human serum albumin), wherein the nanoparticles have an average diameter of no greater than about 150 nm (for example about 100-120 nm, for example about 100 nm). In some embodiments, the average or mean diameter of the nanoparticles is about 10 nm to about 150 nm. In some embodiments, the average or mean diameter of the nanoparticles is about 40 nm to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as a limus drug, e.g., rapamycin or a derivative thereof) stabilized by an albumin (such as human albumin or human serum albumin), wherein the weight ratio of the albumin and the mTOR inhibitor in the composition is no greater than about 9:1 (for example, from about 3:1 to about 9:1, such as about 9:1 or about 8:1). In some embodiments, the mTOR inhibitor nanoparticle compositions described herein comprise nanoparticles comprising an mTOR inhibitor (such as a limus drug. e.g., rapamycin or a derivative thereof) stabilized by an albumin (such as human albumin or human serum albumin), wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., rapamycin or a derivative thereof) stabilized by an albumin (such as human albumin or human serum albumin), wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as a limus drug, e.g., rapamycin or a derivative thereof) stabilized by an albumin (such as human albumin or human serum albumin), wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising rapamycin stabilized by human albumin (such as human serum albumin), wherein the nanoparticles have an average diameter of no greater than about 150 nm (for example about 100-120 nm, for example about 100 nm), wherein the weight ratio of albumin and the rapamycin in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as rapamycin) and an albumin (such as human albumin or human serum albumin), wherein the composition further comprises a saccharide, wherein the nanoparticles have an average diameter of no greater than about 200 nm. In some embodiments, the mTOR inhibitor nanoparticle compositions described herein comprise nanoparticles comprising an mTOR inhibitor (such as rapamycin) and an albumin (such as human albumin or human serum albumin), wherein the composition further comprises a saccharide, wherein the nanoparticles have an average diameter of no greater than about 150 nm. In some embodiments, the mTOR inhibitor nanoparticle compositions described herein comprise nanoparticles comprising an mTOR inhibitor (such as rapamycin) and an albumin (such as human albumin or human serum albumin), wherein the composition further comprises a saccharide, wherein the nanoparticles have an average diameter of no greater than about 150 nm (for example about 100 nm). In some embodiments, the average or mean diameter of the nanoparticles is about 100-120 nm, for example about 100 nm. In some embodiments, the mTOR inhibitor nanoparticle compositions described herein comprise nanoparticles comprising rapamycin and human albumin (such as human serum albumin), wherein the composition further comprises a saccharide, wherein the nanoparticles have an average diameter of no greater than about 150 nm (for example about 100 nm). In some embodiments, the average or mean diameter of the nanoparticles is about 100-120 nm, for example about 100 nm. In some embodiments, the mTOR inhibitor nanoparticle compositions described herein comprise nanoparticles comprising rapamycin and human albumin (such as human serum albumin), wherein the composition further comprises a saccharide, wherein the average or mean diameter of the nanoparticles is about 10 to about 150 nm. In some embodiments, the mTOR inhibitor nanoparticle compositions described herein comprise nanoparticles comprising rapamycin and human albumin (such as human serum albumin), wherein the average or mean diameter of the nanoparticles is about 40 to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as rapamycin) and an albumin (such as human albumin or human serum albumin), wherein the composition further comprises a saccharide, wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) and an albumin (such as human albumin or human serum albumin), wherein the composition further comprises a saccharide, wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) and an albumin (such as human albumin or human serum albumin), wherein the composition further comprises a saccharide, wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising rapamycin and human albumin (such as human serum albumin), wherein the composition further comprises a saccharide, wherein the nanoparticles have an average diameter of no greater than about 150 nm (for example about 100 nm), wherein the weight ratio of albumin and mTOR inhibitor in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as rapamycin) stabilized by an albumin (such as human albumin or human serum albumin), wherein the composition further comprises a saccharide, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) stabilized by an albumin (such as human albumin or human serum albumin), wherein the composition further comprises a saccharide, wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) stabilized by an albumin (such as human albumin or human serum albumin), wherein the composition further comprises a saccharide, wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) stabilized by an albumin (such as human albumin or human serum albumin), wherein the composition further comprises a saccharide, wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising rapamycin stabilized by human albumin (such as human serum albumin), wherein the composition further comprises a saccharide, wherein the nanoparticles have an average diameter of no greater than about 150 nm (for example about 100 nm), wherein the weight ratio of albumin and the rapamycin in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the composition further comprises a saccharide. In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the composition further comprises a saccharide, wherein the nanoparticles have an average diameter of no greater than about 200 nm. In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the composition further comprises a saccharide, wherein the nanoparticles have an average diameter of no greater than about 150 nm. In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the composition further comprises a saccharide, wherein the nanoparticles have an average diameter of about 10 nm to about 150 nm. In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the composition further comprises a saccharide, wherein the nanoparticles have an average diameter of about 40 nm to about 120 nm. In some embodiments, the mTOR inhibitor nanoparticle compositions described herein comprise nanoparticles comprising rapamycin associated (e.g., coated) with human albumin (such as human serum albumin), wherein the composition further comprises a saccharide, wherein the nanoparticles have an average diameter of no greater than about 150 nm (for example about 100 nm). In some embodiments, the mTOR inhibitor nanoparticle compositions described herein comprise nanoparticles comprising rapamycin associated (e.g., coated) with human albumin (such as human serum albumin), wherein the composition further comprises a saccharide, wherein the nanoparticles have an average diameter of about 10 nm to about 150 nm. In some embodiments, the mTOR inhibitor nanoparticle compositions described herein comprise nanoparticles comprising rapamycin associated (e.g., coated) with human albumin (such as human serum albumin), wherein the composition further comprises a saccharide, wherein the nanoparticles have an average diameter of about 40 nm to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the composition further comprises a saccharide, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the composition further comprises a saccharide, wherein the nanoparticles have an average diameter of no greater than about 200 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the composition further comprises a saccharide, wherein the nanoparticles have an average diameter of no greater than about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising an mTOR inhibitor (such as rapamycin) associated (e.g., coated) with an albumin (such as human albumin or human serum albumin), wherein the composition further comprises a saccharide, wherein the nanoparticles have an average diameter of about 150 nm, wherein the weight ratio of the albumin and the mTOR inhibitor in the composition is no greater than about 9:1 (such as about 9:1 or about 8:1). In some embodiments, the mTOR inhibitor nanoparticle compositions described herein comprise nanoparticles comprising rapamycin associated (e.g., coated) with human albumin (such as human serum albumin), wherein the composition further comprises a saccharide, wherein the nanoparticles have an average diameter of no greater than about 150 nm (for example about 100 nm), wherein the weight ratio of albumin and the rapamycin in the composition is about 9:1 or about 8:1. In some embodiments, the average or mean diameter of the nanoparticles is about 10 nm to about 150 nm. In some embodiments, the average or mean diameter of the nanoparticles is about 40 nm to about 120 nm. In some embodiments, the average or mean diameter of the nanoparticles is about 100-120 nm, for example about 100 nm.

In some embodiments, the mTOR inhibitor nanoparticle compositions described herein comprise nanoparticles comprising an mTOR inhibitor (such as rapamycin) stabilized by an albumin (such as human albumin or human serum albumin), wherein the composition further comprises a saccharide. In some embodiments, the mTOR inhibitor nanoparticle compositions described herein comprise nanoparticles comprising an mTOR inhibitor (such as rapamycin) stabilized by an albumin (such as human albumin or human serum albumin), wherein the composition further comprises a saccharide, wherein the nanoparticles have an average diameter of no greater than about 200 nm. In some embodiments, the mTOR inhibitor nanoparticle compositions described herein comprise nanoparticles comprising an mTOR inhibitor (such as rapamycin) stabilized by an albumin (such as human albumin or human serum albumin), wherein the composition further comprises a saccharide, wherein the nanoparticles have an average diameter of no greater than about 150 nm. In some embodiments, the mTOR inhibitor nanoparticle compositions described herein comprise nanoparticles comprising an mTOR inhibitor (such as rapamycin) stabilized by an albumin (such as human albumin or human serum albumin), wherein the composition further comprises a saccharide, wherein the nanoparticles have an average diameter of no greater than about 150 nm (for example about 100 nm). In some embodiments, the mTOR inhibitor nanoparticle compositions described herein comprise nanoparticles comprising rapamycin stabilized by human albumin (such as human serum albumin), wherein the composition further comprises a saccharide, wherein the nanoparticles have an average diameter of no greater than about 150 nm (for example about 100 nm). In some embodiments, the average or mean diameter of the nanoparticles is about 10 nm to about 150 nm. In some embodiments, the average or mean diameter of the nanoparticles is about 40 nm to about 120 nm. In some embodiments, the average or mean diameter of the nanoparticles is about 100-120 nm, for example about 100 nm.

In some embodiments, the mTOR inhibitor nanoparticle composition comprises nab-rapamycin. In some embodiments, the mTOR inhibitor nanoparticle composition is nab-rapamycin. Nab-rapamycin is a formulation of rapamycin stabilized by human albumin USP, which can be dispersed in directly injectable physiological solution. The weight ratio of human albumin and rapamycin is from about 3:1 to about 9:1, for example, about 8:1 to about 9:1. When dispersed in a suitable aqueous medium such as 0.9% sodium chloride injection or 5% dextrose injection, nab-rapamycin forms a stable colloidal suspension of rapamycin. The mean particle size of the nanoparticles in the colloidal suspension is about 100 nanometers. Since HSA is freely soluble in water, nab-rapamycin can be reconstituted in a wide range of concentrations ranging from dilute (0.1 mg/ml rapamycin or a derivative thereof) to concentrated (e.g., 50 mg/ml rapamycin or a derivative thereof), including for example about 2 mg/ml to about 8 mg/ml, or about 5 mg/ml.

Methods of making nanoparticle compositions are known in the art. For example, nanoparticles containing an mTOR inhibitor (such as a limus drug, e.g., rapamycin or a derivative thereof) and an albumin (such as human serum albumin or human albumin) can be prepared under conditions of high shear forces (e.g., sonication, high pressure homogenization, or the like). These methods are disclosed in, for example, U.S. Pat. Nos. 5,916,596; 6,506,405; 6,749,868, 6,537,579, 7,820,788, and 8,911,786, and also in U. S. Pat. Pub. Nos. 2007/0082838, 2006/0263434 and PCT Application WO08/137148.

Briefly, the mTOR inhibitor (such as a limus drug, e.g., rapamycin or a derivative thereof) is dissolved in an organic solvent, and the solution can be added to an albumin solution. The mixture is subjected to high pressure homogenization. The organic solvent can then be removed by evaporation. The dispersion obtained can be further lyophilized. Suitable organic solvent include, for example, ketones, esters, ethers, chlorinated solvents, and other solvents known in the art. For example, the organic solvent can be methylene chloride or chloroform/ethanol (for example with a ratio of 1:9, 1:8, 1:7, 1:6, 1:5, 1:4, 1:3, 1:2, 1:1, 2:1, 3:1, 4:1, 5:1, 6:1, 7:1, 8:1, or 9:1).

In some embodiments, the composition is a dry (such as lyophilized) composition that can be reconstituted, resuspended, or rehydrated to form generally a stable aqueous suspension of the nanoparticles comprising an mTOR inhibitor and an albumin. In some embodiments, the composition is a liquid (such as aqueous) composition obtained by reconstituting or resuspending a dry composition. In some embodiments, the composition is an intermediate liquid (such as aqueous) composition that can be dried (such as lyophilized).

A. mTOR Inhibitor

The methods described herein in some embodiments comprise administration of nanoparticle compositions of mTOR inhibitors. “mTOR inhibitor” used herein refers to an inhibitor of mTOR. mTOR is a serine/threonine-specific protein kinase downstream of the phosphatidylinositol 3-kinase (PI3K)/Akt (protein kinase B) pathway, and a key regulator of cell survival, proliferation, stress, and metabolism. mTOR pathway dysregulation has been found in many human carcinomas, and mTOR inhibition produced substantial inhibitory effects on tumor progression.

The mammalian target of rapamycin (mTOR) (also known as mechanistic target of rapamycin or FK506 binding protein 12-rapamycin associated protein 1 (FRAP1)) is an atypical serine/threonine protein kinase that is present in two distinct complexes, mTOR Complex 1 (mTORC1) and mTOR Complex 2 (mTORC2). mTORC1 is composed of mTOR, regulatory-associated protein of mTOR (Raptor), mammalian lethal with SEC13 protein 8 (MLST8), PRAS40 and DEPTOR (Kim et al. (2002). Cell 110: 163-75; Fang et al. (2001). Science 294 (5548): 1942-5). mTORC1 integrates four major signal inputs: nutrients (such as amino acids and phosphatidic acid), growth factors (insulin), energy and stress (such as hypoxia and DNA damage). Amino acid availability is signaled to mTORC1 via a pathway involving the Rag and Ragulator (LAMTOR1-3) Growth factors and hormones (e.g., insulin) signal to mTORC1 via Akt, which inactivates TSC2 to prevent inhibition of mTORC1. Alternatively, low ATP levels lead to the AMPK-dependent activation of TSC2 and phosphorylation of raptor to reduce mTORC1 signaling proteins.

Active mTORC1 has a number of downstream biological effects including translation of mRNA via the phosphorylation of downstream targets (4E-BP1 and p70 S6 Kinase), suppression of autophagy (Atg13, ULK1), ribosome biogenesis, and activation of transcription leading to mitochondrial metabolism or adipogenesis. Accordingly, mTORC1 activity promotes either cellular growth when conditions are favorable or catabolic processes during stress or when conditions are unfavorable.

mTORC2 is composed of mTOR, rapamycin-insensitive companion of mTOR (RICTOR), GβL, and mammalian stress-activated protein kinase interacting protein 1 (mSIN1). In contrast to mTORC1, for which many upstream signals and cellular functions have been defined (see above), relatively little is known about mTORC2 biology. mTORC2 regulates cytoskeletal organization through its stimulation of F-actin stress fibers, paxillin, RhoA, Rac1, Cdc42, and protein kinase C α (PKCα). It had been observed that knocking down mTORC2 components affects actin polymerization and perturbs cell morphology (Jacinto el al. (2004). Nat. Cell Biol. 6, 1122-1128; Sarbassov et al. (2004). Curr. Biol. 14, 1296-1302). This suggests that mTORC2 controls the actin cytoskeleton by promoting protein kinase Cα (PKCα) phosphorylation, phosphorylation of paxillin and its relocalization to focal adhesions, and the GTP loading of RhoA and Rac1. The molecular mechanism by which mTORC2 regulates these processes has not been determined.

In some embodiments, the mTOR inhibitor (such as a limus drug, e.g., sirolimus or a derivative thereof) is an inhibitor of mTORC1. In some embodiments, the mTOR inhibitor (such as a limus drug, e.g., sirolimus or a derivative thereof) is an inhibitor of mTORC2. In some embodiments, the mTOR inhibitor (such as a limus drug, e.g., sirolimus or a derivative thereof) is an inhibitor of both mTORC1 and mTORC2.

In some embodiments, the mTOR inhibitor is a limus drug, which includes sirolimus and its analogs. Examples of limus drugs include, but are not limited to, temsirolimus (CCI-779), everolimus (RAD00 l), ridaforolimus (AP-23573), deforolimus (MK-8669), zotarolimus (ABT-578), pimecrolimus, and tacrolimus (FK-506). In some embodiments, the limus drug is selected from the group consisting of temsirolimus (CC1-779), everolimus (RAD001), ridaforolimus (AP-23573), deforolimus (MK-8669), zotarolimus (ABT-578), pimecrolimus, and tacrolimus (FK-506). In some embodiments, the mTOR inhibitor is an mTOR kinase inhibitor, such as CC-115 or CC-223.

In some embodiments, the mTOR inhibitor is sirolimus. Sirolimus is macrolide antibiotic that complexes with FKBP-12 and inhibits the mTOR pathway by binding mTORC1.

In some embodiments, the mTOR inhibitor is selected from the group consisting of sirolimus (rapamycin). BEZ235 (NVP-BEZ235), everolimus (also known as RAD001, Zortress, Certican, and Afinitor), AZD8055, temsirolimus (also known as CCI-779 and Torisel), CC-115, CC-223, PI-103, Ku-0063794, INK 128, AZD2014, NVP-BGT226, PF-04691502, CH5132799, GDC-0980 (RG7422), Torin 1, WAY-600, WYE-125132, WYE-687, GSK2126458, PF-05212384 (PKI-587), PP-121, OSI-027, Palomid 529, PP242, XL765, GSK1059615, WYE-354, and ridaforolimus (also known as deforolimus).

BEZ235 (NVP-BEZ235) is an imidazoquilonine derivative that is an mTORC1 catalytic inhibitor (Roper J, et al. PLoS One, 2011, 6(9), e25132). Everolimus is the 40-O-(2-hydroxyethyl) derivative of sirolimus and binds the cyclophilin FKBP-12, and this complex also mTORC1. AZD8055 is a small molecule that inhibits the phosphorylation of mTORC1 (p70S6K and 4E-BP1). Temsirolimus is a small molecule that forms a complex with the FK506-binding protein and prohibits the activation of mTOR when it resides in the mTORC1 complex. PI-103 is a small molecule that inhibits the activation of the rapamycin-sensitive (mTORC1) complex (Knight et al. (2006) Cell. 125: 733-47). KU-0063794 is a small molecule that inhibits the phosphorylation of mTORC1 at Ser2448 in a dose-dependent and time-dependent manner. INK 128, AZD2014. NVP-BGT226, CH5132799, WYE-687, and are each small molecule inhibitors of mTORC1. PF-04691502 inhibits mTORC1 activity. GDC-0980 is an orally bioavailable small molecule that inhibits Class I PI3 Kinase and TORC1. Torin 1 is a potent small molecule inhibitor of mTOR. WAY-600 is a potent, ATP-competitive and selective inhibitor of mTOR. WYE-125132 is an ATP-competitive small molecule inhibitor of mTORC1. GSK2126458 is an inhibitor of mTORC1. PKI-587 is a highly potent dual inhibitor of PI3Kα, PI3Kγ and mTOR. PP-121 is a multi-target inhibitor of PDGFR, Hck, mTOR, VEGFR2, Src and Abl. OSI-027 is a selective and potent dual inhibitor of mTORC1 and mTORC2 with IC50 of 22 nM and 65 nM, respectively. Palomid 529 is a small molecule inhibitor of mTORC1 that lacks affinity for ABCB1/ABCG2 and has good brain penetration (Lin et al. (2013) Int J Cancer DOI: 10.1002/ijc. 28126 (e-published ahead of print). PP242 is a selective mTOR inhibitor. XL765 is a dual inhibitor of mTOR/PI3k for mTOR, p110α, p110β, p110γ and p110δ. GSK1059615 is a novel and dual inhibitor of PI3Kα, PI3Kβ, PI3Kδ, PI3Kγ and mTOR. WYE-354 inhibits mTORC1 in HEK293 cells (0.2 μM-5 μM) and in HUVEC cells (10 nM-1 μM). WYE-354 is a potent, specific and ATP-competitive inhibitor of mTOR. Deforolimus (Ridaforolimus, AP23573, MK-8669) is a selective mTOR inhibitor.

B. Carrier Protein

In some embodiments, the composition comprises an mTOR inhibitor and a carrier protein. The term “proteins” refers to polypeptides or polymers of amino acids of any length (including full length or fragments), which may be linear or branched, comprise modified amino acids, and/or be interrupted by non-amino acids. The term also encompasses an amino acid polymer that has been modified naturally or by intervention; for example, disulfide bond formation, glycosylation, lipidation, acetylation, phosphorylation, or any other manipulation or modification. Also included within this term are, for example, polypeptides containing one or more analogs of an amino acid (including, for example, unnatural amino acids, etc.), as well as other modifications known in the art. The proteins described herein may be naturally occurring, i.e., obtained or derived from a natural source (such as blood), or synthesized (such as chemically synthesized or by synthesized by recombinant DNA techniques). Examples of suitable carrier proteins include proteins normally found in blood or plasma, which include, but are not limited to, albumin, immunoglobulin including IgA, lipoproteins, apolipoprotein B, alpha-acid glycoprotein, beta-2-macroglobulin, thyroglobulin, transferin, fibronectin, factor VII, factor VIII, factor IX, factor X, and the like. In some embodiments, the carrier protein is non-blood protein, such as casein, α-lactalbumin, and β-lactoglobulin. The carrier proteins may either be natural in origin or synthetically prepared.

In some embodiments, the carrier protein is an albumin. In some embodiments, the albumin is serum albumin. In some embodiments, the albumin is human serum albumin.

C. Other Components in the Nanoparticle Composition

The nanoparticles described herein can be present in a composition that includes other agents, excipients, or stabilizers. For example, to increase stability by increasing the negative zeta potential of nanoparticles, certain negatively charged components may be added. Such negatively charged components include, but are not limited to bile salts of bile acids consisting of glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid and others; phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine stearoyloleoylphosphatidylcholine, stearoylarachidoylphosphatidylcholine, and dipalmitoylphosphatidylcholine. Other phospholipids including L-α-dimyristoylphosphatidylcholine (DMPC), dioleoylphosphatidylcholine (DOPC), distearyolphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

In some embodiments, the composition is suitable for administration to a human. In some embodiments, the composition is suitable for administration to a mammal such as, in the veterinary context, domestic pets and agricultural animals. There are a wide variety of suitable formulations of the mTOR inhibitor nanoparticle composition (such as sirolimus/albumin nanoparticle composition) (see, e.g., U.S. Pat. Nos. 5,916,596 and 6,096,331). The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Injectable formulations are preferred.

In some embodiments, the composition is formulated to have a pH range of about 4.5 to about 9.0, including for example pH ranges of about any of 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

D. Albumin-Based Nanoparticle Compositions of Rapamycin

The methods described herein are particularly suitable for albumin-based nanoparticle compositions described herein in more details. The nanoparticle composition in some embodiments includes (a) nanoparticles that include rapamycin and albumin, and (b) a non-nanoparticle portion that includes rapamycin and albumin. The rapamycin and the albumin of the nanoparticles are associated with each other in the nanoparticles. For example, the nanoparticles may include a coating having the albumin, which surrounds a core comprising the rapamycin. In the non-nanoparticle portion of the composition, the rapamycin and the albumin may or may not associated with each other (i.e., the rapamycin may be in a reversible binding equilibrium with the albumin), but do not associate with each other in a manner that forms nanoparticles. That is, the nanoparticle composition may include nanoparticle-bound albumin and nanoparticle-bound rapamycin in the nanoparticle portion of the composition, and non-nanoparticle albumin and non-nanoparticle rapamycin in the non-nanoparticle portion of the composition. As used herein, “in the nanoparticles” is used synonymously with “in the nanoparticle portion.” The albumin of the nanoparticles may be further distinguishable from the albumin in the non-nanoparticle portion of the composition; for example, the oligomeric profile of the albumin in the nanoparticles may differ from the oligomeric profile of the albumin in the non-nanoparticle portion of the composition. The oligomer profile means the percentage of various albumin species compared with the total albumin in the composition. The types of albumin species includes albumin monomers, dimers, trimers, oligomers, and polymers. As used herein, “albumin monomers” or “monomeric albumin” refers to an albumin species having one, and only one, albumin unit; “albumin dimers” or “dimeric albumin” refers to an albumin species having two, and only two, albumin units; “albumin trimers” or “trimeric albumin” refers to albumin species having three, and only three, albumin units; “albumin polymers” refers to albumin species having a higher molecular weight than albumin monomers and albumin dimers; “albumin oligomers” or “oligomeric albumin” refers to lower molecular weight polymeric albumin species associated with a UV-based size-exclusion chromatography peak observed between a peak associated with albumin dimers and higher molecular weight polymeric albumin species.

The albumin of the nanoparticles associates with the rapamycin of the nanoparticles so that a nanoparticle suspension has a high concentration of rapamycin, which allows the composition to be used as a pharmaceutical composition for treating certain diseases, such as cancer. Manufactured nanoparticles (which may be made, for example, using the methods described herein) may be formulated, filtered, or otherwise processed to obtain the pharmaceutical composition, which may be suitable for medical use in a human individual.

Generally, to make the rapamycin pharmaceutical compositions described herein, rapamycin is dissolved in an organic solvent. Suitable organic solvents include, for example, ketones, esters, ethers, chlorinated solvents, and other solvents known in the art. For example, the organic solvent can be a mixture of methylene chloride/ethanol, chloroform/ethanol, or chloroform/tert-butanol (for example with a ratio of about any one of 1:9, 1:8, 1:7, 1:6, 1:5, 1:4, 1:3, 1:2, 1:1, 2:1, 3:1, 4:1, 5:1, 6:1, 7:1, 8:1, or 9:1 or with a ratio of about any one of 3:7, 5:7, 4:6, 5:5, 6:5, 8:5, 9:5, 9.5:5, 5:3, 7:3, 6:4, or 9.5:0.5). In some embodiments, the organic solvent comprises between about 10% and about 50% tert-butanol by volume. In some embodiments, the organic solvent comprises about any of 10%, 15%, 20%, 25%, 30%, 35%, 40%, 45%, or 50% tert-butanol by volume. In some embodiments, the organic solvent comprises about any of 10-15%, 15-20%, 20-25%, 25-30%, 30-35%, 3540%, 40-45%, or 45-50%, or any combination of such ranges, of tert-butanol by volume. In some embodiments, the organic solvent comprises between about 50% and about 90% chloroform by volume. In some embodiments, the organic solvent comprises about any of 50%, 55%, 60%, 65%, 70%, 75%, 80%, 85%, or 90% chloroform by volume. In some embodiments, the organic solvent comprises about any of 50-55%, 55-60%, 60-65%, 65-70%, 70-75%, 75-80%, 80-85%, or 85-90%, or any combination of such ranges, of chloroform by volume. In some embodiments, the organic solvent comprises between about 10% and about 50% tert-butanol by volume and between about 50% and about 90% chloroform by volume. In some embodiments, the organic solvent comprises chloroform and tert-butanol at a volumetric ratio of about 1:1 to about 1:9, such as about any of 1:1, 2:1, 3:1, 41, 5:1, 6:1, 7:1, 8:1, and 9:1.

Albumin (such as recombinant albumin, for example NOVOZYME™ recombinant albumin or INTRIVIA™ recombinant albumin disclosed herein) is dissolved in an aqueous solution (such as water) and combined with the rapamycin solution to form a crude emulsion. The mixture is subjected to high pressure homogenization (e.g., using an Avestin, APV Gaulin, MICROFLUIDIZER™ such as a MICROFLUIDIZER™ Processor M-110EH from Microfluidics, Stansted, or Ultra Turrax homogenizer). The emulsion may be cycled through the high pressure homogenizer for between about 2 to about 100 cycles, such as about 5 to about 50 cycles or about 6 to about 20 cycles (e.g., about any one of 6, 8, 10, 12, 14, 16, 18 or 20 cycles). The organic solvent can then be removed by evaporation utilizing suitable equipment known for this purpose, including, but not limited to, rotary evaporators, falling film evaporators, wiped film evaporators, spray driers, and the like that can be operated in batch mode or in continuous operation. In some embodiments, the evaporator is a wiped film evaporator. The solvent may be removed at reduced pressure (such as at about any one of 25 mm Hg, 30 mm Hg, 40 mm Hg, 50 mm Hg, 100 mm Hg, 200 mm Hg, or 300 mm Hg). The amount of time used to remove the solvent under reduced pressure may be adjusted based on the volume of the formulation. For example, for a formulation produced on a 300 mL scale, the solvent can be removed at about 1 to about 300 mm Hg (e.g., about any one of 5-100 mm Hg, 10-50 mm Hg, 20-40 mm Hg, or 25 mm Hg) for about 5 to about 60 minutes (e.g., about any one of 7, 8, 9, 10, 11, 12, 13, 14, 15 16, 18, 20, 25, or 30 minutes). The dispersion obtained can be further lyophilized.

The nanoparticle compositions described herein (such a pharmaceutical composition) may have distinct characteristics for any one or more (in any combination) of the following: (1) the oligomeric status of the albumin associated with (such as in) the nanoparticles, such as the percentage of albumin monomers, dimers, and/or polymers (or trimers) of the albumin associated with (such as in) the nanoparticles; (2) the oligomeric status of the albumin associated with (such as in) the non-nanoparticle portion of the composition, such as the percentage of albumin monomers, dimers, and/or polymers (or trimers) of the albumin associated with (such as in) the non-nanoparticle portion of the composition; (3) the oligomeric status of the total albumin in the composition, such as the percentage of albumin monomers, dimers, and/or polymers (or trimers) of the total albumin in the composition; (4) the particle size profile of the nanoparticles, such as the average particle size, polydispersity index, and/or size distribution; (5) the portion (e.g., weight percentage) of the nanoparticles that is albumin and/or the portion (e.g., weight percentage) of the nanoparticles that is rapamycin; (6) the weight ratio of the albumin to the rapamycin in the nanoparticles; (7) the weight ratio of the albumin to the rapamycin in the non-nanoparticle portion of the composition; (8) the weight ratio of the albumin to the rapamycin in the non-nanoparticle portion of the composition (9) the weight ratio of the total albumin to the total rapamycin in the composition; (10) the portion (e.g., weight percentage) of rapamycin that is in the nanoparticles (or the non-nanoparticle portion of the composition) compared to the total rapamycin in the composition; (11) the portion (e.g., weight percentage) of albumin that is in the non-nanoparticle portion (or in the nanoparticles) compared to the total albumin in the composition; (12) the concentration of albumin in the composition; (13) the concentration of albumin in the non-nanoparticle portion of the composition; (14) the concentration of albumin in the composition that is associated with (such as in) the nanoparticles; (15) the concentration of rapamycin in the composition; (16) the concentration of rapamycin in the non-nanoparticle portion of the composition; (17) the concentration of rapamycin in the composition that is associated with (such as in) the nanoparticles; (18) the osmolality of the composition; (19) the viscosity of the composition; (20) the pH of the composition; (21) the stability of the nanoparticles in the composition; (22) the amount of residual solvent in the composition; (23) the zeta potential of the nanoparticles in the composition; (24) the crystalline status of the rapamycin in the nanoparticles; (25) the particle morphology of the nanoparticles, such as the shape, sphericity, thickness of the coating, and/or surface-to-volume ratio; (26) the weight percentage of seco-rapamycin in the nanoparticles, as compared to the sum of seco-rapamycin and rapamycin, by weight; (27) the presence, percentage, or concentration of albumin stabilizer (such as sodium caprylate and/or N-acetyltryptophanate) in the composition; (28) the recovery of rapamycin following filtration; (29) in vitro release kinetics of the nanoparticles; (30) the portion of total rapamycin in the composition that is both in the non-nanoparticle portion of the composition and not bound to albumin; and/or (31) the weight percentage of seco-rapamycin in the composition, as compared to the sum of seco-rapamycin and rapamycin, by weight. In some embodiments, the oligomeric status (such as the percentage of albumin monomers, dimers, or polymers (or trimers)) of the nanoparticles, the non-nanoparticles portion, or the total composition is assessed by size-exclusion chromatography using a saline mobile phase coupled with a multiple angle light scattering (MALS) detector).

The nanoparticle compositions described herein (such a pharmaceutical composition) may have distinct characteristics for any one or more (in any combination) of the following: (1) the oligomeric status of the albumin associated with (such as in) the nanoparticles, such as the percentage of albumin monomers, dimers, oligomers, and/or polymers (other than oligomers) of the albumin associated with (such as in) the nanoparticles; (2) the oligomeric status of the albumin associated with (such as in) the non-nanoparticle portion of the composition, such as the percentage of albumin monomers, dimers, oligomers, and/or polymers (other than oligomers) of the albumin associated with (such as in) the non-nanoparticle portion of the composition; (3) the oligomeric status of the total albumin in the composition, such as the percentage of albumin monomers, dimers, oligomers, and/or polymers (other than oligomers) of the total albumin in the composition; (4) the particle size profile of the nanoparticles, such as the average particle size, polydispersity index, and/or size distribution; (5) the portion (e.g., weight percentage) of the nanoparticles that is albumin and/or the portion (e.g., weight percentage) of the nanoparticles that is rapamycin; (6) the weight ratio of the albumin to the rapamycin in the nanoparticles; (7) the weight ratio of the albumin to the rapamycin in the non-nanoparticle portion of the composition; (8) the weight ratio of the albumin to the rapamycin in the non-nanoparticle portion of the composition (9) the weight ratio of the total albumin to the total rapamycin in the composition; (10) the portion (e.g., weight percentage) of rapamycin that is in the nanoparticles (or the non-nanoparticle portion of the composition) compared to the total rapamycin in the composition; (11) the portion (e.g., weight percentage) of albumin that is in the non-nanoparticle portion (or in the nanoparticles) compared to the total albumin in the composition; (12) the concentration of albumin in the composition; (13) the concentration of albumin in the non-nanoparticle portion of the composition; (14) the concentration of albumin in the composition that is associated with (such as in) the nanoparticles; (15) the concentration of rapamycin in the composition; (16) the concentration of rapamycin in the non-nanoparticle portion of the composition; (17) the concentration of rapamycin in the composition that is associated with (such as in) the nanoparticles; (18) the osmolality of the composition; (19) the viscosity of the composition; (20) the pH of the composition; (21) the stability of the nanoparticles in the composition; (22) the amount of residual solvent in the composition; (23) the zeta potential of the nanoparticles in the composition; (24) the crystalline status of the rapamycin in the nanoparticles; (25) the particle morphology of the nanoparticles, such as the shape, sphericity, thickness of the coating, and/or surface-to-volume ratio; (26) the weight percentage of seco-rapamycin in the nanoparticles, as compared to the sum of seco-rapamycin and rapamycin, by weight; (27) the presence, percentage, or concentration of albumin stabilizer (such as sodium caprylate and/or N-acetyltryptophanate) in the composition; (28) the recovery of rapamycin following filtration; (29) in vitro release kinetics of the nanoparticles; (30) the portion of total rapamycin in the composition that is both in the non-nanoparticle portion of the composition and not bound to albumin; and/or (31) the weight percentage of seco-rapamycin in the composition, as compared to the sum of seco-rapamycin and rapamycin, by weight. As used herein, “albumin oligomers” or “oligomeric albumin” refers to lower molecular weight polymeric albumin species associated with a UV-absorbance-based size-exclusion chromatography peak observed between a peak associated with albumin dimers and higher molecular weight polymeric albumin species. In some embodiments, the oligomeric status (such as the percentage of albumin monomers, dimers, oligomers, or polymers (other than oligomers)) of the nanoparticles, the non-nanoparticle portion, or the total composition is assessed by size-exclusion chromatography using a mobile phase containing an aqueous portion and a miscible organic portion (such as an aqueous buffer containing 7.5% methanol) coupled with a UV detector. In some embodiments, the percentage of albumin in the nanoparticle portion that is in the form of monomeric, dimeric, oligomeric, or polymeric albumin (other than oligomeric albumin) is determined by separating the nanoparticles from the non-nanoparticle portion, dissolving the nanoparticles, and subjecting the dissolved nanoparticles to size-exclusion chromatography. In some embodiments, the size-exclusion chromatography uses a mobile phase containing an aqueous portion and a miscible organic portion (such as an aqueous buffer containing 7.5% methanol) coupled with a UV detector.

In some embodiments, the nanoparticle composition has one or more of the following distinct characteristics: (1) about 80% to about 95% (or as further provided herein) of the total albumin in the composition is in the form of monomeric albumin; (2) about 4% to about 15% (or as further provided herein) of the total albumin in the composition is in the form of dimeric albumin; (3) about 0.5% to about 5% (or as further provided herein) of the total albumin in the composition is in the form of polymeric albumin (or trimeric albumin); (4) the weight ratio of the total albumin to the total rapamycin in the composition is about 1:1 to about 10:1 (or as further provided herein); (5) about 90% or more (or as further provided herein) of the total rapamycin in the composition is in the nanoparticles; (6) about 90% or more (or as further provided herein) of the total albumin in the composition is in the non-nanoparticle portion of the nanoparticles; (7) the composition comprises tert-butanol at a concentration of less than about 10 μg/mL or less than about 10 ppm (or as further provided herein); (8) the composition comprises chloroform at a concentration of less than about 5 μg/mL or less than about 5 ppm (or as further provided herein); (9) the composition comprises an albumin stabilizer (such as sodium caprylate and/or N-acetyltryptophanate); (10) at least about 80% or more (or as further provided herein) of the rapamycin in the composition is recoverable after filtering the composition with a 0.2 micron filter; (11) the composition is stable for at least 24 hours; and/or (12) less than about 5% of the total rapamycin in the composition is both in the non-nanoparticle portion of the composition and unbound to albumin in the non-nanoparticle portion of the composition. In some embodiments, the nanoparticle composition may be a nanoparticle suspension, and the nanoparticle composition may have one or more of the following distinct characteristics (in addition to or in alternative to any one of the previously described district characteristics); (1) the concentration of albumin in the composition is about 30 mg/mL to about 100 mg/mL (or as further provided herein); (2) the concentration of rapamycin in the composition is about 1 mg/mL to about 15 mg/mL (or as further provided herein, such as about 1 mg/mL to about 7 mg/mL); (3) the osmolality of the composition is about 300 mOsm/kg to about 350 mOsm/kg (or as otherwise provided herein); (4) the viscosity of the composition is about 1.2 cP to about 1.5 cP (or as otherwise provided herein); and/or (5) the pH of the composition is about 6.0 to about 7.5 (or as otherwise provided herein).

In some embodiments, the nanoparticles of the composition have one or more of the following distinct characteristics: (1) about 70% to about 85% (or as otherwise provided herein) of the albumin in the nanoparticles is in the form of albumin monomers; (2) about 9% to about 20% (or as otherwise provided herein) of the albumin in the nanoparticles is in the form of albumin dimers; (3) about 5% to about 15% (or as otherwise provided herein) of the albumin in the nanoparticles is in the form of albumin polymers (or albumin trimers); (4) the nanoparticles have a volume weighted mean particle size and/or Z-average particle size of about 200 nm or less (or as otherwise provided herein, such as between about 50 nm and about 200 nm); (5) the nanoparticles have a polydispersity index of less than about 0.2 (or as otherwise provided herein, such as between about 0.03 and about 0.2); (6) the span of the particle size distribution ((Dv95−Dv5)/Dv50) is about 0.8 to about 1.2 (or as otherwise provided herein); (7) the nanoparticles are about 25% to about 45% albumin by weight (or as otherwise provided herein); (8) the nanoparticles are about 55% to about 75% rapamycin by weight (or as otherwise provided herein); (9) the weight ratio of albumin to rapamycin in the nanoparticles is about 1:1 to about 1:4 (or as otherwise provided herein); (10) the zeta potential of the nanoparticles in the composition is about −25 mV to about −50 mV (or as otherwise provided herein); (11) the nanoparticles have an amorphous morphology; (12) the rapamycin in the nanoparticles has an amorphous morphology; (13) the vinyl chain of the rapamycin in the nanoparticles interacts with the albumin in the nanoparticles; (14) at least a portion (such as at least 20%, or as otherwise provided herein) of the nanoparticles in the composition are non-spherical; (15) the nanoparticles comprise less than about 2.5% seco-rapamycin (or as otherwise provided herein, such as between about 0.2% and about 2.5%) compared to the sum of seco-rapamycin and rapamycin by weight; and/or (16) the composition comprises less than 3% seco-rapamycin (or as otherwise provided herein, such as between about 0.2% and about 2.5%) compared to the sum of seco-rapamycin and rapamycin by weight. In some embodiments, the nanoparticle composition may be a nanoparticle suspension, and in some embodiments the concentration of the albumin in the nanoparticle suspension that is in the nanoparticles is about 1.8 mg/mL to about 3 mg/mL (or as otherwise provided herein).

In some embodiments, the nanoparticles of the composition have one or more of the following distinct characteristics: (1) about 25% to about 50% (or as otherwise provided herein) of the albumin in the nanoparticles is in the form of albumin monomers; (2) about 5% to about 16% (or as otherwise provided herein) of the albumin in the nanoparticles is in the form of albumin dimers; (3) about 1% to about 4.5% (or as otherwise provided herein) of the albumin in the nanoparticles is in the form of albumin oligomers; (4) about 42% to about 60% (or as otherwise provided herein) of the albumin in the nanoparticles is in the form of albumin polymers (other than oligomers); (5) the nanoparticles have a volume weighted mean particle size and/or Z-average particle size of about 200 nm or less (or as otherwise provided herein, such as between about 50 nm and about 200 nm); (6) the nanoparticles have a polydispersity index of less than about 0.2 (or as otherwise provided herein, such as between about 0.03 and about 0.2); (7) the span of the particle size distribution ((Dv95−Dv5)/Dv50) is about 0.8 to about 1.2 (or as otherwise provided herein); (8) the nanoparticles are about 25% to about 45% albumin by weight (or as otherwise provided herein); (9) the nanoparticles are about 55% to about 75% rapamycin by weight (or as otherwise provided herein); (10) the weight ratio of albumin to rapamycin in the nanoparticles is about 1:1 to about 1:4 (or as otherwise provided herein); (11) the zeta potential of the nanoparticles in the composition is about −25 mV to about −50 mV (or as otherwise provided herein); (12) the nanoparticles have an amorphous morphology; (13) the rapamycin in the nanoparticles has an amorphous morphology; (14) the vinyl chain of the rapamycin in the nanoparticles interacts with the albumin in the nanoparticles; (15) at least a portion (such as at least 20%, or as otherwise provided herein) of the nanoparticles in the composition are non-spherical; (16) the nanoparticles comprise less than about 2.5% seco-rapamycin (or as otherwise provided herein, such as between about 0.2% and about 2.5%) compared to the sum of seco-rapamycin and rapamycin by weight; and/or (17) the composition comprises less than about 3% seco-rapamycin (or as otherwise provided herein, such as between about 0.2% and about 3%) compared to the sum of seco-rapamycin and rapamycin, by weight. In some embodiments, the nanoparticle composition may be a nanoparticle suspension, and in some embodiments the concentration of the albumin in the nanoparticle suspension that is in the nanoparticles is about 1.8 mg/mL to about 3 mg/mL (or as otherwise provided herein).

In some embodiments, the non-nanoparticle portion of the composition has one or more of the following distinct characteristics: (1) about 80% to about 95% (or as otherwise provided herein) of the albumin in the non-nanoparticle portion of the composition is in the form of albumin monomers; (2) about 5% to about 14% (or as otherwise provided herein) of the albumin in the non-nanoparticle portion of the composition is in the form of albumin dimers; and/or (3) about 1% to about 5% (or as otherwise provided herein) of the albumin in the non-nanoparticle portion of the composition is in the form of albumin polymers (or albumin trimers). In some embodiments, the nanoparticle composition may be a nanoparticle suspension, and the non-nanoparticle portion of the nanoparticle suspension may have one or more of the following distinct characteristics (in addition to or in alternative to any one of the previously described district characteristics): (1) the concentration of albumin in the non-nanoparticle portion of the composition is between about 30 mg/mL and about 100 mg/mL (or as otherwise provided herein); and/or (2) the concentration of rapamycin in the non-nanoparticle portion is about 20 μg/mL to about 55 μg/mL (or as otherwise provided herein).

In some embodiments, the non-nanoparticle portion of the composition has one or more of the following distinct characteristics: (1) about 80% to about 95% (or as otherwise provided herein) of the albumin in the non-nanoparticle portion of the composition is in the form of albumin monomers; (2) about 5% to about 16% (or as otherwise provided herein) of the albumin in the non-nanoparticle portion of the composition is in the form of albumin dimers; about 0.5% to about 4% (or as otherwise provided herein) of the albumin in the non-nanoparticle portion of the composition is in the form of albumin oligomers; and/or (4) about 0.5% to about 3% (or as otherwise provided herein) of the albumin in the non-nanoparticle portion of the composition is in the form of albumin polymers (other than oligomers). In some embodiments, the nanoparticle composition may be a nanoparticle suspension, and the non-nanoparticle portion of the nanoparticle suspension may have one or more of the following distinct characteristics (in addition to or in alternative to any one of the previously described district characteristics): (1) the concentration of albumin in the non-nanoparticle portion of the composition is between about 30 mg/mL and about 100 mg/mL (or as otherwise provided herein); and/or (2) the concentration of rapamycin in the non-nanoparticle portion is about 20 μg/mL to about 55 μg/mL (or as otherwise provided herein).

The compositions (such as pharmaceutical compositions) described herein can be in liquid (e.g., as a nanoparticle suspension) or powder forms. For example, in some embodiments, the composition is a liquid nanoparticle suspension (for example prior to lyophilization). In some embodiments, the composition is a reconstituted suspension (e.g., in an aqueous solution such as a saline solution). In some embodiments, the composition is dried, such as lyophilized. In some embodiments, the composition is sterile. In some embodiments, the composition is contained in a sealed container, such as a sealed vial (e.g., a glass vial) or sealed bag.

In some embodiments, the nanoparticle composition comprises (a) nanoparticles comprising rapamycin and albumin (such as human albumin), and (b) a non-nanoparticle portion comprising albumin (such as human albumin) and rapamycin. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising a coating comprising albumin (such as human albumin) and a core comprising rapamycin, and (b) a non-nanoparticle portion comprising albumin (such as human albumin) and rapamycin. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising rapamycin and albumin (such as human albumin), wherein about 70% to about 85% of the albumin in the nanoparticles is in the form of monomeric albumin; and (b) a non-nanoparticle portion comprising albumin (such as human albumin) and rapamycin. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising rapamycin and albumin (such as human albumin), wherein about 25% to about 50% of the albumin in the nanoparticles is in the form of monomeric albumin; and (b) a non-nanoparticle portion comprising albumin (such as human albumin) and rapamycin. In some embodiments, about 0.3% to about 4% of the albumin in the non-nanoparticle portion or the total albumin in the nanoparticle composition is in the form of oligomeric albumin. In some embodiments, about 0.5% to about 7% of the albumin in the non-nanoparticle portion or the total albumin in the nanoparticle composition is in the form of polymeric albumin (other than oligomeric albumin). In some embodiments, about 4% to about 15% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the percentage of albumin monomers, dimers, oligomers, or polymers (other than oligomers) is determined using size exclusion chromatography using a mobile phase containing an aqueous portion and a miscible organic portion (such as an aqueous buffer containing 7.5% methanol) coupled with a UV detector. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising a coating comprising albumin (such as human albumin) and a core comprising rapamycin, wherein about 70% to about 85% of the albumin in the nanoparticles is in the form of monomeric albumin; and (b) a non-nanoparticle portion comprising albumin (such as human albumin) and rapamycin. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising rapamycin and albumin (such as human albumin), wherein about 5% to about 15% of the albumin in the nanoparticles is in the form of polymeric albumin (or trimeric albumin); and (b) a non-nanoparticle portion comprising albumin (such as human albumin) and rapamycin. In some embodiments, the nanoparticle composition comprises less than 10 μg/mL tert-butanol and/or comprises less than 5 μg/mL chloroform. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising rapamycin and albumin (such as human albumin), wherein about 25% to about 50% of the albumin in the nanoparticles is in the form of polymeric albumin (other than oligomeric albumin); and (b) a non-nanoparticle portion comprising albumin (such as human albumin) and rapamycin. In some embodiments, the nanoparticle composition comprises less than 10 μg/mL tert-butanol and/or comprises less than 5 μg/mL chloroform. In some embodiments, about 0.5% to about 7% of the albumin in the non-nanoparticle portion or the total albumin in the nanoparticle composition is in the form of polymeric albumin (other than oligomeric albumin). In some embodiments, about 4% to about 15% of the albumin in the non-nanoparticle portion or the total albumin in the nanoparticle composition is in the form of dimeric albumin. In some embodiments, about 0.3% to about 4% of the albumin in the non-nanoparticle portion or the total albumin in the nanoparticle composition is in the form of oligomeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising a coating comprising albumin (such as human albumin) and a core comprising rapamycin, wherein about 5% to about 15% of the albumin in the nanoparticles is in the form of polymeric albumin (or trimeric albumin); and (b) a non-nanoparticle portion comprising albumin (such as human albumin) and rapamycin. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising rapamycin and albumin (such as human albumin), wherein about 9% to about 20% of the albumin in the nanoparticles is in the form of dimeric albumin, and (b) a non-nanoparticle portion comprising albumin (such as human albumin) and rapamycin. In some embodiments, the nanoparticle composition comprises less than 10 μg/mL tert-butanol and/or comprises less than 5 μg/mL chloroform. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising rapamycin and albumin (such as human albumin), wherein about 5% to about 16% of the albumin in the nanoparticles is in the form of dimeric albumin; and (b) a non-nanoparticle portion comprising albumin (such as human albumin) and rapamycin. In some embodiments, the nanoparticle composition comprises less than 10 μg/mL tert-butanol and/or comprises less than 5 μg/mL chloroform. In some embodiments, about 0.5% to about 7% of the albumin in the non-nanoparticle portion or the total albumin in the nanoparticle composition is in the form of polymeric albumin (other than oligomeric albumin). In some embodiments, about 0.3% to about 4% of the albumin in the non-nanoparticle portion or the total albumin in the nanoparticle composition is in the form of oligomeric albumin. In some embodiments, about 4% to about 15% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising a coating comprising albumin (such as human albumin) and a core comprising rapamycin, wherein about 9% to about 20% of the albumin in the nanoparticles is in the form of dimeric albumin; and (b) a non-nanoparticle portion comprising albumin (such as human albumin) and rapamycin. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising rapamycin and albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising rapamycin and albumin (such as human albumin), wherein about 25% to about 50% of the albumin in the nanoparticles is in the form of monomeric albumin, about 1% to about 4.5% of the albumin in the nanoparticles is in the form of oligomeric albumin, about 5% to about 16% of the albumin in the nanoparticles is in the form of dimeric albumin, and about 25% to about 50% of the albumin in the nanoparticles is in the form of polymeric albumin (other than oligomeric albumin); and (b) a non-nanoparticle portion comprising albumin (such as human albumin) and rapamycin. In some embodiments, about 0.5% to about 7% of the albumin in the non-nanoparticle portion or the total albumin in the nanoparticle composition is in the form of polymeric albumin (other than oligomeric albumin). In some embodiments, about 0.3% to about 4% of the albumin in the non-nanoparticle portion or the total albumin in the nanoparticle composition is in the form of oligomeric albumin. In some embodiments, about 4% to about 15% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising a coating comprising albumin (such as human albumin) and a core comprising rapamycin, wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 200 nm or less (such as about 50 nm to about 200 nm), comprising rapamycin and albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 200 nm or less (such as about 50 nm to about 200 nm), comprising a coating comprising albumin (such as human albumin) and a core comprising rapamycin, wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 200 nm or less (such as about 50 nm to about 200 nm), comprising about 55% to about 65% (by weight) rapamycin and about 25% to about 45% (by weight) albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 200 nm or less (such as about 50 nm to about 200 nm), comprising a coating comprising albumin (such as human albumin) and a core comprising rapamycin, wherein the albumin comprises about 25% to about 45% of the nanoparticles by weight and the rapamycin comprises about 55% to about 75% of the nanoparticles by weight, wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 200 nm or less (such as about 50 nm to about 200 nm), comprising about 55% to about 75% (by weight) rapamycin and about 25% to about 45% (by weight) albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL). In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 200 nm or less (such as about 50 nm to about 200 nm), comprising a coating comprising albumin (such as human albumin) and a core comprising rapamycin, wherein the albumin comprises about 25% to about 45% of the nanoparticles by weight and the rapamycin comprises about 55% to about 75% of the nanoparticles by weight, wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL). In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 200 nm or less (such as about 50 nm to about 200 nm) and a zeta potential of about −25 mV to about −50 mV, comprising about 55% to about 75% (by weight) rapamycin and about 25% to about 45% (by weight) albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL). In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 200 nm or less (such as about 50 nm to about 200 nm) and a zeta potential of about −25 mV to about −50 mV, comprising a coating comprising albumin (such as human albumin) and a core comprising rapamycin, wherein the albumin comprises about 25% to about 45% of the nanoparticles by weight and the rapamycin comprises about 55% to about 75% of the nanoparticles by weight, wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL). In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 200 nm or less (such as about 50 nm to about 200 nm) and a zeta potential of about −25 mV to about −50 mV, comprising about 55% to about 75% (by weight) rapamycin and about 25% to about 45% (by weight) albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein about 3% or less of the rapamycin in the nanoparticle composition is free rapamycin. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 200 nm or less (such as about 50 nm to about 200 nm) and a zeta potential of about −25 mV to about −50 mV, comprising a coating comprising albumin (such as human albumin) and a core comprising rapamycin, wherein the albumin comprises about 25% to about 45% of the nanoparticles by weight and the rapamycin comprises about 55% to about 75% of the nanoparticles by weight, wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein about 3% or less of the rapamycin in the nanoparticle composition is free rapamycin. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 200 nm or less (such as about 50 nm to about 200 nm) and a zeta potential of about −25 mV to about −50 mV, comprising about 55% to about 75% (by weight) rapamycin and about 25% to about 45% (by weight) albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein the sum of seco-rapamycin and rapamycin in the nanoparticles is less than 3% (such as about 0.2% to about 3%) seco-rapamycin, by weight. In some embodiments, the sum of seco-rapamycin and rapamycin in the composition is less than 3% (such as about 0.2% to about 3%) seco-rapamycin, by weight. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 200 nm or less (such as about 50 nm to about 200 nm) and a zeta potential of about −25 mV to about −50 mV, comprising a coating comprising albumin (such as human albumin) and a core comprising rapamycin, wherein the albumin comprises about 25% to about 45% of the nanoparticles by weight and the rapamycin comprises about 55% to about 75% of the nanoparticles by weight, wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein the sum of seco-rapamycin and rapamycin in the nanoparticles is less than 3% (such as about 0.2% to about 3%) seco-rapamycin, by weight. In some embodiments, the seco-rapamycin is less than 3% (such as about 0.2% to about 3%) of the sum of seco-rapamycin and rapamycin in the composition. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising rapamycin and albumin (such as human albumin), wherein about 74% to about 80% of the albumin in the nanoparticles is in the form of monomeric albumin; and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising a coating comprising albumin (such as human albumin) and a core comprising rapamycin, wherein about 74% to about 80% of the albumin in the nanoparticles is in the form of monomeric albumin; and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising rapamycin and albumin (such as human albumin), wherein about 7% to about 11% of the albumin in the nanoparticles is in the form of polymeric albumin (or trimeric albumin); and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising a coating comprising albumin (such as human albumin) and a core comprising rapamycin, wherein about 7% to about 11% of the albumin in the nanoparticles is in the form of polymeric albumin (or trimeric albumin); and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising rapamycin and albumin (such as human albumin), wherein about 12% to about 17% of the albumin in the nanoparticles is in the form of dimeric albumin, and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising a coating comprising albumin (such as human albumin) and a core comprising rapamycin, wherein about 12% to about 17% of the albumin in the nanoparticles is in the form of dimeric albumin; and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising rapamycin and albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising a coating comprising albumin (such as human albumin) and a core comprising rapamycin, wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 85 nm to about 95 nm, comprising rapamycin and albumin (such as human albumin); and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 85 nm to about 95 nm, comprising a coating comprising albumin (such as human albumin) and a core comprising rapamycin; and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 85 nm to about 95 nm, comprising rapamycin and albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 85 nm to about 95 nm, comprising a coating comprising albumin (such as human albumin) and a core comprising rapamycin, wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a zeta potential of about −33 mV to about −39 mV, comprising rapamycin and albumin (such as human albumin); and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a zeta potential of about −33 mV to about −39 mV, comprising a coating comprising albumin (such as human albumin) and a core comprising rapamycin; and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a zeta potential of about −33 mV to about −39 mV, comprising rapamycin and albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a zeta potential of about −33 mV to about −39 mV, comprising a coating comprising albumin (such as human albumin) and a core comprising rapamycin, wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 85 nm to about 95 nm and a zeta potential of about −33 mV to about −39 mV, comprising rapamycin and albumin (such as human albumin); and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 85 nm to about 95 nm and a zeta potential of about −33 mV to about −39 mV, comprising a coating comprising albumin (such as human albumin) and a core comprising rapamycin; and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 85 nm to about 95 nm and a zeta potential of about −33 mV to about −39 mV, comprising rapamycin and albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 85 nm to about 95 nm and a zeta potential of about −33 mV to about −39 mV, comprising a coating comprising albumin (such as human albumin) and a core comprising rapamycin, wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 85 nm to about 95 nm, comprising about 62% to about 68% (by weight) rapamycin and about 32% to about 38% (by weight) albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin), and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 85 nm to about 95 nm, comprising a coating comprising albumin (such as human albumin) and a core comprising rapamycin, wherein the albumin comprises about 32% to about 38% of the nanoparticles by weight and the rapamycin comprises about 62% to about 68% of the nanoparticles by weight, wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 85 nm to about 95 nm, comprising about 62% to about 68% (by weight) rapamycin and about 32% to about 38% (by weight) albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL). In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 85 nm to about 95 nm, comprising a coating comprising albumin (such as human albumin) and a core comprising rapamycin, wherein the albumin comprises about 32% to about 38% of the nanoparticles by weight and the rapamycin comprises about 62% to about 68% of the nanoparticles by weight, wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL). In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 85 nm to about 95 nm and a zeta potential of about −33 mV to about −39 mV, comprising about 62% to about 68% (by weight) rapamycin and about 32% to about 38% (by weight) albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL). In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 85 nm to about 95 nm and a zeta potential of about −33 mV to about −39 mV, comprising a coating comprising albumin (such as human albumin) and a core comprising rapamycin, wherein the albumin comprises about 32% to about 38% of the nanoparticles by weight and the rapamycin comprises about 62% to about 68% of the nanoparticles by weight, wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin), and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL). In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 85 nm to about 95 nm and a zeta potential of about −33 mV to about −39 mV, comprising about 62% to about 68% (by weight) rapamycin and about 32% to about 38% (by weight) albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein about 1% or less of the rapamycin in the nanoparticle composition is free rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 85 nm to about 95 nm and a zeta potential of about −33 mV to about −39 mV, comprising a coating comprising albumin (such as human albumin) and a core comprising rapamycin, wherein the albumin comprises about 32% to about 38% of the nanoparticles by weight and the rapamycin comprises about 62% to about 68% of the nanoparticles by weight, wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein about 1% or less of the rapamycin in the nanoparticle composition is free rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 85 nm to about 95 nm and a zeta potential of about of about −33 mV to about −39 mV, comprising about 62% to about 68% (by weight) rapamycin and about 32% to about 38% (by weight) albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein the sum of seco-rapamycin and rapamycin in the nanoparticles is less than 1% (such as about 0.5% to about 1%) seco-rapamycin, by weight. In some embodiments, seco-rapamycin is greater than about 0.2% (such as about 0.2% to about 3%) of the sum of seco-rapamycin and rapamycin in the composition. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles having a Z-average particle size of about 85 nm to about 95 nm and a zeta potential of about −33 mV to about −39 mV, comprising a coating comprising albumin (such as human albumin) and a core comprising rapamycin, wherein the albumin comprises about 32% to about 38% of the nanoparticles by weight and the rapamycin comprises about 62% to about 68% of the nanoparticles by weight, wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein the sum of seco-rapamycin and rapamycin in the nanoparticles is less than 1% (such as about 0.5% to about 1%) seco-rapamycin, by weight. In some embodiments, seco-rapamycin is greater than 0.2% (such as about 0.2% to about 3%) of the sum of seco-rapamycin and rapamycin in the composition. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin of the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL. In some embodiments, the osmolality of the composition is about 325 mOsm/kg to about 340 mOsm/kg. In some embodiments, the viscosity of the composition is about 1.3 cP to about 1.35 cP. In some embodiments, the pH of the composition is about 6.7 to about 6.8. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

Also provided herein are commercial batches of the nanoparticle compositions (such as the pharmaceutical compositions) for use of any one of the treatment methods described here. “Commercial batch” as used herein refers to a batch size that is at least about 20 grams (by mass of rapamycin). Commercial batches are produced at a larger scale than experimental or bench-scale batches. The increased scale is associated with longer production times, including longer steps (such as evaporation steps) or longer hold times between steps.

The commercial batches described herein, in some embodiments, comprise nanoparticle compositions (such as pharmaceutical compositions) that may have distinct characteristics for any one or more (in any combination) of the following: (1) the oligomeric status of the albumin associated with (such as in) the nanoparticles, such as the percentage of albumin monomers, dimers, oligomers, and/or polymers (or polymers other than oligomers) of the albumin associated with (such as in) the nanoparticles; (2) the oligomeric status of the albumin associated with (such as in) the non-nanoparticle portion of the composition, such as the percentage of albumin monomers, dimers, oligomers, and/or polymers (or polymers other than oligomers) of the albumin associated with (such as in) the non-nanoparticle portion of the composition; (3) the oligomeric status of the total albumin in the composition, such as the percentage of albumin monomers, dimers, oligomers, and/or polymers (or polymers other than oligomers) of the total albumin in the composition; (4) the particle size profile of the nanoparticles, such as the average particle size, polydispersity index, and/or size distribution; (5) the portion (e.g., weight percentage) of the nanoparticles that is albumin and/or the portion (e.g., weight percentage) of the nanoparticles that is rapamycin; (6) the weight ratio of the albumin to the rapamycin in the nanoparticles; (7) the weight ratio of the albumin to the rapamycin in the non-nanoparticle portion of the composition; (8) the weight ratio of the albumin to the rapamycin in the non-nanoparticle portion of the composition (9) the weight ratio of the total albumin to the total rapamycin in the composition; (10) the portion (e.g., weight percentage) of rapamycin that is in the nanoparticles (or the non-nanoparticle portion of the composition) compared to the total rapamycin in the composition; (11) the portion (e.g., weight percentage) of albumin that is in the non-nanoparticle portion (or in the nanoparticles) compared to the total albumin in the composition; (12) the concentration of albumin in the composition; (13) the concentration of albumin in the non-nanoparticle portion of the composition; (14) the concentration of albumin in the composition that is associated with (such as in) the nanoparticles; (15) the concentration of rapamycin in the composition; (16) the concentration of rapamycin in the non-nanoparticle portion of the composition; (17) the concentration of rapamycin in the composition that is associated with (such as in) the nanoparticles; (18) the osmolality of the composition; (19) the viscosity of the composition; (20) the pH of the composition; (21) the stability of the nanoparticles in the composition; (22) the amount of residual solvent in the composition; (23) the zeta potential of the nanoparticles in the composition; (24) the crystalline status of the rapamycin in the nanoparticles; (25) the particle morphology of the nanoparticles, such as the shape, sphericity, thickness of the coating, and/or surface-to-volume ratio; (26) the weight percentage of seco-rapamycin in the nanoparticles, as compared to the sum of seco-rapamycin and rapamycin, by weight; (27) the presence, percentage, or concentration of albumin stabilizer (such as a caprylic acid derivative e.g., sodium caprylate and/or a tryptophan derivative e.g., N-acetyltryptophanate) in the composition; (28) the recovery of rapamycin following filtration; (29) in vitro release kinetics of the nanoparticles; and/or (30) the portion of total rapamycin in the composition that is both in the non-nanoparticle portion of the composition and not bound to albumin. The physicochemical parameters discussed above can affect drug release and delivery of the albumin-based rapamycin nanoparticle compositions (such as pharmaceutical compositions), and thus constitute unique properties to the compositions in the commercial batches.

The commercial batches described herein, in some embodiments, comprise nanoparticle compositions (such as pharmaceutical compositions) that may have distinct characteristics for any one or more (in any combination) of the following: (1) the oligomeric status of the albumin associated with (such as in) the nanoparticles, such as the percentage of albumin monomers, dimers, and/or trimers of the albumin associated with (such as in) the nanoparticles; (2) the oligomeric status of the albumin associated with (such as in) the non-nanoparticle portion of the composition, such as the percentage of albumin monomers, dimers, and/or trimers of the albumin associated with (such as in) the non-nanoparticle portion of the composition; (3) the oligomeric status of the total albumin in the composition, such as the percentage of albumin monomers, dimers, and/or trimers of the total albumin in the composition; (4) the particle size profile of the nanoparticles, such as the average particle size, polydispersity index, and/or size distribution; (5) the portion (e.g., weight percentage) of the nanoparticles that is albumin and/or the portion (e.g., weight percentage) of the nanoparticles that is rapamycin; (6) the weight ratio of the albumin to the rapamycin in the nanoparticles; (7) the weight ratio of the albumin to the rapamycin in the non-nanoparticle portion of the composition; (8) the weight ratio of the albumin to the rapamycin in the non-nanoparticle portion of the composition (9) the weight ratio of the total albumin to the total rapamycin in the composition; (10) the portion (e.g., weight percentage) of rapamycin that is in the nanoparticles (or the non-nanoparticle portion of the composition) compared to the total rapamycin in the composition; (11) the portion (e.g., weight percentage) of albumin that is in the non-nanoparticle portion (or in the nanoparticles) compared to the total albumin in the composition; (12) the concentration of albumin in the composition; (13) the concentration of albumin in the non-nanoparticle portion of the composition; (14) the concentration of albumin in the composition that is associated with (such as in) the nanoparticles; (15) the concentration of rapamycin in the composition; (16) the concentration of rapamycin in the non-nanoparticle portion of the composition; (17) the concentration of rapamycin in the composition that is associated with (such as in) the nanoparticles; (18) the osmolality of the composition; (19) the viscosity of the composition; (20) the pH of the composition; (21) the stability of the nanoparticles in the composition; (22) the amount of residual solvent in the composition; (23) the zeta potential of the nanoparticles in the composition; (24) the crystalline status of the rapamycin in the nanoparticles; (25) the particle morphology of the nanoparticles, such as the shape, sphericity, thickness of the coating, and/or surface-to-volume ratio; (26) the weight percentage of seco-rapamycin in the nanoparticles, as compared to the sum of seco-rapamycin and rapamycin, by weight; (27) the presence, percentage, or concentration of albumin stabilizer (such as a caprylic acid derivative e.g., sodium caprylate and/or a tryptophan derivative e.g., N-acetyltryptophanate) in the composition; (28) the recovery of rapamycin following filtration; (29) in vitro release kinetics of the nanoparticles; and/or (30) the portion of total rapamycin in the composition that is both in the non-nanoparticle portion of the composition and not bound to albumin. The physicochemical parameters discussed above can affect drug release and delivery of the albumin-based rapamycin nanoparticle compositions (such as pharmaceutical compositions), and thus constitute unique properties to the compositions in the commercial batches.

In some embodiments, the commercial batch size is at least about any of 30 grams, 40 grams, 50 grams, 60 grams, 70 grams, 80 grams, 90 grams, 100 grams, 150 grams, 200 grams, 250 grams, 300 grams, 350 grams, 400 grams, 450 grams, 500 grams, 550 grams, 600 grams, 650 grams, 700 grams, 750 grams, 800 grams, 850 grams, 900 grams, 1000 grams, 1500 grams, 2000 grams, 2500 grams, 3000 grams, 3500 grams, 4000 grams, 4500 grams, 5000 grams, or 10000 grams (by amount of rapamycin). In some embodiments, the commercial batch comprises a plurality of containers, such as vials, comprising any of the compositions (such as pharmaceutical compositions) described herein. In some embodiments, the commercial batch comprises at least about any of 100 vials, 150 vials, 200 vials, 250 vials, 300 vials, 350 vials, 400 vials, 450 vials, 500 vials, 550 vials, 600 vials, 650 vials, 700 vials, 750 vials, 800 vials, 850 vials, 900 vials, 1000 vials, 1500 vials, 2000 vials, 2500 vials, 3000 vials, 3500 vials, 4000 vials, 4500 vials, 5000 vials, 10000 vials, 12000 vials, 14000 vials, 16000 vials, 18000 vials, 20000 vials, 22000 vials, 24000 vials, 26000 vials, 28000 vials, 30000 vials, 32000 vials, 34000 vials, 36000 vials, 38000 vials, 40000 vials, 42000 vials, 44000 vials, 46000 vials, 48000 vials, or 50000 vials. For example, each vial contains about any of 10 mg, 50 mg, 100 mg, 200 mg, 300 mg, 400 mg, 500 mg, 600 mg, 700 mg, 800 mg, 900 mg, or 1000 mg of the composition (such as a pharmaceutical composition). In some embodiments, each vial contains about any of 10 mg, 50 mg, 100 mg, 200 mg, 300 mg, 400 mg, 500 mg, 600 mg, 70) mg, 800 mg, 900 mg, or 1000 mg rapamycin. In some embodiments, the pharmaceutical composition in the commercial batch is a liquid suspension. In some embodiments, the pharmaceutical composition in the commercial batch is in a dried form, such as a lyophilized powder.

Thus, the present application in some embodiments provides a commercial batch of a composition (such as a pharmaceutical composition), for use in any of the described methods, comprising any one of the compositions or pharmaceutical compositions described herein (see more details in the sections above). For example, in some embodiments, there is provided a commercial batch of a pharmaceutical composition comprising: a) nanoparticles comprising rapamycin associated (such as coated) with albumin, and b) a non-nanoparticle portion comprising albumin and rapamycin. The characteristics and properties of the compositions contained with the commercial batch are described and defined throughout this application. Those characteristics and properties may be assessed for the commercial batch by assessment of a sample of the commercial batch.

### Cancer

The cancer treated by the methods complemented in the application can be any cancer that harbors one or more (such as one, two, three, four, five, or six) mTOR-activating aberration at any of the genes selected from the group consisting of TSC1, TSC2, TP53, RB1, ATRX, FAT1, PTEN, and RPS6. In some embodiments, the cancer harbors one or more mTOR-activating aberration at any one of genes selected from the group consisting of TSC, TSC2, TP53, and RPS6. In some embodiments, the cancer harbor at least one mTOR-activating aberration at RPS6 and at least one mTOR-activating aberration at TSC1, TSC2, or TP53. In some embodiments, the cancer harbor at least one mTOR-activating aberration at RPS6 and at least one mTOR-activating aberration at TSC1, or TSC2.

In some embodiments, the cancer is a solid tumor. In some embodiments, the cancer is a hematologic cancer.

In some embodiments, the cancer is advanced. In some embodiments, the cancer is malignant. In some embodiments, the cancer is an inoperable locally advanced cancer.

In some embodiments, the cancer is selected from the group consisting of pancreatic neuroendocrine cancer, endometrial cancer, breast cancer, lymphangioleiomyomatosis (LAM), prostate cancer, hepatocellular carcinoma, melanoma, renal cell carcinoma, bladder cancer, endometrial cancer, ovary cancer, gynecologic cancer, sarcoma, perivascular epithelioid cell neoplasms (PEComa), Hodgkin's lymphoma and multiple myeloma.

In some embodiments, the cancer is Ewing's sarcoma, PEComa, epithelioid sarcoma, desmoid tumor, chordoma, non-small cell lung cancer, small cell lung cancer, urethelial carcinoma, melanoma, renal cell carcinoma, squamous cell carcinoma of head and neck, hepatocellular carcinoma, classical Hodgkin's lymphoma, MSI-H/dMMR metastatic colorectal cancer, or a tumor with one or more genetic mutation sensitive to mTOR inhibitors. In some embodiments, the cancer is undifferentiated pleomorphic sarcoma. In some embodiments, the cancer is malignant. In some embodiments, the cancer is advanced. In some embodiments, the cancer is metastatic. In some embodiments, the cancer is metastatic or locally advanced. In some embodiments, surgery is not a recommended option for the cancer.

In some embodiments, the cancer is a PEComa. In some embodiments, the cancer is advanced PEComa. In some embodiments, the cancer is advanced and malignant PEComa. In some embodiments, the PEComa is a uterine primary PEComa. In some embodiments, the PEComa is retroperitoneal primary PEComa. In some embodiments, the PEComa is kidney primary PEComa. In some embodiments, the PEComa is lung primary PEComa. In some embodiments, the PEComa is pelvis primary PEComa.

In some embodiments, the tumor tissue is characterized with a TSC1 aberration (such as a TSC1 mutation). In some embodiments, the tumor tissue is characterized with a PTEN aberration (such as a PEN loss). In some embodiments, the tumor tissue is characterized with a TSC2 aberration (such as a TSC2 mutation). In some embodiments, the tumor tissue is characterized with a RB1 aberration (such as a RB1 loss). In some embodiments, the tumor tissue is characterized with a TP53 aberration (such as a TP53 mutation, such as a TP53 frameshift mutation). In some embodiments, the tumor tissue is characterized with an ATRX aberration (such as an ATRX mutation, such as an ATRX frameshift mutation). In some embodiments, the tumor tissue is characterized with an FA 1 aberration. In some embodiments, the tumor tissue is characterized with one, two, three, four, or five different aberrations selected from the group consisting of a PTEN aberration (such as a PTEN loss), a TSC2 aberration (such as a TSC2 mutation), a RB1 aberration (such as a RB1 loss), a TP53 aberration (such as a TP53 mutation, such as a TP53 frameshift mutation) and an ARX aberration (such as an ATRX mutation, such as an ATRX frameshift mutation).

In some embodiments, the tumor tissue is characterized with stable micro satellite status.

In some embodiments, the tumor tissue is characterized with low tumor mutation burden.

In some embodiments, the tumor tissue is characterized with both stable micro satellite status and low tumor mutation burden. In some embodiments, the tumor tissue is further characterized with a TSC1 aberration (such as a TSC1 mutation). In some embodiments, the tumor tissue is further characterized with a PTEN aberration (such as a PTEN loss). In some embodiments, the tumor tissue is further characterized with a TSC2 aberration (such as a TSC2 mutation). In some embodiments, the tumor tissue is further characterized with a RB1 aberration (such as a RB1 loss). In some embodiments, the tumor tissue is further characterized with a TP53 aberration (such as a TP53 mutation, such as a TP33 frameshift mutation). In some embodiments, the tumor tissue is further characterized with an ATRX aberration (such as an ATRX mutation, such as an ATRX frameshift mutation). In some embodiments, the tumor tissue is further characterized with an FAT1 aberration. In some embodiments, the tumor tissue is further characterized with one, two, three, four, or five different aberrations selected from the group consisting of a PTEN aberration (such as a PTEN loss), a TSC2 aberration (such as a TSC2 mutation), a RB1 aberration (such as a RB1 loss), a TP53 aberration (such as a TP53 mutation, such as a TP53 frameshift mutation) and an ATRX aberration (such as an ATRX mutation, such as an ATRX frameshift mutation).

### Individuals

In some embodiments, the individual did not respond to a prior therapy. In some embodiments, the individual did not respond to one, two, three, four or more prior therapies.

In some embodiments, the prior therapy comprises the administration of an mTOR inhibitor. In some embodiments, the mTOR inhibitor is everolimus.

In some embodiments, the prior therapy comprises the administration of an immune checkpoint inhibitor. In some embodiments, the immune checkpoint inhibitor is an anti-PD-1 antibody. Exemplary anti-PD-1 antibodies include nivolumab, pembrolizumab, cemiplimab, avelumab, durvalumab, and atezolizumab.

In some embodiments, the prior therapy comprises a chemotherapy. In some embodiments, the chemotherapy comprises the administration of doxorubicin. In some embodiments, the chemotherapy comprises the administration of an anti-neoplastic agent. In some embodiments, the chemotherapy comprises the administration of ifosfamide. In some embodiments, the chemotherapy comprises the administration of high-dose ifosfamide (such as a dose of 12 g/m² every four weeks). See Nielsen et al., Eur J Cancer. 2000 January; 36(1):61-7.

In some embodiments, the prior therapy further comprises a concurrent radiotherapy (for example, with administration of an anti-PD-1 antibody).

In some embodiments, the individual is a human. In some embodiments, the individual is at least about 12 years old, or at least about 18 years old.

In some embodiments, the individual is a female. In some embodiments, the individual is a post-menopausal female. In some embodiments, the individual is a male.

### Dosing and Method of Administering the Nanoparticle Compositions

The dose of the mTOR nanoparticles (such as a limus nanoparticle compositions) administered to an individual (such as a human) may vary with the particular composition, the mode of administration, and the kind of cancer being treated. In some embodiments, the amount of the composition is effective to result in an objective response (such as a partial response or a complete response). In some embodiments, the amount of the mTOR nanoparticle composition (such as a limus nanoparticle composition) is sufficient to result in a complete response in the individual. In some embodiments, the amount of the mTOR nanoparticle composition (such as a limus nanoparticle composition) is sufficient to result in a partial response in the individual. In some embodiments, the amount of the mTOR nanoparticle composition (such as a limus nanoparticle composition) administered (for example when administered alone) is sufficient to produce an overall response rate of more than about any of 20%, 30%, 40%, 50%, 60%, or 64% among a population of individuals treated with the mTOR nanoparticle composition (such as a limus nanoparticle composition). Responses of an individual to the treatment of the methods described herein can be determined, for example, based on RECIST levels, cystoscopy (with or without biopsy), biopsy, cytology, and CT imaging.

In some embodiments, the amount of the mTOR nanoparticle composition (such as a limus nanoparticle composition) is sufficient to produce a negative biopsy in the individual.

In some embodiments, the amount of the composition is sufficient to prolong progression-free survival of the individual. In some embodiments, the amount of the composition is sufficient to prolong overall survival of the individual. In some embodiments, the amount of the composition (for example when administered alone) is sufficient to produce clinical benefit of more than about any of 50%, 60%, 70%, or 77% among a population of individuals treated with the mTOR nanoparticle composition (such as a limus nanoparticle composition).

In some embodiments, the amount of the composition is an amount sufficient to decrease the size of a tumor, decrease the number of cancer cells, or decrease the growth rate of a tumor by at least about any of 10%, 20%, 30%, 40%, 50%, 60%, 70%, 80%, 90%, 95% or 100% compared to the corresponding tumor size or tumor growth rate in the same subject prior to treatment or compared to the corresponding activity in other subjects not receiving the treatment. Standard methods can be used to measure the magnitude of this effect, such as in vitro assays with purified enzyme, cell-based assays, animal models, or human testing.

In some embodiments, the amount of the mTOR inhibitor (such as a limus drug, for example sirolimus) in the composition is below the level that induces a toxicological effect (i.e., an effect above a clinically acceptable level of toxicity) or is at a level where a potential side effect can be controlled or tolerated when the composition is administered to the individual.

In some embodiments, the amount of the composition is close to a maximum tolerated dose (MTD) of the composition following the same dosing regime. In some embodiments, the amount of the composition is more than about any of 80%, 90%, 95%, or 98% of the MTD.

In some embodiments, the effective amounts of an mTOR inhibitor (e.g., a limus drug) in the nanoparticle composition include, but are not limited to, at least about any of 25 mg/m², 30 mg/m², 50 mg/m², 60 mg/m², 75 mg/m², 80 mg/m², 90 mg/m², 100 mg/m², 120 mg/m², 125 mg/m², 150 mg/m², 160 mg/m², 175 mg/m², 180 mg/m², 200 mg/m², 210 mg/m², 220 mg/m², 250 mg/m², 260 mg/m², 300 mg/m², 350 mg/m², 400 mg/m², 500 mg/m², 540 mg/m², 750 mg/m², 1000 mg/m², or 1080 mg/m² of an mTOR inhibitor (e.g., sirolimus). In various embodiments, the composition includes less than about any of 350 mg/m², 300 mg/m², 250 mg/m², 200 mg/m², 150 mg/m², 120 mg/m², 100 mg/m², 90 mg/m², 50 mg/m², or 30 mg/mⁿ of an mTOR inhibitor (e.g., sirolimus). In some embodiments, the amount of the mTOR inhibitor (e.g., sirolimus) per administration is less than about any of 25 mg/n, 22 mg/m², 20 mg/m², 18 mg/m², 15 mg/m², 14 mg/m², 13 mg/m², 12 mg/m², 11 mg/m², 10 mg/m², 9 mg/m², 8 mg/m², 7 mg/m², 6 mg/m², 5 mg/m², 4 mg/m², 3 mg/m², 2 mg/m², or 1 mg/m². In some embodiments, the effective amount of an mTOR inhibitor (e.g., sirolimus) in the composition is included in any of the following ranges: about 1 to about 5 mg/m², about 5 to about 10 mg/m², about 10 to about 25 mg/m², about 25 to about 50 mg/m², about 50 to about 75 mg/m², about 75 to about 100 mg/m², about 100 to about 125 mg/m², about 125 to about 150 mg/m², about 150 to about 175 mg/m², about 175 to about 200 mg/m², about 200 to about 225 mg/m², about 225 to about 250 mg/m², about 250 to about 300 mg/m², about 300 to about 350 mg/m², or about 350 to about 400 mg/m². In some embodiments, the effective amount of an mTOR inhibitor (e.g., sirolimus) in the composition is about 5 to about 300 mg/n, such as about 100 to about 150 mg/n, about 120 mg/m², about 130 mg/m², or about 140 mg/m². In some embodiments, the effective amount of an mTOR inhibitor (e.g., sirolimus) in the composition is about 50 mg/m² to about 100 mg/m².

In some embodiments of any of the above aspects, the effective amount of an mTOR inhibitor (e.g., sirolimus) in the composition includes at least about any of 1 mg/kg, 2.5 mg/kg, 3.5 mg/kg, 5 mg/kg, 6.5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, or 60 mg/kg. In various embodiments, the effective amount of an mTOR inhibitor (e.g., sirolimus) in the composition includes less than about any of 350 mg/kg, 300 mg/kg, 250 mg/kg, 200 mg/kg, 150 mg/kg, 100 mg/kg, 50 mg/kg, 25 mg/kg, 20 mg/kg, 10 mg/kg, 7.5 mg/kg, 6.5 mg/kg, 5 mg/kg, 3.5 mg/kg, 2.5 mg/kg, or 1 mg/kg of an mTOR inhibitor (e.g., sirolimus).

In some embodiments, the dosing frequencies for the administration of the nanoparticle compositions include, but are not limited to, daily, every two days, every three days, every four days, every five days, every six days, weekly without break, three out of four weeks, once every three weeks, once every two weeks, or two out of three weeks. In some embodiments, the composition is administered about once every 2 weeks, once every 3 weeks, once every 4 weeks, once every 6 weeks, or once every 8 weeks. In some embodiments, the composition is administered at least about any of 1×, 2×, 3×, 4×, 5×, 6×, or 7× (i.e., daily) a week. In some embodiments, the intervals between each administration are less than about any of 6 months, 3 months, 1 month, 20 days, 15, days, 14 days, 13 days, 12 days, 11 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

In some embodiments, the dosing frequency is once every two days for one time, two times, three times, four times, five times, six times, seven times, eight times, nine times, ten times, and eleven times. In some embodiments, the dosing frequency is once every two days for five times. In some embodiments, the mTOR inhibitor (e.g., sirolimus) is administered over a period of at least ten days, wherein the interval between each administration is no more than about two days, and wherein the dose of the mTOR inhibitor (e.g., sirolimus) at each administration is about 0.25 mg/m² to about 250 mg/m², about 0.25 mg/m² to about 150 mg/m², about 0.25 mg/m² to about 75 mg/m², such as about 0.25 mg/m² to about 25 mg/m², or about 25 mg/m² to about 50 mg/m².

In some embodiments, the dose of the mTOR inhibitor (e.g., sirolimus) for each administration is at least about 10 mg/m² to 100 mg/m² (such as about 25 mg/m² to 100 mg/m², 50 mg/m² to 100 mg/m², 75 mg/m² to 100 mg/m²).

In some embodiments, the average weekly dose of the mTOR inhibitor (e.g., sirolimus) in a cycle (counting in the rest period) is no more than 100 mg/m² (such as no more than about 90 mg/m², 80 mg/m², or 70 mg/m²).

The administration of the composition can be extended over an extended period of time, such as from about a month up to about seven years. In some embodiments, the composition is administered over a period of at least about any of 2, 3, 4, 5, 6, 7, 8, 9, 10, 11, 12, 18, 24, 30, 36, 48, 60, 72, or 84 months.

In some embodiments, the dosage of an mTOR inhibitor (e.g., sirolimus) in a nanoparticle composition can be in the range of 5-400 mg/m² when given on a 3 week schedule, or 5-250 mg/m² (such as 80-150 mg/m², for example 100-120 mg/m²) when given on a weekly schedule. For example, the amount of an mTOR inhibitor (e.g., sirolimus) is about 60 to about 300 mg/m² (e.g., about 260 mg/m²) on a three week schedule.

In some embodiments, the exemplary dosing schedules for the administration of the nanoparticle composition (e.g., sirolimus/albumin nanoparticle composition) include, but are not limited to, 100 mg/m², weekly, without break; 100 mg/m², weekly, 2 out of 3 weeks; 100 mg/m², weekly, 3 out of 4 weeks; 75 mg/m², weekly, without break; 75 mg/m², weekly, 2 out of 3 weeks; 75 mg/m², weekly, 3 out of 4 weeks; 56 mg/m², weekly, without break; 56 mg/m², weekly, 2 out of 3 weeks; 56 mg/m², weekly, 3 out of 4 weeks. The dosing frequency of the composition may be adjusted over the course of the treatment based on the judgment of the administering physician.

In some embodiments, the individual is treated for at least about any of one, two, three, four, five, six, seven, eight, nine, or ten treatment cycles.

The compositions described herein allow infusion of the composition to an individual over an infusion time that is shorter than about 24 hours. For example, in some embodiments, the composition is administered over an infusion period of less than about any of 24 hours, 12 hours, 8 hours, 5 hours, 3 hours, 2 hours, 1 hour, 30 minutes, 20 minutes, or 10 minutes. In some embodiments, the composition is administered over an infusion period of about 30 minutes.

In some embodiments, the exemplary dose of the mTOR inhibitor (in some embodiments a limus drug, for example, sirolimus) in the nanoparticle composition include, but is not limited to, about any of 50 mg/m², 60 mg/m², 75 mg/m², 80 mg/m², 90 mg/m², 100 mg/m², 120 mg/m², 160 mg/m², 175 mg/m², 200 mg/m², 210 mg/m², 220 mg/m², 260 mg/m², and 300 mg/m². For example, the dosage of an mTOR inhibitor in a nanoparticle composition can be in the range of about 100-400 mg/m² when given on a 3 week schedule, or about 50-250 mg/m when given on a weekly schedule.

The mTOR nanoparticle composition (such as a limus nanoparticle composition) can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, sustained continuous release formulation of the composition may be used. In some embodiments, the composition is administered intravenously. In some embodiments, the composition is administered subcutaneously. In some embodiments, the composition is administered intravesicularly. In some embodiments, the composition is administered intraarterially. In some embodiments, the composition is administered intraperitoneally.

In some embodiments when the limus nanoparticle composition is administered intravesicularly, the dosage of an mTOR inhibitor (such as a limus drug, e.g., sirolimus) in a nanoparticle composition can be in the range of about 30 mg to about 400 mg in volume of about 20 to about 150 ml, for example retained in the bladder for about 30 minutes to about 4 hours. In some embodiments, the nanoparticle composition is retained in the bladder for about 30 minutes to about 4 hours, including for example about 30 minutes to about 1 hour, about 1 hour to about 2 hours, about 2 hours to about 3 hours, or about 3 hours to about 4 hours.

In some embodiments, the dosage of an mTOR inhibitor (such as a limus drug, e.g., sirolimus) is about 100 to about 400 mg, for example about 100 mg, about 200 mg, about 300 mg, or about 400 mg. In some embodiments, the limus drug is administered at about 100 mg weekly, about 200 mg weekly, about 300 mg weekly, about 100 mg twice weekly, or about 200 mg twice weekly. In some embodiments, the administration is further followed by a monthly maintenance dose (which can be the same or different from the weekly doses).

In some embodiments when the limus nanoparticle composition is administered intravenously, the dosage of an mTOR inhibitor (such as a limus drug, e.g., sirolimus) in a nanoparticle composition can be in the range of about 30 mg to about 400 mg. The compositions described herein allow infusion of the composition to an individual over an infusion time that is shorter than about 24 hours. For example, in some embodiments, the composition is administered over an infusion period of less than about any of 24 hours, 12 hours, 8 hours, 5 hours, 3 hours, 2 hours, 1 hour, 30 minutes, 20 minutes, or 10 minutes. In some embodiments, the composition is administered over an infusion period of about 30 minutes to about 40 minutes.

### Combination Therapy

The methods described herein for treating cancer can be used in combination therapy with a second agent. The second agent may be a chemotherapeutic agent or an antibody. In some embodiments, the other therapeutic agent is selected from the group consisting of an alkylating agent, an anthracycline antibiotic, a DNA crosslinking agent, an antimetabolite, an indolequinone, a taxane, or a platinum-based agent.

In some embodiments, the second agent comprises an immune checkpoint inhibitor. In some embodiments, the immune checkpoint inhibitor specifically targets PD-1 or PD-L1.

In some embodiments, the immune checkpoint inhibitor is an anti-PD-1 antibody. In some embodiments, the anti-PD-1 antibody is administered at a dose of about 1 mg/kg to about 10 mg/kg (such as about 3 mg/kg) for a human individual. In some embodiments, the anti-PD-1 antibody is administered once a week, once every two weeks, or once every three weeks. In some embodiments, the anti-PD-1 antibody is administered at a dose of about 3 mg/kg for a human individual once every three weeks.

### Kits, Medicines and Compositions

The present application also provides kits, medicines, compositions, and unit dosage forms for use in any of the methods described herein.

In some embodiments, there is provided a kit comprising (a) a composition comprising nanoparticles comprising an mTOR inhibitor (such as a limus drug) and a carrier protein (e.g., albumin); and (b) one or more agents for assessing an mTOR-activating aberration at one or more (such as one, two, three, four, five, or six) of genes selected from the group consisting of TSC1, TSC2, RPS6, PTEN, 1P53, RB, ATRX, and FAT1). In some embodiment, the one or more (such as one, two or three) genes is selected from TSC1, TSC2, and RPS6. In some embodiments, there is provided a kit comprising (a) a composition comprising nanoparticles comprising an mTOR inhibitor (such as a limus drug) and a carrier protein (e.g., albumin); (b) a first agent for assessing mutation of a gene selected from the group consisting of 1C, 15C2, PTEN, TP53, RB1, ATRX, and FAT1, c) a second agent for assessing phosphorylation level of a protein encoded by RPS6. In some embodiments, there is provided a kit comprising (a) a composition comprising nanoparticles comprising an mTOR inhibitor (such as a limus drug) and a carrier protein (e.g., albumin), (b) a first agent for assessing TSC2 mutation, c) a second agent for assessing phosphorylation level of a protein encoded by RPS6. In some embodiments, there is provided a kit comprising (a) a composition comprising nanoparticles comprising an mTOR inhibitor (such as a limus drug) and a carrier protein (e.g., albumin); (b) a first agent for assessing TSC1 mutation, c) a second agent for assessing phosphorylation level of a protein encoded by RPS6.

In some embodiments, the agent comprises a nucleic acid specific for the mTOR-associated gene. In some embodiments, the agent comprises an antibody that specifically recognizes a protein encoded by the mTOR-associated gene. In some embodiments, the kit further comprises instructions for use in accordance with any of the methods described herein including methods for treating, assessing responsiveness, monitoring, identifying individuals, and selecting patients for treatment of a cancer using the mTOR inhibitor nanoparticle composition based upon the status of the mTOR-activating aberration.

In some embodiments, the kit further comprises an agent for assessing the mutational status of a resistance biomarker, such as TFE3. In some embodiments, the kit further comprises instructions for using the mutational status of the resistance biomarker for selecting individuals for treatment of a cancer based on the mutational status of the resistance biomarker alone or in combination with at least one mTOR-activating aberration.

Kits of the invention may include one or more containers comprising the mTOR inhibitor (such as limus drug) nanoparticle compositions (or unit dosage forms and/or articles of manufacture), and one or more containers comprising the agent for assessing the mTOR-activating aberration.

In some embodiments, the kit comprises a second therapeutic agent. The nanoparticle compositions and the second therapeutic agent can be present in separate containers or in a single container. For example, the kit may comprise one distinct composition or two or more compositions wherein one composition comprises nanoparticles and one composition comprises the second therapeutic agent.

The kits of the invention are in suitable packaging. Suitable packaging include, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the nanoparticle compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of the mTOR inhibitor (such as a limus drug. e.g., sirolimus) as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the mTOR inhibitor (such as a limus drug) and pharmaceutical compositions and instructions for use and packaged in quantities sufficient for storage and use in pharmacies, for example, hospital pharmacies and compounding pharmacies.

Also provided are medicines, compositions, and unit dosage forms useful for the methods described herein. In some embodiments, there is provided a medicine (or composition) for use in treating a cancer comprising nanoparticles comprising an mTOR inhibitor (such as a limus drug) and a carrier protein (such as an albumin).

In some embodiments, there is a pharmaceutical composition comprising an mTOR inhibitor (such as a limus drug) and a carrier protein (such as an albumin) for use in any of the methods described herein for treating a cancer.

In some embodiments, the pharmaceutical compositions further comprise an agent or agents for enhancing dissolution of dried forms of the compositions and/or enhancing the stability of the composition. In some embodiments, the additional agent or agents comprise a saccharide. The saccharide may be, but is not limited to, monosaccharides, disaccharides, polysaccharides, and derivatives or modifications thereof. The saccharide may be, for example, any of mannitol, sucrose, fructose, lactose, maltose, dextrose, or trehalose. In some embodiments, the additional agent or agents comprise glycine. The present application therefore in one aspect provides a pharmaceutical composition suitable for subcutaneous administration to an individual comprising a) nanoparticles comprising an mTOR inhibitor (such as rapamycin) and an albumin, and b) a saccharide.

In some embodiments, the saccharide is present in an amount that is effective to increase the stability of the nanoparticles in the composition as compared to a nanoparticle composition without the saccharide. In some embodiments, the saccharide is in an amount that is effective to improve filterability of the nanoparticle composition as compared to a composition without the saccharide.

In some embodiments, the saccharide is present in an amount effective to enhance the solubility of the pharmaceutical composition. In some embodiments, the enhanced solubility comprises improved rate of dissolution of a dried form of the nanoparticle composition after addition of a reconstituting solution.

In some embodiments, the saccharide is present in an amount that reduces the incidence or severity of post-administration side effects when the nanoparticle composition is administered subcutaneously. For example, in some embodiments, the side effect is rash and the composition comprises nanoparticles comprising an mTOR inhibitor and an albumin and the saccharide is present in an amount that reduces the incidence of rash after subcutaneous administration of the nanoparticle composition.

### Exemplary Embodiments

Embodiment 1. A method of treating a cancer in an individual comprising administering to the individual an effective amount of a composition comprising nanoparticles comprising an mTOR inhibitor and a carrier protein, wherein the individual is selected for treatment on the basis of having an mTOR-activating aberration at TSC2 or RPS6.

Embodiment 2. The method of embodiment 1, wherein the individual is selected for treatment on the basis of having an mTOR-activating aberration at TSC2 and RPS6.

Embodiment 3. The method of embodiment 1 or embodiment 2, wherein the mTOR-activating aberration at TSC2 comprises a mutation in TSC2.

Embodiment 4. The method of any one of embodiment 1-3, wherein the mTOR-activating aberration at TSC2 comprises a single-nucleotide variant (SNV).

Embodiment 5. The method of embodiment 4, wherein the SNV comprises a mutation selected from the group consisting of C1503T, C2743G, C5383T, C3755G, G760T, C3442T, G880A, T707C, A4949G, or a deletion of any one or more of the amino acids at the position of 1405-1409, 1960-1970, 4999, 5002, 3521, 5208, 5238-5255.

Embodiment 6. The method of any one of embodiments 1-5, wherein the mTOR-activating aberration at TSC2 comprises a copy number variation of TSC2.

Embodiment 7. The method of any one of embodiments 1-6, wherein the mTOR-activating aberration at TSC2 is a loss of function mutation.

Embodiment 8. The method of any one of embodiments 1-7, wherein the mTOR-activating aberration at TSC2 comprises an aberrant expression level of TSC2.

Embodiment 9. The method of any one of embodiments 1-8, wherein the mTOR-activating aberration at TSC2 comprises an aberrant activity level of a protein encoded by TSC2.

Embodiment 10. The method of any one of embodiments 1-9, wherein the mTOR-activating aberration at TSC2 comprises a loss of heterozygosity of TSC2.

Embodiment 11. A method of treating a cancer in an individual comprising administering to the individual an effective amount of a composition comprising nanoparticles comprising an mTOR inhibitor and a carrier protein, wherein the individual is selected for treatment on the basis of having an mTOR-activating aberration at TSC1 or RPS6.

Embodiment 12. The method of any one of embodiments 1-11, wherein the mTOR-activating aberration at RPS6 comprises an aberrant phosphorylation level of the protein encoded by RPS6.

Embodiment 13. The method of any one of embodiments 1-12, wherein the mTOR-activating aberration at RPS6 comprises an aberrant expression level of RPS6.

Embodiment 14. The method of any one of embodiments 1-13, wherein the cancer is advanced and/or malignant.

Embodiment 15. The method of any one of embodiments 1-14, wherein the cancer is a solid tumor.

Embodiment 16. The method of any one of embodiments 1-14, wherein the cancer is a hematologic cancer.

Embodiment 17. The method of any one of embodiments 1-16, wherein the cancer is selected from the group consisting of pancreatic neuroendocrine cancer, endometrial cancer, breast cancer, lymphangioleiomyomatosis (LAM), prostate cancer, hepatocellular carcinoma, melanoma, renal cell carcinoma, bladder cancer, endometrial cancer, ovary cancer, gynecologic cancer, sarcoma, perivascular epithelioid cell neoplasms (PEComa), Hodgkin's lymphoma and multiple myeloma.

Embodiment 18. The method of any one of embodiments 1-17, wherein the nanoparticles in the composition comprises the mTOR inhibitor associated with the carrier protein.

Embodiment 19. The method of any one of embodiments 1-18, wherein the nanoparticles in the composition have an average diameter of no greater than about 200 nm.

Embodiment 20. The method of any one of embodiments 1-19, wherein the ratio of the mTOR inhibitor to the carrier protein in the nanoparticles is from about 1:1 to about 9:1.

Embodiment 21. The method of any one of embodiments 1-20, wherein the carrier protein is an albumin.

Embodiment 22. The method of embodiment 21, wherein the albumin is human serum albumin.

Embodiment 23. The method of any one of embodiments 1-22, wherein the mTOR inhibitor is a limus drug.

Embodiment 24. The method of embodiment 23, wherein the limus drug is rapamycin.

Embodiment 25. The method of any one of embodiments 1-24, wherein the dose of the mTOR inhibitor in the composition for each administration is from about 10 mg/m² to about 100 mg/m².

Embodiment 26. The method of any one of embodiments 1-25, wherein nanoparticle composition is administered at a frequency of about once a week to about once every two weeks.

Embodiment 27. The method of any one of embodiments 1-26, wherein the method comprises administering the nanoparticle composition to the individual weekly for about two weeks followed by a rest period of about one week.

Embodiment 28. The method of any one of embodiments 1-27, wherein the individual is resistant or refractory to a prior therapy.

Embodiment 29. The method of any one of embodiments 1-28, wherein the method further comprises administering a second agent.

Embodiment 30. The method of any one of embodiments 1-29, wherein the individual is a human.

Embodiment 31. The method of any one of embodiments 1-10 and 12-30, wherein the individual does not comprise a mutation in TSC1.

Embodiment 32. The method of any one of embodiments 1-31, wherein the method further comprises assessing the mTOR-activating aberration at TSC1, TSC2, or RPS6 in the individual.

Embodiment 33. The method of any one of embodiments 1-32, wherein the method further comprises selecting the individual for treatment based on the individual having the mTOR-activating aberration at TSC1, TSC2 or RPS6.

### EXAMPLES

The examples below are intended to be purely exemplary of the invention and should therefore not be considered to limit the invention in any way. The following examples and detailed description are offered by way of illustration and not by way of limitation.

### Example 1. Phase 11 Study Multi-Center Study with Patients Receiving ABI-009 Treatment

Patients with advanced malignant PEComa (a rare, aggressive sarcoma, with no approved treatment available) who previously have not been treated with an mTOR inhibitor were enrolled in a phase II study, single arm, open label, multi-institutional study to assess the efficacy and safety profile of intravenous ABI-009 (also referred to herein as nab-sirolimus or nab-rapamycin, produced as described in Example 7).

Key eligibility requirements include that patients a) were at least 18 years old at the time of enrollment, b) had Eastern Cooperative Oncology Group (ECOG) performance status 0 or 1, c) had histological confirmation of a PEComa; d) had locally advanced inoperable or metastatic disease; and 3) had no prior treatment with an mTOR inhibitor.

Patients received ABI-009 at a dose of 100 mg/m² for two of every 3 weeks by IV infusion over 30 minutes. Two dose reduction levels were allowed: 75 mg/m² and 56 mg/m². Patients continued the treatment until disease progression, unacceptable toxicity, until in the opinion of the investigator the patient was no longer benefiting from therapy, or at the patients discretion.

Primary endpoints include ORR by independent assessment CT/MRI (RECIST v1.1) every 6 weeks. Secondary endpoints include DOR, PFS at 6 months, median PFS, median OS and safety. Exploratory endpoints included multiple biomarkers: mutational analysis (oncopanel) was by next-generation sequencing of a 500-gene panel, including TSC1, TSC2, TP53, PTEN, and FAT1, TFE3 translocation analysis was done via FISH. Immunohistochemistry included phosphorylated S6, 4EBP1, and AKT and percentage Ki67. Sample size: based on an estimated ORR of 30% in 30 efficacy-evaluable patients, the lower bound of the 95% CI will exclude values less than 14.7%. The primary analysis was prospectively planned when all patients were treated ≥6 months. Efficacy Evaluable Patients must receive ≥1 dose of nab-sirolimus and must have centrally confirmed PEComa.

### Demographics and Characteristics

See Table 1 below for an analysis of demographics and characteristics.

TABLE 1

| Variable | All Patients (N = 34) |
|---|---|
| Age, median (range), years | 60 (range: 27-78) |
| ≥65 years, n (%) | 15 (44%) |
| Female, n (%) | 28 (82%) |
| Race, n (%) | |
| White | 24 (71%) |
| Black | 3 (9%) |
| Asian | 3 (9%) |
| Pacific Islander/Hawaiian | 1 (3%) |
| Unknown | 3 (9%) |
| ECOG 0, n (%) | 26 (76%) |
| ECOG 1, n (%) | 8 (24%) |
| Metastatic, n (%) | 29 (85%) |
| Locally Advanced, inoperable, n (%) | 5 (15%) |
| Prior Systemic Rx for Advanced PEComa,* n (%) | 4 (12%) |

*docetaxel, doxorubicin, gemcitabine, ifosfamide, olaratumab

### Primary Sites of the Diseases and Most Comment Metastatic Sites

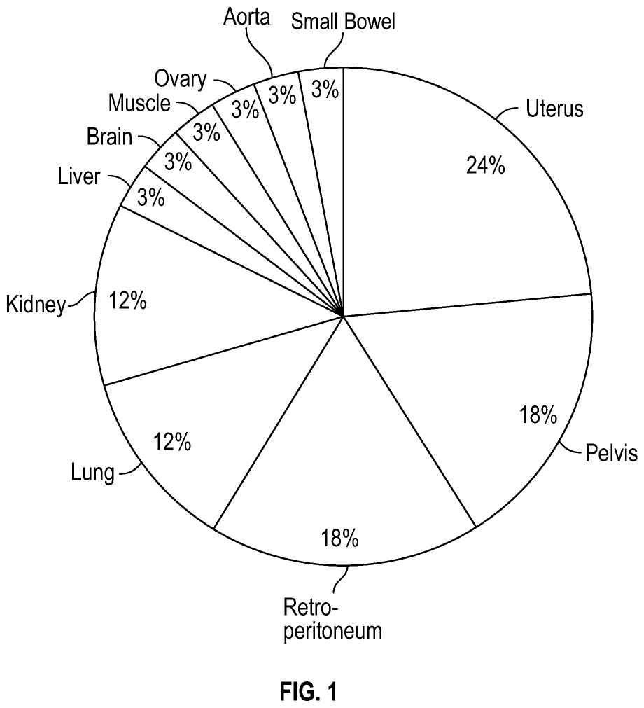

Primary sites of the diseases were shown in FIG. 1. Table 2 lists most common metastatic sites. Specifically, the most common primary site of PEComa was the uterus (24%), pelvis (18%), and retroperitoneum (18%).

TABLE 2

| Most Common Metastatic Sites | N = 29 |
|---|---|
| Lung | 21 (72%) |
| Liver | 6 (21%) |
| Abdomen * | 8 (28%) |
| Pelvis | 5 (17%) |

* Includes abdomen, colon, omentum, perigastric area, peritoneum, serosa

### Safety

Summaries of treatment-related adverse events (TR AEs) and treatment-related serious adverse effects were shown in Tables 3 and 4 below.

TABLE 3

| | Any Grade > 25% | Grade 3* |
|---|---|---|
| TR AEs | n (%) | n (%) |
| Patients with Any TR AEs | 34 (100) | |

Hematologic TRAEs

| | | |
|---|---|---|
| Anemia | 16 (47) | 4 (12) |
| Thrombocy topenia | 11(32) | 1 (3) |

Nonhematologic TRAEs

| | | |
|---|---|---|
| Stomatitis/Mucositis | 27 (79) | 6 (18) |
| Rash | 19 (56) | — |
| Fatigue | 20 (59) | 1 (3) |
| Nausea | 16 (47) | — |
| Diarrhea | 13 (38) | — |
| Weight Decreased | 13 (38) | — |
| Hyperglycemia | 12 (35) | 3 (9) |
| Hypertriglyceridemia | 11 (32) | 1 (3) |
| Hy percholesterol emia | 11 (32) | — |
| Decreased Appetite | 11 (32) | — |
| Dermatitis | 10 (29) | — |
| Dysgeusia | 10 (29) | — |
| Headache | 10 (29) | — |
| Peripheral Edema | 9 (26) | — |

*Additional G3 TRAEs were 6% hypokalemia, and 3% each of AST/ALT, amylase ↑, hypophosphatemia, insomnia, lipase ↑, lymphocyte ↓, skin infection, vomiting.

TABLE 4

| TR Serious AEs | n (%) |
|---|---|
| Patients with Any TR SAE | 8 (24) |
| Dehydration (G3) | 2 (6) |
| Abdominal pain (G2) | 1 (3) |
| Diarrhea (G2.) | 1 (3) |
| Edema (3) | 1 (3) |
| Enteritis (G3) | 1 (3) |
| Pancytopenia (G3) | 1 (3) |
| Acute Coronary Syndrome (G3) | 1 (3) |
| Acute Kidney Injun' (G3) | 1 (3) |

As shown in Tables 3 and 4, no grade 4 or grade 5 treatment-related adverse events. Grade 1 or Grade 2 pneumonitis was seen in six out of thirty-four patients (about 18%). No unexpected AEs were shown. Two out of thirty-four patients had an adverse event that resulted in discontinuation (which was Grade 2 anemia and Grade 1 cystitis, respectively). Additional Grade 3 adverse events were: hypokalemia (6%), AST/ALT (3%), increased amylase (3%), hypophosphatemia (3%), insomnia (3%), increased lipase (3%), decreased lymphocyte (3%), skin infection (3%), and vomiting (3%).

### Treatment Exposure

The enrollment closed in November 2018. Ten out of thirty-four patients were still on treatment as of the cutoff date on May 22, 2019. See Table 5.

TABLE 5

| | nab-sirolimus |
|---|---|
| Variable | (N = 34) |
| Median Follow-up, median months (min, max) | 11.5 (1, 37+) |
| Number of Treatment Cycles, median (Min, max) | 8.5 (1, 46+) |
| Patients with a dose reduction, n (%) | 13 (38) |
| 1 dose reduction | 11 (32) |
| 2 dose reductions | 2 (6) |
| Patients with a dose delay, n (%) | 24 (71) |
| % of Protocol Dose, median mg/m² (min, max) | 92 (45, 100) |
| Average Dose Intensity, median mg/m²/week (min, max)* | 62 (30, 67) |

### Response Assessment

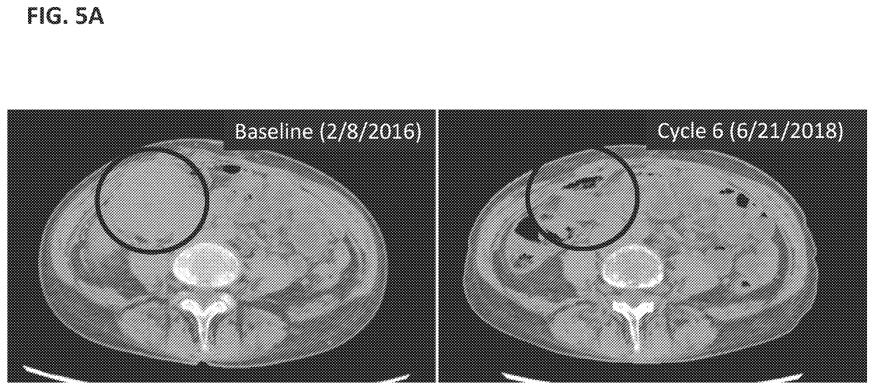

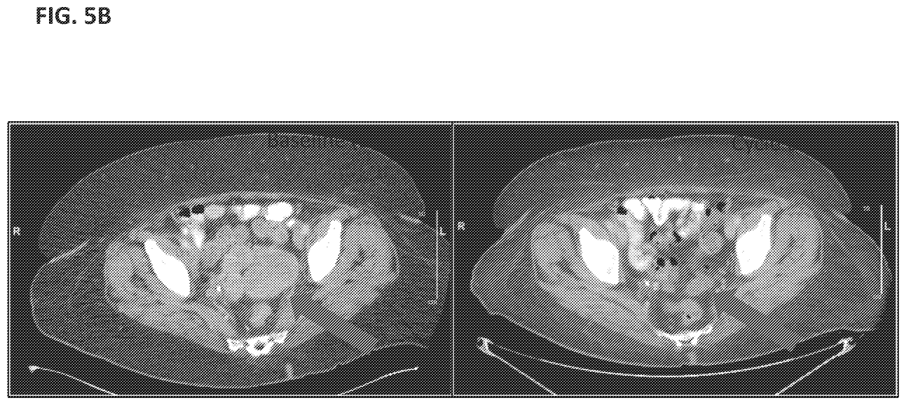

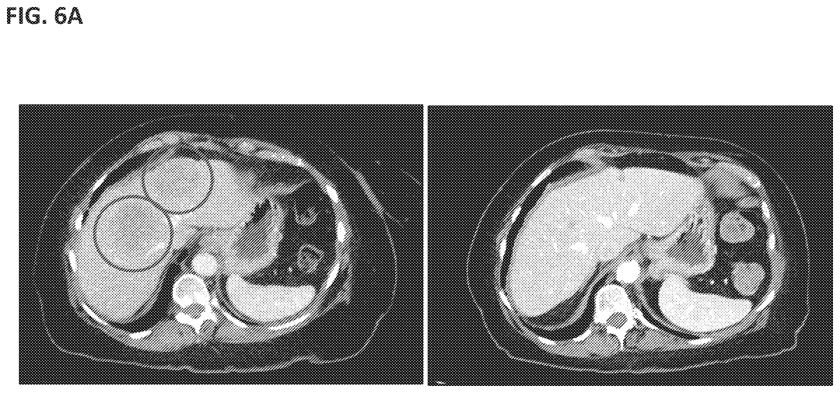

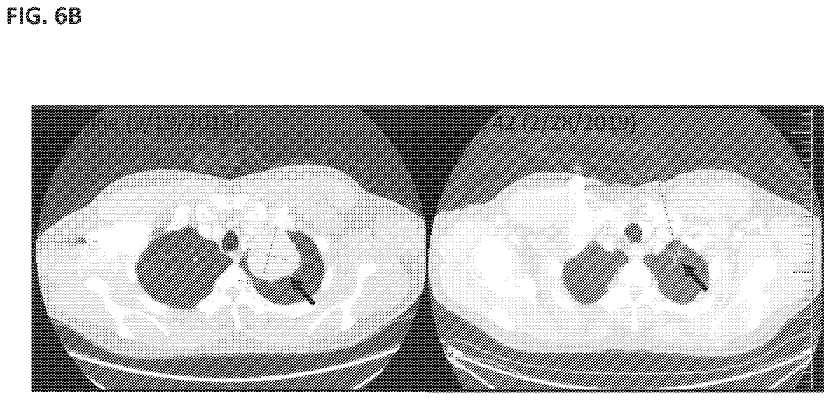

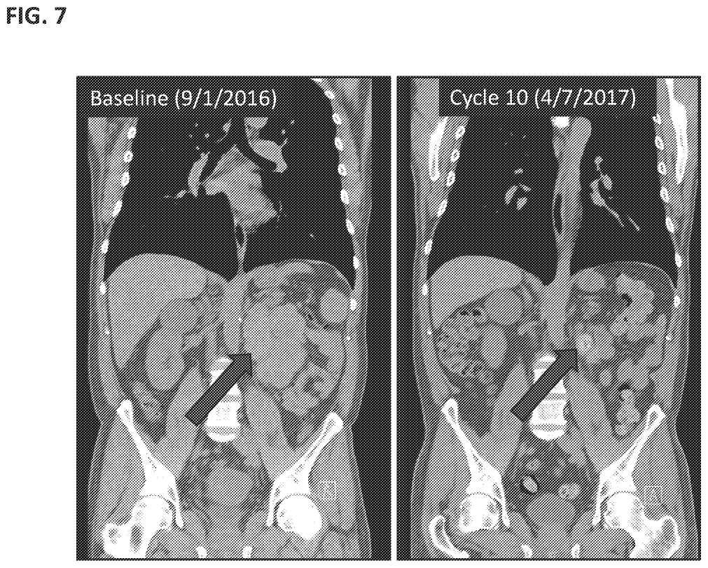

As shown in Table 6, nab-sirolimus is highly active in advanced malignant PEComa with overall response rate (ORR) of 39% by independent radiology review, durable responses, and acceptable safety profile. Patients that showed a confirmed response had PEComa with various primary sites. See representative images of tumors in PEComa with various primary site before and after treatment in FIGS. 5A-5B, 6A-6B, and 7. Specifically, 43% evaluable patients with uterine primary PEComa, a hard to treat subset, had a partial response. No new safety signals were observed despite relatively high doses of nab-sirolimus compared to other mTOR inhibitors. Additionally, 92% (28/31) patients had a best response of PR or SD.

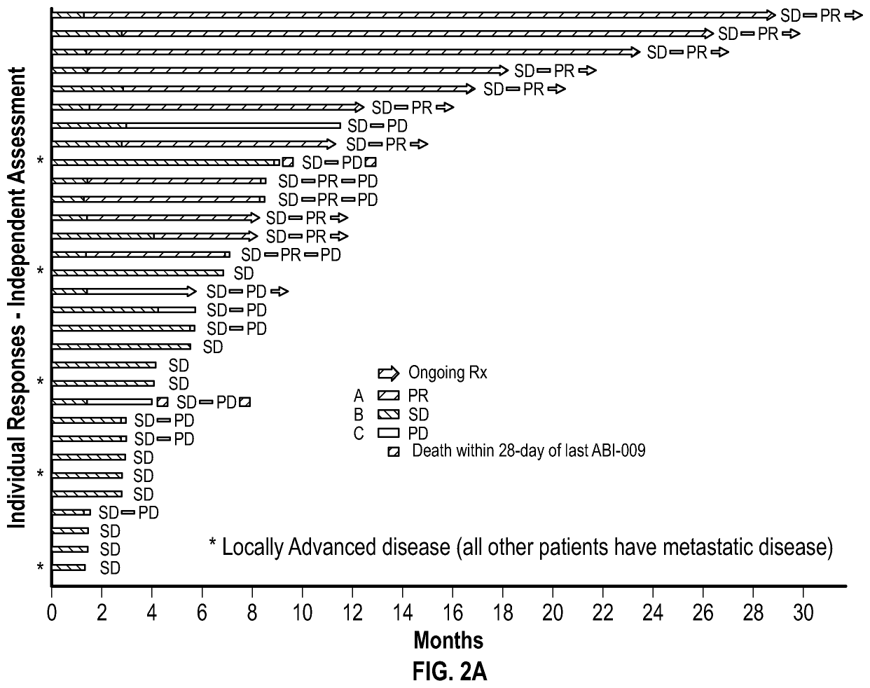

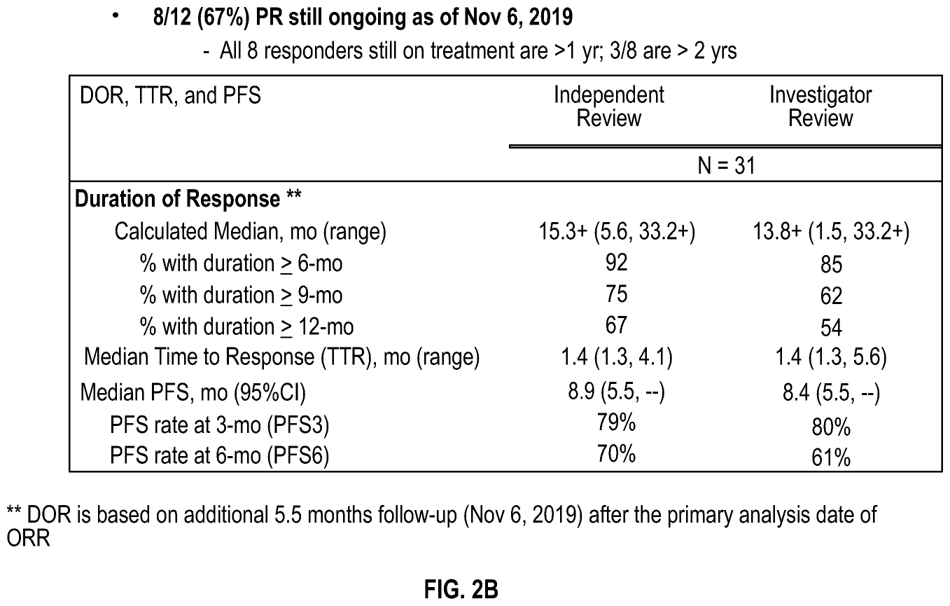

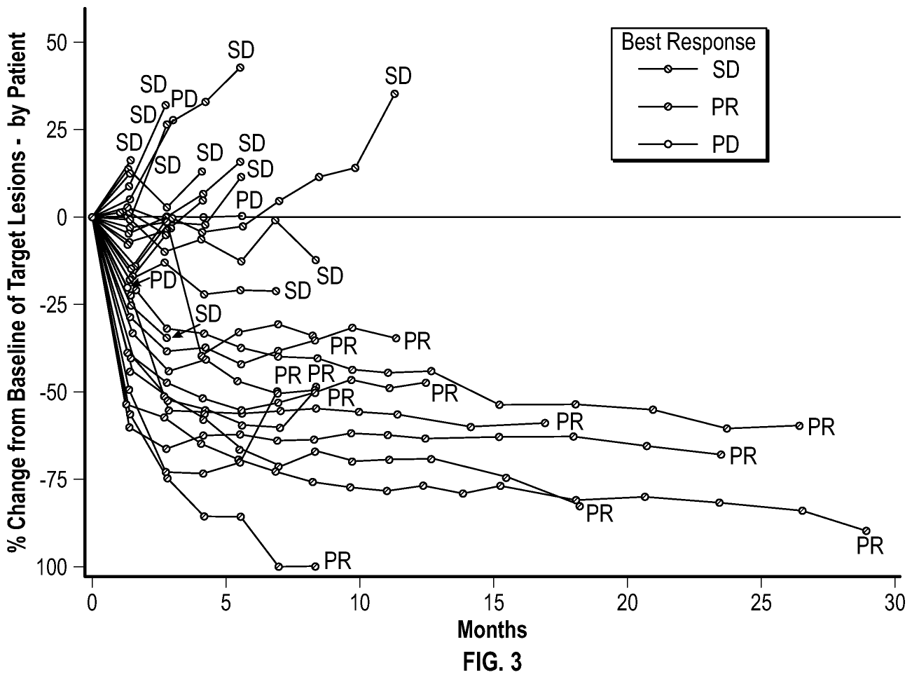

Individual responses and various parameters were listed in Table 9 and analyzed in FIG. 2A and FIGS. 3-4. As of Nov. 6, 2019, eight out of the twelve patients who had shown partial response are still on treatment. The duration of response, median time to response and median PFS were analyzed in FIG. 2B. Ninety percent of patients achieved a PR or SD. Disease control (PR+SD≥12 weeks) was achieved in 71% of patients.

As of Nov. 6, 2019, 75% (9/12) of responders had been on therapy for more than 1 year and 42% (5/12) for more than 2 years, with 67% (8/12) still on treatment. Median DOR has not been reached (range [5.6-33.2+ months] and 50% of the responders have a response duration that is 15.3 months or longer; the median time to response was 1.4 months (95% CI: 1.3, 2.7).

Median PFS is 8.9 months (95% CI: 5.5, -), PFS rate at 3 months (PFS3) is 79%, PFS6 is 70%, and 26% (9/34) of all patients enrolled remain on treatment. For reference, per a meta-analysis of 10 years of phase 2 trials in advanced soft tissue sarcomas (STS) published by the EORTC STS and Bone Sarcoma Group (Wagner et al. 2010. J Clin Oncol 28(5): 835-840), the PFS3 and PFS6 are widely accepted as a meaningful measure of activity of drugs in STS and may be utilized to determine acceptable criteria of benefit. Drugs yielding a PFS rate of ≥40% at 3 months and >14% at 6 months are considered to be ‘potentially active’ in advanced STS (Penel et al. 2011. Ann Oncol 22(6): 1266-1272.)

Mutational status of the suspect genes TSC1 or TSC2 in the mTOR pathway were analyzed for association with patient response outcomes. See Table 7. Mutation or deletion of TSC1 or TSC2 (no overlap) occurred in 5 (20%) and 9 (36%) patients respectively, while 11 (44%) patients had no alterations in TSC1 or TSC2. Specifically, patients with TSC mutations have a) deletions in 4999A and 5002T; b) deletion in 3521G and a mutation in 2743C>G; c) a deletion from 1405C to 1409C; d) deletion in 5208C; e) a mutation in 4949A>G; f) a mutation in 707T>C; g) a deletion from 1960G to 1970A; h) a mutation from 1513C>T. Responses occurred in 9/9 (100%, 8 confirmed responses (89%), 1 unconfirmed response (11%)) patients with TSC2 mutations, 1/5 (20/%) patients with TSC1 mutations and 1/11 (9%) of patients with no mutations in TSC1 or TSC2. Moreover, as shown in Table 8, phosphorylated S6 expression by IHC was significantly associated with response, while absence of phosphorylated S6 was associated with no response.

Eleven patients with TSC1 or TSC2 mutations were analyzable for pS6 expression status. Ten out of eleven patients (91%) expressed pS6. In contrast, only 5/11 (45%) without TSC1 or TSC2 mutation expressed pS6. All patients with a TSC2 mutations and a positive pS6 responded to the treatment, which suggests patients with TSC2 mutation and a positive pS6 status are particularly suitable for the treatment.

Additionally, TFE3 translocation (2/22, both patients SD) was infrequent, and was not associated with pS6 status. Mutations in TP53 were present in a) those that showed at least a partial response (3/10, 30%), b) those that showed a stable disease or a progression of disease (9/15, 6(%).

In conclusion, TSC2 mutations were significantly associated with response (89% of patients) to nab-sirolimus in this cohort of 31 efficacy evaluable patients with PEComa. Responses were also seen in patients with TSC1 mutations (20%) or no TSC1/TSC2 mutations (9%) although at much lower frequency than for TSC2 mutations indicating nab-sirolimus is active regardless of mutational status. Lack of pS6 expression was a negative predictor of response. The first prospective study in advanced malignant PEComa suggests that nab-sirolimus may offer an important benefit in a rare and aggressive sarcoma for which there are no approved therapies. A prospective tumor agnostic trial of nab-sirolimus for patients with tumor mutations in TSC2 is warranted.

TABLE 6

| | | Investigator |
|---|---|---|
| | Independent Review | Review' |
| Response Assessment | N = 31¹ | |
| Confirmed Response Rate (CR + PR)² | 12/31 (39%) | 13/31 (42%) |
| 95% CI | (21.8%, 57.8%) | (24.5%, 60.9%) |
| 2 | 16/31 (52%) | 15/31 (48%) |
| Stable Disease (SD)² | 10/31 (32%) | 10/31 (32) |
| Confirmed SD (12 weeks) | 3/31 (10%) | 3/31 (10%) |
| Progressive Disease (PD) | | |

¹3/34 treated patients were not evaluable—2 pts confirmed as ‘not PEComa’ (misdiagnosis), 1 patient had no tissue for central confirmation of PEComa
²All confirmed responses are PR
*1 patient had an unconfirmed PR and thus best response is an SD as per RECIST v1.1
**Patient with CR in target lesion had a nonCR/nonPD nontarget lesion, thus overall assessment is a PR as per RECIST v1.1

TABLE 7

| | Partial response | Stable disease | Progressive disease |
|---|---|---|---|
| TSC2+ only | 8/9 | 1/9 | 0/9 |
| TSC1+ only | 1/5 | 3/5 | 1/5 |
| No TSC2+ or TSC1+ | 1/11 | 8/11 | 2/11 |

TABLE 8

| | Partial response | Stable disease | Progressive disease |
|---|---|---|---|
| pS6+ | 10/17 | 4/17 | 3/17 |
| pS6− | 0/8 | 8/8 | 0/8 |

TABLE 9

| | a | b | c | d | e | f | g | h | i | j | k | l | m |
|---|---|---|---|---|---|---|---|---|---|---|---|---|---|
| 1. | F | K | − | M | PR | PR | FM | − | FM | − | − | − | − |
| 2. | F | O | − | M | PR | PR | FM | − | SSM | − | − | − | − |
| 3. | F | U | − | M | PR | PR | FM* | − | − | − | − | MM | − |
| 4. | F | K | − | M | PR | PR | HD | − | − | − | − | − | − |
| 5. | F | R | − | M | PR | PR | FM* | − | − | − | − | − | − |
| 6. | F | U | − | M | PR | PR | FM* | − | − | − | − | − | − |
| 7. | F | R | NE | M | PR | SD | FM | − | − | − | − | − | − |
| 8. | M | R | NE | M | PR | PR | NM | − | − | − | − | − | − |
| 9. | F | O | NE | M | PR | PR | FM* | − | − | − | − | − | − |
| 10. | F | U | − | M | PR | PR | − | FM | − | − | NM | − | − |
| 11. | F | L | − | I | PR | SD | − | − | MM | − | − | MM | − |
| 12. | M | K | − | M | PR | PR | NE | NE | NE | NE | NE | NE | NE |
| 13. | F | P | NE | M | PR | PR | NE | NE | NE | NE | NE | NE | NE |
| 14. | M | P | − | M | SD | SD | − | MM | FM | SSM | − | MM | − |
| 15. | M | O | − | M | SD | PD | − | NM | NM | FM | − | − | MM |
| 16. | F | R | − | I | SD | SD | − | SSM | − | − | − | − | − |
| 17. | F | U | − | M | SD | SD | − | − | FM | HD | NM* | − | − |
| 18. | F | U | − | M | SD | PD | − | − | SSM | FM | − | − | − |
| 19. | F | R | − | M | SD | SD | − | − | MM | − | FM | − | − |
| 20. | F | U | NE | M | SD | SD | − | − | MM* | − | − | − | − |
| 21. | F | P | − | M | SD | PR | − | − | MM | − | − | − | − |
| 22. | F | R | − | I | SD | SD | − | − | − | − | − | − | − |
| 23. | M | P | + | M | SD | SD | − | − | − | − | − | − | − |
| 24. | F | O | NE | I | SD | SD | NE | NE | NE | NE | NE | NE | NE |
| 25. | M | P | NE | I | SD | SD | NE | NE | NE | NE | NE | NE | NE |
| 26. | F | L | NE | M | SD | SD | NE | NE | NE | NE | NE | NE | NE |
| 27. | F | L | − | M | SD | SD | − | − | − | HD | SSM | − | − |
| 28. | F | P | − | M | SD | SD | NE | NE | NE | NE | NE | NE | NE |
| 29. | F | R | NE | M | PD | SD | − | SSM | HD | − | − | − | − |
| 30. | F | U | − | M | PD | PD | − | − | MM | HD | FM | − | − |
| 31. | F | O | + | M | PD | SD | − | − | − | − | − | − | − |

Annotations for Table 9:
a-Gender. F = female; M = male
b-Site of primary tumor. K = kidney; L = lung; P = pelvis; R = retroperitoneum; U = uterus; O = others.
c-TFE translocation. [+] = positive; [−] = negative; NE = not evaluable.
d-Metastatic or inoperable locally advanced. M = metastatic; I = inoperable locally advanced.
e-f: e-Investigator assessed response; f-Central review response. PR = partial response; SD = stable response; PD = progression of disease.
g-m: g-TSC2 mutation; h-TSC1 mutation; i-TP53 mutation; j-RB1 mutation; k-ATRX mutation; l-FAT1 mutation; m-PTEN mutation. SSM = Splice site mutation; NM = nonsense mutation; FM = frameshift mutation; MM = missense mutation; HD = homozygous deletion; NE = not evaluable; [−] = no mutation;
*Bi-allelic mutations.

One-Year Follow-Up after the Primary Analysis for DOR, PFS, and OS:

### Reponses and Duration of Response

One year of follow-up after the primary analysis date, 7 patients were still receiving treatment and the median DOR was still not reached (DOR range 5.6, 42.4+ months, calculated median 25.8+ months). Notably, one patient with a primary renal PEComa metastatic to the lungs and lymph nodes had a PR for 10 months that converted to a CR, and the response is ongoing at 21.6+ months.

### Progression-Free Survival and Progression-Free Rate

Median PFS was 8.9 months (95% CI: 5.5 months, not reached). At 6 months, 69% of patients remained progression-free. The progression-free rate was 43% at 12 and 24 months.

### Biomarkers

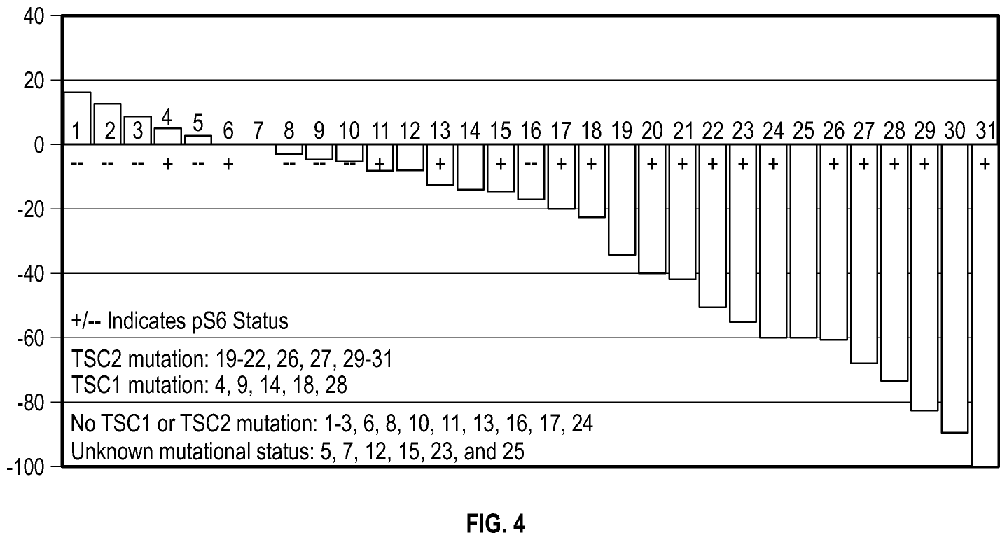

Inactivating mutations in TSC1 (n=5, 20%) or TSC2 (n=9, 36%) were identified in tumor specimens of 25 patients with sufficient PEComa tissue for genetic analysis. TSC1 and TSC2 mutations were mutually exclusive. Confirmed PR occurred in 8/9 (89%) patients with a TSC2 mutation (the 1 additional patient with TSC2 mutation had an unconfirmed PR), 1/5 (20%) patients with TSC1 mutation, and 1/11 (9%) without an identified mutation in TSC1 or TSC2. See FIG. 4 and Table 9. Also, 8/9 (89%) patients with a TSC2 mutation achieved a response vs 2/16 (13%) without a TSC2 mutation (P<0.001, Fisher's exact test). Stable disease ≥12 weeks occurred in patients in each of the above subgroups (i.e., either TSC1 or TSC2 mutations or neither TSC1 or TSC2 mutations). Six patients had tumors with an unknown mutational status; responses occurred in 2 patients (33%) of this group.

The median DOR had not been reached for patients with TSC2 mutations at the 1-year follow-up after the primary analysis (8 patients, range: 6.5 to 42.4+ months). One patient with a TSC1 mutation and 1 patient with no TSC1 or 7S′C2 mutations had a DOR of 5.6 months and 28.4+ months respectively. Anatomic site was not associated with TSC2 mutations; the primary site of tumors for the 9 patients with TSC2 mutations were retroperitoneum (3), kidney (2), uterus (2), liver (1) and small bowel (1).

FIG. 13 presents a Kaplan-Meier curve for PFS and OS for the mutation subtypes.

The absence of pS6 IHC staining was significantly associated with lack of response to nab-sirolimus treatment. In 25 patients whose pS6 status by IHC was available, responses occurred in 10/17 (59%) patients with pS6+ tumors versus 0 of 8 patients with pS6− tumors (P=0.008, Fisher exact test, See FIG. 4 and Table 9).

TFE3 translocations were identified in 2/22 patients evaluable for FISH; both had SD as best response. The tumors were pS6− and without mutations in TSC1 or TSC2.

One of 7 patients with RB1 mutation responded to nab-sirolimus, while 9 of 18 patients without RB1 mutation responded (P=0.18). Interestingly, this patient with a PR also had TSC1 and TP53 mutations.

Mutations in other genes (ATRX, FAT1, PTEN) were not associated with response.

Further analysis of mutations in TSC1 or TSC2 patients shown in Table 10.

TABLE 10

| Patient | Re- | TSC1/ | Bi- | TSC1/TSC2 | | |
|---|---|---|---|---|---|---|
| # | sponse | TSC2 | allelic | mutation | pS6 | Other mutations |
| 2 | PR | TSC1 | Y | F462Lfs*65 | positive | ATRX, CDKN2C, TP53, |
| | | | | in 5% of 387 | | PTEN, BUB1B, CDH4, |
| | | | | reads | | MCL1, RIT1, NTRK1, |
| | | | | | | PVRL4, TLX3, , CEBPA, |
| | | | | | | MUTYH, NOTCH3, |
| | | | | | | RBBP8, SDHA, |
| | | | | | | SMARCA4, TET2, |
| 3 | PR | TSC2 | Y | TSC2 | positive | C17orf70, CDH4, EZH2, |
| | | | | c.4999delA | | FGFR4, RIF1 |
| | | | | and | | |
| | | | | c.5002delT | | |
| | | | | (fs) each in | | |
| | | | | 18-22% | | |
| | | | | reads in trans | | |
| 4 | PR | TSC2 | Y | TSC2 | positive | CDKN2C, ERBB3, FAT1, |
| | | | | c.3521delG | | ASXL1, BLM, CCNE1, |
| | | | | and c.2743- | | EPCAM, FLT1, FLT4, |
| | | | | 3 C > G (fs) | | JAK2, KDM6A, MGA, |
| | | | | each in 19- | | NRG1, PDGFRB, PMS2, |
| | | | | 22% reads | | PRKDC, PTCH1, RAD50, |
| | | | | | | RET, SETBP1, SETD2, |
| | | | | | | TRIM37 |
| 7 | CR | TSC2 | N | TSC2 | positive | TP53, PRKDC |
| | | | | c.1405_1409 | | |
| | | | | delCTGTC | | |
| | | | | (p.S470Cfs* | | |
| | | | | 10), exon 14 - | | |
| | | | | in 26% of | | |
| | | | | 141 reads | | |
| 10 | SD | TSC2 | N | single copy | positive | CDKN2C, FANCD2, |
| | | | | loss of TSC2 | | PDGFRA, PTCH1, WRN |
| 11 | SD | TSC1 | Y | *TSC1 | NE | BRCA2, ERBB3, TP53, |
| | | | | c.2813 + 1 G > | | C19orf40, EXO1, FAN1, |
| | | | | A ( ) - in 46% | | KIT, MAP3K1, |
| | | | | of 255 reads | | MCM8, POLQ, |
| | | | | # | | WHSC1L1, XPA, KAT6B |
| 12 | PR | TSC2 | N | TSC2 | positive | TP53, NPM1, TLX3, |
| | | | | c.5208delC | | UIMC1, JAZF1, RSPO2, |
| | | | | (p.S1738Pfs* | | ATR |
| | | | | 88), exon 41 - | | |
| | | | | in 43% of | | |
| | | | | 64 reads | | |
| 14 | PR | TSC2 | Y | homozygous | positive | CDKN1A, DAXX, EXT1, |
| | | | | del TSC2 | | FANCA, GLI2, NR0B1, |
| | | | | | | SOCS1, TLX3 |
| 17 | PD | TSC1 | N | single copy | positive | TP53, RB1, DICER1, |
| | | | | loss of TSC1 | | DMC1, FANCB, GATA2, |
| | | | | | | GLI1, KMT2A, DNMT3A, |
| | | | | | | GEN1, MYCN, FOXL2, |
| | | | | | | ROS1 |
| 21 | SD | TSC1 | Y | *TSC1 | negative | TP53, RB1, FAT1, BRD4, |
| | | | | c.913 G > A | | CHEK1, EP300, ERCC5, |
| | | | | (p.G305R), | | NSD1, TP53BP1, TSHR, |
| | | | | last nt of | | CCNE1 |
| | | | | exon 9, | | |
| | | | | splice | | |
| | | | | mutation - in | | |
| | | | | 18% of 365 | | |
| | | | | reads | | |
| 22 | PR | TSC2 | Y | TSC2 | positive | GNAS, KLF4 |
| | | | | c.4949 A > G | | |
| | | | | (p.Y1650C), | | |
| | | | | exon 38 - in | | |
| | | | | 13% of 191 | | |
| | | | | reads; | | |
| | | | | c.209delC | | |
| | | | | (p.K71Rfs*3 | | |
| | | | | 5), exon 3 - | | |
| | | | | in 25% of | | |
| | | | | 311 reads | | |
| 24 | PD | TSC1 | N | TSC1 | positive | TP53, RB1, PTEN, CTCF, |
| | | | | c.1525 C > T | | CYLD, EXT1, GLI2, |
| | | | | (p.R509*), | | KMT2A, KMT2D, MEN1, |
| | | | | exon 15 - in | | MSH2, RIF1, RPTOR, |
| | | | | 51% of 361 | | SMARCA4, SUFU, |
| | | | | reads | | TCEB1, XPA |
| 25 | PR | TSC2 | Y | TSC2 | positive | FGFR3, GNAS, H19, PMS2 |
| | | | | c.707 T > C | | |
| | | | | (p.L236P), | | |
| | | | | exon 8 - in | | |
| | | | | 49% of 308 | | |
| | | | | reads; | | |
| | | | | c.5006 T > A | | |
| | | | | (p.V1669D), | | |
| | | | | exon 39 - in | | |
| | | | | 34% of 201 | | |
| | | | | reads; | | |
| | | | | c.1721_1739 | | |
| | | | | delAGCTGT | | |
| | | | | ACACCCTG | | |
| | | | | CCTGC | | |
| | | | | (p.L575Afs* | | |
| | | | | 117), exon | | |
| | | | | 17 - in 13% | | |
| | | | | of 208 reads | | |
| 27 | SD | TSC1 | Y | *TSC1 | positive | ARID1B, ETV4, FANCF, |
| | | | | c.664-1 G > A | | GLI1, NSD1, RNF43 |
| | | | | ( ) - in 91% of | | |
| | | | | 92 reads | | |
| 29 | SD | TSC2 | N | TSC2 | NE | VHL, BRIP1, BUB1B, |
| | | | | c.1966_1970 | | FLT4, RIF1 |
| | | | | delGAGAA | | |
| | | | | (p.K657Dfs* | | |
| | | | | 44), exon 19 - | | |
| | | | | in 25% of | | |
| | | | | 210 reads | | |
| 31 | PR | TSC2 | Y | TSC2 | NE | BRCA2, CIC, ETV1, |
| | | | | c.1513 C > T | | FANCL, HELQ, PIK3C2B, |
| | | | | (p.R505*), | | WRN |
| | | | | exon 15 - in | | |
| | | | | 69% of 2.62 | | |
| | | | | reads | | |

NE = not evaluated.

### Example 2. Malignant PEComa Patient Who had Failed Prior mTOR Inhibitor Responded to AB1-009

A 58-year-old post-menopausal female with family history of lymphoma in her father and breast, ovarian cancer in a paternal aunt, presented with abnormal uterine bleeding in July 2018. Endometrial biopsy revealed a neoplastic process and further work up with CT scan showed a 7 cm mass. Following this, a laparoscopic hysterectomy with bilateral salpingo-oophorectomy was performed and pathology was consistent with malignant PEComa which stained positive for smooth muscle actin, HMB-45 and Melan-A (59 mitoses per 10 hpf). FoundationOne genomic testing revealed a TSC1 mutation with stable micro satellite status and low tumor mutation burden.

### Treatment History

### Chemotherapy

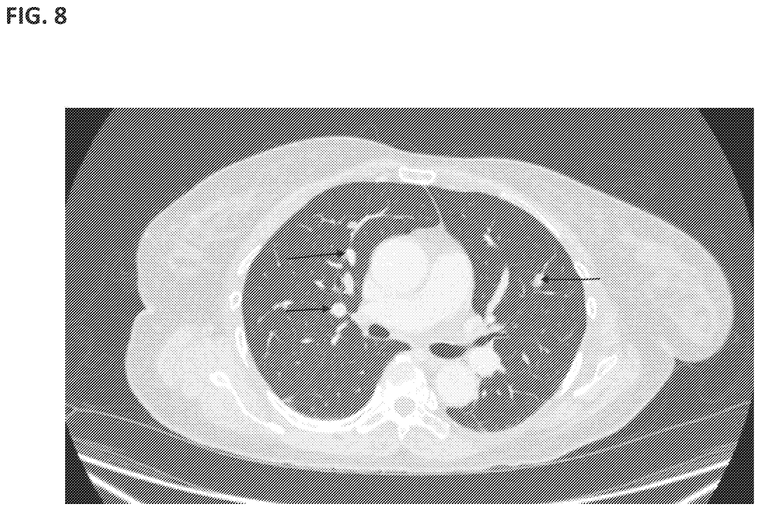

The primary tumor was locally advanced, and no metastatic disease was present at the time of diagnosis, adjuvant chemotherapy was not administered, and the patient was monitored with serial scans. A CT scan at 6 months in February of 2019 (FIG. 8) following surgery showed multiple pulmonary nodules bilaterally, consistent with metastatic disease.

mTOR Inhibitor, Everolimus

Upon disease progression, the patient was started on 10 mg everolimus orally daily. Three weeks after beginning treatment, patient was hospitalized due to fever and headache, related to everolimus and dose was reduced to 5 mg orally every other day which was gradually uptitrated to 5 mg daily in 4 weeks.

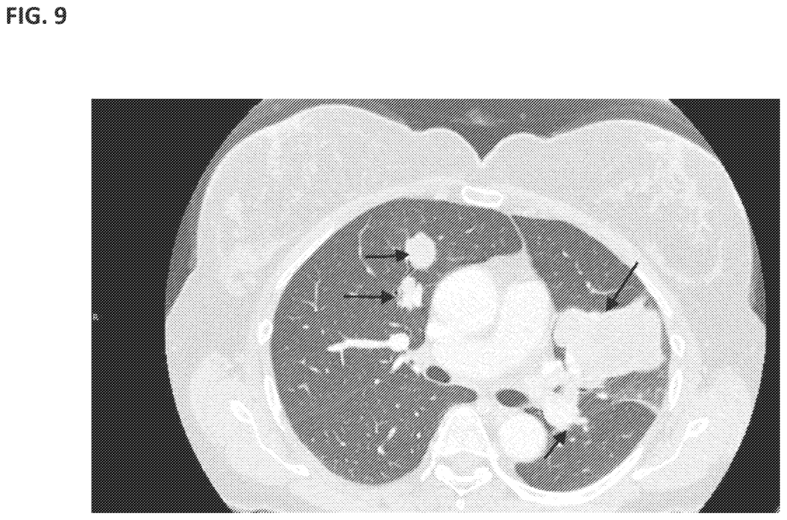

CT scan at 2 months after starting everolimus in April of 2019, demonstrated marked interval enlargement of all pulmonary lesions seen on prior imaging, along with new lesions, indicative of progressive disease (FIG. 9). Additionally, brain imaging performed for evaluation of dizziness showed new enhancing lesion in the periphery of left occipital lobe. Prior scans were negative for any intracranial lesions.

Investigational mTOR Inhibitor, Nab-Sirolimus:

After failure of treatment with everolimus, the patient was treated with nab-sirolimus at 100 mg/m² on day 1 and day 8 of a 21 day cycle started in July of 2019. She also received stereotactic radiosurgery to the metastatic lesion in her brain. The 6-week restaging following 2 cycles of therapy showed marked decrease (50%) in target tumor lesion in her chest, indicating partial response which were confirmed by the week 12 scans. The MRI brain also showed reduction in size of the cranial lesions.

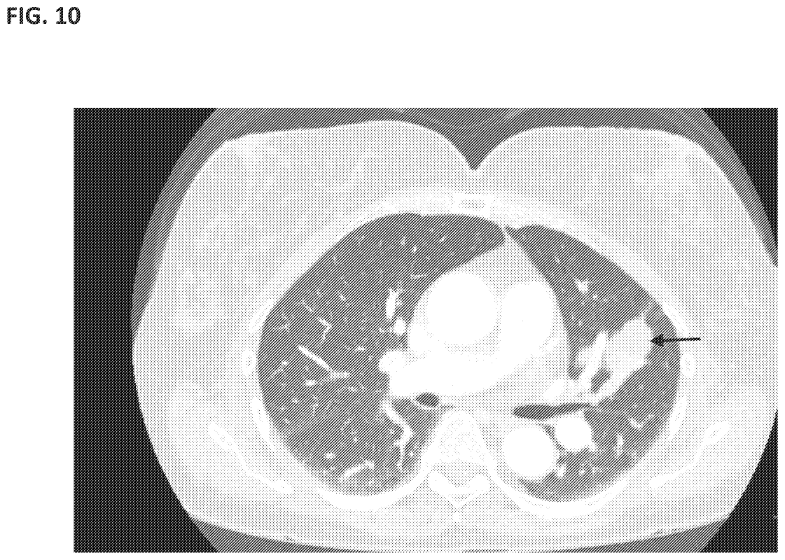

Clinical symptoms prior to nab-sirolimus included coughing-up blood, which ceased after 2 cycles, enabling her to run 2 miles without “getting winded”. Patient developed grade 2 thrombocytopenia after cycle 2 for which dose was reduced to 75 mg/m². Other treatment-related adverse events were elevated lipids, maculopapular rash (grade 2) which were manageable. The patient had a sustained response to nab-sirolimus for 3 months based on scans done on October of 19 (FIG. 10).

### Example 3A. PEComa Patient Who Failed Sirolimus Achieved a Stable Disease after Administration of ABI-009

A patient with PEComa metastatic to lung previously treated with sirolimus and progressed. A mutational analysis on the tumor sample (Left diaphragmatic mass with greater omentum) using the IMPACT NGS panel revealed the following somatic mutations:
1, TSC2 Nonsense Mutation Y648* (c. 1944C>A) exon 18 Mutant allele frequency (MAF): 82.3%
2. TP53 Missense Mutation Y220C (c.659A>G) exon 6 MAF: 81.2%
3. ATRX Frameshift Deletion K1646Mfs*10 (c.4937_4940del) exon 18 MAF: 72.1%
4. The estimated tumor mutation burden (TMB) for this sample is 4.4 mutations per megabase (mt/Mb).
5. MSI Status: MICROSATELLITE STABLE (MSS).

Additionally the following somatic mutation was detected in the blood
1. DNMT3A Splicing X492_splice (c.1474+1G>A) exon 12 MAF: 2.3%
2. DNMT3A Splicing X492_splice (c.1474+ldel) exon 12 MAF: 1.4%

The patient was started on nab-sirolimus 100 mg/m2 IV over 30 minutes for twice every three weeks. Patient disease has been stable and treatment ongoing for more than 15 months since initiation of therapy inspite of progression on prior sirolimus.

### Example 3B. Patient with Undifferentiated Pleomorphic Sarcoma Who had Failed Various Prior Therapies Responded to ABI-009

A 36-year old male patient presented with undifferentiated pleomorphic sarcoma of left thigh with bilateral pulmonary metastases. Prior treatment history of the patient was as follows. After initial diagnosis, the patient first received multiple cycles of neoadjuvant pembrolizumab with concurrent radiotherapy. Amid of the treatment the patient underwent a radical resection of the lower left extremity mass. Subsequent CT scan revealed new pulmonary nodules, which indicated metastasis of undifferentiated pleomorphic sarcoma. The patient was then treated with doxorubicin (75 mg/m²), which was discontinued due to disease progression. After that, the patient was treated with high dose ifosfamide, which was also discontinued due to disease progression.

After failing to respond to multiple regimens, the patient was treated with AB1-009 (100 mg/m² IV over 30 minutes for twice every three weeks, three weeks per cycle) in combination with nivolumab (3 mg/kg IV over 30 minutes once every three weeks).

A genomic profiling test (FoundationOne Heme) was perform on tumor tissue from the patient. The test revealed that the patient had PTEN loss and TSC2 mutation which involves a rearrangement of exon 16. Moreover, the patient had RB1 loss, a TP33 frameshift mutation, and an ATRX frameshift mutation. The patient also had a FAS loss and a KDM6A loss. Other than the above, he also had a FGFR1 amplification, a CKS1B amplification, a MYST3 amplification, a NTRK1 amplification. The patient's microsatellite status was stable and his tumor mutational burden was low.

The patient responded after two cycles (three weeks per cycle) of treatment with ABI-009 and nivolumab. Compared to the baseline CT, tumor size (measured by sum of longest diameters of tumors) decreased by 31%.

### Example 4A

A study was undertaken to compare the antitumor activity of rapamycin by oral route (Rapamune) and intravenous or subcutaneous route (nab-rapamycin) in a human hepatocellular carcinoma xenograft mouse model.

Human cancer cells were prepared for injection in mice by thawing frozen (by liquid nitrogen) SNU-398 (TSC2-deficient human liver hepatocellular carcinoma cells) obtained from ATCC® (CRL-2233™). Cells were dispersed into a 75 cm² flask containing RPMI 1640 media supplemented with 10% fetal bovine serum and incubated at 37° C. in humidified 5% CO₂. At 80% cell confluence, cells were expanded to 150 cm² flasks with fresh culture media. Cells were grown to obtain a target of 1×10⁷ cells per mouse flank (2×10⁷ per mouse).

20 athymic nude mice were housed in filter-topped cages. Cancer cells were injected subcutaneously into both flanks (1×10⁷ per flank) in 0.1 ml phosphate-buffered saline with 20% Matrigel®.

Treatment Day 1 began with the presence of tumors (tumor average ˜100-150 mm³). Animals were sorted into 4 groups.

Group 1, comprising 5 mice, received saline by intravenous route 2× weekly for 6 weeks.

Group 2, comprising 5 mice, received ABI-009 at 7.5 mg/kg by intravenous route 2× weekly for 6 weeks. Total rapamycin dose was 15 mg/kg/wk.

Group 3, comprising 5 mice, received rapamune at 3 mg/kg 5× weekly for 6 weeks by oral administration. Total rapamycin dose was 15 mg/kg/wk.

Group 4, comprising 3 mice, received ABI-009 at 7.5 mg/kg by subcutaneous route 2× weekly for 6 weeks. Total rapamycin dose was 15 mg/kg/wk.

Measurements (mouse weight and tumor measurements) are made three-times weekly (Monday, Wednesday, and Friday) until predefined sacrifice time points and termination 6 weeks later or when tumors reach maximum volume of 2,000 mms. Signs of distress will be recorded daily. Tumors will be harvested and stored. Blood samples will be collected at the same time with tumor harvest.

### Results.

The study is ongoing. Preliminary tumor volume results (mean and standard error of mean, SEM) of each group are summarized in Table 11, below. The tumor growth inhibition (TGI) compared to saline (group 1) and P-value of the TGI vs. saline are reported in Table 11, as well.

TABLE 11
Tumor Growth During Treatment

| | Group 1 | | | | | | | |
|---|---|---|---|---|---|---|---|---|
| Treatment | (control) | | Group 2 | | Group 3 | | Group 4 | |
| Day | Mean | SEM | Mean | SEM | Mean | SEM | Mean | SEM |
| 1 | 149.2 | 16.8 | 134.6 | 10.9 | 122.6 | 14.5 | 115.9 | 22.3 |
| 3 | 253.6 | 28.3 | 202.0 | 29.7 | 182.9 | 20.0 | 142.0 | 43.6 |
| 5 | 323.5 | 37.0 | 222.4 | 39.7 | 276.7 | 43.2 | 167.6 | 67.2 |
| 8 | 530.6 | 62.9 | 185.9 | 30.2 | 367.9 | 68.6 | 126.2 | 47.9 |
| 10 | 789.4 | 87.8 | 274.5 | 48.4 | 537.4 | 94.6 | 162.8 | 68.8 |
| 12 | 1010.8 | 118.8 | 381.7 | 55.2 | 666.1 | 104.0 | 195.1 | 95.0 |
| 15 | 1142.9 | 136.1 | 465.7 | 68.9 | 786.6 | 120.2 | 217.5 | 106.3 |
| TGI | NA | — | 66.7% | — | 33.2% | — | 89.8% | — |
| P-value vs. | NA | — | 0.0006 | — | NS | — | 0.0001 | — |
| Group 1 | | | | | | | | |

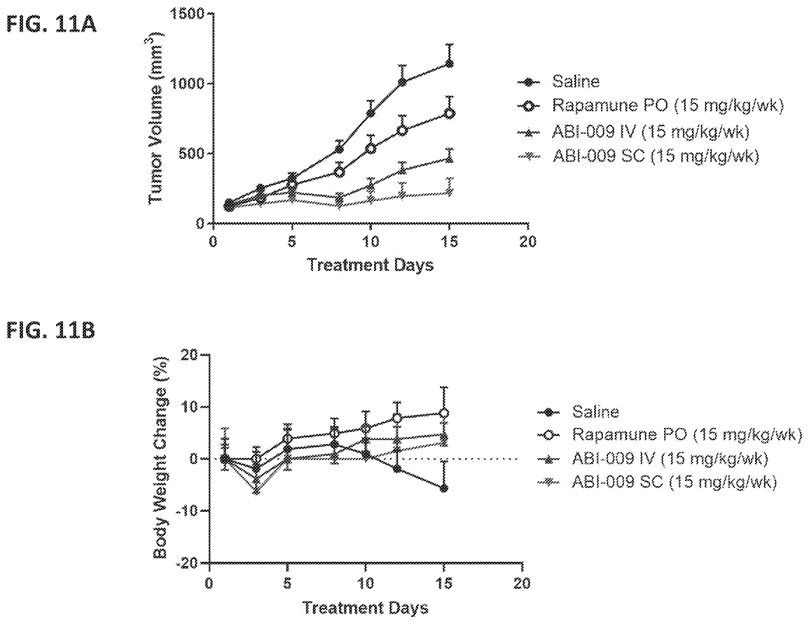

Rapamune oral solution (group 3) at 15 mg/kg/wk resulted in modest tumor growth inhibition (TGI 33.2%, P=not significant) compared with saline control. Equal weekly doses of ABI4009 intravenously (group 2) resulted in significantly greater TG than oral Rapamune (TGI 66.7% vs saline control. P=0.0016 vs oral Rapamune). However. ABI-009 by subcutaneous route (group 4) produced the most profound tumor growth inhibition (TGI 89.8%, P=0.0001 vs. saline control, P<0.0001 vs oral Rapamune). See Table 11 and FIG. 11A.

No signs of toxicity were observed in any treatment group. No major weight loss (>10%) were observed in any treatment group. Slight weight loss was observed in the saline control group (group 1) by Day 15, while each treatment group (groups 2-4) maintained body weight or gained weight by Day 15. See FIG. 11B.

In conclusion, ABI-009 administered by intravenous or subcutaneous route resulted in significantly greater antitumor activity compared with equal weekly dose of oral Rapamune in a TSC2-deficient SNU-398 human hepatocellular carcinoma xenograft mouse model. ABI-009 by subcutaneous route was surprisingly effective even compared to ABI-009 by intravenous route. No major toxicity or weight loss were observed in any treatment group.

### Example 4B

The objective of the study was to evaluate the antitumor effect of ABI-009 delivered IV or SC in comparison to oral Rapamune against TSC2-null SNU-398 tumor xenografts. Tumor volume, body weight measurements, and survival time were assessed.

A total of 20 immunodeficient female athymic nude mice (Strain: Hsd:Athymic Nude-Foxn1^{nu}, Supplier: ENVIGO, East Millstone, N.J., US, R #: 4300) were used in this study. Mice were 5 to 6 weeks old.

ABI-009 100 mg per vial was supplied by Aadi Bioscience, Inc (Lot #C345-001, produced by methods described in Example 7). ABI-009 is a lyophilized powder for injection containing 100 mg sirolimus and approximately 850 mg albumin (human) and stored refrigerated (2 to 8° C./36 to 46° F.). ABI-009 was reconstituted with 0.9% sodium chloride to produce a suspension. Rapamune (Oral Rapamycin Solution or Sirolimus, 1 mg/mL, Lot #: CBFTD, Expiration Date: Dec. 31, 2020) was purchased from Pharmaceutical Buyers (New Hyde Park, N.Y., USA) and stored at 2 to 8° C. protected from light.

The SNU-398 cell line was obtained from American Type Culture Collection (ATCC, Manassas, Va., US, Catalog #CRL-2233™).

### Study Design

Mice received a subcutaneous injection of 10×10⁶ SNU-398 cells into both flanks. Tumor measurements were recorded 3 times per week post-injection until tumors were approximately 50 to 180 mm³.

Tumors were measured with a digital caliper and the following formula was used to calculate tumor volume:

Tumor volume=length×width×width×½.

Mice were divided into 4 treatment groups with 3 to 5 mice in each group based on similar tumor size. All groups were treated for 4 weeks with the appropriate agent and dose frequency as described in Table 12. The dose level and dosing frequency selected for each agent were based on previous nonclinical studies. During the treatment period body weight and tumor measurements were recorded 3 times a week. The animals were observed for signs of distress daily. Body weight, tumor measurements and signs of distress were assessed until the end of the study or until tumor size exceeded the maximum of 200 mm. Mice were sacrificed and tumors were harvested at the end of the study or when the maximum tumor size was exceeded.

TABLE 12
Treatment Groups

| | # | | | | Volume | | Fre- |
|---|---|---|---|---|---|---|---|
| Group | Mice | Tumor | Material | Dosing | (mL/kg) | ROA | quency |
| 1 | 5 | SNU- | Saline | 0 | 10 | IV* | 2×/week |
| | | 398 | | | | | |
| 2 | 5 | SNU- | ABI-009 | 7.5 | 10 | IV | 2×/week |
| | | 398 | | mg/kg | | | |
| 3 | 5 | SNU- | Rapamune | 3 | 3 | PO** | 5×/week |
| | | 398 | | mg/kg | | | |
| 4 | 3 | SNU- | ABI-009 | 7.5 | 10 | SC*** | 2×/week |
| | | 398 | | mg/kg | | | |

Abbreviations: IV = intravenous; PO = oral; SC = subcutaneous.
*IV = intravenous injection,
**PO = oral administration,
***SC = subcutaneous administration

### Experimental Procedures

SNU-398 cells were cultured in 75 cm² flask containing RPMI 1640 media supplemented with 10% fetal bovine calf serum (FBS) and incubate at 37° C. in humidified atmosphere of 5% CO₂. As cells became 80% confluent, cultures were expanded to 150 cm² flasks, and expanded further until sufficient cells were available for injection.

SNU-398 cells were subcutaneously injected into mice (both flanks, 1 Ox 10¹ cells in 0.1 mL phosphate-buffered saline [PBS] with 20% Matrigel per flank, 20 million per mouse).

Test solutions were prepared and dosed as described below. All solutions, with the exception of saline, were stored at −20° C. for further use.

Group 1: Saline—0.9% saline was used directly.

Groups 2 and 4: ABI-009—100 mg of ABI-009 was dissolved in 20 mL of saline to make a solution of 5 mg/mL. The solution was aliquot into 20 Eppendorf tubes and stored at −20° C. Each aliquot was diluted with 5.67 mL of saline to make a solution of 0.75 mg/mL before use.

Group 3: Rapamune—a solution of 1 mg/mL was used as supplied, without further preparation. The 1 mg/mL Rapamune oral formulation is a marketed product.

Mice were divided into treatment groups as described in Table 12, when tumor volume was approximately 50 to 180 mm³. Weight and tumor volumes were recorded, and dosing commenced on Day 0 for all groups. The treatment period was 4 weeks for all groups.

Groups 1, 2, and 4 were dosed twice a week. Group 3 was dosed once daily 5 times per week.

Body weights and tumor volume measurements were performed 3 times a week and animals were observed for signs of distress daily until the end of the study. Mice were sacrificed and tumors harvested after at the end of the study or when the maximum tumor size of 2000 mm³ was exceeded. Tumors of the right side were flash frozen and stored at −80° C. Tumors of the left side were fixed in 10% formalin.

### Statistical Analysis

Tumor growth inhibition (TGI) was calculated based on average tumor volumes of each group compared against the tumor volumes of the saline or the indicated control group. TGI is calculated using the formula 100×(ΔC−ΔT)/ΔC, where ΔT and ΔC are the changes in the mean tumor volumes between the last day when all animals in the saline or control group were alive and the first day of measurement for the treatment and control groups, respectively.

Tumor sizes and body weights were analyzed using analysis of variance (ANOVA; GraphPad Prism 9.0.0, GraphPad Software, San Diego, Calif., US). Animal survival was analyzed using a Log-rank Test (GraphPad Prism 9.0.0). P values <0.05 were considered statistically significant.

### Results

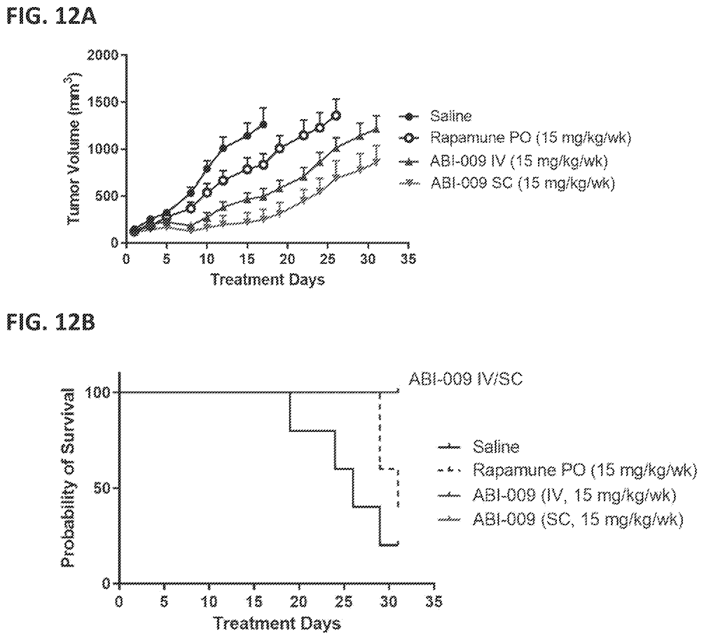

Tumor volumes of each group are summarized in Table 13 and FIG. 12A. Rapamune oral solution at 15 mg/kg/week resulted in modest tumor growth inhibition (TGI) compared with saline control (TGI 36.2%, P=0.0566 vs saline at Day 17, ANOVA). Equal weekly dose of ABI-009 delivered IV resulted in significantly greater TGI than saline (TGI 67.8%; P=0.0004 vs saline control at Day 17) and oral Rapamune (P=0.0408 vs Rapamune P0 at Day 26). Equal weekly dose of ABI-W09 delivered SC also resulted in significantly greater TGI than saline (TGI 87.9%, P=0.0005 vs saline control at Day 17) and oral Rapamune (P=0.0102 vs Rapamune P0 at Day 26). The antitumor effect of ABI-009 SC administration was greater than ABI-009 IV administration although not statistically significant (P=NS at Day 31).

TABLE 13
Tumor Growth following Treatment.

| | | | | ABI-009 IV | | | Rapamune PO | | | ABI-009 SC | | |
|---|---|---|---|---|---|---|---|---|---|---|---|---|
| Treatment | Saline | | | (15 mg/kg/week) | | | (15 mg/kg/week) | | | (15 mg/kg/week) | | |
| Days | Mean | SEM | N | Mean | SEM | N | Mean | SEM | N | Mean | SEM | N |
| 1 | 149.2 | 16.8 | 10 | 134.6 | 10.9 | 10 | 122.6 | 14.5 | 10 | 115.9 | 22.3 | 6 |
| 3 | 253.6 | 28.3 | 10 | 202.0 | 29.7 | 10 | 182.9 | 20.0 | 10 | 142.0 | 43.6 | 6 |
| 5 | 323.5 | 37.0 | 10 | 222.4 | 39.7 | 10 | 276.7 | 43.2 | 10 | 167.6 | 67.2 | 6 |
| 8 | 530.6 | 62.9 | 10 | 185.9 | 30.2 | 10 | 367.9 | 68.6 | 10 | 126.2 | 47.9 | 6 |
| 10 | 789.4 | 87.8 | 10 | 274.5 | 48.4 | 10 | 537.4 | 94.6 | 10 | 162.8 | 68.8 | 6 |
| 12 | 1010.8 | 118.8 | 10 | 381.7 | 55.2 | 10 | 666.1 | 104.0 | 10 | 195.1 | 95.0 | 6 |
| 15 | 1142.9 | 136.1 | 10 | 465.7 | 68.9 | 10 | 786.6 | 120.2 | 10 | 217.5 | 106.3 | 6 |
| 17 | 1262.8 | 175.0 | 10 | 493.6 | 87.7 | 10 | 833.6 | 116.7 | 10 | 250.3 | 108.6 | 6 |
| 19 | | | | 582.1 | 85.8 | 10 | 1006.9 | 136.5 | 10 | 312.6 | 119.4 | 6 |
| 22 | | | | 707.1 | 97.2 | 10 | 1147.6 | 162.5 | 10 | 447.7 | 122.2 | 6 |
| 24 | | | | 864.1 | 97.7 | 10 | 1227.1 | 161.9 | 10 | 543.9 | 143.8 | 6 |
| 26 | | | | 1014.0 | 107.4 | 10 | 1357.4 | 175.8 | 10 | 688.2 | 186.9 | 6 |
| 29 | | | | 1140.7 | 135.0 | 10 | | | | 776.6 | 173.6 | 6 |
| 31 | | | | 1213.5 | 141.5 | 10 | | | | 857.7 | 179.5 | 6 |
| TGI | NA | | | 67.8% | | | 36.2% | | | 87.9% | | |
| P vs | NA | | | 0.0004 | | | NS | | | 0.0005 | | |
| Saline | | | | | | | | | | | | |
| P vs Rapa | NA | | | 0.0408 | | | NA | | | 0.0102 | | |

Abbreviations:
IV = intravenous;
NA = not applicable;
NS = not significant;
PO = oral;
Rapa = Rapamune Oral Solution I mg/mL;
SC = subcutaneous;
SEM = standard error of the mean.

Consistent with antitumor activity of mTOR inhibitors, animal survival was prolonged with treatment (FIG. 12B). At the end of the study (Day 31), only 1 out of 5 animals survived in the saline group, compared to 2/5 alive in the Rapamune group, and all animals alive in ABI-009 TV (5/5) and SC (3/3) groups. Rapamune oral solution at 15 mg/kg/week resulted in longer animal survival compared with saline control (median survival: 31 days vs 26 days for saline, P=NS, Log-rank test). Equal weekly dose of ABI-009 delivered IV and SC resulted in longer survival than oral Rapamune (median survival: not reached).

No signs of toxicity were observed in any treatment group. No major weight loss (>10%) were observed in any treatment groups with mTOR inhibitors. (Data not shown)

### Conclusions

ABI-009 demonstrated antitumor activity against a TSC2-null tumor cell line, supporting the clinical investigation of ABI-009 in patients with solid tumors harboring inactivating mutations in TSC2 gene. ABI-009 administered IV or SC resulted in significantly greater antitumor activity compared with equal weekly dose of oral Rapamune against TSC2-deficient SNU-398 human hepatocellular carcinoma xenografts and longer animal survival. No major weight loss or signs of toxicity were observed in any treatment group. ABI-009 SC delivery is a feasible route of administration for treatment of oncology indications.

### Example 5A. Phase 2 Multi-Center Open-Label Basket Trial of ABI-009 (Nab-Sirolimus) for Adult and Adolescent Patients with Solid Tumors Harboring TSC1 or TSC2 Pathogenic Inactivating Mutations

### Objectives

Primary objective is to determine clinical benefit as described by the overall response rate (ORR) of ABI-009 (produced as described in Example 7) in patients with pathogenic TSC1 (TSC1 Arm) or TSC2 (TSC2 Arm) inactivating mutation-positive solid tumors via independent radiographic review (IRR).

Secondary objectives include a) to evaluate duration of response (DOR), disease control rate (DCR), progression-free survival (PFS) via IRR, and overall survival (OS) of ABI-009 in the TSC1 Arm and TSC2 Arm; b) to evaluate Quality-of-Life (QoL) and c) to describe the safety and tolerability of ABI-009 in the TSC1 Arm and TSC2 Arm and both Arms together.

Exploratory objectives include a) to evaluate ORR, DOR, DCR, time on treatment, and PFS via investigator-assessed responses; b) to evaluate the rate of surgical resection with curative intent for patients with inoperable locally advanced disease; c) evaluate baseline genomics, cfDNA, functional analyses of variants, and the association between genomic mutations and clinical outcome in the TSC1 Arm and TSC2 Arm.

### Endpoints

Endpoints were evaluated for patients in the TSC Arm (pathogenic inactivating TSC1) and TSC2 Arm (pathogenic inactivating TSC2) and by tumor types within the TSC1 Arm and TSC2 Arm.

Primary endpoint is best overall response (BOR) of confirmed partial response (PR) or complete response (CR) from the time of study treatment initiation until disease progression as determined by independent radiologic assessment using Response Evaluation Criteria in Solid Tumors (RECIST) v1.1 or Response Assessment in Neuro-Oncology (RANO), as applicable.

Secondary endpoints include the following: a) DOR: Determined for patients with BOR of confirmed CR or PR (independent radiologic assessment); b) DCR: BOR of confirmed CR or PR (either of any duration) or stable disease (SD) ≥16 weeks following study treatment initiation (independent radiologic assessment); c) PFS: Number of months from study treatment initiation to the date of disease progression or death due to any cause (independent radiologic assessment); d) OS: Number of months from study treatment initiation to the date of death due to any cause; e) evaluating the European Organization for Research and Treatment of Cancer QoL Questionnaire v3.0 (EORTC-QOL-C30); and f) incidence and severity of treatment-emergent and treatment-related adverse events (AEs) as assessed by the National Cancer Institute Common Terminology Criteria for Adverse Events (NCI CTCAE) v5.0 (in the TSC1 Arm and TSC2 Arm and both Arms together).

Exploratory endpoints include: a) investigator assessed ORR, DOR, DCR, and PFS; b) rate of surgical resection with curative intent for patients with inoperable locally advanced disease at baseline; c) time on treatment (including patients treated beyond progression); d) baseline tumor tissue (archival or fresh biopsy) and blood (peripheral blood mononuclear cells, PBMCs) samples are required from all patients: i) to characterize TSC1 and TSC2 mutations as germline vs somatic (PBMCs, using next generation sequencing. NGS); ii) to understand the concomitant alterations and allele frequency via a standardized method (secondary confirmation) (tissue, using NGS); iii) to identify correlation between genomic mutations and clinical outcome; iv) pS6 via immunohistochemistry; e) baseline and during treatment blood collection to identify dynamic clonal changes.

### Study Design and Treatment

This trial is a prospective phase 2, open-label, multi-institutional basket trial to determine the efficacy and safety profile of ABI-009 administered by intravenous (IV) infusion to patients with pathogenic inactivating TSC1 or TSC2 mutations, studied in two independent cohorts: a) Patients with advanced solid tumors bearing TSC1 inactivating mutations (TSC1 Arm); b) Patients with advanced solid tumors bearing TSC2 inactivating mutations (TSC2 Arm).

It is highly unlikely that pathogenic TSC1 and pathogenic TSC2 mutations co-exist, but if such case occurs, that patient would be assigned to the TSC2 Arm.

A cycle consists of 21 days. Patients receive ABI-009 by IV infusion over 30 minutes (+10 mins window allowed, ie 30-40 mins infusion) weekly for 2 weeks followed by a week of rest (qw2/3). The starting dose of ABI-009 is 100 mg/m², with the dose capped at a body surface area (BSA) of 2 m². Four dose reductions are allowed: 75, 60, 45, and 30 mg/m².

Patients will continue treatment until disease progression, or unacceptable toxicity, or until in the opinion of the investigator the patient is no longer benefiting from therapy, or at patient discretion.

The study will be conducted in compliance with International Conference on Harmonisation (ICH) Good Clinical Practices (GCPs).

### Number of Patients

The prevalence of pathogenic TSC1 and TSC2 inactivating mutations is relatively low but they are detected in a wide variety of malignancies. Solid tumors where TSC2 mutations are most frequent include hepatocellular carcinoma, melanoma, renal cell carcinoma, gynecologic cancers, and sarcoma. For TSC1 mutations, bladder cancer, melanoma, renal cancer, and endometrial cancer are the most frequent tumor types.

The expected enrollment is approximately 60 patients in TSC1 Arm and TSC2 Arm each (up to 120 patients in total). Tumor types will be capped at 15 patients to avoid over-enrolling in any one type of cancer.

### Sample Size Estimate

Sample size estimation is based on the primary endpoint of BOR (proportion of patients that achieved a confirmed objective response) evaluated separately for TSC1 Arm and TSC2 Arm.

A sample size of approximately 60 patients in each TSC1 Arm and TSC2 Arm is planned. If the observed ORR is 40% in each Arm, then an N=60 will exclude a lower bound of the 95% confidence interval (CI) of 25%.

### Inclusion Criteria

A patient will be eligible for inclusion in this study only if all of the following criteria are met at screening:

Patients must have a ‘definite’ or ‘likely’ pathogenic inactivating TSC1 (TSC1 Arm) or TSC2 (TSC2 Arm) mutation that confers a loss-of-function within a solid tumor. Mutations should be identified in tumor tissue using NGS (ie, not by liquid biopsy alone).

Patients will be enrolled after the central evaluation of NGS reports confirm eligibility.

Patients must provide baseline tumor tissue samples.

Patients must have solid tumors that are metastatic or locally advanced where surgical resection is not an option or likely to result in severe morbidity.

Patients have must have received all standard therapies appropriate for their tumor type and stage of disease (including targeted therapies), or in the opinion of the Investigator, would be unlikely to tolerate or derive clinically meaningful benefit from appropriate standard of care therapy, or have no satisfactory alternative treatments.

Patients must have one or more measurable target lesions by computed tomography (CT) scan or magnetic resonance imaging (MRI) (RECIST v1.1 or RANO, as applicable for their tumor type).

Age: 12 years or older

Eastern Cooperative Oncology Group (ECOG) performance status 0, 1, or 2 or Karnofsky Performance Status (KPS) ≥70

Adequate liver function: Total bilirubin ≤1.5×upper limit of normal (ULN) mg/dL. Aspartate aminotransferase (AST) ≤2.5≤ULN (≤5×ULN if attributable to liver metastases)

Adequate renal function: Creatinine clearance >50 mL/min (Cockcroft-Gault).

Adequate hematologic parameters: Absolute neutrophil count (ANC) ≥1.0×109/L; Platelet count ≥100,000/mm3 (100×109/L) (transfusion and/or growth factors allowed); Hemoglobin ≥8.0 g/dL (transfusion and/or growth factors allowed); Fasting serum triglyceride ≤5300 mg/dL; fasting serum cholesterol ≤350 mg/dL.

Minimum of 4 weeks since any major surgery, completion of radiation, or completion of all prior systemic anticancer therapy, or at least 5 half-lives if the prior therapy is a single agent small-molecule therapeutic, and adequately recovered from the acute toxicities of any prior therapy, including neuropathy, to grade ≤1.

Male or non-pregnant and non-breast feeding female: Females of child-bearing potential must agree to use effective contraception or abstinence without interruption from 28 days prior to starting investigational product (IP) throughout 3 months after last dose of IP and have a negative serum pregnancy test (beta human chorionic gonadotropin, β-hCG) result at screening and agree to ongoing pregnancy testing during the course of the study, and after the end of study treatment. A second form of birth control is required even if she has had a tubal ligation.

Male patients must practice abstinence or agree to use a condom during sexual contact with a pregnant female or a female of childbearing potential while participating in the study and throughout 3 months after last dose of IP. A second form of birth control is required even if he has undergone a successful vasectomy.

The patient or the patient's parent(s) or legal guardian(s) understand(s) and sign(s) the informed consent.

Willingness and ability to comply with scheduled visits, laboratory tests, and other study procedures.

### Exclusion Criteria

A patient will not be eligible for inclusion in this study if any of the following criteria apply:

Prior treatment with a mammalian target of rapamycin inhibitor (mTOR inhibitor), including ABI-009.

Recent infection requiring systemic anti-infective treatment, either ongoing or completed ≤14 days prior to enrollment (except for uncomplicated urinary tract infection or upper respiratory tract infection).

Patients who have any severe and/or uncontrolled medical or psychiatric conditions or other conditions that could affect their participation.

Use of strong inhibitors and inducers of CYP3A4 at least 1 week or 5 half-lives of the inducers (whichever is longer) prior to receiving the first dose of ABI-009. Additionally, use of any known CYP3A4 substrates with a narrow therapeutic window (such as fentanyl, alfentanil, astemizole, cisapride, dihydroergotamine, pimozide, quinidine, or terfenadine) within 5 half-lives prior to receiving the first dose of ABI-009.

### TSC1 and TSC2 Inactivating Mutations Pathogenicity Classification

TSC1 and TSC2 mutations should be identified in tumor tissue using analytically validated NGS from a Clinical Laboratory Improvement Amendments (CLIA)-certified laboratory. The NGS reports for each patient will be evaluated centrally to ensure eligibility.

Pathogenic inactivating mutations (loss-of-function) of TSC1 and TSC2 genes will be determined by review of experimental evidence within the published scientific literature and review of critical regions that may be disrupted, including but not limited to frameshift, missense mutations, truncating mutations, deletions, copy number variations, or nonsense mutations. A pathogenic mutation of the TSC1 and TSC2 is inferred as inactivating.

Pathogenicity Classifications

Definite Pathogenic: includes but not limited to homozygous deletions, bi-allelic (double hit), 2nd splice site, frameshift, and nonsense mutation in coding region, missense mutation with confirmed impact

Likely Pathogenic: includes but not limited to missense without confirmed pathologic impact

Unlikely Pathogenic: mutations with unknown functional significance

Not Pathogenic: mutation in noncoding regions

### Duration of Treatment and Study Participation

The study will enroll patients in approximately 10-15 US sites and is expected to take approximately 50 months from first patient enrolled to last patient follow-up, including approximately 24 months of enrollment period, an estimated 24 months of treatment, a 28-day screening and a 28-day (4 week) safety follow-up after the last dose.

End of Treatment (EOT) for a patient is defined as the date of the last dose of ABI-009. The End of Treatment Visit (EOT Visit) for a patient is a safety follow-up visit; safety assessments and procedures are performed at least 4 weeks (+7 days) after the last dose of ABI-009 is administered.

The End of Study (EOS) is defined as either the date of the last visit of the last patient to complete the study, or the date of receipt of the last data point from the last patient that is required for primary, secondary, and/or exploratory analysis, as pre-specified in the protocol.

The Follow-up period begins after the EOT Visit. All patients that discontinue study drug and have not withdrawn full consent to participate in the study will continue in the follow-up phase for survival and initiation of new anticancer therapy. Follow up will continue approximately every 12 weeks (±3 weeks), until death, withdrawal of consent, or the study closes, whichever is the earliest. This evaluation may be made by record review and/or telephone contact.

Key Efficacy Assessments

Efficacy will be assessed by investigators and independent radiologic review using CT or MRI scans using RECIST v1.1 or RANO, as applicable.

Patients will be evaluated for CR, PR, SD, or progressive disease (PD) by CT imaging or contrast enhanced MRI can also be used. The same modality of imaging should be used throughout the study. Baseline scan results can be accepted from outside institutions but must be done within 4 weeks of starting therapy and must include (as clinically indicated), chest, abdominal, and pelvic CT or MRI. The first response assessment by CT or MRI scans documenting target lesions will be done 8 weeks after first treatment and should be repeated every 8 weeks (±7 days) for the first year, then every 12 weeks (±7 days) thereafter until disease progression. If an initial observation of objective response (CR or PR) is made, a confirmation scan should be done 4 weeks (±1 week) after the initial observation. Scans should continue on schedule regardless of delays in ABI-009 dosing.

The BOR and DCR will be reported along with exact 95% CIs computed by the Clopper-Pearson method.

Definitions:

DOR is defined as the number of months from the start of CR or PR (whichever response is recorded first) and subsequently confirmed to the first date of documented PD or death.

DCR is defined as BOR of confirmed CR or PR (either of any duration) or SD ≥16 weeks following study treatment initiation.

PFS is defined as the time from the first dose to the first observation of a disease progression or death due to any cause.

OS is defined as the time of first dose to the date of death due to any cause.

For PFS, OS, and DOR, the Kaplan-Meier (KM) estimates and corresponding two-sided 95% CIs for the median and quartiles will be provided. The KM plot also may be provided.

All patients will be analyzed together across tumor types within each Arm. Tumor types within Arms may also be analyzed separately:

If ≥5 patients enroll with the same tumor types, they will be grouped together for analysis; ≤4 patients per tumor types will be grouped as “other”.

### Key Safety Assessments

Safety and tolerability will be monitored through continuous reporting of treatment-emergent and treatment-related AEs, AEs of special interest, laboratory abnormalities, and incidence of patients experiencing dose modifications, dose delay/dose not given, dose interruptions, and/or premature discontinuation of IP due to an AE. All AEs will be recorded by the investigator from the time the patient signs informed consent until 28 days after the last dose of IP. Adverse events will be graded by NC CTCAE v5.0.

Physical examination, vital signs, laboratory assessments (eg, serum chemistry, hematology), and ECOG performance status will be monitored. All serious AEs (regardless of relationship to IP) will be followed until resolution. Local laboratory analysis will be performed as per study schedule.

### Example 6. Clinical Evidence with Single-Agent ABI-009 in Consecutive Non-PEComa Patients with Relevant mTOR Pathway Mutations

Seven patients were enrolled under ABI-009 Expanded Access Protocol. See Table 14 below for information about their tumor type, relevant mutation, failed prior therapy and response to ABI-009. ABI-009 was produced according to Example 7. All five patients (#1, #2, #3, #5, #6) without prior mTOR inhibitor treatment showed significant anti-tumor activity. Among those, patients #1, #2, #5 and #6 who satisfied the key inclusion criteria of the TSC1, TSC2 pan tumor registration study discussed in Example 5, i.e., must have pathologic inactivating TSC1 or TSC2 mutation; must have no satisfactory alternative treatments or have progressed following a standard treatment; must not be previously treated with an mTOR inhibitor, were all responding.

TABLE 14

| | | TSC1 or | | |
|---|---|---|---|---|
| Patient | | TSC2 | | Response to |
| # | Tumor Type | mutation | Failed Prior Therapy | ABI-009 |
| 1 | Metastatic | TSC2 | Anti-estrogen therapy | Responding* |
| | Endometrial | | | |
| | Cancer (Stromal | | | |
| | Sarcoma) (002- | | | |
| | 006) | | | |
| 2 | Metastatic | TSC1 | Cisplatin/paclitaxel, | Responding* |
| | Epithelial Ovarian | | bevacizumab, carboplatin, | |
| | Cancer(002-007) | | liposomal doxorubicin, | |
| | | | gemcitabine | |
| 3 | Metastatic | mTOR exon | liposomal doxorubicin, | Tumor shrinkage |
| | Angiosarcoma | 43 | paclitaxel, gemcitabine, | and necrosis* |
| | (002-008) | | Vinorelbine, pazopanib, anti- | |
| | | | PD-1 on clinical trial | |
| 4 | Metastatic | TSC2 | liposomal doxorubicib, | No follow up |
| | Epithelial Ovarian | | carboplatin, bevacizumab, | scan |
| | Cancer(002-009) | | gemcitabine, enzalutamide, | |
| | | | MLN0128 (mTORi) | |
| 5 | Metastatic | TSC1 | 1^{st} line—doxorubicin, | Responding* |
| | Angiosarcoma | | ifosfamide, mesna; 2^{nd} line— | |
| | (002-010) | | paclitaxel [both unresponsive | |
| | | | to Rx] | |
| 6 | Metastatic High | TSC2 | Adriamycin + ifosfamide, | Responding* |
| | Grade Sarcoma | | gemcitabine + Taxotere, | |
| | (009-002) | | surgery; Adjuvant gemcitabine; | |
| | | | pazopanib, pembrolizumab | |
| | | | plus denosumab | |

* Based upon investigator's assessment.

More specific information about patients were provided below.

### Patient #1

Patient #1 is a 64 year old female. She has low grade endometrial stromal sarcoma metastatic to liver and peritoneum. She has been treated with Exemestane, Letrozole, Fulvestrant. The patient is positive for the following somatic alterations: TSC2 (NM_000548) exon18 p.C646′* (c.1938C>A); TSC2 (NM_000548) exon30 p.W1194* (c. 3581G>A); NTRK1 (NM_:0025 29—1q23.1) Amplification (Fold Change: 2.0); AR (NM_000044) exon1 p.H41Q (c.123C>A); IL7R (NM 002185) exon8 p.K395R (c.1184A>G). Patient #1 started treatment of ABI-009 with a dose of 100 mg/m². She has completed five cycles.

The patient developed some AEs including mucositis, diarrhea and mild skin rash all of which have resolved. No SAE or dose limiting events.

Radiology report about 1-2 months after initiation of the treatment showed a decrease in size of peritoneal tumor implant and a decrease in size of hepatic metastases. Radiology report 3-5 months after initiation of the treatment confirmed prior findings. The investigator noted that this patient had excellent response to nab-sirolimus at week 6 with substantial decrease in liver and peritoneal metastases.

Patient #2

Patient #2 is a 70 year old female. She has stage IIB high grade serious ovarian cancer with retroperitoneal and pelvic metastases. Her prior treatment includes: cisplatin/paclitaxel, bevacizumab, olaparib, carboplatin, liposomal doxorubicin and gemcitabine. The patient is positive for the following somatic alterations: TSC1 (NM_000368—9q34.13) Deletion (Fold Change: −3.3); Other: TP53 (NM_000546) exon4 splicing variant p.X125_splice (c.375+2T>A); RB 1 (NM_000321—13q14.2) Loss (Fold Change: −1.7); MEF2B (NM_001145785) exon5 p.P169S (c.505C>T); NF1 (NM_WO1042492) exon13 p Y489C (c.1466A>G); RAF1 (NM_002880) exon5 p.K171 R (c.5 12A>G).

The patient started treatment of ABI-009 with a dose of 100 mg/m². She has completed five cycles. The patient developed a grade 2 Mucositis. No SAE or dose limiting events was developed. Radiology report about 2 months after initiation of treatment showed decreased retroperitoneal and pelvic nodal metastases. Radiology report one month later showed that retroperitoneal/pelvic lymph node metastases were unchanged and noted slightly increased size of some small retroperitoneal lymph nodes. The investigator noted that this patient has excellent response with decreasing peritoneal metastases and lymph nodes.

### Patient #3

Patient #3 is a 67 year old Female, who has metastatic high grade angiosarcoma in lower extremity with soft tissue and nodal metastasis. Her prior treatment includes: liposomal doxorubicin, paclitaxel, gemcitabine, vinorelbine, IL1 TNF, pazopanib, NKTR and nivolumab on clinical trial. She is positive for the following somatic alterations: MTOR (NM_004958) exon43 p.V2006F (c.6016G>T); other: TP53 exon4 p.P36Afs*7 (c.102dupC); MYC Amplification (Fold Change: 11.2); CDKN1 B Loss (Fold Change: −1.6); BRCA1 exon10 p.A887P (c.2659G>C); INPP4A exon22 p.V772F (c.2314G>T); RPS6KA4 exon13 p.H500R (c.1499A>G); IDH2 Rearrangement: c.988:IDH2_c.-2253 KIM0101 inv.

The patient started treatment of ABI-009 with a dose of 100 mg/m². She has completed five cycles. Some of her doses were delayed. Due to AE (rash), dose was reduced to 75 mg/m². She did not develop any SAE.

Radiology report about 1-2 months after initiation of the treatment showed increased central necrosis of left thigh subcutaneous mass and a decrease in size of left groin and pelvic subcutaneous tumor implants.

Radiology report one month later showed increased necrotic subcutaneous tumor mass anterior left thigh, no substantial change in metastatic soft tissue implants/nodes in the left groin and right anterior pelvis, and enhancement within the right vastus medialis with probable intramuscular edema.

The investigator noted that scans demonstrate decrease in tumor burden/stability in most areas. The investigator believed that the patient tolerated this dose without any new AEs. The investigator also noted that 10% with increased central necrosis was shown after 6 weeks of nab-sirolimus, and believed that the angiosarc response is remarkable. The central necrosis suggests tumor is dying.

### Patient #4

Patient #4 is an 89 year old Female. She has metastatic epithelial ovarian carcinoma.

Prior treatment includes liposomal doxorubicin, carboplatin, bevacizumab, gemcitabine, enzalutamide, MLN0128 (an mTOR inhibitor). The best response shown prior to ABI-009 treatment was seen after treatment of MLN0128 with a SD. The patient is positive for the following somatic alterations: TSC2 (NM_000548) exon42 p.C1755* (c.5265C>A); other: TP53 (NM_000546) exon6 p.Y220* (c.660T>G); SMARCA4 Amplification (Fold Change: 4.8); DNMT1 Amplification (Fold Change: 3.6); KEAP1 Amplification (Fold Change: 3.6); CARM1 Amplification (Fold Change: 3.6); FOXO1 Deletion (Fold Change: −2.4); BCL2L11 exon2 p.RI03Efs*8 (c.307_308delAG); CDKN1B exon1 p.L70* (c.208delC); EPHA5 exon3 p.D269N (c.805G>A).

The patient started treatment of ABI-009 at a dose of 100 mg/m². She has completed one cycle. No notable AEs were observed.

The Investigator noted that the patient withdrew consent for further treatment on the protocol after cycle 1 due to rise in CA 125 (from approx. 1000 to 1800) suggesting clinical progression. No follow up scan was available.

### Patient #5

Patient #5 is a 36 year old male with metastatic angiosarcoma in involving rt atrium, pericardium and bilateral lungs. Prior treatment includes first line AIM—doxorubicin, ifosfamide, mesna (unresponsive), new; and 2^{nd} line Taxol unsuccessful in stabilizing disease. He was positive for the following somatic alterations: TSC1 loss; other: CKS1B Amplification; POT1 (178T. G274E) (NM_015450) (233T>C. 821G>A); Other variants: APH1A amplification; CD22 (G655C); FAM123B (E385_E387del); FANCD2 (5612F); KDR (L743 G744insCSVL); MAP3K6 (V269G); TGFBR2 amplification; YY1AP1 amplification; CRLF2 (F107fs*9); FLT1 (P1201L); NTRK1 (G18E); ZNF217 (E519Q); ETS1 amplification; IL7R (166A); PDGFRB (V316M).

The patient started the treatment of ABI-009 at a dose of 100 mg/m². He has completed 1.5 cycles. Notable AEs include fasciitis, hyperglycemia; SAEs include hyperglycemia, hospitalization for infection.

Radiology report showed decreased size of right atrial angiosarcoma and lung metastases as compared to baseline. Investigator noted that CT scan done early at week 5 showed impressive response in the cardiac tumor and lung mets.

### Patient #6

Patient #6 is a 43 year old male, with metastatic high grade Sarcoma with metastasis to lung, bone and soft tissue and Li-Fraumeni syndrome. Prior treatment includes adriamycin and ifosfamide (4 Cycles); gemcitabine and taxotere (3 Cycles); surgery; Adjuvant gemcitabine (4½ cycles); pazopanib; pembrolizumab plus denosumab (7 cycles); and radiation. He was positive for the following somatic alterations: TSC2 (splice site 848+1G>C) (NM_000548); and other: DAXX (H300fs*70); RB1 (1297fs*13); TP53 (G245S); ASMTL (R525Q); ERBB3 (L1177I); FLT1 (T377I); RAD21 (T294A); YY1AP1 (S47P).

The patient started ABI-009 treatment at a dose of 100 mg/m. He has completed two cycles of treatment. Notable AEs include rash, oral ulcers. No SAEs or dose limiting events was shown.

Radiology report showed a dramatic response to therapy with significant interval improvement in hypermetabolic metastatic sarcoma involving the lungs, bones, lymph nodes, and skeletal muscles as compared to baseline. The investigated noted that the patient's PET/CT are consistent with a near complete response with complete de-activation of all of his tumor sites.

See Table 15 below for an analysis of mutations in the patients. In view of Table 9 and Table 15, at least two or more patients with TSC1 or TSC2 mutation that responded to ABI-009 have an aberration at any of FLT1, IL7R, RB1, TP53, PTEN, and YY1AP1.

TABLE 15

| | Patient #1 | Patient #2 | Patient #4 | | | |
|---|---|---|---|---|---|---|
| | Endometrial | Epithelial | Epithelial | Patient #5 | Patient #6 | Patient #7 |
| | stromal | ovarian | ovarian | angio- | High grade | Endometrial |
| Tumor | sarcoma | cancer | cancer | sarcoma | sarcoma | cancer |
| TSC1 | | Deletion, | | loss | | |
| | | Fold | | | | |
| | | change: −3.3 | | | | |
| TSC2 | Exon 18, | | Exon 42, | | splice site | exon22 |
| | C646*; | | C1755* | | 848 + 1 | p.E787*; |
| | Exon 30, | | | | G > C | exon27 |
| | W1194* | | | | | p.H1019Qfs* |
| | | | | | | 135 |
| MSI | Stable | Stable | Stable | | | stable |
| Status | | | | | | |
| APH1A | | | | Ampli- | | |
| | | | | fication | | |
| AR | Exon 1 | | | | | |
| | H41Q | | | | | |
| ASMTL | | | | | R525Q | |
| BCL2L11 | | | Exon 2, | | | |
| | | | R103Efs*8 | | | |
| CARM1 | | | Amplifi- | | | |
| | | | cation, fold | | | |
| | | | change 3.6 | | | |
| CD22 | | | | G655C | | |
| CDKN1B | | | Exon 1, | | | |
| | | | L70* | | | |
| CKS1B | | | | Ampli- | | |
| | | | | fication | | |
| CRLF2 | | | | F107fs*9 | | |
| DAXX | | | | | H300fs*70 | |
| DNMT1 | | | Amplifica- | | | |
| | | | tion, Fold | | | |
| | | | change: 3.6 | | | |
| EPHA5 | | | Exon 3, | | | |
| | | | D269N | | | |
| ERBB3 | | | | | L1177I | |
| ETS1 | | | | Ampli- | | |
| | | | | fication | | |
| FAM123B | | | | E385_E38 | | |
| | | | | 7del | | |
| FANCD2 | | | | 5612F | | |
| FLT1 | | | | P1201L | T377I | |
| FOXO1 | | | Deletion, | | | |
| | | | fold | | | |
| | | | change: −2.4 | | | |
| IL7R | Exon 8, | | | I66A | | |
| | K395R | | | | | |
| KDR | | | | L743_G74 | | |
| | | | | 4insCSVL | | |
| KEAP1 | | | Amplifi- | | | |
| | | | cation, fold | | | |
| | | | change: 3.6 | | | |
| MAP3K6 | | | | V269G | | |
| MEF2B | | Exon 5, | | | | |
| | | P169S | | | | |
| NF1 | | Exon 13, | | | | |
| | | Y489C | | | | |
| NTRK1 | Amplifi- | | | G18E | | |
| | cation, fold | | | | | |
| | change: 2.0 | | | | | |
| PDGFRB | | | | V316M | | |
| PTEN | | | | | | exon7 |
| | | | | | | p.K260Nfs*6 |
| POT1 | | | | 178T, | | |
| | | | | G274E | | |
| RAD21 | | | | | T294A | |
| RAF1 | | Exon 5, | | | | |
| | | K171R | | | | |
| RB1 | | Loss, fold | | | I297fs*13 | |
| | | change: −1.7 | | | | |
| SMARCA4 | | | Amplifi- | | | |
| | | | cation, Fold | | | |
| | | | change: 4.8 | | | |
| TGFBR2 | | | | Ampli- | | |
| | | | | fication | | |
| TP53 | | Exon 4: | Exon 6, | | G245S | |
| | | splicing | Y220* | | | |
| | | variant | | | | |
| YY1AP1 | | | | Ampli- | S47P | |
| | | | | fication | | |
| ZNF217 | | | | E519Q | | |
| TMB | | | | | | 2.6 mutations |
| Tumor | | | | | | per megabase |
| mutation | | | | | | (mt/Mb) |
| burden | | | | | | |

The above results with nab-sirolimus in patients with TSC1 or TSC2 mutations are particularly striking in view of low response rate seen in Kwiatkowski et al. (Clin Cancer Res. 2016, 22:2445-52). According to RECIST, standard definition for a response requires 30% tumor shrinkage. In Kwiatkowski et al, only 2/32 (6.25%) patients with TSC1 mutations or copy number loss and 0% patients with TSC2 mutations or copy number loss that were treated with an mTOR inhibitor (e.g., temsirolimus or everolimus) responded. In addition, in another study (Kwiatkowski. NCT02201212) only 2/30 (7%) responses were seen in patients with TSC1 or TSC2 mutations that were treated with everolimus.

### Example 7. Manufacturing and Characterization of ABI-009

This example demonstrates a method of making the ABI-009 composition of the preceding examples. More details can be found in PCT/US2020/057710, and U.S. Provisionals 62/927,047 and 62/936,212, which are hereby incorporated by reference in their entirety.

Emulsions were prepared to form albumin-rapamycin nanoparticles. The emulsions were optimized by testing different organic solvents at different ratios. An organic phase comprising chloroform and alcohol was tested at a 6:4 ratio of chloroform:ethanol or chloroform:isopropanol. An organic phase comprising chloroform and tert-butanol was tested at ratios of 6:4, 9:1, and 7:3 chloroform:tert-butanol. Samples were also tested in the presence or absence of 0.6 M NaCl or 10% sucrose. An aqueous solution comprising 30 mg/ml human albumin (HA) was prepared. The albumin contained the stabilizers sodium caprylate (0.08 mM/g) and N-acetyltryptophanate (0.08 mM/g). The aqueous solution and various organic solutions were mixed at a 96:4 ratio of aqueous solution:organic solution in a high-shear homogenizer to form the crude emulsion. Crude emulsions were fed into a high-pressure homogenizer coupled to a wiped film evaporator. The post-evaporate (PE) suspension was pooled and held at about 2° C. to about 8° C. After holding and pooling, the PE was assayed for rapamycin (by RP-HPLC) and HA (by SEC-UV). Based on assay values, the PE suspension was diluted with a 20% HA solution to yield a rapamycin concentration of about 7 mg/ml rapamycin and 56 mg/ml albumin. The different preparation conditions were assayed for particle size (before and after 0.2 μm filtration) and for filterability through a 0.2 μm filter. The results are summarized in Table 16, below.

TABLE 16
Bench scale manufacturing experiments.

| | | Z- | Z- | 0.2 μm |
|---|---|---|---|---|
| | | average (nm) | average (nm) | Filterability |
| Sample ID | Solvents | (unfiltered) | (filtered) | (ml per filter) |
| Sample 1 | CHCl3:EtOH | 193.5 | 175.8 | 7 |
| Sample 2 | CHCl3:EtOH | 195.9 | 171.2 | 4-5 |
| Sample 3 | CHCl3:EtOH | 178.6 | 159.9 | 7 |
| Sample 4 | CHCl3:EtOH | 154.7 | 135.9 | 10 |
| Sample 5 | CHCl3:EtOH | 183.6 | 169.1 | 10 |
| Sample 6 | CHCl3:EtOH | 194.9 | 179.1 | 7 |
| Sample 7 | CHCl3:tBa | 191.4 | 175.6 | 10 |
| Sample 8 | CHCl3:IPA | 199.7 | 178.8 | 7-8 |
| Sample 9 | CHCl3:EtOH | 212.5 | 189.5 | 7.5 |
| Sample 10 | CHCl3:tBa | 134.6 | 83.3 | 10 |
| Sample 11 | CHCl3:tBa | 155.1 | 138.2 | 12-15 |
| Sample 12 | CHCl3:tBa | 224.0 | 153.9 | 2-3 |
| Sample 13 | CHCl3:EtOH | 174.1 | 148.2 | 5-7 |

Sample 11 demonstrated the best filterability based on volume per filter and low average particle size. Further, Sample 11 had reduced fibers as determined by light microscope, compared to the other samples. The optimized conditions of Sample 11 were used to prepare ABI-009.

The optimized conditions of Sample 11 are used to prepare commercial batches of the pharmaceutical composition. Diluted PE of the commercial batch are filtered through a 0.2 μm filter. Filtered product are aliquoted into approximately 5000-60(0) depyrogenated vials and plugged with sterilized stoppers to yield sealed vials of the final product comprising lyophilized cake of about 100 mg rapamycin and about 800 mg albumin each. The atmosphere of each vile is replaced with nitrogen NF before stoppering. Each vial contains about 0.068 mM/vial of each of sodium caprylate and N-acetyltryptophanate and only trace or undetectable amounts of chloroform and tert-butanol. Each vial may be reconstituted with 20 ml of 0.9% NaCl to yield an injection of 5 mg/ml rapamycin.

A study was undertaken using size exclusion chromatography with multi-angle light scattering and refractive index detection (SEC-MALS-RI) to characterize the albumin oligomer status of albumin in ABI-009. Manufactured lots of the lyophilized product (vials comprising 100 mg of rapamycin in rapamycin protein-bound particles) were reconstituted with 20 ml saline to yield 5 mg/ml rapamycin. Samples were centrifuged at 14,000 rpm in a Beckman Coulter Microfuge 22R centrifuge for 1 hour at 24° C. Samples could be aliquoted and frozen, but only one freeze/thaw cycle was allowed. Normalization constants were determined with U.S.P. Albutein®, 25% (Lot No. B3ALC00082) standard at 4 mg/ml concentration in saline. 100 μl of each sample was injected in a BioSep-S3000 (<7×10⁵ Da; 5 μm) column with a saline mobile phase at a flow rate of 1 ml/min. Wyatt DAWN HELEOS II and Wyatt Optilab T-rEX detectors were used. Nanoparticle samples were diluted 10-fold in saline before injection. Reconstituted stock samples, pellets from centrifugation and supernatants from centrifugation were tested. As a control for centrifugation, samples were also resuspended without separating supernatant to test stability of the oligomer profile from centrifugation.

ABI-009 lots designated lot #1, lot #2, lot #3, lot #8, lot #10, lot #14, and lot #16 were assessed. Samples were tested without centrifugation (stock) or after centrifugation (pellet and supernatant). As a control, samples were also resuspended after pelleting, to demonstrate the pelleting did not substantially alter the oligomer profile.

TABLE 17
SEC-MALS-RI Oligomer study

| Sample | Monomer (%) | Dimer (%) | Trimer (%) |
|---|---|---|---|
| Lot #1 before centrifugation | 89.0 | 9.2 | 1.8 |
| Lot #1 after centrifugation | 88.3 | 9.6 | 2.3 |
| Lot #1 pellet | 77.0 | 13.5 | 9.5 |
| Lot #1 supernatant | 89.3 | 9.2 | 1.5 |
| Lot #2 before centrifugation | 87.7 | 9.9 | 2.4 |
| Lot #2 after centrifugation | 87.8 | 9.9 | 2.3 |
| Lot #2 pellet | 74.1 | 15.2 | 10.6 |
| Lot #2 supernatant | 88.9 | 9.3 | 1.7 |
| Lot #3 before centrifugation | 89.1 | 8.9 | 2.0 |
| Lot #3 after centrifugation | 89.2 | 8.8 | 2.0 |
| Lot #3 pellet | 80.0 | 12.4 | 7.6 |
| Lot #3 supernatant | 90.1 | 8.4 | 1.5 |
| Lot #8 before centrifugation | 86.3 | 10.9 | 2.9 |
| Lot #8 after centrifugation | ND | ND | ND |
| Lot #8 pellet | 77.7 | 13.9 | 8.4 |
| Lot #8 supernatant | 87.3 | 10.3 | 2.3 |
| Lot #10 before centrifugation | 89.1 | 8.8 | 2.1 |
| Lot #10 after centrifugation | ND | ND | ND |
| Lot #10 pellet | 74.0 | 15.5 | 10.5 |
| Lot #10 supernatant | 89.9 | 8.4 | 1.7 |
| Lot #14 before centrifugation | 90.7 | 7.9 | 1.4 |
| Lot #14 after centrifugation | ND | ND | ND |
| Lot #14 pellet | 78.5 | 12.9 | 8.6 |
| Lot #14 supernatant | 89.6 | 8.7 | 1.7 |
| Lot #16 before centrifugation | 89.1 | 8.8 | 2.1 |
| Lot #16 after centrifugation | 89.2 | 8.8 | 2.0 |
| Lot #16 pellet | 74.2 | 16.2 | 9.6 |
| Lot #16 supernatant | 90.0 | 8.4 | 1.6 |

Additional characterization of the oligomer profile of human albumin in ABI-009 was performed with an alternative method. Samples from ABI-009 lots designated Lot #1, Lot #2, Lot #5, and Lot #15 were assessed. Lyophilized samples from each lot were reconstituted in saline to yield a reconstituted pharmaceutical suspension with approximately 5 mg/mL rapamycin.

To assess the total albumin oligomeric profile, a Stock Sample Suspension was prepared at a target concentration of 1.8 mg/mL rapamycin by quantitatively transferring each reconstituted sample suspension into a 500 mL volumetric flask using water and then diluting to volume with water. The Stock Sample Suspension was stored at 2-8° C. A Working Sample Suspension was prepared at a target concentration of 0.18 mg/mL by diluting 5.0 mL of the Stock Sample Suspension to 50 mL with water. The Working Sample Suspension was stored at 2-8° C. Size exclusion chromatography was used with a column of appropriate separation capability for albumin, with UV detection at 228 nm. The mobile phase comprised 0.10 M K2HPO4 in 7.5% methanol. The peaks in the chromatogram were integrated to determine the composition of the different oligomeric species and the total albumin in the composition.

To determine the albumin oligomeric profile of the nanoparticle portion and non-nanoparticle portion of the compositions, 4 mL of the 5 mg/mL rapamycin reconstituted suspensions were transferred into ultra-centrifugation tubes and centrifuged at 50,000 rpm for 41 minutes. The supernatants were separated using a micro-pipette without disturbing the pellet and analyzed by SEC with UV detector with a mobile phase comprising 0.10 M K2HPO4 in 7.5% methanol as above. The pellets (the nanoparticle portion) were washed carefully with 2-3 mL of purified water. The rinsate was decanted. 2 mL of ethanol was added to the pellet. The pellet in ethanol was then sonicated in a water bath until fully dispersed. The dispersed pellet was transferred by pipette to a new ultra-centrifugation tube. An additional 3 mL of ethanol was added and the tubes were centrifuged at 10,000 rpm for 20 minutes. Following centrifugation, the supernatant was decanted without disturbing the pellet. 3 mL of saline was added to the pellet and allowed to dissolve for 15 minutes. Using a glass Pasteur pipette, the mixtures were transferred into a 10 mL volumetric flask. Saline was used to transfer the remaining material into the 10 mL volumetric flask. The samples were diluted to 10 mL with saline and sonicated until completely dissolved. The samples were analyzed by SEC with UV detector with a mobile phase comprising 0.10 M K2HPO4 in 7.5% methanol to determine the oligomeric profile of the nanoparticle portion.

The oligomeric profiles for Lots #1, #2, #5, and #15 for the total composition, the non-nanoparticle portion, and the nanoparticle portions are summarized in Table 18, below.

TABLE 18
Composition of Human Albumin in ABI-009

| | Human Albumin Composition (%) | | | |
|---|---|---|---|---|
| Lot #/Portion | Monomer | Dimer | Oligomer | Polymer |
| Lot #1/Total | 85.06 | 8.53 | 2.14 | 4.27 |
| Lot #1/Non-nanoparticle | 89.23 | 8.16 | 1.77 | 0.83 |
| Lot #1/Nanoparticle | 36.99 | 10.96 | 3.47 | 48.58 |
| Lot #2/Total | 85.08 | 8.89 | 2.13 | 3.89 |
| Lot #2/Non-nanoparticle | 89.01 | 8.52 | 1.72 | 0.75 |
| Lot #2/Nanoparticle | 38.5 | 11.23 | 3.72 | 46.55 |
| Lot #5/Total | 86.94 | 7.41 | 1.6 | 4.05 |
| Lot #5/Non-nanoparticle | 90.38 | 6.99 | 1.59 | 1.04 |
| Lot #5/Nanoparticle | 39.13 | 10.34 | 2.93 | 47.60 |
| Lot #15/Total | 85.49 | 8.34 | 2.05 | 4.11 |
| Lot #15/Non-nanoparticle | 89.13 | 8.09 | 1.86 | 0.92 |
| Lot #15/Nanoparticle | 38.56 | 9.72 | 2.65 | 49.07 |

A study was also undertaken to analyze rapamycin drug release from 12 lots of ABI-009 (Lots #1-10 and Lots 14-15) using a stable isotope tracer ultrafiltration assay (SITUA) (see Skoczen et al., Stable Isotope Method to Measure Drug Release from Nanomedicines, J. Control Release, 220(A):169-174 (2015). Drug release was examined at 10 μg/ml and 500 μg/ml of rapamycin following 10 minutes of incubation. Briefly, stable, isotope-labeled rapamycin was spiked into 4.5% human serum albumin (25% Albutein HSA diluted in 0.9% saline). MeOH-solvent rapamycin (as a control) or fresh reconstituted samples of each lot at 10 μg/ml or 500 μg/ml were added. After 10 minutes of equilibration at 29° C., a portion of the sample is taken and filtered using Vivacon®) 10 kDa MWCO centrifuge devices prewarmed to 29° C. The sample and the ultrafiltrate are analyzed by LC-MS to determine the concentrations of normal rapamycin and isotope-labelled rapamycin. The ultrafilterable fraction of isotope-labeled rapamycin represents a measurement of free unbound fraction.

The encapsulated and unencapsulated nanoparticle fractions can also be calculated.

TABLE 19
Lot comparison of drug release

| | 10 μg/ml | | 500 μg/ml | |
|---|---|---|---|---|
| | Avg. | | Avg. | |
| | Release | SD/% | Release | SD/% |
| Lot | (%) | CV | (%) | CV |
| Free | 97.2 | 5.0/5.1 | 18.3 | 2.1/11.8 |
| rapamycin | | | | |
| Lot #1 | 94.7 | 2.0/2.2 | 16.7 | 1.2/7.5 |
| Lot #2 | 89.8 | 3.4/3.8 | 15.6 | 0.6/3.8 |
| Lot #3 | 89.7 | 3.2/3.5 | 15.1 | 0.7/4.6 |
| Lot #4 | 107.5 | 1.5/1.4 | 23.3 | 1.0/4.3 |
| Lot #5 | 107.7 | 3.1/2.9 | 24.7 | 0.7/2.8 |
| Lot #6 | 104.5 | 3.4/3.3 | 23.2 | 2.0/8.7 |
| Lot #7 | 99.9 | 4.3/4.3 | 19.5 | 1.0/5.3 |
| Lot #8 | 96.0 | 2.2/2.3 | 18.5 | 0.8/4.6 |
| Lot #9 | 99.1 | 2.3/2.3 | 19.3 | 1.5/7.8 |
| Lot #10 | 100.6 | 9.0/8.9 | 15.4 | 2.1/13.6 |
| Lot #14 | 100.7 | 5.2/5.2 | 16.1 | 0.9/5.4 |
| Lot #15 | 106.3 | 2.5/2.4 | 17.7 | 1.1/5.9 |

As summarized in Table 19, all lots displayed 89-106% calculated release at 10 μg/ml and 15-25% release at 500 μg/ml, similar to a free drug control, supporting solubility-dependent drug release and indicating a consistent formulation. Standard deviations and coefficients of variation are also indicated.

### Example 8

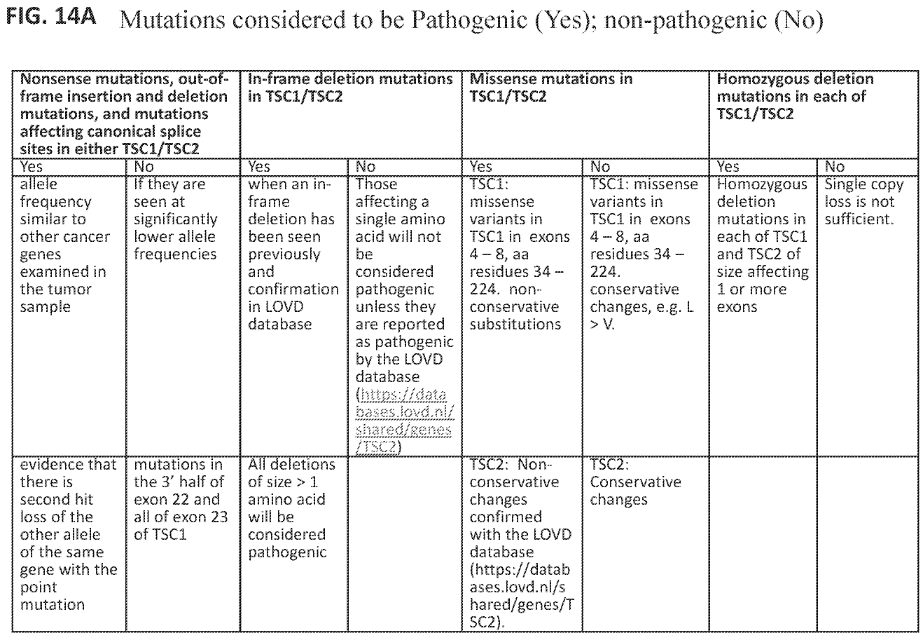

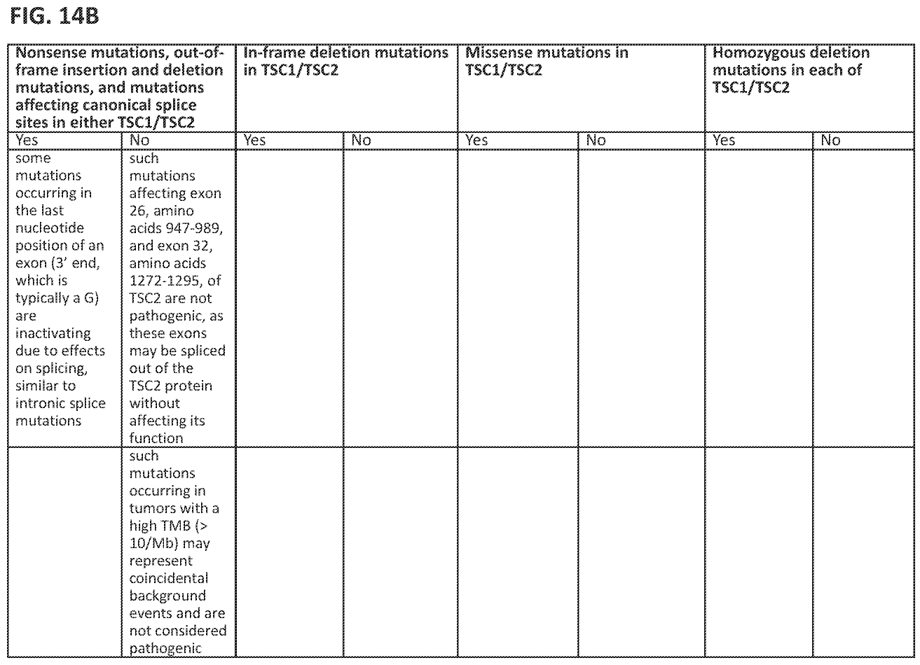

An algorithm was designed to assess whether a particular mutation is pathogenic. See FIGS. 14A-14B.

### Example 9

An analysis was conducted to study additional aberrations seen in other genes in the patients with a TSC1 or TSC2 mutation (including inactivating mutation, a loss or deletion of the gene) based upon results discussed in the application (such as in Examples 1, 2, 3A, 3B and 5) and a few references. See Wagle, et al, N Engl J Med 2014; 371:1426-1433; Perini et al Blood Cancer Journal 6, e420 (2016); Alsidawi and Kasi 2018 Cold Spring Harb Mol Case Stud 4: a00222, Dickson et al., Int J Cancer. 2013 Apr. 1:132(7):1711-7; Wagner et al., J Clin Oncol. 2010 Feb. 10:28(5):835-40; Lyer et al., Science. 2012 Oct. 12; 338(6104):221; Lim et al., Oncotarget. 2016; 7:24172-24178; Kwiatkowski et al. Clin Cancer Res. 2016 May 15:22(10):2445-2452; Voss et al. Clin Cancer Res Jan. 15, 2019 (25) (2) 506-514; Roldan-Romero et al., Int. J. Cancer, 146: 1435-1444; and Huynh et al., Mol Cancer Ther. 2015 May; 14(5):1224-35).

We found that the following mutations occurs in patients with a TSC1 or TSC2 mutation: AKT1, ALK, APC, APH1A, AR, ARID1A, ARID1B, ARID2, ASMTL, ASXL1, ATR, ATRX, AXIN1, AXL, BAP1, BARD1, BCL11A, BCL2L11, B2M BLM, BRCA1, BRCA2, BRD4, BRIP1, BUB1B, CASC5, C17orf70, C19orf40, CARM1, CCND3, CCNE1, CD22, CD36 CD274, CDC73, CDH4, CDK12, CDKN1A, CDKN1B, CDKN2A, CDKN2B, CDKN2C, CEBPA, CHEK1, CIC, CSF1R, CKS1B, CREBBP, CRLF2, CTCF, CYLD, DAXX, DCC, DDR1, DDR2, DICER1, DMC1, DNMT1, DNMT3A, EP300, EPCAM, EPHA3, EPHA5, ERCC5, ERBB3, ERBB4, ERRF11, ETS, ETV1, ETV4, EXO1, EXT1, EZH2, FAM123B, PAN), FANCA, FANCB, FANCD2, FANCF, FANCL, FAS, FAT1, FBX011, FGF6, FGFR3, FGFR4, FLCN, FLT, FLT3, FLT4, FOXL2, FOXO1, GATA1, GATA2, GATA6, GEN1, GLI1, GLI2, GNAS, H19, HELQ, HGF, HNF1A, IL7R, JAK1, JAK2, JAK3, JAZF1, KAT6B, KDM4C, KDM5C, KDM6A, KDR, KEAP1, KIT, KLF4, KMT2A, KMT2D, KRAS, MAP2K2, MAP3KL, MAP3K6, MCL1, MCM8, MEF2B, MEN, MET, MGA, MLLT10, MSH2, MSH3, MSH6, mTOR, MUTYH, MYCN, NBN, NF1, NF2, NPM1, NOTCH1, NOTCH2, NOTCH3, NRG1, NR0B1, NSD1, NTRK1, PARP1, PRKDC, PBRM1, PDCD1LG2, PDGFRA, PDGFRB, PIK3C2B, PIK3C2G, PIK3CG, PIK3RI, PMS2, POLD1, POLE, POLQ, POT1, PRKDC, PTCH1, PTEN, PTPRD, PVRL4, RAD21, RAD50, RAD51C, RANBP2, RAF1, RB1, RBBP8, RBM10, RET, RICTOR, RIF1, RIT1, RNF43, ROS1, RPTOR, RSPO2, SDHA, SETBP1, SETD2, SMAD2, SMAD4, SMARCA4, SMO, SNCAIP, SOCS1, SOX9, SUFU, TAF, TCEB1, TET2, TGFBR2, TIPARP, TLX3, TNFAIP3, TP53, TP53BP1, TRIM37, TSHR, TYRO, UIMC1, VHL, WHSC1L1, WRN, XPA, XPO1, YY1AP1, ZNF217.

Among those genes, an aberration in ARID1A, ARID1B, AXIN1, BAP1, BRCA2, BUB1B, CDH4, CDKN2C, ERBB3, EXT1, FANCD2, FAT1, FLT1, FLT4, FOXL2, GLI1, GLI2, GNAS, IL7R, KDM6A, KIT, NOTCH3, NSD1, NTRK1, PARP1, PBRM1, PIK3CG, PMS2, POLDI, POLE, PTCH1, PTEN, RB1, RET, RIF1, SETD2, SMARCA4, TLX3, WRN, XPO1, or YY1AP1 was seen in more than 5% of the patients analyzed.

Aberration in ARID1A, BAP1, CDKN2C, ERBB3, FLT1, NTRK1, PBRM1, PTEN, RB1, RIF1, SETD2, SMARCA4, TLX3, TP53, or VHL was seen in more than 10% of the patients analyzed.

Aberration in RB1 and PTEN were seen in more than 20% of the patients analyzed.

Mutations in any of APH1A. ASXL1, BCL2L11, BRD4, BUB1B, C17orf70, C19orf40, CARM1, CCNE1, CD22, CDKN1A, CDKN1B, CDKN2C, CEBPA, CHEK1, CIC CKS1B, CRLF2, CTCF, CYLD, DAXX, DMC1, DNMT1, EPCAM, ERBB3, ETS, ETV1, ETV4, EXO1, EXT17, FAM123B, FANCA, FANCB, FGFR4, FLT1, FLT4, FOXO1, GATA2, GEN1, GLI1, GLI2, H19, HELQ, IL7R JAK3, JAZF7, KAT6B, KDR, KEAP1, KMT24, MAP3K6, MCL1, MCM8, MEF2B, MEN1, MYCN, NF1, NPM1, NRG1, NR0B1, NSD1, NTRK1, PRKDC, PDGFRA, POLQ, POT1, PRKDC, PVRL4, RAD21, RAF1, RIT1, RNF43, ROS1, RPTOR, SDHA, SETBP1, SMARCA4, SOCS1, TCEB1, TET2, TSHR UIMC1, WHSC1L1, XPA, YY1AP1, and ZNF217 were observed in patients with a TSC1 or TSC2 mutation based upon the Examples described herein. None of those mutations were observed in patients with a TSC1 or TSC2 mutation described in any of the referenced discussed above.

Mutations in any of AR, APH1A, ATRX, ARID1B, BRD4, BRCA2, BUB1B, CCNE1, C19orf40, CDH4, CDKN2C, CD22, CEBPA, CHEK1, CKS1B, CRLF2, CTCF, CYLD, DICER1, DMC1, DNMT3A, EP300, ERCC5, ERBB3, ETV4, ETS1, EXO1, EXT1, FAM123B, FANCB, FANCF, FANCD2, FAN1, FLT1, FOXL2, GATA2, GEN1, GLI1, GLI2, IL7R, KAT6B, KDR, KIT, KMT2A, KMT2D, MAP3K6, MCL1, MAP3KL, MCM8, MEF2B, MEN1, MSH2, MUTYH, MYCN, NOTCH3, NSD1, NF1, NTRK1, PDGFRB, POT1, POLQ, PVRL4, RAF1, RB1, RBBP8, RIF1, RIT1, RNF43, RPTOR, ROS1, SDHA, SMARCA4, SUFU, TCEB1, TET2, TGFBR2, TLX3, TP53, TP53BP1, TSHR, WHSC1L1, XPA, YY1AP1, and ZNF217 was observed in patients with a TSC1 mutation based upon the Examples described herein, Mutations in APH1A, BRD4, BUB1B, CCNE1, C19orf40, CDKN2C, CD22, CEBPA, CHEK1, CKS1B, CRLF2, CTCF, CYLD, DMC1, ERBB3, ETV4, ETS1, EXO1, EXT1, FAM123B, FANCB, FLT1, GATA2, GEN1, GLI1, GLI2, IL7R, KAT6B, KDR KMT2A, MAP3K6, MCL1, MCM8, MEF2B, MEN1, MYCN, NSD1, NF1, NTRK1, POT1, POLQ, PVRL4, RAF1, RIT1, RNF43, RPTOR, ROS1, SDHA, SMARCA4, TCEB1, TET2, TSHR, WHSC1L1, XPA, YY1AP1, and ZNF217 were not described in any of the references discussed above.

Mutations in any of ATR, AR, ASMTL, ASXL1, BCL2L11, BLM, BRCA2, BRIP1, BUB1B, CARM1, C17orf70, C19orf40, CIC, CCNE1, CDH4, CDKN2C, CDKN1A, CDKN1B, DAXX, DNMT1, EPHA5 EPCAM, ERBB3, ETV1, EXO1, EXT1, EZH2, FAT1, FAN1, FANCA, FANCL, FANCD2, FGFR3, FGFR4, FAS, FAT1, FLT1, FOXO1, FLT4, GNAS, GLI2, H19, HELQ, IL7R, JAK2, JAZF1, KAT6B, KDM6A, KEAP1, KIT KLF4, MAP3KL, MCM8, MGA, NPM1, NRG1, NR0B1, NTRK1, PDGFRA, PDGFRB, PIK3C2B, PMS2, POLQ, PRKDC, PTEN, PTCH1, PRKDC, RAD21, RAD50, RB1, RET, RIF1, RSPO2, SETBP1, SETD2, SMARCA4, SOCS1, TLX3, TP53, TRIM37, UIMC1, VHL, WHSC1L1, XPA, WRN, and YY1AP1 was observed in patients with a TSC2 mutation based upon the Examples described herein.

Mutations in any of ASMTL, ASXL1, BCL2L11, BUB1B, CARM1, C17orf70, C19orf40, CIC, CCNE1, CDKN2C, CDKN1A, CDKN1B, DAXX, DNMT1, EPCAM, ERBB3, ETV1, EXO1, EXT1, FANCA, FGFR4, FLT7, FOXO1, FLT4, GLI2, H19, HELQ, IL7R, JAK2, JAZF1, KAT6B, KEAP1, MCM8, NPM1, NRG1, NR0B1, NTRK1, PDGFRA, POLQ, PRKDC, RAD21, SETBP1, SMARCA4, SOCS1, UIMC1, WHSC1L1, XPA, and YY1AP1 were not described in any of the references discussed above.

Based upon information from references discussed above, and results discussed in the application (such as in Examples 1, 2, 3A, 3B and 5) (total 92 patients), one or more mutations in any one or more of TP53, RB1, VHL, PBRM1, PTEN, SETD2, BAP1, BRCA2, FANCD2, ARID1A, ARID1B, CDKN2A, FAT1, KDM6A, KIT, PDGFRB, RIF1 were observed in at least about 5.7% of the patients who had a TSC1 or TSC2 mutation. One or more mutations in any one or more of TP53, RB1. VHL were observed in at least about 11.5% of the total patients who had a TSC1 or TSC2 mutation. Among those, Mutation in TP53 was observed in at least about 49.4% of the patients who had a TSC1 or TSC2 mutation. Mutation in RB1 or VHL was observed in at least 17.2% or 11.5%, respectively, of the total patients who had a TSC1 or TSC2 mutation. See Table 20 below.

Based upon results discussed in the application (such as in Examples 1, 2, 3A, 3B and 5) (total 25 patients), one or more mutations in any one or more of TP53, RB1, TLX3, SMARCA4, RIF1, PTEN, NTRK1, FLT1, ERBB3, CDKN2C, ATRX YY1AP1, XPA, WRN, PTCH1 PMS2, PDGFRB, NSD1, KMT2A, KDM6A, IL7R, GNAS, GLI2, GLI1, FLT4, FAT1, FANCD2, EXT1, DNMT3A, DAXX, CDH4, CCNE1, and BUB1B were observed in at least about 8% of the patients who had a TSC1 or TSC2 mutation. Among those, one or more mutations in any one or more of TP53, RB1, TLX3, SMARCA4, RIF, PTEN NTRK1, FLT1, ERBB3, CDKN2C, and ATRX were observed in at least about 12% of the patients who had a TSC1 or TSC2 mutation. Mutation in TP53 or RB1 was observed in at least about 48% or 28%, respectively of the patients who had a TSC1 or TSC2 mutation. See Table 20 below.

TABLE 20
Mutation frequencies in patients with TSC1 or TSC2 mutation.

| | All | | ABI-009 | | Literature |
|---|---|---|---|---|---|
| Gene | Data | Gene | pts only | Gene | only |
| TP53 | 49.4% | TP53 | 48.0% | TP53 | 50.0% |
| MSS | 29.9% | RB1 | 28.0% | MSS | 32.3% |
| TMB < 10 | 18.4% | MSS | 24.0% | TMB < 10 | 22.6% |
| RB1 | 17.2% | PTEN | 12.0% | VHL | 14.5% |
| VHL | 11.5% | RIF1 | 12.0% | RB1 | 12.9% |
| PTEN | 9.2% | ATRX | 12.0% | PBRM1 | 12.9% |
| PBRM1 | 9.2% | TLX3 | 12.0% | TMB > 40 | 12.9% |
| TMB > 10 | 9.2% | CDKN2C | 12.0% | SETD2 | 9.7% |
| SETD2 | 8.0% | ERBB3 | 12.0% | BAP1 | 9.7% |
| BRCA2 | 6.9% | FLT1 | 12.0% | PTEN | 8.1% |
| FANCD2 | 6.9% | NTRK1 | 12.0% | ARID1A | 8.1% |
| BAP1 | 6.9% | SMARCA4 | 12.0% | CDKN2A | 8.1% |
| RIF1 | 5.7% | TMB < 10 | 8.0% | BRCA2 | 6.5% |
| FAT1 | 5.7% | BRCA2 | 8.0% | FANCD2 | 6.5% |
| KDM6A | 5.7% | FANCD2 | 8.0% | ARID1B | 6.5% |
| PDGFRB | 5.7% | FAT1 | 8.0% | KIT | 6.5% |
| ARID1B | 5.7% | KDM6A | 8.0% | AXIN1 | 6.5% |
| KIT | 5.7% | PDGFRB | 8.0% | POLD1 | 6.5% |
| ARID1A | 5.7% | CCNE1 | 8.0% | FAT1 | 4.8% |
| CDKN2A | 5.7% | DAXX | 8.0% | KDM6A | 4.8% |
| ATRX | 4.6% | GNAS | 8.0% | PDGFRB | 4.8% |
| TLX3 | 4.6% | PMS2 | 8.0% | FGFR3 | 4.8% |
| CCNE1 | 4.6% | CDH4 | 8.0% | FOXL2 | 4.8% |
| DAXX | 4.6% | DNMT3A | 8.0% | KMT2D | 4.8% |
| GNAS | 4.6% | PTCH1 | 8.0% | NOTCH3 | 4.8% |
| PMS2 | 4.6% | WRN | 8.0% | RET | 4.8% |
| FGFR3 | 4.6% | BUB1B | 8.0% | CDKN2B | 4.8% |
| FOXL2 | 4.6% | EXT1 | 8.0% | PARP1 | 4.8% |
| KMT2D | 4.6% | FLT4 | 8.0% | PIK3CG | 4.8% |
| NOTCH3 | 4.6% | GLI1 | 8.0% | POLE | 4.8% |
| RET | 4.6% | GLI2 | 8.0% | XPO1 | 4.8% |
| AXIN1 | 4.6% | IL7R | 8.0% | RIF1 | 3.2% |
| POLD1 | 4.6% | KMT2A | 8.0% | CCNE1 | 3.2% |
| CDKN2C | 3.4% | NSD1 | 8.0% | DAXX | 3.2% |
| ERBB3 | 3.4% | XPA | 8.0% | GNAS | 3.2% |
| FLT1 | 3.4% | YY1AP1 | 8.0% | PMS2 | 3.2% |

MSS stable microsatellite status.

For analysis in this example, patients with a complete response, partial response or achieving stable disease were deemed responding to the mTOR inhibitor or ABI-009.

Based upon information from references discussed above, and results discussed in the application (such as in Examples 1, 2, 3A, 3B and 5) (total about 51 patients), one or more mutations in any one or more of TP53, VHL, RB1, PBRM1, ATRX, KDM6A, RET, SETD2, ARID1A, BAP1, FLT1, NTRK1, TLX3, and BRCA2 were observed in at least about 5.9% of the responding patients to an mTOR inhibitor (e.g., ABI-009) who had a TSC1 or TSC2 mutation. Among those, one or more mutations in any one or more of TP53, VHL, RB1, PBRM1 were observed in at least about 11.8% of the responding patients to an mTOR inhibitor (e.g., ABI-009) who had a TSC1 or TSC2 mutation. See Table 21 below.

Based upon the results discussed in the application (such as in Examples 1, 2, 3A, 3B and 5) (total about 18 patients), one or more mutations in any one or more of TP53, RB1, ATRX, FLT1, NTRK1, TLX3, KDM6A, CDH4, CDKN2C, DAXX, ERBB3, GNAS, IL7R, PDGFRB, PMS2. PTEN. SMARCA4, and YY1AP1 were observed in at least about 11.1% of the responding patients to an mTOR inhibitor (e.g., ABI-009) who had a TSC1 or TSC2 mutation. Among those, one or more mutations in any one or more of TP53, RB1, ATRX, FLT1, NTRK1, and TLX3 were observed in at least about 16.7% of the responding patients to an mTOR inhibitor (e.g., ABI-009) who had a TSC1 or TSC2 mutation. See Table 21 below.

TABLE 21
Mutation frequencies in mTOR responding patients
with TSC1 or TSC2 mutation

| | All | | ABI-009 | | Literature |
|---|---|---|---|---|---|
| Gene | Data | Gene | pts only | Gene | only |
| TP53 | 43.1% | TP53 | 44.4% | TP53 | 42.4% |
| VHL | 17.6% | MSS | 33.3% | VHL | 27.3% |
| MSS | 11.8% | RB1 | 22.2% | PBRM1 | 18.2% |
| RB1 | 11.8% | ATRX | 16.7% | ARID1A | 12.1% |
| PBRM1 | 11.8% | FLT1 | 16.7% | BAP1 | 12.1% |
| ATRX | 7.8% | NTRK1 | 16.7% | RET | 9.1% |
| KDM6A | 7.8% | TLX3 | 16.7% | SETD2 | 9.1% |
| RET | 7.8% | KDM6A | 11.1% | RB1 | 6.1% |
| SETD2 | 7.8% | CDH4 | 11.1% | KDM6A | 6.1% |
| ARID1A | 7.8% | CDKN2C | 11.1% | BRCA2 | 6.1% |
| BAP1 | 7.8% | DAXX | 11.1% | ATRX | 3.0% |
| FLT1 | 5.9% | ERBB3 | 11.1% | AR | 3.0% |
| NIKK1 | 5.9% | GNAS | 11.1% | ARID2 | 3.0% |
| TLX3 | 5.9% | IL7R | 11.1% | ASXL1 | 3.0% |
| BRCA2 | 5.9% | PDGFRB | 11.1% | ATR | 3.0% |
| CDH4 | 3.9% | PMS2 | 11.1% | DNMT3A | 3.0% |
| CDKN2C | 3.9% | PTEN | 11.1% | FANCD2 | 0.030303 |
| DAXX | 3.9% | SMARCA4 | 11.1% | FGFR3 | 3.0% |
| ERBB3 | 3.9% | YY1AP1 | 11.1% | JAK2 | 3.0% |
| GNAS | 3.9% | TMB < 10 | 11.1% | PTCH1 | 3.0% |

Table 22 below shows mutation frequencies in non-responders to an mTOR inhibitor who had a TSC1 or TSC2 mutation. Mutations in GLI1, KMT2A, NSD1, RIF1, or XPA were seen in non-responders at a frequency higher than the responders to an mTOR inhibitor (e.g., ABI-009).

TABLE 22
Mutation frequencies in mTOR non-responders
with TSC1 or TSC2 mutation.

| | All | | ABI-009 | | Literature |
|---|---|---|---|---|---|
| Gene | Data | Gene | pts only | Gene | only |
| TP53 | 50.0% | TP53 | 57.1% | TP53 | 33.3% |
| RB1 | 30.0% | RB1 | 42.9% | FGFR3 | 33.3% |
| GLI1 | 20.0% | GLI1 | 28.6% | KDM6A | 33.3% |
| KMT2A | 20.0% | KMT2A | 28.6% | | |
| NSD1 | 20.0% | NSD1 | 28.6% | | |
| RIF1 | 20.0% | RIF1 | 28.6% | | |
| XPA | 20.0% | XPA | 28.6% | | |

Table 23 below shows mutation frequencies in patients who had a TSC1 mutation. Based upon the information from references discussed above, and results discussed in the application (such as in Examples 1, 2, 3A, 3B and 5) (total 44 patients), one or more mutations in any one or more of TP53, RB1, VHL, and PBRM1 were observed in at least about 16.3% of the total patients who had a TSC1 mutation. Based upon the results discussed in the application (such as in Examples 1, 2, 3A, 3B and 5) (total 9), one or more mutations in any one or more of TP53, RB1, GLI1, KMT2A, NSD1, NTRK1, SMARCA4 and XPA were observed in at least about 22.2% of the total patients who had a TSC1 mutation.

TABLE 23
Mutation frequencies in patients with TSC1 mutation.

| | All | | ABI-009 | | Literature |
|---|---|---|---|---|---|
| Gene | Data | Gene | pts only | Gene | only |
| TP53 | 48.8% | TP53 | 66.7% | TP53 | 44.1% |
| RB1 | 20.9% | RB1 | 55.6% | VHL | 23.5% |
| MSS | 18.6% | GLI1 | 22.2% | MSS | 20.6% |
| VHL | 18.6% | KMT2A | 22.2% | PBRM1 | 20.6% |
| PBRM1 | 16.3% | NSD1 | 22.2% | RB1 | 11.8% |
| ARID1B | 9.3% | NTRK1 | 22.2% | BAP1 | 11.8% |
| FANCD2 | 9.3% | SMARCA4 | 22.2% | POLD1 | 11.8% |
| FAT1 | 9.3% | XPA | 22.2% | SETD2 | 11.8% |
| FOXL2 | 9.3% | APH1A | 11.1% | TMB < 10 | 11.8% |
| KIT | 9.3% | ARID1B | 11.1% | TMB > 10 | 11.8% |
| NOTCH3 | 9.3% | ATRX | 11.1% | ARID1B | 8.8% |
| PDGFRB | 9.3% | BRCA2 | 11.1% | FANCD2 | 0.088235 |
| PTEN | 9.3% | BRD4 | 11.1% | FAT1 | 8.8% |
| BAP1 | 9.3% | BUB1B | 11.1% | FOXL2 | 8.8% |
| POLD1 | 9.3% | C19orf40 | 11.1% | KIT | 8.8% |
| SETD2 | 9.3% | CCNE1 | 11.1% | NOTCH3 | 8.8% |
| TMB < 10 | 9.3% | CD22 | 11.1% | PDGFRB | 8.8% |
| TMB > 10 | 9.3% | CDH4 | 11.1% | PTEN | 8.8% |
| FAN1 | 7.0% | CDKN2C | 11.1% | ARID1A | 8.8% |
| KMT2D | 7.0% | CEBPA | 11.1% | AXIN1 | 8.8% |
| RIF1 | 7.0% | CHEK1 | 11.1% | KDM6A | 8.8% |
| ARID1A | 7.0% | CKS1B | 11.1% | PARP1 | 8.8% |
| AXIN1 | 7.0% | CRLF2 | 11.1% | PIK3CG | 8.8% |
| KDM6A | 7.0% | CTCF | 11.1% | POLE | 8.8% |
| PARP1 | 7.0% | CYLD | 11.1% | FAN1 | 5.9% |
| PIK3CG | 7.0% | DICER1 | 11.1% | KMT2D | 5.9% |
| POLE | 7.0% | DMC1 | 11.1% | RIF1 | 5.9% |
| GLI1 | 4.7% | DNMT3A | 11.1% | AR | 5.9% |
| KMT2A | 4.7% | EP300 | 11.1% | BCL11A | 5.9% |
| NSD1 | 4.7% | ERCC5 | 11.1% | BUM | 5.9% |
| NTRK1 | 4.7% | ERBB3 | 11.1% | CDK12 | 5.9% |
| SMARCA4 | 4.7% | ETS | 11.1% | ERRFI1 | 5.9% |
| XPA | 4.7% | ETV4 | 11.1% | FANCL | 5.9% |
| ATRX | 4.7% | EXO1 | 11.1% | FGFR3 | 5.9% |
| CCNE1 | 4.7% | EXT1 | 11.1% | GNAS | 5.9% |
| CDH4 | 4.7% | FAM123B | 11.1% | HNF1A | 5.9% |
| EP300 | 4.7% | FAN1 | 11.1% | MGA | 5.9% |
| ERCC5 | 4.7% | FANCB | 11.1% | MSH3 | 5.9% |
| FANCF | 4.7% | FANCD2 | 0.111111 | NOTCH2 | 5.9% |
| MAP3K1 | 4.7% | FANCF | 11.1% | PMS2 | 5.9% |
| MSH2 | 4.7% | FAT1 | 11.1% | RAD50 | 5.9% |
| MUTYH | 4.7% | FLT1 | 11.1% | SMO | 5.9% |
| RBBP8 | 4.7% | FOXL2 | 11.1% | XPO1 | 5.9% |
| SUFU | 4.7% | GATA2 | 11.1% | ATRX | 2.9% |
| TGFBR2 | 4.7% | GEN1 | 11.1% | CCNE1 | 2.9% |
| TLX3 | 4.7% | GLI2 | 11.1% | CDH4 | 2.9% |
| TP53BP1 | 4.7% | IL7R | 11.1% | EP300 | 2.9% |
| AR | 4.7% | KAT6B | 11.1% | ERCC5 | 2.9% |
| BCL11A | 4.7% | KDR | 11.1% | FANCF | 2.9% |
| BLM | 4.7% | KIT | 11.1% | MAP3K1 | 2.9% |
| CDK12 | 4.7% | KMT2D | 11.1% | MSH2 | 2.9% |
| ERRFI1 | 4.7% | MAP3K1 | 11.1% | MUTYH | 2.9% |
| FANCL | 4.7% | MAP3K6 | 11.1% | RBBP8 | 2.9% |
| FGFR3 | 4.7% | MCL1 | 11.1% | SUFU | 2.9% |
| GNAS | 4.7% | MCM8 | 11.1% | TGFBR2 | 2.9% |
| HNF1A | 4.7% | MEF2B | 11.1% | TLX3 | 2.9% |
| MGA | 4.7% | MEN1 | 11.1% | TP53BP1 | 2.9% |
| MSH3 | 4.7% | MSH2 | 11.1% | AKT1 | 2.9% |
| NOTCH2 | 4.7% | MUTYH | 11.1% | ALK | 2.9% |
| PMS2 | 4.7% | MYCN | 11.1% | APC | 2.9% |
| RAD50 | 4.7% | NF1 | 11.1% | ASXL1 | 2.9% |
| SMO | 4.7% | NOTCH3 | 11.1% | AXL | 2.9% |
| XPO1 | 4.7% | PDGFRB | 11.1% | BARD1 | 2.9% |
| APH1A | 2.3% | POLQ | 0.111111 | BRCA1 | 2.9% |
| BRCA2 | 2.3% | POT1 | 11.1% | BRIP1 | 2.9% |
| BRD4 | 2.3% | PTEN | 11.1% | CASC5 | 2.9% |
| BUB1B | 2.3% | PVRL4 | 11.1% | C17orf39 | 2.9% |
| C19orf40 | 2.3% | RAF1 | 11.1% | CREBBP | 2.9% |
| CD22 | 2.3% | RBBP8 | 11.1% | DCC | 2.9% |
| CDKN2C | 2.3% | RIF1 | 11.1% | DDR1 | 2.9% |
| CEBPA | 2.3% | RIT1 | 11.1% | DDR2 | 2.9% |
| CHEK1 | 2.3% | RNF43 | 11.1% | EPHA3 | 2.9% |
| CKS1B | 2.3% | ROS1 | 11.1% | EPHA5 | 2.9% |
| CRLF2 | 2.3% | RPTOR | 11.1% | EZH2 | 2.9% |
| CTCF | 2.3% | SDHA | 11.1% | FAS | 2.9% |
| CYLD | 2.3% | SUFU | 11.1% | FGF6 | 2.9% |
| DICER1 | 2.3% | TCEB1 | 11.1% | GATA1 | 2.9% |
| DMC1 | 2.3% | TET2 | 11.1% | GATA6 | 2.9% |
| DNMT3A | 2.3% | TGFBR2 | 11.1% | HGF | 2.9% |
| ERBB3 | 2.3% | TLX3 | 11.1% | JAK1 | 2.9% |
| ETS | 2.3% | TP53BP1 | 11.1% | JAK2 | 2.9% |
| ETV4 | 2.3% | TSHR | 11.1% | KDM5C | 2.9% |
| EXO1 | 2.3% | WHSC1L1 | 11.1% | KLF4 | 2.9% |
| EXT1 | 2.3% | YY1AP1 | 11.1% | KRAS | 2.9% |
| FAM123B | 2.3% | ZNF217 | 11.1% | MAP2K2 | 2.9% |
| FANCB | 2.3% | MSS | 11.1% | MET | 2.9% |

Among the patients with TSC1 mutation, Tables 24 and 25 below show the mutation frequencies in responding patients and non-responding patients to an mTOR inhibitor (e.g., ABI-009), respectively. As shown in Table 24, based upon the information from references discussed above, and results discussed in the application (such as in Examples 1, 2, 3A, 3B and 5) (total about 23 patients), one or more mutations in any one or more of VHL, TP53, PBRM1, BAP1, NTRK1, RB1, ATRX, FANCD2, ARID1A, KDM6A were observed in at least about 8.7% of the total responding patients who had a TSC1 mutation. Based upon the results discussed in the application (such as in Examples 1, 2, 3A, 3B and 5) (total 4 patients), one or more mutations in any one or more of NTRK1, RB1, TP53, APH1A. ATRX, BUB1B. CD22, CDH4, CDKN2C, CEBPA. CKS1B, CRLF2, ETS, FAM123B, FANCD2, FLT1, IL7R, KDR, MAP3K6. MCL1, MEF2B, MUTYH, NF1, NOTCH3, PDGFRB, POT1, PVRL4, RAF1, RBBP8, RIT1, SDHA, SMARCA4, TET2, TGFBR2, TLX3, YY1AP1, and ZNF217 were observed in at least about 25% of the total responding patients who had a TSC1 mutation. Among those, one or more mutations in any one or more of NTRK1, RB1, and TP53 were observed in at least about 50% of the total responding patients who had a TSC1 mutation.

TABLE 24
Mutation frequencies in mTOR inhibitor
responders with a TSC1 mutation.

| | All | | ABI-009 | | Literature |
|---|---|---|---|---|---|
| Gene | Data | Gene | pts only | Gene | only |
| VHL | 34.8% | NTRK1 | 50.0% | VHL | 42.1% |
| TP53 | 26.1% | RB1 | 50.0% | PBRM1 | 31.6% |
| PBRM1 | 26.1% | TP53 | 50.0% | TP53 | 21.1% |
| BAP1 | 13.0% | APH1A | 25.0% | BAP1 | 15.8% |
| NTRK1 | 8.7% | ATRX | 25.0% | ARID1A | 10.5% |
| RB1 | 8.7% | BUB1B | 25.0% | KDM6A | 10.5% |
| ATRX | 8.7% | CD22 | 25.0% | ATRX | 5.3% |
| FANCD2 | 8.7% | CDH4 | 25.0% | FANCD2 | 5.3% |
| ARID1A | 8.7% | CDKN2C | 25.0% | AR | 5.3% |
| KDM6A | 8.7% | CEBPA | 25.0% | BCL11A | 5.3% |
| APH1A | 4.3% | CKS1B | 25.0% | CASC5 | 5.3% |
| BUB1B | 4.3% | CRLF2 | 25.0% | DCC | 5.3% |
| CD22 | 4.3% | ETS | 25.0% | FGFR3 | 5.3% |
| CDH4 | 4.3% | FAM123B | 25.0% | GATA1 | 5.3% |
| CDKN2C | 4.3% | FANCD2 | 25.0% | KDMSC | 5.3% |
| CEBPA | 4.3% | FLT1 | 25.0% | MLLT10 | 5.3% |
| CKS1B | 4.3% | IL7R | 25.0% | NF2 | 5.3% |
| CRLF2 | 4.3% | KDR | 25.0% | PARP1 | 5.3% |
| ETS | 4.3% | MAP3K6 | 25.0% | PIK3CG | 5.3% |
| FAM123B | 4.3% | MCL1 | 25.0% | PTPRD | 5.3% |
| FLT1 | 4.3% | MEF2B | 25.0% | RET | 5.3% |
| IL7R | 4.3% | MUTYH | 25.0% | SETD2 | 5.3% |
| KDR | 4.3% | NF1 | 25.0% | SMAD2 | 5.3% |
| MAP3K6 | 4.3% | NOTCH3 | 25.0% | TAF | 5.3% |
| MCL1 | 4.3% | PDGFRB | 25.0% | TRIM37 | 5.3% |
| MEF2B | 4.3% | POT1 | 25.0% | | |
| MUTYH | 4.3% | PVRL4 | 25.0% | | |
| NF1 | 4.3% | RAF1 | 25.0% | | |
| NOTCH3 | 4.3% | RBBP8 | 25.0% | | |
| PDGFRB | 4.3% | RIT1 | 25.0% | | |
| POT1 | 4.3% | SDHA | 25.0% | | |
| PVRL4 | 4.3% | SMARCA4 | 25.0% | | |
| RAF1 | 4.3% | TET2 | 25.0% | | |
| RBBP8 | 4.3% | TGFBR2 | 25.0% | | |
| RIT1 | 4.3% | TLX3 | 25.0% | | |
| SDHA | 4.3% | YY1AP1 | 25.0% | | |
| SMARCA4 | 4.3% | ZNF217 | 25.0% | | |
| TET2 | 4.3% | MSS | 25.0% | | |

TABLE 25
Mutation frequencies in mTOR inhibitor non-
responders with a TSC1 mutation

| | All | | ABI-009 | | Literature |
|---|---|---|---|---|---|
| Gene | Data | Gene | pts only | Gene | only |
| TP53 | 83.3% | TP53 | 80.0% | TP53 | 100.0% |
| RB1 | 50.0% | RB1 | 60.0% | FGFR3 | 100.0% |
| GLI1 | 33.3% | GLI1 | 40.0% | KDM6A | 100.0% |
| KMT2A | 33.3% | KMT2A | 40.0% | | |
| NSD1 | 33.3% | NSD1 | 40.0% | | |
| XPA | 33.3% | XPA | 40.0% | | |
| ARID1B | 16.7% | ARID1B | 20.0% | | |
| BRCA2 | 16.7% | BRCA2 | 20.0% | | |
| BRD4 | 16.7% | BRD4 | 20.0% | | |
| C19orf40 | 16.7% | C19orf40 | 20.0% | | |
| CCNE1 | 16.7% | CCNE1 | 20.0% | | |
| CHEK1 | 16.7% | CHEK1 | 20.0% | | |
| CTCF | 16.7% | CTCF | 20.0% | | |
| CYLD | 16.7% | CYLD | 20.0% | | |
| DICER1 | 16.7% | DICER1 | 20.0% | | |
| DMC1 | 16.7% | DMC1 | 20.0% | | |
| DNMT3A | 16.7% | DNMT3A | 20.0% | | |
| EP300 | 16.7% | EP300 | 20.0% | | |
| ERCC5 | 16.7% | ERCC5 | 20.0% | | |
| ERBB3 | 16.7% | ERBB3 | 20.0% | | |
| ETV4 | 16.7% | ETV4 | 20.0% | | |
| EXO1 | 16.7% | EXO1 | 20.0% | | |
| EXT1 | 16.7% | EXT1 | 20.0% | | |
| FAN1 | 16.7% | FAN1 | 20.0% | | |
| FANGB | 16.7% | FANCB | 20.0% | | |
| FANCF | 16.7% | FANCF | 20.0% | | |
| FAT1 | 16.7% | FAT1 | 20.0% | | |
| FOXL2 | 16.7% | FOXL2 | 20.0% | | |
| GATA2 | 16.7% | GATA2 | 20.0% | | |
| GEN1 | 16.7% | GEN1 | 20.0% | | |
| GLI2 | 16.7% | GLI2 | 20.0% | | |
| KAT6B | 16.7% | KAT6B | 20.0% | | |
| KIT | 16.7% | KIT | 20.0% | | |
| KMT2D | 16.7% | KMT2D | 20.0% | | |
| MAP3K1 | 16.7% | MAP3K1 | 20.0% | | |
| MCM8 | 16.7% | MCM8 | 20.0% | | |
| MEN1 | 16.7% | MEN1 | 20.0% | | |
| MSH2 | 16.7% | MSH2 | 20.0% | | |
| MYCN | 16.7% | MYCN | 20.0% | | |
| POLQ | 16.7% | POLQ | 20.0% | | |
| PTEN | 16.7% | PTEN | 20.0% | | |
| RIF1 | 16.7% | RIF1 | 20.0% | | |
| RNF43 | 16.7% | RNF43 | 20.0% | | |
| ROS1 | 16.7% | ROS1 | 20.0% | | |
| RPTOR | 16.7% | RPTOR | 20.0% | | |
| SMARCA4 | 16.7% | SMARCA4 | 20.0% | | |
| SUFU | 16.7% | SUFU | 20.0% | | |
| TCEB1 | 16.7% | TCEB1 | 20.0% | | |
| TP53BP1 | 16.7% | TP53BP1 | 20.0% | | |
| TSHR | 16.7% | TSHR | 20.0% | | |
| WHSC1L1 | 16.7% | WHSC1L1 | 20.0% | | |

### TSC2 Analysis

Table 26 below shows mutation frequencies in patients who had a TSC2 mutation. Based upon the information from references discussed above, and results discussed in the application (such as in Examples 1, 2, 3A, 3B and 5) (total 47 patients), one or more mutations in any one or more of TP53, RB1, PTEN, BRCA2 and CDKN2A were observed in at least about 10.9% of the total patients who had a TSC2 mutation. Based upon the results discussed in the application (such as in Examples 1, 2, 3A, 3B and 5) (total about 16 patients), one or more mutations in an one or more of TP3, MSS, ATRX, CDKN2C, DAXX, ERBB3, FLT1, FLT4, GNAS, KDM6A, PMS2 PTCH1, PTEN, RB20 RIF. TLX3, and RN were observed in at least about 12.5% of the total patients who had a TSC2 mutation.

TABLE 26
Mutation frequencies in patients with TSC2 mutation.

| | All | | ABI-009 | | Literature |
|---|---|---|---|---|---|
| Gene | Data | Gene | pts only | Gene | only |
| TP53 | 52.2% | TP53 | 37.5% | TP53 | 60.0% |
| MSS | 37.0% | MSS | 31.3% | MSS | 40.0% |
| TMB < 10 | 26.1% | ATRX | 12.5% | TMB < 10 | 33.3% |
| RB1 | 17.4% | CDKN2C | 12.5% | RB1 | 20.0% |
| PTEN | 13.0% | DAXX | 12.5% | CDKN2A | 16.7% |
| BRCA2 | 10.9% | ERBB3 | 12.5% | PTEN | 13.3% |
| CDKN2A | 10.9% | FLT1 | 12.5% | BRCA2 | 13.3% |
| DAXX | 8.7% | FLT4 | 12.5% | ARID1B | 10.0% |
| PTCH1 | 6.5% | GNAS | 12.5% | AXIN1 | 10.0% |
| CIC | 6.5% | KDM6A | 12.5% | CDKN2B | 10.0% |
| FANCD2 | 6.5% | PMS2 | 12.5% | KIT | 10.0% |
| RET | 6.5% | PTCH1 | 12.5% | DAXX | 6.7% |
| SETD2 | 6.5% | PTEN | 12.5% | CIC | 6.7% |
| ARID1B | 6.5% | RB1 | 12.5% | FANCD2 | 6.7% |
| AXIN1 | 6.5% | RIF1 | 12.5% | RET | 6.7% |
| CDKN2B | 6.5% | TLX3 | 12.5% | SETD2 | 6.7% |
| KIT | 6.5% | TMB < 10 | 12.5% | ALK | 6.7% |
| ATRX | 4.3% | WRN | 12.5% | ARID1A | 6.7% |
| CDKN2C | 4.3% | AR | 6.3% | ATM | 6.7% |
| ERBB3 | 4.3% | ARID2 | 6.3% | BAP1 | 6.7% |
| FLT1 | 4.3% | ASMTL | 6.3% | ERRFI1 | 6.7% |
| FLT4 | 4.3% | ASXL1 | 6.3% | PARP1 | 6.7% |
| GNAS | 4.3% | ATR | 6.3% | PDE4DIP | 6.7% |
| KDM6A | 4.3% | BCL2L11 | 6.3% | POLD1 | 6.7% |
| PMS2 | 4.3% | BLM | 6.3% | SMO | 6.7% |
| RIF1 | 4.3% | BRCA2 | 6.3% | TMB > 10 | 6.7% |
| TLX3 | 4.3% | BRIP1 | 6.3% | PTCH1 | 3.3% |
| WRN | 4.3% | BUB1B | 6.3% | ARID2 | 3.3% |
| ARID2 | 4.3% | C17orf70 | 6.3% | ASXL1 | 3.3% |
| ASXL1 | 4.3% | CARM1 | 6.3% | ATR | 3.3% |
| ATR | 4.3% | CCNE1 | 6.3% | CCNE1 | 3.3% |
| CCNE1 | 4.3% | CDH4 | 6.3% | DNMT3A | 3.3% |
| DNMT3A | 4.3% | CDKN1A | 6.3% | ETV1 | 3.3% |
| ETV1 | 4.3% | CDKN1B | 6.3% | FGFR3 | 3.3% |
| FGFR3 | 4.3% | CIC | 6.3% | JAK2 | 3.3% |
| JAK2 | 4.3% | DNMT1 | 6.3% | PDGFRA | 3.3% |
| PDGFRA | 4.3% | DNMT3A | 6.3% | RAD21 | 3.3% |
| RAD21 | 4.3% | EPCAM | 6.3% | RAD50 | 3.3% |
| RAD50 | 4.3% | EPHA5 | 6.3% | SOCS1 | 3.3% |
| SOCS1 | 4.3% | ETV1 | 6.3% | VHL | 3.3% |
| VHL | 4.3% | EXT1 | 6.3% | APC | 3.3% |
| ALK | 4.3% | EZH2 | 6.3% | B2M | 3.3% |
| ARID1A | 4.3% | FANCA | 6.3% | BRAF | 3.3% |
| ATM | 4.3% | FANCD2 | 6.3% | BRCA1 | 3.3% |
| BAP1 | 4.3% | FANCL | 6.3% | CCND3 | 3.3% |
| ERRFI1 | 4.3% | FAS | 6.3% | CD274 | 3.3% |
| PARP1 | 4.3% | FAT1 | 6.3% | CD36 | 3.3% |
| PDE4DIP | 4.3% | FGFR3 | 6.3% | CDC73 | 3.3% |
| POLD1 | 4.3% | FGFR4 | 6.3% | CSF1R | 3.3% |
| SMO | 4.3% | FOXO1 | 6.3% | DICER1 | 3.3% |
| TMB > 10 | 4.3% | GLI2 | 6.3% | EP300 | 3.3% |
| AR5 | 2.2% | H19 | 6.3% | ERBB4 | 0.033333 |
| ASMTL | 2.2% | HELQ | 6.3% | ERCC5 | 3.3% |
| BCL2L11 | 2.2% | IL7R | 6.3% | ERRC4 | 3.3% |
| BLM | 2.2% | JAK2 | 6.3% | FBX011 | 3.3% |
| BRIP1 | 2.2% | JAZF1 | 6.3% | FLCN | 3.3% |
| BUB1B | 2.2% | KEAP1 | 6.3% | FLT3 | 3.3% |
| C17orf70 | 2.2% | KLF4 | 6.3% | HNF1A | 3.3% |
| CARM1 | 2.2% | MGA | 6.3% | KDM4C | 3.3% |
| CDH4 | 2.2% | NPM1 | 6.3% | KDM5C | 3.3% |
| CDKN1A | 2.2% | NROB1 | 6.3% | KMT2D | 3.3% |
| CDKN1B | 2.2% | NRG1 | 6.3% | KRAS | 3.3% |
| DNMT1 | 2.2% | NTRK1 | 6.3% | MAP3K1 | 3.3% |
| EPCAM | 2.2% | PDGFRA | 6.3% | MAP3K6 | 3.3% |
| EPHA5 | 2.2% | PDGFRB | 6.3% | MLH1 | 3.3% |
| EXT1 | 2.2% | PIK3C2B | 6.3% | MSH6 | 3.3% |
| EZH2 | 2.2% | PRKDC | 6.3% | mTOR | 3.3% |
| FANCA | 2.2% | PRKDC | 6.3% | MYC | 3.3% |
| FANCL | 2.2% | RAD21 | 6.3% | MYCN | 3.3% |
| FAS | 2.2% | RAD50 | 6.3% | NBN | 0.033333 |
| FAT1 | 2.2% | RET | 6.3% | NF1 | 3.3% |
| FGFR4 | 2.2% | RSPO2 | 6.3% | PBRM1 | 3.3% |
| FOXO1 | 2.2% | SETBP1 | 6.3% | PDCD1LG2 | 3.3% |
| GLI2 | 2.2% | SETD2 | 6.3% | PIK3CA | 3.3% |
| H19 | 2.2% | SMARCA4 | 6.3% | RANBP2 | 3.3% |
| HELQ | 2.2% | SOCS1 | 6.3% | RICTOR | 3.3% |
| IL7R | 2.2% | TRIM37 | 6.3% | WHSC1L1 | 3.3% |
| JAZF1 | 2.2% | UIMC1 | 6.3% | XPO1 | 3.3% |
| KEAP1 | 2.2% | VHL | 6.3% | | |
| KLF4 | 2.2% | YY1AP1 | 6.3% | | |

Among the patients with TSC2 mutation, Tables 27 below show the mutation frequencies in responding patients to an mTOR inhibitor (e.g., ABI-009), respectively. As shown in Table 27, based upon the information from references discussed above, and results discussed in the application (such as in Examples 1, 2, 3A, 3B and 5) (total 28 responding patients with TSC2 mutation), one or more mutations in any one or more of TP53, RB1, BRCA2, RET and SETD2 were observed in at least about 10.7% of the total responding patients who had a TSC2 mutation. Based upon the results discussed in the application (such as in Examples 1, 2, 3A, 3B and 5) (total about 14 responding patients with TSC2 mutation), one or more mutations in any one or more of TP53, ATRX, DAXX, ERBB3, FLT1, GNAS, KDM6A, PMS2, PTEN, RB1, and TLX3 were observed in at least about 14.3% of the total responding patients who had a TSC2 mutation. Mutations in BRIP1, BUB1B, CDKN2C, FANCD2, FLT4, PDGFRA, PTCH1, RIF1, VHL, WRN were observed in mTOR non-responders who had a TSC2 mutation.

TABLE 27
Mutation frequencies in mTOR inhibitor
responders with TSC2 mutation

| | All | | ABI-009 | | Literature |
|---|---|---|---|---|---|
| Gene | Data | Gene | pts only | Gene | only |
| TP53 | 57.1% | TP53 | 42.9% | TP53 | 71.4% |
| MSS | 17.9% | MSS | 35.7% | RB1 | 14.3% |
| RB1 | 14.3% | ATRX | 14.3% | BRCA2 | 14.3% |
| BRCA2 | 10.7% | DAXX | 14.3% | RET | 14.3% |
| RET | 10.7% | ERBB3 | 14.3% | SETD2 | 14.3% |
| SETD2 | 10.7% | FLT1 | 14.3% | ARID1A | 14.3% |
| ATRX | 7.1% | GNAS | 14.3% | ARID2 | 7.1% |
| DAXX | 7.1 % | KDM6A | 14.3% | ASXL1 | 7.1% |
| ERBB3 | 7.1% | PMS2 | 14.3% | ATR | 7.1% |
| FLT1 | 7.1% | PTEN | 14.3% | DNMT3A | 7.1% |
| GNAS | 7.1% | RB1 | 14.3% | JAK2 | 7.1% |
| KDM6A | 7.1% | TLX3 | 14.3% | PTCH1 | 7.1% |
| PMS2 | 7.1 % | TMB < 10 | 14.3% | APC | 7.1% |
| PTEN | 7.1% | AR | 7.1% | ARID1B | 7.1% |
| TLX3 | 7.1% | ARID2 | 7.1% | AXIN1 | 7.1% |
| TMB < 10 | 7.1% | ASMTL | 7.1% | BAP1 | 7.1% |
| ARID2 | 7.1% | ASXL1 | 7.1% | B2M | 7.1% |
| ASXL1 | 7.1% | ATR | 7.1% | CCND3 | 7.1% |
| ATR | 7.1% | BCL2L11 | 7.1% | CD36 | 7.1% |
| DNMT3A | 7.1% | BLM | 7.1% | CD274 | 7.1% |
| JAK2 | 7.1% | BRCA2 | 7.1% | CDC73 | 7.1% |
| PTCH1 | 7.1% | C17orf70 | 7.1% | CDKN2A | 7.1% |
| ARID1A | 7.1% | CARM1 | 7.1% | CDKN2B | 7.1% |
| AR | 3.6% | CCNE1 | 7.1% | CSF1R | 7.1% |
| ASMTL | 3.6% | CDH4 | 7.1% | DICER1 | 7.1% |
| BCL2L11 | 3.6% | CDKN1A | 7.1% | ERBB4 | 7.1% |
| BLM | 3.6% | CDKN1B | 7.1% | FBX011 | 7.1% |
| C17orf70 | 3.6% | CDKN2C | 7.1% | FLCN | 7.1% |
| CARM1 | 3.6% | CIC | 7.1% | FLT3 | 7.1% |
| CCNE1 | 3.6% | DNMT1 | 7.1% | KDM4C | 7.1% |
| CDH4 | 3.6% | DNMT3A | 7.1% | KRAS | 7.1% |
| CDKN1A | 3.6% | EPCAM | 7.1% | MAP3K1 | 7.1% |
| CDKN1B | 3.6% | EPHA5 | 7.1% | MSH6 | 7.1% |
| CDKN2C | 3.6% | ETV1 | 7.1% | mTOR | 7.1% |
| CIC | 3.6% | EXT1 | 7.1% | NBN | 7.1% |
| DNMT1 | 3.6% | EZH2 | 7.1% | PDCD1LG2 | 7.1% |
| EPCAM | 3.6% | FANCA | 7.1% | RANBP2 | 7.1% |
| EPHA5 | 3.6% | FANCL | 7.1% | RICTOR | 7.1% |
| ETV1 | 3.6% | FAS | 7.1% | VHL | 7.1% |
| EXT1 | 3.6% | FAT1 | 7.1% | XPO1 | 7.1% |
| EZH2 | 3.6% | FGFR3 | 7.1% | | |
| FANCA | 3.6% | FGFR4 | 7.1% | | |
| FANCL | 3.6% | FLT4 | 7.1% | | |
| FAS | 3.6% | FOXO1 | 7.1% | | |
| FAT1 | 3.6% | GLI2 | 7.1% | | |
| FGFR3 | 3.6% | H19 | 7.1% | | |
| FGFR4 | 3.6% | HELQ | 7.1% | | |
| FLT4 | 3.6% | IL7R | 7.1% | | |
| FOXO1 | 3.6% | JAK2 | 7.1% | | |
| GLI2 | 3.6% | JAZF1 | 7.1% | | |
| H19 | 3.6% | KEAP1 | 7.1% | | |
| HEIQ | 3.6% | KLF4 | 7.1% | | |
| IL7R | 3.6% | MGA | 7.1% | | |
| JAZF1 | 3.6% | NPM1 | 7.1% | | |
| KEAP1 | 3.6% | NRG1 | 7.1% | | |
| KLF4 | 3.6% | NR0B1 | 7.1% | | |
| MGA | 3.6% | NTRK1 | 7.1% | | |
| NPM1 | 3.6% | PRKDC | 7.1% | | |
| NRG1 | 3.6% | PDGFRB | 7.1% | | |
| NR0B1 | 3.6% | PIK3C2B | 7.1% | | |
| NTRK1 | 3.6% | PRKDC | 7.1% | | |
| PRKDC | 3.6% | PTCH1 | 7.1% | | |
| PDGFRB | 3.6% | RAD21 | 7.1% | | |
| PIK3C2B | 3.6% | RAD50 | 7.1% | | |
| PRKDC | 3.6% | RET | 7.1% | | |
| RAD21 | 3.6% | RIF1 | 7.1% | | |
| RAD50 | 3.6% | RSPO2 | 7.1% | | |
| RIF1 | 3.6% | SETBP1 | 7.1% | | |
| RSPO2 | 3.6% | SETD2 | 7.1% | | |
| SETBP1 | 3.6% | SMARCA4 | 7.1% | | |
| SMARCA4 | 3.6% | SOCS1 | 7.1% | | |
| SOCS1 | 3.6% | TRIM37 | 7.1% | | |
| TRIM37 | 3.6% | UIMC1 | 7.1% | | |
| UIMC1 | 3.6% | WRN | 7.1% | | |
| WRN | 3.6% | YY1AP1 | 7.1% | | |

### Bi-Allelic Analysis

Based upon the information from references discussed above, and results discussed in the application (such as in Examples 1, 2, 3A, 3B and 5) (total 25 patients with bi-allelic mutations in TSC11 or TSC2), one or more mutations in any one or more of MSS, TP53, RB11, BRCA2, ARID1B, CCNE1, KIT, PTEN, CDKN2A were observed in at least about 13.6;% of the total patients who had a TSC1 or TSC2 bi-allelic mutation. Based upon the results discussed in the application (such as in Examples 1, 2, 3A, 3B and 5) (total about 12 patients with bi-allelic mutations in TSC1 or TSC2), one or more mutations in any one or more of TP53, BRCA2, CCNE1, CDH4, CDKN2C, ERBB3, FAT1, GNAS, NSD1, NTRK1, PMS2, RB1, TLX3 were observed in at least about 16.7% of the total patients who had a TSC1 or TSC2 bi-allelic mutation. See Table 28 below.

TABLE 28
Mutation frequencies in patients with TSC1 or TSC2
bi-allelic (i.e., two-point) mutations

| | All | | ABI-009 | | Literature |
|---|---|---|---|---|---|
| Gene | Data | Gene | pts only | Gene | only |
| MSS | 40.9% | TP53 | 25.0% | MSS | 70.0% |
| TP53 | 36.4% | BRCA2 | 16.7% | TMB < 10 | 70.0% |
| RB1 | 31.8% | CCNE1 | 16.7% | TP53 | 50.0% |
| TMB < 10 | 31.8% | CDH4 | 16.7% | RB1 | 50.0% |
| BRCA2 | 18.2% | CDKN2C | 16.7% | ARID1B | 30.0% |
| ARID1B | 18.2% | ERBB3 | 16.7% | CDKN2A | 30.0% |
| CCNE1 | 13.6% | FAT1 | 16.7% | BRCA2 | 20.0% |
| KIT | 13.6% | GNAS | 16.7% | KIT | 20.0% |
| PTEN | 13.6% | MSS | 16.7% | PTEN | 20.0% |
| CDKN2A | 13.6% | NSD1 | 16.7% | AXIN1 | 20.0% |
| CDH4 | 9.1% | NTRK1 | 16.7% | CDKN2B | 0.2 |
| CDKN2C | 9.1% | PMS2 | 16.7% | ERRFI1 | 20.0% |
| ERBB3 | 9.1% | RB1 | 16.7% | FANCD2 | 20.0% |
| FAT1 | 9.1% | TLX3 | 16.7% | PARP1 | 20.0% |
| GNAS | 9.1% | AR | 8.3% | POLD1 | 20.0% |
| NSD1 | 9.1% | ARID1B | 8.3% | SMO | 20.0% |
| NTRK1 | 9.1% | ARID2 | 8.3% | CCNE1 | 10.0% |
| PMS2 | 9.1% | ASXL1 | 8.3% | ARID2 | 10.0% |
| TLX3 | 9.1% | ATRX | 8.3% | MAP3K1 | 10.0% |
| ARID2 | 9.1% | BLM | 8.3% | RAD50 | 10.0% |
| MAP3K1 | 9.1% | BRD4 | 8.3% | ALK | 10.0% |
| RAD50 | 9.1% | BUB1B | 8.3% | BAP1 | 10.0% |
| AXIN1 | 9.1% | C17orf70 | 8.3% | C17orf39 | 10.0% |
| CDKN2B | 9.1% | C19orf40 | 8.3% | DICER1 | 10.0% |
| ERRFI1 | 9.1% | CDKN1A | 8.3% | FLT3 | 10.0% |
| FANCD2 | 9.1% | CEBPA | 8.3% | HNF1A | 10.0% |
| PARP1 | 9.1% | CHEK1 | 8.3% | MAP3K6 | 10.0% |
| POLD1 | 9.1% | CIC | 8.3% | PBRM1 | 10.0% |
| SMO | 9.1% | DAXX | 8.3% | RANBP2 | 10.0% |
| AR | 4.5% | EP300 | 8.3% | RICTOR | 10.0% |
| ASXL1 | 4.5% | EPCAM | 8.3% | | |
| ATRX | 4.5% | ERCC5 | 8.3% | | |
| BLM | 4.5% | ETV1 | 8.3% | | |
| BRD4 | 4.5% | ETV4 | 8.3% | | |
| BUB1B | 4.5% | EXO1 | 8.3% | | |
| C17orf70 | 4.5% | EXT1 | 8.3% | | |
| C19orf40 | 4.5% | EZH2 | 8.3% | | |
| CDKN1A | 4.5% | FAN1 | 8.3% | | |
| CEBPA | 4.5% | FANCA | 8.3% | | |
| CHEK1 | 4.5% | FANCF | 8.3% | | |
| CIC | 4.5% | FANCL | 8.3% | | |
| DAXX | 4.5% | FGFR3 | 8.3% | | |
| EP300 | 4.5% | FGFR4 | 8.3% | | |
| EPCAM | 4.5% | FLT1 | 8.3% | | |
| ERCC5 | 4.5% | FLT4 | 8.3% | | |
| ETV1 | 4.5% | GLI1 | 8.3% | | |
| ETV4 | 4.5% | GLI2 | 8.3% | | |
| EXO1 | 4.5% | H19 | 8.3% | | |
| EXT1 | 4.5% | HELQ | 8.3% | | |
| EZH2 | 4.5% | IL7R | 8.3% | | |
| FAN1 | 4.5% | JAK2 | 8.3% | | |
| FANCA | 4.5% | KAT6B | 8.3% | | |
| FANCF | 4.5% | KDM6A | 8.3% | | |
| FANCL | 4.5% | KIT | 8.3% | | |
| FGFR3 | 4.5% | KLF4 | 8.3% | | |
| FGFR4 | 4.5% | MAP3K1 | 8.3% | | |
| FLT1 | 4.5% | MCL1 | 8.3% | | |
| FLT4 | 4.5% | MCM8 | 8.3% | | |
| GLI1 | 4.5% | MGA | 8.3% | | |
| GLI2 | 4.5% | MUTYH | 8.3% | | |
| H19 | 4.5% | NOTCH3 | 8.3% | | |
| HELQ | 4.5% | NR0B1 | 8.3% | | |
| IL7R | 4.5% | NRG1 | 8.3% | | |
| JAK2 | 4.5% | PDGFRB | 8.3% | | |
| KAT6B | 4.5% | PIK3C2B | 8.3% | | |
| KDM6A | 4.5% | POLQ | 8.3% | | |
| KLF4 | 4.5% | PRKDC | 8.3% | | |
| MCL1 | 4.5% | PTCH1 | 8.3% | | |
| MCM8 | 4.5% | PTEN | 8.3% | | |
| MGA | 4.5% | PVRL4 | 8.3% | | |
| MUTYH | 4.5% | RAD50 | 8.3% | | |
| NOTCH3 | 4.5% | RBBP8 | 8.3% | | |
| NROB1 | 4.5% | RET | 8.3% | | |
| NRG1 | 4.5% | RIF1 | 8.3% | | |
| PDGFRB | 4.5% | RIT1 | 8.3% | | |
| PIK3C2B | 4.5% | RNF43 | 8.3% | | |
| POLQ | 4.5% | SDHA | 8.3% | | |
| PRKDC | 4.5% | SETBP1 | 8.3% | | |
| PTCH1 | 4.5% | SETD2 | 8.3% | | |
| PVRL4 | 4.5% | SMARCA4 | 8.3% | | |
| RBBP8 | 4.5% | SOCS1 | 8.3% | | |
| RET | 4.5% | TET2 | 8.3% | | |
| RIF1 | 4.5% | TP53BP1 | 8.3% | | |
| RIT1 | 4.5% | TRIM37 | 8.3% | | |
| RNF43 | 4.5% | TSHR | 8.3% | | |
| SDHA | 4.5% | WHSC1L1 | 8.3% | | |
| SETBP1 | 4.5% | WRN | 8.3% | | |
| SETD2 | 4.5% | XPA | 8.3% | | |

Because the sequencing panels for detecting mutations across those studies not the same, it is possible that some of the genes may not be tested in every patient in the analysis. Accordingly the frequency of the mutations discussed above merely indicate a minimum frequency.

1: A method of treating a cancer in an individual comprising administering to the individual an effective amount of a composition comprising nanoparticles comprising sirolimus and an albumin, wherein the individual is selected for treatment on the basis of a) having an mTOR inactivating mutation at TSC1 or TSC2, b) having an aberration at any of the genes selected from the group consisting of VHL, RB1, PBRM1, KDM6A, RET, SETD2, ARID1A, BAP1, BRCA2, TP53, RB1, ATRX, FLT1, NTRK1, TLX3, KDM6A, CDH4, CDKN2C, DAXX, ERBB3, GNAS, IL7R, PDGFRB, PMS2, PTEN, SMARCA4, and YY1AP1.

2: The method of claim 1, wherein the individual has not been treated with an mTOR inhibitor.

3: The method of claim 1, wherein the individual has failed a prior therapy.

4. (canceled)

5: The method of claim 1, wherein the inactivating mutation in TSC1 or TSC2 comprises a homozygous deletion, bi-allelic mutations, a splice site mutation, a frameshift mutation, nonsense mutation in coding region, missense mutation with confirmed impact, or a loss or deletion of TSC1 or TSC2.

6: The method of claim 5, wherein the inactivating mutation in TSC1 or TSC2 comprises bi-allelic mutations.

7: The method of claim 1, wherein the individual is selected for treatment on the basis of having an aberration at any of the genes selected from the group consisting of TP53, RB1, ATRX, FLT1, NTRK1, TLX3, KDM6A, CDH4, CDKN2C, DAXX, ERBB3, GNAS, IL7R, PDGFRB, PMS2, PTEN, SMARCA4, and YY1AP1.

8: The method of claim 1, wherein the individual the individual is selected for treatment on the basis of having an inactivating mutation in TSC1.

9: The method of claim 8, wherein the individual is selected for treatment on the basis of having an aberration at any of the genes selected from the group consisting of NTRK1, RB1, TP53, and PBRM1.

10: The method of claim 1, wherein the individual the individual is selected for treatment on the basis of having an inactivating mutation in TSC2.

11: The method of claim 10, wherein the individual is selected for treatment on the basis of having an aberration at any of the genes selected from the group consisting of TP53, ATRX, DAXX, ERBB3, FLT1, GNAS, KDM6A, PMS2, PTEN, RB1, and TLX3.

12: The method of claim 1, wherein the individual has a tumor mutational burden less than about 10.

13: The method of claim 1, wherein the individual has a stable microsatellite status.

14: The method of claim 1, wherein the individual does not comprise any of a) a deletion mutation in EGFR exon 19; b) EGFR exon 21 L858R alteration; c) EGFR exon 20 T790M alteration; d) ALK rearrangement; e) BRAF V600E or V600K; f) MET single nucleotide variant or indel that leads to MET exon 14 skipping; g) ERBB2 amplification; h) any of C420R, E542K, E545A, E545D, E545G, E545K, Q546E, Q546R, H1047L, H1047R, and H1047Y in PIK3CA; i) BRCA1/2 alteration; j) a FGFR2 fusion and/or rearrangement; and k) a mutation in any of BRCA1, BRCA2, ATM, BARD1, BRIP1, CDK12, CHEK1, CHEK2, FANCL, PALB2, RAD51B, RAD51C, RAD5JD and RAD54L.

15: The method of claim 1, wherein the individual has an mTOR-activating aberration at RPS6.

16: The method of claim 15, wherein the mTOR-activating aberration at RPS6 comprises an aberrant phosphorylation level of the protein encoded by RPS6 or an aberrant expression level of RPS6.

17: The method of claim 1, wherein the cancer is advanced and/or malignant.

18-20. (canceled)

21: The method of claim 1, wherein the nanoparticles in the composition comprises the mTOR inhibitor associated with the carrier protein.

22: The method of claim 1, wherein the nanoparticles in the composition have an average diameter of no greater than about 200 nm.

23: The method of claim 1, wherein the ratio of the mTOR inhibitor to the carrier protein in the nanoparticles is from about 1:1 to about 9:1.

24: The method of claim 1, wherein the individual is a human.

25: The method of claim 24, wherein the composition is administered at a dose of about 30 mg/m² to about 100 mg/m² for two out of every three weeks a cycle for one or more cycles.

26. (canceled)

27: The method of claim 1, wherein the composition is administered intravenously or subcutaneously.

28: The method of claim 1, wherein the composition comprises (a) nanoparticles comprising rapamycin and albumin, and (b) a non-nanoparticle portion comprising albumin and rapamycin;
wherein about 80% to about 95% of the albumin in the composition is in the form of monomeric albumin, about 4% to about 15% of the albumin in the composition is in the form of dimeric albumin, and about 0.5% to about 5% of the albumin in the composition is in the form of polymeric albumin when the percentage of albumin in the composition that is in the form of monomeric albumin, dimeric albumin, or polymeric albumin is determined by subjecting the composition to size-exclusion chromatography (SEC) using a saline mobile phase coupled with a multiple angle light scattering (MALS) detector.

29: The method of claim 1, wherein the nanoparticle composition comprising: (a) nanoparticles comprising rapamycin and albumin, and (b) a non-nanoparticle portion comprising albumin and rapamycin;
wherein about 42% to about 60% of the albumin in the nanoparticles is in the form of polymeric albumin other than oligomeric albumin when the percentage of albumin in the nanoparticles that is in the form of polymeric albumin other than oligomeric albumin is determined by separating the nanoparticles from the non-nanoparticle portion, dissolving the nanoparticles, and subjecting the dissolved nanoparticles to size-exclusion chromatography.

30-32. (canceled)
